(19) United States
(12) Patent Application Publication
(10) Pub. No.: US 2026/0166113 A1
(43) Pub. Date: Jun. 18, 2026

(54) COMPOSITIONS AND METHODS FOR TREATING RENAL CANCER

(71) Applicant: The Regents of the University of Michigan, Ann Arbor, MI (US)

(72) Inventors: Arul M. Chinnaiyan, Ann Arbor, MI (US); Aleksey Nesvizhskiy, Ann Arbor, MI (US); Saravana M. Dhanasekaran, Ann Arbor, MI (US); Rahul Mannan, Ann Arbor, MI (US); Rohit Mehra, Ann Arbor, MI (US); Yuping Zhang, Ann Arbor, MI (US); Xiaoming Wang, Ann Arbor, MI (US)

(21) Appl. No.: 19/124,431

(22) PCT Filed: Oct. 26, 2023
(86) PCT No.: PCT/US2023/036017

§ 371 Date: Apr. 25, 2025

Related U.S. Application Data
(60) Provisional application No. 63/420,058, filed on Oct. 27, 2022.
(60) Provisional application No. 63/449,796, filed on Mar. 3, 2023.

(51) Int. Cl. A61K 38/00

(52) U.S. Cl. CPC A61K 38/00

(57) ABSTRACT

Provided herein are compositions and methods for cancer diagnosis, research and therapy, including but not limited to, cancer markers. In particular, provided herein are methods of treating renal cancer based on expression levels of cancer markers.

### STATEMENT OF RELATED APPLICATIONS

This application claims priority to U.S. Provisional Patent Application No. 63/449,796, filed Mar. 3, 2023, and to U.S. Provisional Patent Application No. 63/420,058, filed Oct. 27, 2022, the entire contents of which are incorporated herein by reference for all purposes.

### STATEMENT REGARDING FEDERALLY SPONSORED RESEARCH OR DEVELOPMENT

This invention was made with government support under CA210967 awarded by the National Institutes of Health. The government has certain rights in the invention.

### FIELD OF THE DISCLOSURE

Provided herein are compositions and methods for cancer diagnosis, research and therapy, including but not limited to, cancer markers. In particular, provided herein are methods of treating renal cancer based on expression levels of cancer markers.

### BACKGROUND OF THE DISCLOSURE

Renal tumors are the third most common urologic malignancy and can originate from the renal parenchyma or urinary collecting system. Renal cell carcinoma, arising from the renal parenchyma, is the most common malignant renal tumor associated with an incidence of 64,000 cases and approximately 14,000 deaths yearly in the United States. From the urinary collecting system, urothelial cell carcinoma is the most common malignancy representing approximately 10-15% of all renal tumors. The overall incidence of malignant renal tumors is increasing and currently is the third most common form of genitourinary cancer. Both malignant and benign renal tumors are increasingly diagnosed in incidental fashion with the use of advanced cross-sectional imaging. Accurate diagnosis of benign versus malignant tumor types is lacking and accordingly patients may be subjected to overtreatment. Furthermore, there are currently no diagnostic tests from needle biopsy, urine or blood that accurately characterize renal tumors or identify patients at risk for renal tumors. The diagnostic and therapeutic approach to renal tumors is complicated by the presence of multiple benign renal tumor types and the fact that many small malignant renal parenchymal tumors can be observed rather than definitively treated.

At the present time, there are no accurate, user-friendly, and widely accessible screening tools at the tissue, blood or urinary level for ideal clinical management of renal tumors. Additional methods to determine and administer appropriate treatments for renal cancer are needed.

### SUMMARY OF THE DISCLOSURE

Provided herein are compositions and methods for cancer diagnosis, research and therapy, including but not limited to, cancer markers. In particular, provided herein are methods of treating renal cancer based on expression levels of cancer markers.

The present disclosure provides cancer markers useful in the diagnosis, prognosis, and treatment of renal cancer. The markers improve renal cancer treatment by identifying subjects with aggressive tumors and by providing targeted therapy to such individuals.

Accordingly, in some embodiments, provided herein is a method of treating renal cell carcinoma (RCC), comprising: a) assaying the level of expression of ubiquitin C-terminal hydrolase L1 (UCHL1) in a sample from a subject (e.g., a subject diagnosed with RCC); and b) administering an UCHL1 inhibitor to a subject identified as having increased levels of expression of UCHL1.

Also provided is a method of characterizing or prognosing RCC, comprising: a) assaying a level of expression UCHL1 in a sample from a subject diagnosed with RCC; and b) identifying the subject as at an increased risk of death or metastatic RCC when the subject is identified as having increased levels of expression of UCHL1. In some embodiments, the method further comprises administering an UCHL1 inhibitor to the subject.

Further embodiments provide a method of treating renal cell carcinoma (RCC), comprising: a) assaying the level of expression of pyrroline-5-carboxylate reductase 1 (PYCR1) and/or IGF2BP3 in a sample from a subject (e.g., a subject diagnosed with RCC); and b) administering an PYCR1 and/or IGF2BP3 inhibitor to a subject identified as having increased levels of expression of PYCR1 and/or IGF2BP3.

Additional embodiments provide a method of characterizing or prognosing RCC, comprising: a) assaying the level of expression of PYCR1 and/or IGF2BP3 in a sample from a subject diagnosed with RCC; and b) identifying the subject as at an increased risk of death or metastatic RCC when the subject is identified as having increased levels of expression of PYCR1 and/or IGF2BP3.

Certain embodiments provide a method of characterizing or prognosing RCC, comprising: a) assaying the level of expression of UCHL1 and IGF2BP3 in a sample from a subject diagnosed with RCC; and b) identifying the subject as at an increased risk of death or metastatic RCC when the subject is identified as having increased levels of expression of UCHL1 and IGF2BP3 in the sample.

Certain embodiments utilize one or more additional markers in combination with UCHL1 or PYCR1. For example, in some embodiments, the markers are PYCR1 and/or IGF2BP3 and one or more (e.g., all) of dihydropyrimidinase like 3 (DPYSL3), Inhibitor of Nuclear Factor Kappa-B Kinase-Interacting Protein (IKBIP), or Fatty Acid Binding Protein 6 (FABP6).

The present disclosure is not limited to particular UCHL1, IGF2BP3 or PYCR1 inhibitors. In some embodiments, the inhibitor is a small molecule (e.g., CAS-668467-91-2), a nucleic acid, or an antibody.

In some embodiments, a MEK inhibitor (e.g., trametinib), adjuvant chemotherapy, and/or immunotherapy is administered to the subject.

In some embodiments, the RCC is clear cell RCC (ccRCC) or non ccRCC. In some embodiments, the RCC exhibits high genomic instability.

The present disclosure is not limited to particular methods of assaying the level of expression of UCHL1, IGF2BP3 or PYCR1. In some embodiments, the expression is the level of mRNA or protein expressed by a UCHL1, IGF2BP3 or PYCR1 gene. In some embodiments, the sample is, for example, urine, tissue, blood, plasma, serum, kidney tissue, kidney cells, or renal cancer cells. In some embodiments, the assaying is carried out utilizing a method selected from the group, for example, an immunological technique (e.g., including but not limited to, immunohistochemistry, ELISA, or a Western blot), a sequencing technique, a nucleic acid hybridization technique, or a nucleic acid amplification technique (e.g., including but not limited to, polymerase chain reaction, reverse transcription polymerase chain reaction, transcription-mediated amplification, ligase chain reaction, strand displacement amplification, or nucleic acid sequence based amplification). Exemplary reagents for detecting the level of UCHL1, IGF2BP3 or PYCR1 include but are not limited to, one or more antibodies, a pair of amplification oligonucleotides, a sequencing primer, or an oligonucleotide probe. In some embodiments, the reagent comprises one or more labels.

Additional embodiments are described herein.

### DESCRIPTION OF THE FIGURES

FIG. 1. Examples of nodularity from the sample cohort and various morphologic architectures. (A) microcystic and tubular architecture in spatially defined regions with a high magnification inset of the tubular growth pattern, (B) shows a nested tumor background with a nodule of cystic/eosinophilic architecture that is highlighted by the higher magnification inset, and (C) shows nested and solid growth patterns juxtaposed to each other with a higher magnification inset of the solid growth pattern.

FIG. 2. Representative images from Case #3 representing cytoplasmic BAP1 expression and alterations of different spatially defined areas. (A) H&E image showing defined tubular and microcystic areas of tumor. (B) BAP1 IHC stained section demonstrating areas of intense cytoplasmic staining within the tumor at the interface of these two distinct nodules. (C) UCHL1 IHC stained section of same region highlighting intense staining of the tumor cells with cytoplasmic BAP1 staining. (D) SETD2 stained section showing the nodule of tubular tumor with no expression and the interface area of UCHL1 expression with strong nuclear staining. (E) CAIX IHC stained section showing weakened membranous expression in the tubular nodular area which correlates to SETD2 loss of expression.

FIG. 3. Representative images from Case #20. (A) H&E image at 5× magnification showing a nodule of eosinophilic and pseudopapillary cells forming a tumor nodule. (B) CAIX IHC stained section showing weakened membranous staining within the tumor nodule. (C) BAP1 IHC stained section showing same tumor nodule with weakened and loss of expression. (D) UCHL1 stained section showing strong cytoplasmic expression in all the tumor cells. (E) SETD2 IHC stained section showing tumor cells within tumor nodule negative for staining with positive inflammatory cells in the background.

FIG. 4. Representative images from Case #27. (A) H&E image showing the interface of two distinctive nodules. The upper half of the tumor showing a nested pattern and the bottom half showing a papillary and eosinophilic architecture (as shown by the high magnification inset). (B) CAIX IHC stained section showing weakened expression in the papillary nodule of tumor with the high magnification inset showing the interface of normal expression and weakened expression. (C) BAP1 IHC stained section showing nuclear retention of staining in the nested component of the tumor and loss of expression in the papillary component. (D) UCHL1 IHC stained section showing expression in the papillary/eosinophilic component. (E) SETD2 IHC stained section showing weakened expression in the papillary component and retained expression in the nested component.

FIG. 5. Representative images from case #19 and case #41. (A) H&E image of case #19 showing a central nodule of high-grade tumor in a bleeding follicle pattern. (B) CAIX IHC stained section showing a nodule of CAIX negative tumor which encompasses the high-grade bleeding follicle area. (C) SETD2 IHC stained section showing the bleeding follicle nodule with loss of nuclear expression. (D) H&E image of case #41 showing an eosinophilic solid tumor with areas of clear cells as depicted by high magnification inset. (E) CAIX IHC stained section showing a gradient of expression across the area of tumor. (F) SETD2 stained section showing similar gradient of expression across the same area of tumor shown in parts D & E.

FIG. 6. Representative biomarker panel in a single CCRCC case (A-C) at different magnifications (5X, 10X and 20X) where matched tumor areas show loss of BAP1 (D-F) with corresponding expression of AMACR (G-I) and UCHL1 (J-L).

FIG. 7. Molecular underpinnings of ccRCC histopathologic heterogeneity. A) Overview of the sample cohorts and various data types generated in this study. Top panel: stacked bar plots represent the total numbers of cases, tumor samples, matched NATs, and peripheral blood samples. Bottom panel: bar plot represents various genomics, proteomic, metabolomic, kinase inhibitor, and image analysis data types generated. B) Distribution of ccRCC cohort. Representative H&E images of the 4 histopathologic subtypes based on nuclear grade and cytological features. Top left to right: Low-grade ccRCC (CL), Highgrade ccRCC (CH); bottom left to right: CH with sarcomatoid (CH-S), and CH with rhabdoid (CHR). Scale bar=200 microns. C) Heatmap of proteogenomic features associated with histological grade. D) Upset plot represents the association between tumor grade and heterogeneity.

FIG. 8. ccRCC proteogenomic and TME ITH revealed by comprehensive multi-segment integrative analysis. A) Schematic representation of the ITH cohort workflow. B) Proteogenomic aberration and histological features landscape of ITH cohort samples. Upper panel: Various segment-wise histopathologic feature annotations obtained from pathology review, methylation subtype, immune subtype, and wGII category. Bottom panel: Intratumoral variation in genomic aberrations, starting with the number of SV, total somatic mutations followed by known key drivers with variant allele frequency, and finally select CNV. C) Bar plots represent the frequency of heterogeneity features (left) and the heterogeneity count in the ITH cohort. D) Box plots indicate the distributions of xCell CD8+ T signature, overall immune signature, and endothelial signature between the groups with and without immune heterogeneity (w-ITH vs., w/o-ITH). E) 6 representative cases for the comparison between the w-ITH group and the w/o-ITH group (three for each group). F) Panoptes-based multi-resolution neural network models were trained to predict immune subtypes based on H&E images displayed on the left (Hong et al., 2021). Scale bar=3 mm.

FIG. 9. Single-nuclei RNA-seq atlas identifies distinct intra-tumor epithelial populations. A) Top panel: snRNA-seq analysis workflow schematic. Bottom panel: Cell atlas was generated from multi-segment snRNA-seq, from 12 segments (4 cases). B) Schematic tracks present the heterogeneity observed in the 12 segments at the histological and molecular feature level characterizations besides tumor cells based on H&E image review and bulk sequencing data, respectively. C) Stacked bar plot shows the frequency and composition of non-tumoral cell types found in the TME of cases C3L-01287 and C3N-00148. D) UMAP shows the tumor sub-clusters (C0A, COB, C1-6) and the corresponding ITH found in the 4 segments obtained from case C3N-00148. E) UMAP shows the tumor sub-clusters and the corresponding ITH in the 2 segments of case C3L-01287 is shown in the top panel. Bottom panel: C3L-01287 HE image reveals the presence of classic clear cell and mutually exclusive rhabdoid regions in this tumor as indicated.

FIG. 10. Single-nuclei RNA-seq atlas further refines sarcomatoid and rhabdoid histology associated gene expression signatures. A) Bubble plot presents the expression profiles of the top markers associated with the C0A tumor cluster in C3N-00148. B) UMAPs show the integration among six segments of case C3N-00148 and two additional cases C3L-00079 and C3L-00968 with the sarcomatoid feature. C) Bubble plots present the expression profiles of the top markers associated with the C0A tumor cluster in snRNA-seq at the integration level and individual case level. D) Corresponding high and low expression of TGFBI in two representative cases with high and diffuse staining intensity noted in sarcomatoid area with the absence of staining in nested clear cell area. Scale bar=200 microns. E) Bubble plot presents the expression profiles of the top markers associated with C0. F) UMAPs show the integration among three segments of case C3L-01287 and one additional case C3L-02551 with the rhabdoid feature. G) Bubble plots present the expression profiles of the top markers associated with the C0 tumor cluster in snRNA-seq at the integration level and individual case level. H) Corresponding high and low expression of KIF2A in two representative cases with high and diffuse staining intensity noted in rhabdoid area with the absence of staining in nested clear cell area. Scale bar=200 microns.

FIG. 11. DNA hypermethylated Methyl1 subtype is associated with BAP1 mutations and various other features linked to poor survival. A) Heatmap depicts patient classification according to the 3 DNA methylation subtypes identified by consensus clustering analysis and the various features associated with each of the three methylation subtypes. B) Kaplan Meier plot indicates the association between overall survival and the three methylation subtypes in CPTAC ccRCC cohort. C) The scatterplot correlates methylation difference (beta value) and RNA expression difference of the top Methyl1 signature probes (genes). D) Scatter plots show the differentially expressed proteins (DEPs) associated with Methyl1 (vs.Methyl2&3) and enriched pathways based on DEPs up in Methyl1. E) Scatter plot presents the DEGs and DEPs associated with BAP1 mutation status. F) Scatter plot presents the DEGs and DEPs associated with wGII category. G) Kaplan Meier plot indicates the association between overall survival and UCHL1 expression in CPTAC ccRCC cohort. H) Comparative high and uniform expression of UCHL1 in tumor of a Methyl1 ccRCC case (a, b) with absence of UCHL1 expression in tumor of a Methyl3 ccRCC case (c, d). I) Characterization of UCHL1 in a morphologically heterogeneous ccRCC case. Scale bar=3 mm.

FIG. 12. Identification of key phospho signaling pathways, kinase-substrate interactions in ccRCC tissues, and integration of ex-vivo kinase drug inhibition data from RCC cell lines. A) Top 50 signaling pathways between kinases and phospho-substrates across all tumors. B) Heatmap depicting the classification of ccRCC tumors using phosphorylation events with CV >25% quartile into four phospho groups (P1-4). C) Pathways and kinase activities are inferred from phosphoproteome data for each phosphor subtype. NES: normalized enrichment score from PTM-SEA. D) Schematic representation summarizing the kinase inhibition experiment conducted in 5 RCC cell lines targeting the kinases identified in the initial cohort.

FIG. 13. Alteration of protein glycosylation specific to ccRCC and high-grade ccRCC. A) Volcano plot shows the intact glycopeptides (IGP) differentially expressed between tumors and NATs. B) Performance of glyco-signatures individually and as a multi-signature panel for differentiating tumor and non-tumor tissues. C) Glycan type distribution in 51 upregulated and 131 downregulated intact glycopeptides (tumors compared to NATs). D) Scatter plot represents the glycosylation changes (y-axis) compared to global protein expression (x-axis) changes in tumors relative to NATs. E) Heatmap displays the classification of ccRCC tumors by consensus clustering of glycoproteomics data into three glycoproteomic subtypes (Glyco 1-3) and overlaid with 14 variables represented by individual tracks immediately below. F) The expression profile of HYOU1 between low-grade (CL-light red) and high-grade (CH-dark red) tumors. Asterisks represent significant differences between two groups: * FDR <0.05; ** FDR<0.01. G) Kaplan Meier plot compares protein expression of HYOU1 between High (upper quartile) and Low (lower quartile) groups in the CPTAC cohort.

FIG. 14. Dysregulated metabolism in high-grade and low-grade ccRCC. A) PCA plot shows the distribution of 50 tumors (colored and shaped based on their histopathologic subtypes) with metabolome characterization. B) Differentially expressed metabolites (DEMs) between high-grade tumor (CH) and low-grade tumor (CL) were identified. C) Enriched metabolic pathways corresponding to CH and CL, respectively. D) Sankey diagram visualizes the distribution of metabolic pathways and super pathways for the 183 metabolites used for metabolomic subtyping. E) Heatmap shows the four metabolomic subtypes that were identified among the 50 tumors and 7 NATs. F) Network plot of M00844 Arginine biosynthesis, M00029 Urea cycle, and M00009 Citrate cycle demonstrates the connection of metabolites and enzymes, and the expression fold change of metabolites and direction of enzymes in tumors compared with NATs. G) Among the 213 cases, the fractions of high expression of GLUL and GLS are higher in highgrade tumors (CH, CH—S, CH-R) compared with those of low-grade tumors (CL) as indicated by these stacked bar plots. H) The distribution of 50 tumors with multi-level profiling of histological, BAP1 mutation, wGII, methylation, immune, multi-omic CNV+RNA+Protein), phospho, glyco, and metabolome subtypes.

FIG. 15. Association between high-grade feature count (HGFC) and overall survival, immune and multi-omic clusters. A) Overall survival difference among the four histopathologic subtypes indicated by Kaplan Meier plot indicates the association between overall survival and histopathologic subtypes. B) The hazard ratios of the histopathologic subtypes CH, CH-S, and CH-R compared to the CL reference. C) Scatter plot shows the correlation between the wGII score and ploidy values of all the tumor samples. D & E) Representative H&E images showing the seven high-grade features in schema: solid, eosinophilic/granular change, thick trabeculae, alveolar, and papillary/pseudopapillary pattern, respectively. Scale bar=200 microns. F) Proteomic changes associated with tumor architecture (pattern) and cytology subtypes. G) Kaplan Meier plot shows the association between integrative signature score (based on protein abundance of LRRC5, RPN2, and SERPINH1 weighted by length) and overall survival (cases divided into the upper quartile and lower quartile for comparison). H) Kaplan Meier plot shows the association between HGFC and overall survival (cases divided into those containing less than 3 or 3 and above HGFC features). I) Principal component analysis (PCA) of proteomics data obtained from tumors (red dots) and normal adjacent tissues (NATs, blue dots). J) Box plots representing immune cell type (10 different cell types) abundance as assessed by CIBERSORTx deconvolution tool using Leukocyte signature Matrix 10 (LM10). K) Heatmap represents the xCell cell-type-enrichment-based immune subtyping performed for the entire cohort. L) Multi-omic subtyping based on non-negative matrix factorization (NMF).

FIG. 16. Integrated proteogenomic analysis of ccRCC genetic and TME heterogeneity. A) Landscape of driver mutations and their clonality across the ITH cohort segments. B) Heatmap depicts the frequency of segment-specific (green), shared-subclonal (pink), and shared-clonal (red) somatic mutational events found across tumors and/or even across segments of a given tumor. C) Heatmap of the absolute copy number determined from whole exome/whole genome sequencing data for 132 tumor segments obtained from 40 ccRCC patients in the ITH cohort (cases clustered by CNV pattern and segments ordered by case numbers. D) Absolute copy number (CN) plots reveal the copy number heterogeneity found across segments for a given case (C3N-00573). E) Dosage effects of CNV observed at protein and RNA expression levels. F) t-SNE of the data obtained from Panoptes-based multi-resolution neural network models that were trained to predict immune subtypes based on H&E images. G) High concordance was observed between the sample immune annotations rendered by pathology team review and by data-driven delineation for the overall immune infiltration level (ESTIMATE immune score and xCell immune score). H) Overall survival difference among the cases with vs. without heterogeneity in wGII status or driver mutations (in VHL, PBRM1, KDM5C, SETD2, BAP1).

FIG. 17. Single-nuclei RNA-seq analysis identified sarcomatoid and rhabdoid expression signatures. A) The UMAP dimensionality reduction plot displays 10 different cell types (based on biomarker expression and colored accordingly) identified from 104,654 nuclei (with anchoring process) split by cases. B) The main tumor cluster includes 9 tumor sub-clusters being enriched in different cases colored by different colors. C) Trajectory analysis of the four cases is shown and colored by segment ID and predicted pseudotime on the first and second rows, respectively. D) Tumor sub-cluster trajectory obtained from pseudotime gene expression analysis of case C3N-00148 that shows the relationship among the various tumor sub-clusters. E) CNV mapping to UMAP shows that segment-3-enriched sub-clusters (C3N-00148) were featured with CNV events including chr9q loss. In these clusters, 9p (e.g., JAK2) and 13 loss (e.g., RB1) was also detected. F) H&E image of the selected region of C3N-00148 segment-3 showing distinct sarcomatoid differentiation and spindle cell proliferation (˜25-30%, right side box) juxtaposed to a region with clear cell morphology (left box). Scale bar=300 microns. G) Inferred copy number variation (CNV) of select chromosomes mapped to UMAP shows that sub-cluster C0 was characterized by 8q and 3q gains while 5q gain was prevalent in other tumor clusters. H) Copy number characterization using WES of macro-dissection confirmed the distinct CNV features in the clear cell area (seg1-CC) and rhabdoid area (seg1-Rh). I) Heatmaps indicate the expression profiles of candidate feature-associated markers (e.g., sarcomatoid, rhabdoid) at the bulk RNA and protein levels.

FIG. 18. Methylation subtype associated with BAP1 mutations and poor survival. A) Box plots present the distributions of ploidy, wGII score, and stemness score among the methylation subtypes. B) Kaplan Meier plot indicates the association between overall survival and the three methylation subtypes in TCGA clear cell RCC KIRC cohort. C) Panoptes-based multi-resolution neural network models were trained to predict methylation subtypes based on H&E images (Hong et al., 2021). D) The distribution of UCHL1 immunohistochemistry score among methylation subtypes represented by boxplot and annotated by wGII category and BAP1 mutation status. E) Kaplan Meier plot indicates the association between overall survival and UCHL1 expression in TCGA KIRC cohort. High UCHL1 expression (upper quartile of the cohort) is significantly associated with poor survival as indicated by the p-value. F) High correlation between quantified UCHL1 protein abundance versus UCHL1 IHC score is represented by scatter plot. BAP1 mutation type (deleterious-red, missense-green) and status (wild-type-blue, mutant-red, or green) of the evaluated tumors are indicated. G) Representative H&E and immunohistochemistry (IHC) images from CAIX, BAP1, UCHL1 IHC validations performed on BAP1 wild-type and mutated tumors. UCHL1 is overexpressed in most BAP1 mutated tumors while BAP1 is variably downregulated in BAP1 mutated tumors. H) Characterization of UCHL1 in a patient with primary renal mass (positive UCHL1 expression, stronger in higher grade than lower grade tumor area) (a, b). High and uniform UCHL1 expression in metastatic sarcomatoid differentiated ccRCC tubo-ovarian mass in the same patient (c, d). Scale bar=200 microns. I) Strong homogeneous expression of UCHL1 in primary tumors of four different clinically aggressive patients (who developed metastasis) despite varying morphological patterns in conventional nested (a, b), alveolar (c, d), acinar (e, f), and rhabdoid (g, h) tumors. Scale bar=200 microns. J) Plots indicate the IC50 of UCHL1 inhibition (CAS 668467-91-2) in Caki-1, HK-2, and 786-O, respectively. K) Morphology of Control and CAS 668467-91-2 (30 μM, 24 hr) treated 786-O renal cancer cells. L) Impact of UCHL-1 inhibition on Akt signaling in 786-O renal cancer cells evaluated by Western blot analysis.

FIG. 19. Phosphoproteomic analysis. A) An expanded view of the phospho-signaling network. B) Heatmap showing K-means clustering results of the phosphoproteomic features among the phosphoproteomic groups. C) Stacked bar plots highlight the association between select important ccRCC disease variables and the different phosphoproteomic groups. D) Phosphorylation events upregulated in BAP1 mutant versus BAP1 WT tumors comparison as revealed by DIA-based profiling in the tissue cohort and the status of those sites upon various treatments in RCC cell lines. E) ROC analysis of BAP1 mutant and WT tumors based on phospho-substrates.

FIG. 20. Alteration of protein glycosylation specific to ccRCC and high-grade ccRCC. A) Significant concepts in the KEGG pathway enrichment analysis using the glycoproteins that exhibit differential intact glycopeptides expression in tumors compared to NATs. B) Volcano plot depicts the differentially expressed glycosylation enzymes (n=35) at the global protein level. C) K-means clustering of the glycoproteomic features in three glycoproteomic subtypes for tumors. D) Distribution of each glycoproteomic subtype in association with IPCs shown as boxplots. E) Bubble plots represent upregulated intact glycopeptides in association with the glyco subtypes. F) Performance of HYOU1 for differentiating low-grade and high-grade tumors. G) Immunochemistry staining of HYOU1 protein expression in high-grade (C3N-01648) versus low-grade (C3N-01905) tumor. Scale bar=200 microns. H) Kaplan Meier plot compares HYOU1 between High (upper quartile) and Low (lower quartile) groups at the RNA level in the CPTAC cohort. I) Kaplan Meier plot compares HYOU1 between High (upper quartile) and Low (lower quartile) groups at the RNA level in the TCGA cohort. J) Forest plot for Cox Proportional Hazards Model adjusting by age and sex (comparing HYOU1 high and low at the protein level in the CPTAC cohort).

FIG. 21. Dysregulated metabolism in high-grade ccRCC and low-grade ccRCC. A) Enriched metabolic pathways in the comparison of tumors and NATs. B) Volcano plot shows the upregulated DEMs in CH-S compared with CL. C) Enriched metabolic pathways corresponding to CH-S. D) The distributions of histopathologic, methylation, and immune subtypes, wGII status, sex, and tumor grade among the metabolomic subtypes. E) Upregulated metabolites associated with each of the methylation subtypes. F) Expression profiles of key metabolites and enzymes presented in FIG. 8F. G) Plots indicate the IC50 of GLUL inhibition in skrc42.EV and HK-2, respectively. H) 2-HG and MYC expressions were significantly higher in Methyl1 (methylation subtype associated with the worse prognosis).

FIG. 22. Characterization of renal cell carcinoma proteogenomic aberration landscape reveals association between survival and copy number-based genome instability, and identifies prognostic biomarkers. A) Proteogenomic aberration landscape of ccRCC and non ccRCC tumors. Top panel: various sample histo-molecular annotations are represented as tracks. Middle panel: Recurrent 3p driver gene (VHL, PBRM1, BAP1, SETD2) mutations and 3p copy loss in ccRCC, in contrast non-ccRCC lacks 3p drivers, but display distinct recurrent events such as chr 1loss, CCND1 rearrangements in RO, TP53 mutations in chRCC, chr 7/17 gains in pRCC among others. Bottom panel: Heatmaps show the top 10 differentially expressed mRNA transcripts and proteins enriched in each biological process annotated. B) Bubble plot shows various RNA and proteins differentially enriched pathways among the various RCC subtypes. C) Density plots show distribution of prediction immune composition for ccRCC and non-ccRCC samples. D) Heatmap shows the absolute copy number (CN) variation deduced from CNVEX output for non ccRCC tumors (Top) and ccRCC tumors (bottom). E) Pie charts show the distribution of BAP1 mutation status, wGII grouping, immune subtype, tumor classes and NMF clustering in 5 methylation subgroups. F) UMAP visualization mapping RNAseq data of CPTAC ccRCC samples onto TCGA KIRC and CPTAC non-ccRCC samples onto TCGA KIRP samples separately. G) Heatmap shows fold changes of gene abundance in log 2 scale between high wGII group and low wGII group in TCGA KIRP, CPTAC non-ccRCC RNA-seq and protein datasets. H) Network visualization of the wGII markers in non-ccRCC subgroup and the biological functions they are enriched with.

FIG. 23. non-ccRCC prognostic markers and delineation of tumor transcriptomic heterogeneity, immune infiltration status and tumor cell of origin by single nuclei RNA sequencing. A) Histograms show the distribution of median Concordance Index (CI) in all the genes modeled as survival predictors in TCGA KIRP (left) and KIRC (right) cohorts. B) K-M curves show differences in survival outcomes between groups separated by higher expression and lower expression (relative to averaged expression of the nominated markers that results in best separation) in CPTAC non-ccRCC (left) and ccRCC (right) samples. C) IHC images show PYCR1 expression in 2 high wGII non-ccRCC cases and 1 low wGII non-ccRCC case. D) PYCR1 inhibitors pargyline and compound 4 had no effect on cell viability (left) in metastatic papillary RCC cell line ACHN1, while the UCHL1 inhibitor showed dramatic effects in both ACHN1 (IC50 value 0.65 μM, middle) and NB-1 cell lines (IC50 value, right). E) Two-dimensional Uniform Manifold Approximation and Projection (UMAP) visualization of snRNA-seq data from 8 non-ccRCC tumors. F) Projection of snRNA-seq data from tumor nuclei to the first 3 principal components of 6 tumors (AML excluded), colored by tumor types. G) Radar plot shows the probability of cell-of-origin predicted by random forest classifier for different tumor subclusters for each of the RCC subtypes. H. Heatmaps represent the averaged abundance of differentially expressed protein (top) and mRNA (bottom) markers from each RCC subtype versus benign kidney tissue among the epithelial cell types identified from normal kidney tissues single cell RNAseq data.

FIG. 24. Phosphoproteomic changes in non ccRCC and genome unstable tumors. A) Heatmap shows differentially expressed kinases across major subtypes. B) Boxplots highlight 4 subtype-specific up-regulated kinases. From top to bottom: FLT1 in ccRCC, MET in pRCC type1, KIT in oncocytic tumors and MYLK in AML. C) Pathways enriched among the differentially regulated phosphorylation sites across subtypes. D) Kinases that are enriched with down- or up-regulated phosphorylation in high wGII compared to low wGII non-ccRCC samples. E) Significantly co-regulated kinases-substrate pairs in high wGII tumors (fdr<0.05, abs (lo2 fc of kinase)>0.05, abs (log 2fc of substrates >0.5)). F) Protein 3D structure of CDK2. Highlighted residues are significantly up-regulated phosphorylation clusters identified by CLUMPS-PTM.

FIG. 25. RCC glycoproteome reflects tumor immune infiltration and angiogenesis. A) Venn diagram shows the glycoprotein overlap between glyco search on glyco-enriched samples (glyco enrichment) and glyco search on phospho-enriched samples (phosphor enrichment). B) Distribution of various glycoforms found in the glyco-enriched samples C) Distribution of differentially expressed glycoforms D) Stacked bar plots summarizing the differentially expressed glycoproteins (left) and proteins (right) in glyco-enriched samples and their cell type annotation, delineated by cell type specific expression from previous single cell RNAseq data. E) Cell type enrichment analysis for glycoproteins markers in oncocytoma (left) and pRCC (right) in glyco-enriched samples. F) Heatmap of differentially expressed cell type specific glycoprotein markers in glyco-enriched samples. G) Selected glycoprotein markers validated from Human Protein Atlas H) Fucosyltransferase 8 (FUT8) protein expression across different RCC subtypes and benign kidney tissue in the study cohort. I) FUT8 RNA expression pattern among different cell types identified in the type1 pRCC sample (C3N-00439) subjected to snRNA-seq. J) GSEA for putative FUT8 glycoprotein targets expression in pRCC. K. Differentially expressed glycoproteins between high wgii versus low wgii non-ccRCC samples.

FIG. 26. Metabolomic aberrations across RCC subtypes. A) Barplot represents the total number of filtered metabolites used in the analysis and their distribution across functional categories. B) PCA plot shows the clustering of metabolomics data from different non-ccRCC tumors and benign kidney samples. C) Bubble plots showing differentially enriched pathways between tumor subtypes, bubble size represents the number of compounds in each pathway. D) Sketch of key pathways. E) Bar plot shows the distribution of tumor subtypes stratified by high and low wGII groups. F) Volcano plot shows metabolites with significant differential abundance (abs (log 2fc)>1 and p value <0.05) between high and low wGII tumors.

FIG. 27. Identification and validation of proteogenomic biomarkers that distinguish papillary RCC from mucinous tubular spindle cell carcinoma (MTSCC). A) Scatter plot shows significantly differentially (abs (log 2fc)>2 and q value <0.05) genes in protein expression (x axis) and RNA expression (y axis) between pRCC type1 and other tumors. B) Boxplots show the specificity of nominated pRCC type1 protein markers PIGR and SOSTDC1. C) Boxplots show the expression of nominated pRCC type1 protein markers PIGR and SOSTDC1 in the proteomics data from Xu et al 2022. D) H&E images, Protein IHC and RNA-ISH images (top to bottom) of nominated marker PIGR in normal kidney tissue, pRCC, MTSCC tumors (upper panels from left to right) and SOSTDC1 in chRCC, pRCC and MTSCC (lower panels from left to right). E) Boxplots show RNA-ISH comparative scores of PIGR and SOSTDC1 in different tumor types. F) Location of missense mutations on MET across TCGA cohorts are colored on the MET protein domain diagram. G) PTM-SEA analysis concludes pathways such as EGFR is significantly enriched with increased phosphorylation in MET mutant pRCC samples. H) Enrichment in chromosome 7 and chromosome 17 gene sets are tested with RNA expression and protein expression difference between chromosome 7 gain samples and no gain samples in non-ccRCC groups.

FIG. 28. Identification and validation of proteogenomic biomarkers that distinguish

Oncocytomas (RO) from Chromophobe RCC. A) Box plots showing the protein abundance (top) and RNA expression (bottom) of known biomarkers of RO and chRCC across different kidney tumors. B) PyScenic analysis identifies transcriptional modules commonly enriched in RO and chromophobe RCCs (left), and those specifically enriched in chromophobe RCCs (right). C) Venn diagrams indicate the overlap between differentially expressed proteins identified in this study (CPTAC) and PXD007633 dataset in RO (left) and chromophobe RCC (right). D) Scatter plot represents differentially expressed proteins (x-axis) and mRNA (y-axis) between RO and chromophobe RCC. E) Box plots show the Chromophobe RCC specific marker GPNMB′ (left) and RO specific biomarker MAPRE3 (right) protein abundance (top) and RNA expression (bottom) in different tumor subtypes. F) Immunohistochemistry images of nominated markers seen in representative tumor sections of different renal tumors. G) Box plots showing the protein abundance (top) and RNA expression (bottom) of THSD4 across different kidney tumors, which potentially distinguishes RO type 1 from RO type 2.

FIG. 29. Characterization of renal cell carcinoma proteogenomic aberration landscape reveals association between survival and copy number-based genome instability and identifies prognostic biomarkers. A) Sample availability of bulk experiments included in this study. B) ProTrack modules include Sample Dashboard, Histology Viewer, Proteomic QC, Two-gene Correlation, Interactive Heatmap and Expression Boxplots. C) PCA plots show distribution of samples with regard to PC1 (x axis) and PC2 (y axis) in global proteome, phosphoproteome, phosphosites, phospho-enriched glycoproteome and glyco-enriched glycoproteome, stratified by tumor/normal condition and ccRCC/non-ccRCC cohorts, colored by TMT plexes. D) CNV for samples in TCGA studies. E) Distribution of tumor class in high and low wGII and ploidy subgroups. F) Survival analysis comparing high wGII versus low wGII samples in this cohort (CPTAC) and TCGA (KIRC and KIRP). G) Enrichment plots show genes ranked by signed-log 10 (p value derived from survival regression model), the higher the metric is the stronger the gene is associated with worse survival. H) GSEA analysis comparing high and low wGII groups using HALLMARK gene sets. I) Survival analysis comparing hyper methylated group with the rest (left) and myeloid-lymphoid high group with the rest (right).

FIG. 30. non-ccRCC prognostic markers and delineation of tumor transcriptomic heterogeneity, immune infiltration status and tumor cell of origin by single nuclei RNA sequencing. A) Upregulated genes in KIRP high wGII samples compared to low wGII samples. B) Two-dimensional UMAP visualization of snRNA-seq data for each of the 8 tumors, colored by clusters. C) Stacked bar plot shows the cell type composition of the different samples. D) Two-dimensional UMAP visualization of snRNA-seq data for 3 ccRCC samples from the discovery cohort, colored by cell types. E) Two-dimensional UMAP visualization of snRNA-seq data for each of the 8 tumors, colored by tumor subclusters. F) Two-dimensional UMAP visualization of snRNA-seq data for the RO type1 tumor, colored by cell cycle classification predicted based on expression of phase specific markers. G) Box plots display fold change (log 2 scale) of snRNA-seq expression of genes located in selected chromosomal arms between tumor subcluster and other cells of the pRCC type1 sample (top) and the RO type2 sample (bottom) H) Radar plot shows the probability of cell-of-origin predicted by random forest classifier for different tumor subclusters for each of the RCC subtypes using bulk RNAseq data. I) Heatmap shows overlaps of renal epithelial cell types identified in Lake et al (snRNA-seq) and Zhang et al. (scRNA-seq). J) Heatmap shows scores of subtype-specific gene signatures derived from top 50 most up-regulated transcripts for each tumor subtype.

FIG. 31. Phosphoproteomic changes in non ccRCC tumors. A) Boxplots show phosphorylation intensity stratified in tumor subtypes. B) Network shows member proteins and phosphorylation in the Leptin pathway. C) Heatmap shows the log 2 fold change of kinases between high wGII samples and low wGII samples in ccRCC and non-ccRCC.

FIG. 32. RCC glycoproteome reflects tumor immune infiltration and angiogenesis. A) Distribution of glycoforms found in phospho-enriched samples B) Glycosylation change versus its corresponding protein (fold change, glyco). Right: RO. Left: pRCC. C) Glycosylation changes versus its corresponding protein (signed p-value, glyco). Right: RO. Left: pRCC. D) Volcano plot for differential expressed glycoproteins. E) Stacked plots summarizing differential expressed cell type specific glycoproteins (left) and proteins (right) in phospho-enriched samples. F) Heatmap of differentially expressed expressed cell type specific glycoprotein markers in phospho-enriched samples. G) Protein (left) and RNA (right) expression of glycosylation enzymes in kidney tumors. H) FUT8 RNA expression across kidney cancers in combined TCGA and MCTP cohort. I) Volcano plot for glycosylation changes of putative FUT8 targets in pRCC. J) MET glycosylation in glyco-enriched samples K. MET_N785 glycosylation in glyco-enriched samples.

FIG. 33. Metabolomic aberrations across RCC subtypes. A) Barplot shows the top 10 metabolic pathways with the greatest number of compounds identified. B) Volcano plots show up-regulated compounds in pRCC type1 (left), AML (middle) and ROs (right), with compounds of different categories colored. C) Metabolograms depicting select metabolic pathways to show the coordinated regulation of compounds (metabolites abundance; within green border) and the corresponding enzymes (proteins abundance; within orange border) in pRCC type1 (outer circle), AML (middle circle) and ROs (inner circle). D) Boxplots compare fold changes of metabolites (M), proteins (P) and mRNAs (R) zcross tumor subtypes within each pathway.

FIG. 34. Identification and validation of proteogenomic biomarkers that distinguish papillary RCC from mucinous tubular spindle cell carcinoma (MTSCC). A) Boxplots highlight nominated pRCC type1 mRNA markers PIGR and SOSTDC1 B) Volcano plot shows significant, differentially (abs (log 2fc)>0.5 and p value <0.05) regulated phosphorylation sites between missense MET mutated (n=2) and wild type MET (n=5) pRCC tumors. C) Violin plots show distribution of gene expression fold changes between chr7 gain and no gain samples separately derived from ccRCC and non-ccRCC subgroups. D) Enrichment in chromosome 7 and chromosome 17 gene sets are tested with RNA expression and protein expression difference between chromosome 7 gain samples and no gain samples in ccRCC groups. E) Scatter plot shows the signed log 2 fold change of genes's protein expression (x axis), RNA expression (y axis) derived from DE analysis comparing chromosome 7 gain with no gain samples in ccRCC (upper) and non-ccRCC (lower) subgroup. F) Hallmark pathway enrichment analysis based on expression fold changes between chromosome 7 gain and no gain samples stratified by ccRCC and non-ccRCC subgroups with both protein and RNA expression data. G) Kinases with enriched up-regulated phosphorylation (red) and down-regulated phosphorylation (blue) in Chr 7 gain samples compared to Chr7 no gain samples.

FIG. 35. Scatter plot showing that UCHL1 and IGF2BP3 collectively detect most tumors with adverse molecular features.

### DEFINITIONS

To facilitate an understanding of the present disclosure, a number of terms and phrases are defined below:

As used herein, the terms “detect”, “detecting” or “detection” may describe either the general act of discovering or discerning or the specific observation of a composition. Detecting a composition may comprise determining the presence or absence of a composition. Detecting may comprise quantifying a composition. For example, detecting comprises determining the expression level of a gene. The composition may comprise a nucleic acid molecule. For example, the composition may comprise at least a portion of a nucleic acid encoding the cancer markers disclosed herein. Alternatively, or additionally, the composition may be a detectably labeled composition.

As used herein, the term “subject” refers to any organisms that are screened, prognosed, or treated using methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The nucleic acid molecule may comprise one or more nucleotides. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “label” as used herein refers to any atom or molecule that can be used to provide a detectable (preferably quantifiable) effect, and that can be attached to a nucleic acid or protein. Labels include but are not limited to dyes; radiolabels such as 32P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

As used herein, the term “sample” is used in its broadest sense. In one sense, it is meant to include a specimen or culture obtained from any source, as well as biological and environmental samples. Biological samples may be obtained from animals (including humans) and encompass fluids (e.g., urine, blood, blood products, etc), solids, tissues (e.g., kidney tissue or cells), and gases. Biological samples include blood products, such as plasma, serum and the like. Such examples are not however to be construed as limiting the sample types applicable to the present disclosure.

### DETAILED DESCRIPTION OF THE DISCLOSURE

Provided herein are compositions and methods for cancer diagnosis, research and therapy, including but not limited to, cancer markers. In particular, provided herein are methods of treating renal cancer based on expression levels of cancer markers.

Renal cell carcinoma (RCC) is a common and deadly disease with a worldwide estimate of 431,288 new cases and 179,368 deaths (Global Cancer Observatory) in 2020. Of the many RCC subtypes, clear cell RCCs (ccRCCs) account for up to 80% of all kidney cancer cases and the majority of kidney cancer-related deaths, while non-ccRCCs comprise several different subtypes. Although therapeutic modalities such as surgical resection for localized ccRCC are widely available, options for treating advanced cases are initially limited due to resistance to conventional chemotherapy. Despite a variety of (often targeted) experimental treatments, combinations of tyrosine kinase inhibitors (TKIs) such as axitinib, cabozantinib, and lenvatinib and immune checkpoint inhibitors (IMIs) such as pembrolizumab, nivolumab, and ipilimumab in use, many patients develop resistance to therapy. Therefore, prognostic markers to detect aggressive disease earlier and new therapeutic options to supplement existing treatment modalities will have significant impact on disease diagnosis and management.

Experiments described herein identified several proteins associated with cell cycle proliferation, coagulation pathway and others as upregulated in ccRCC. UCHL1, a deubiquitinase enzyme, was significantly associated with high grade ccRCC. UCHL1 is overexpressed at both protein and RNA levels in BAP1 mutant ccRCC samples, a sub type of ccRCC known to be associated with aggressive disease and poor survival. Renal cancer ex-vivo cell lines treated with a small molecule inhibitor against UCHL 1 showed a decrease in cell viability. Thus, UCHL1 is a prognostic biomarker for predicting poor clinical outcomes and a therapeutic target for patients with aggressive renal tumors.

In addition to ccRCC, further experiments described herein identified markers in heterogeneous non clear cell renal cell carcinomas (non-ccRCC), which encompass malignant and benign tumors. Further refinement of differential diagnosis biomarkers under biopsy setting, prognostic markers for early aggressive disease detection and treatments to complement immunotherapy are current clinical needs in non-ccRCC. Multi-omics analyses of 48 non-ccRCC with 103 ccRCCs revealed proteogenomic, phosphorylation, glycosylation and metabolic aberrations in RCC subtypes and in non-ccRCC tumors with genome instability, a feature associated with poor survival. Expression of PYCR1, IGF2BP3, DPYSL3, IKBIP, and FABP6 genes is highly associated with genome instability and comprises a four-gene non-ccRCC signature useful in prognostic and therapeutic applications.

Insulin-like growth factor 2 mRNA-binding protein 3 (IGF2BP3) was further identified as a prognostic marker for poor prognosis in RCC, as well as a clinical target for treating RCC. The protein encoded by this gene is primarily found in the nucleolus, where it can bind to the 5′ UTR of the insulin-like growth factor II leader 3 mRNA and may repress translation of insulin-like growth factor II during late development. The encoded protein contains several KH domains, which are important in RNA binding and are known to be involved in RNA synthesis and metabolism.

Accordingly, provided herein are diagnostic, prognostic, screening, and therapeutic methods for use in treating patients with RCC. Exemplary methods are described herein.

### I. Methods of Assaying Marker Expression

As described herein, embodiments of the present disclosure provide diagnostic, screening, and therapeutic methods that utilize the detection of the expression level of cancer markers including but not limited to, UCHL1 and/or one or more of PYCR1, DPYSL3, IKBIP, or FABP6. Exemplary, non-limiting methods are described herein.

In some embodiments, the cancer markers of the present disclosure are detected using a variety of nucleic acid techniques, including but not limited to: nucleic acid sequencing; nucleic acid hybridization; and, nucleic acid amplification.

In some embodiments, nucleic acid sequencing methods are utilized (e.g., for detection of amplified nucleic acids). In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92:255 (2008), herein incorporated by reference in its entirety. Those of ordinary skill in the art will recognize that because RNA is less stable in the cell and more prone to nuclease attack experimentally RNA is usually reverse transcribed to cDNA before sequencing.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts (e.g., cancer markers) within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, cancer markers are detected using fluorescence in situ hybridization (FISH). In some embodiments, FISH assays utilize bacterial artificial chromosomes (BACs). These have been used extensively in the human genome sequencing project (see Nature 409:953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present disclosure further provides a method of performing a FISH assay on the patient sample. The methods disclosed herein may comprise performing a FISH assay on one or more cells, tissues, organs, or fluids surrounding such cells, tissues and organs. In some instances, the methods disclosed herein further comprise performing a FISH assay on human kidney cells, human kidney tissue or on the fluid surrounding said human kidney cells or human kidney tissue. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, MD). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

The one or more cancer markers may be detected by conducting one or more hybridization reactions. The one or more hybridization reactions may comprise one or more hybridization arrays, hybridization reactions, hybridization chain reactions, isothermal hybridization reactions, nucleic acid hybridization reactions, or a combination thereof. The one or more hybridization arrays may comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., cancer markers) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or electrochemistry on microelectrode arrays.

The methods disclosed herein may comprise conducting one or more amplification reactions. Nucleic acids (e.g., cancer markers) may be amplified prior to or simultaneous with detection. Conducting one or more amplification reactions may comprise one or more PCR-based amplifications, non-PCR based amplifications, or a combination thereof. Illustrative non-limiting examples of nucleic acid amplification techniques include, but are not limited to, polymerase chain reaction (PCR), reverse transcription polymerase chain reaction (RT-PCR), nested PCR, linear amplification, multiple displacement amplification (MDA), real-time SDA, rolling circle amplification, circle-to-circle amplification transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, proteins expressed by cancer marker genes are detected (e.g., using an immonassay). Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present disclosure provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present disclosure contemplates any method capable of receiving, processing, and transmitting the information to and from laboratories conducting the assays, information providers, medical personnel, and subjects. For example, in some embodiments of the present disclosure, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by one or more medical personnel (e.g., a treating clinician, physician assistant, nurse, or pharmacist). For example, rather than providing raw expression data, the prepared format may represent a diagnosis or risk assessment (e.g., levels of the cancer markers described herein) for the subject, along with recommendations for particular treatment options. The data may be displayed to the medical personnel by any suitable method. For example, in some embodiments, the profiling service generates a report that can be printed for the medical personnel (e.g., at the point of care) or displayed to the medical personnel on a computer monitor.

In some embodiments, the information is first analyzed at the point of care or at a regional facility. The raw data is then sent to a central processing facility for further analysis and/or to convert the raw data to information useful for medical personnel or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the medical personnel, the subject, or researchers.

In some embodiments, the subject is able to directly access the data using the electronic communication system. The subject may choose further intervention or counseling based on the results.

In some embodiments, the data is used for research use. For example, the data may be used to further optimize the inclusion or elimination of markers as useful indicators of a particular condition or stage of disease or as a companion diagnostic to determine a treatment course of action.

Compositions for use in the diagnostic methods described herein include, but are not limited to, antibodies, probes, amplification oligonucleotides, and the like.

The compositions and kits may comprise 1 or more, 2 or more, 3 or more, or 4 or more antibodies, probes, pairs of probes, pairs of amplification oligonucleotide, or sequencing primers.

The probes or primers may hybridize to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 21 or more target molecules. The target molecules may be RNA, DNA, cDNA, mRNA, a portion or fragment thereof or a combination thereof. In some instances, at least a portion of the target molecules are cancer markers. The probes may hybridize to 1 or more or 2 or more cancer markers disclosed herein.

Typically, the probes or primers comprise a target specific sequence. The target specific sequence may be complementary to at least a portion of the target molecule. The target specific sequence may be at least about 50% or more, 55% or more, 60% or more, 65% or more, 70% or more, 75% or more, 80% or more, 85% or more, 90% or more, 95% or more, 97% or more, 98% or more, or 100% complementary to at least a portion of the target molecule.

The target specific sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more nucleotides in length. In some instances, the target specific sequence is between about 8 to about 20 nucleotides, 10 to about 18 nucleotides, or 12 to about 16 nucleotides in length.

The compositions and kits may comprise a plurality of probes or primers, wherein the two or more probes of the plurality of probes comprise identical target specific sequences. The compositions and kits may comprise a plurality of probes, wherein the two or more probes of the plurality of probes comprise different target specific sequences.

The probes may further comprise a unique sequence. The unique sequence is noncomplementary to the cancer marker. The unique sequence may comprise a label, barcode, or unique identifier. The unique sequence may comprise a random sequence, nonrandom sequence, or a combination thereof. The unique sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 22 or more, 24 or more, 26 or more, 28 or more, 30 or more nucleotides in length. In some instances, the unique sequence is between about 8 to about 20 nucleotides, 10 to about 18 nucleotides, or 12 to about 16 nucleotides in length.

The probes may further comprise a universal sequence. The universal sequence may comprise a primer binding site. The universal sequence may enable detection of the target sequence. The universal sequence may enable amplification of the target sequence. The universal sequence may enable transcription or reverse transcription of the target sequence. The universal sequence may enable sequencing of the target sequence.

The probe or primer compositions of the present disclosure may also be provided on a solid support. The solid support may comprise one or more beads, plates, solid surfaces, wells, chips, or a combination thereof. The beads may be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

The compositions and kits may comprise primers and primer pairs capable of amplifying target molecules, or fragments or subsequences or complements thereof. The nucleotide sequences of the target molecules may be provided in computer-readable media for in silico applications and as a basis for the design of appropriate primers for amplification of one or more target molecules.

Primers based on the nucleotide sequences of target molecules can be designed for use in amplification of the target molecules. For use in amplification reactions such as PCR, a pair of primers can be used. The exact composition of the primer sequences is not critical to the disclosure, but for most applications the primers may hybridize to specific sequences of the target molecules or the universal sequence of the probe under stringent conditions, particularly under conditions of high stringency, as known in the art. The pairs of primers are usually chosen so as to generate an amplification product of at least about 15 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, or 1000 or more nucleotides. Algorithms for the selection of primer sequences are generally known and are available in commercial software packages. These primers may be used in standard quantitative or qualitative PCR-based assays to assess transcript expression levels of target molecules. Alternatively, these primers may be used in combination with probes, such as molecular beacons in amplifications using real-time PCR.

The nucleotide sequence of the entire length of the primer does not need to be derived from the target sequence. Thus, for example, the primer may comprise nucleotide sequences at the 5′ and/or 3′ termini that are not derived from the target molecule. Nucleotide sequences which are not derived from the nucleotide sequence of the target molecule may provide additional functionality to the primer. For example, they may provide a restriction enzyme recognition sequence or a “tag” that facilitates detection, isolation, purification or immobilization onto a solid support. Alternatively, the additional nucleotides may provide a self-complementary sequence that allows the primer to adopt a hairpin configuration. Such configurations may be necessary for certain primers, for example, molecular beacon and Scorpion primers, which can be used in solution hybridization techniques.

The probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer to allow detection and/or quantitation of a target polynucleotide representing the target molecule of interest. The target polynucleotide may be the expressed target molecule RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a target polynucleotide may comprise a biotin-binding species, and an optically detectable label may be conjugated to biotin and then bound to the labeled target polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the disclosure. A variety of different coding schemes are known in the art, including fluorophores, including SCNCs, deposited metals, and RF tags.

In some embodiments, is a kit for analyzing a cancer comprising (a) a probe set comprising a plurality of probes comprising target specific sequences complementary to one or more target molecules, wherein the one or more target molecules comprise one or more cancer markers; and (b) a computer model or algorithm for analyzing an expression level and/or expression profile of the one or more target molecules in a sample. The target molecules may comprise one or more of those described herein or a combination thereof.

In some embodiments, is a kit for analyzing a cancer comprising (a) a probe set comprising a plurality of probes comprising target specific sequences complementary to one or more target molecules of a biomarker library; and (b) a computer model or algorithm for analyzing an expression level and/or expression profile of the one or more target molecules in a sample. Control samples and/or nucleic acids may optionally be provided in the kit. Control samples may include tissue and/or nucleic acids obtained from or representative of tumor samples from a healthy subject, as well as tissue and/or nucleic acids obtained from or representative of tumor samples from subjects diagnosed with a cancer.

Instructions for using the kit to perform one or more methods of the disclosure can be provided, can be provided in any fixed medium. The instructions may be located inside or outside a container or housing, and/or may be printed on the interior or exterior of any surface thereof. A kit may be in multiplex form for concurrently detecting and/or quantitating one or more different target polynucleotides representing the expressed target molecules.

Devices useful for performing methods of the disclosure are also provided. The devices can comprise means for characterizing the expression level of a target molecule of the disclosure, for example components for performing one or more methods of nucleic acid extraction, amplification, and/or detection. Such components may include one or more of an amplification chamber (for example a thermal cycler), a plate reader, a spectrophotometer, capillary electrophoresis apparatus, a chip reader, and or robotic sample handling components. These components ultimately can obtain data that reflects the expression level of the target molecules used in the assay being employed.

The devices may include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Exemplary excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid-state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices typically comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory that contains a sample designation and a record of expression levels obtained from the sample. Linkage of the results to the sample can also include a linkage to a particular sample receptacle in the device, which is also linked to the sample identity.

The devices also comprise a means for correlating the expression levels of the target molecules being studied with a prognosis of disease outcome. Such means may comprise one or more of a variety of correlative techniques, including lookup tables, algorithms, multivariate models, and linear or nonlinear combinations of expression models or algorithms. The expression levels may be converted to one or more likelihood scores, reflecting a likelihood that the patient providing the sample may exhibit a particular disease outcome. The models and/or algorithms can be provided in machine readable format and can optionally further designate a treatment modality for a patient or class of patients.

The device also comprises output means for outputting the disease status, prognosis and/or a treatment modality. Such output means can take any form which transmits the results to a patient and/or a healthcare provider, and may include a monitor, a printed format, or both. The device may use a computer system for performing one or more of the steps provided.

Samples for use with the compositions and kits and in the methods of the present disclosure comprise nucleic acids suitable for providing RNA expression information. In principle, the biological sample from which the expressed RNA is obtained and analyzed for target molecule expression can be any material suspected of comprising cancer tissue or cells. The sample can be a biological sample used directly in a method of the disclosure. Alternatively, the sample can be a sample prepared from a biological sample.

In one embodiment, the sample or portion of the sample comprising or suspected of comprising cancer tissue or cells can be any source of biological material, including cells, tissue, secretions, or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. Alternatively, or additionally, the source of the sample can be urine, bile, excrement, sweat, tears, vaginal fluids, spinal fluid, and stool. In some instances, the sources of the sample are secretions. In some instances, the secretions are exosomes.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example, an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.TM, Paramat, Paramat Extra, Paraplast, Paraplast X-tra, Paraplast Plus, Peel Away Paraffin Embedding Wax, Polyester Wax, Carbowax Polyethylene Glycol, PolyfinTM, Tissue Freezing Medium TFMFM, Cryo-GefTM, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

### II. Prognosis and Treatment

The methods, compositions, and kits disclosed herein may be used for the prognosis, predication, monitoring and/or treatment of cancer (e.g., RCC) in a subject. In some embodiments, the predicting, and/or monitoring the status or outcome of a cancer includes assessing the risk of cancer recurrence. In some embodiments, predicting, and/or monitoring the status or outcome of a cancer may comprise determining the efficacy of treatment.

In some embodiments, predicting, and/or monitoring the status or outcome of a cancer may comprise determining a therapeutic regimen. Determining a therapeutic regimen may comprise administering an anti-cancer therapeutic. Alternatively, determining the treatment for the cancer may comprise modifying a therapeutic regimen. Modifying a therapeutic regimen may comprise increasing, decreasing, or terminating a therapeutic regimen.

For example, in some embodiments, the methods described herein are used to identify subjects with a high risk of recurrence of RCC. Such subjects are offered adjuvant and/or neoadjuvant chemotherapy. In some embodiments, the adjuvant chemotherapy is a platinum-based chemotherapy (e.g., carboplatin or cisplatin) and/or immune checkpoint therapy. Specific agents for adjuvant chemotherapy are described below.

Conversely, in some embodiments, subjects identified as having a low risk of recurrence based on the levels of expression of the described markers are given the option to avoid adjuvant chemotherapy.

In some embodiments, the level of expression of UCHL1, IGF2BP3 and/or PYCR1 is used to identify subjects for treatments that target these genes and/or proteins expressed from the genes (e.g., small molecules (e.g., LDN-57444

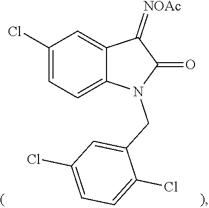

nucleic acids, or antibodies) that target these genes.

In some embodiments, the inhibitor is selected from, for example, a nucleic acid (e.g., siRNA, shRNA, miRNA or an antisense nucleic acid), a small molecule, a peptide, or an antibody.

In some embodiments, the inhibitor is a nucleic acid. Exemplary nucleic acids suitable for inhibiting UCHL1, IGF2BP3 and/or PYCR1 (e.g., by preventing expression of UCHL1 and/or PYCR1) include, but are not limited to, antisense nucleic acids and RNAi. In some embodiments, nucleic acid therapies are complementary to and hybridize to at least a portion (e.g., at least 5, 8, 10, 11, 12, 13, 14, 15, 16, 17, 18, 19, or 20 nucleotides) of UCHL1, IGF2BP3 and/or PYCR1.

In some embodiments, compositions comprising oligomeric antisense compounds, particularly oligonucleotides are used to modulate the function of nucleic acid molecules encoding UCHL1 and/or PYCR1, ultimately modulating the amount of UCHL1, IGF2BP3 and/or PYCR1 expressed. This is accomplished by providing antisense compounds that specifically hybridize with one or more UCHL1, IGF2BP3 and/or PYCR1 nucleic acids. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is decreasing the amount of UCHL1, IGF2BP3 and/or PYCR1 proteins in the T-cell.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which include, without limitation, siRNA, ssRNA and microRNA mechanisms; and occupancy based mechanisms. Certain antisense compounds may act through more than one such mechanism and/or through additional mechanisms.

In certain embodiments, antisense compounds, including those particularly suitable for ssRNA comprise one or more type of modified sugar moieties and/or naturally occurring sugar moieties. In certain embodiments, antisense compounds, including those particularly suited for use as ssRNA comprise modified internucleoside linkages. Exemplary modifications are described, for example, in Geary et al., Adv Drug Deliv Rev. 2015 Jun. 29; 87:46-51; herein incorporated by reference in its entirety.

In some embodiments, nucleic acids are RNAi nucleic acids. “RNA interference (RNAi)” is the process of sequence-specific, post-transcriptional gene silencing initiated by a small interfering RNA (siRNA), shRNA, or microRNA (miRNA). During RNAi, the RNA induces degradation of target mRNA with consequent sequence-specific inhibition of gene expression.

In “RNA interference,” or “RNAi,” a “small interfering RNA” or “short interfering RNA” or “siRNA” or “short hairpin RNA” or “shRNA” molecule, or “miRNA” an RNAi (e.g., single strand, duplex, or hairpin) of nucleotides is targeted to a nucleic acid sequence of interest, for example, UCHL1, IGF2BP3 and/or PYCR1.

An “RNA duplex” refers to the structure formed by the complementary pairing between two regions of a RNA molecule. The RNA using in RNAi is “targeted” to a gene in that the nucleotide sequence of the duplex portion of the RNAi is complementary to a nucleotide sequence of the targeted gene. In certain embodiments, the RNAi is targeted to UCHL1, IGF2BP3 and/or PYCR1 nucleic acids. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure (e.g., shRNA). In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter (e.g., testes specific promoter; See e.g., Wang et al., DNA Cell Biol. 2008 June; 27 (6): 307-14.; herein incorporate by reference in its entirety). The nucleic acid sequence can also include a polyadenylation signal. In some embodiments, the polyadenylation signal is a synthetic minimal polyad n certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, at least 16, at least 17, at least 18, at least 19, or at least 20 contiguous nucleobases complementary to an equal length portion of UCHL1, IGF2BP3 and/or PYCR1.

In some embodiments, hybridization occurs between an antisense compound disclosed herein and a UCHL1, IGF2BP3 and/or PYCR1 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules. Hybridization can occur under varying conditions. Stringent conditions are sequence-dependent and are determined by the nature and composition of the nucleic acid molecules to be hybridized.

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a UCHL1, IGF2BP3 and/or PYCR1 nucleic acid).

Non-complementary nucleobases between an antisense compound and a UCHL1 and/or PYCR1 nucleic acid may be tolerated provided that the antisense compound remains able to specifically hybridize to a target nucleic acid. Moreover, an antisense compound may hybridize over one or more segments of a UCHL1, IGF2BP3 and/or PYCR1 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a UCHL1, IGF2BP3 and/or PYCR1 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a UCHL1 and/or PYCR1 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a UCHL1, IGF2BP3 and/or PYCR1 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a UCHL1, IGF2BP3 and/or PYCR1 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. In certain embodiments, the antisense compounds are complementary to at least an 18 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

The present disclosure contemplates the use of any genetic manipulation for use in modulating the expression of UCHL1, IGF2BP3 and/or PYCR1. Examples of genetic manipulation include, but are not limited to, gene knockout (e.g., removing the UCHL1, IGF2BP3 and/or PYCR1 genes from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present disclosure, vectors are administered into tumors or tissue associated with tumors using direct injection. In other embodiments, administration is via the blood or lymphatic circulation (See e.g., PCT publication 1999/02685 herein incorporated by reference in its entirety). Exemplary dose levels of adenoviral vector are preferably 10⁸ to 10¹¹ vector particles added to the perfusate.

In some embodiments, CRISPR/Cas9 systems are used to delete or knock out genes or express an inhibitor (e.g., nucleic acid). Clustered regularly interspaced short palindromic repeats (CRISPR) are segments of prokaryotic DNA containing short, repetitive base sequences. These play a key role in a bacterial defense system, and form the basis of a genome editing technology known as CRISPR/Cas9 that allows permanent modification of genes within organisms.

In some embodiments, candidate UCHL1, IGF2BP3 and/or PYCR1 inhibitors are screened for activity (e.g., using the methods described herein or another suitable assay).

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

Dosing is dependent on severity and responsiveness of the disease state to be treated, with the course of treatment lasting from several days to several months, or until a cure is affected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual therapies and can generally be estimated based on EC50s found to be effective in in vitro and in vivo animal models. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the agent is administered in maintenance doses, ranging from 0.01 μg to 100 g per kg of body weight, once or more daily, to once every 20 years.

In some embodiments, additional anti-cancer therapies are administered in combination with the targeted therapies described above. In some embodiments, subjects are treated with immune checkpoint therapy. Examples of anti-cancer therapies include targeting cancer therapy (e.g., targeting the cancer markers described herein or other cancer markers), surgery, chemotherapy, radiation therapy, cryoablation, radio ablation, immunotherapy/biological therapy, and photodynamic therapy.

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, 5-fluorouracil, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Additional targeted therapies include but are not limited to, sunitinib, sorafenib, pazopanib, cabozantinib, lenvatinib, bevacizumab, axitinib, tivozanib, temsirolimus, belzutifan, and everolimus.

Biological therapy (sometimes called immunotherapy, biotherapy, or biological response, modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the biological therapy is immune checkpoint therapy. Immune checkpoint inhibitors target CTLA-4, PD-1, or PD-L1. Examples include but are not limited to, ipilimumab, nivolumab, pembrolizumab, spartalizumab, and atezolizumab.

### Experimental

The following examples are provided in order to demonstrate and further illustrate certain preferred embodiments and aspects of the present disclosure and are not to be construed as limiting the scope thereof.

### Example 1

### Methods

A case retrieval from the pathology archives of partial and total nephrectomy slides were reviewed from 2014-2021 with a diagnosis of clear cell renal cell carcinoma. Cases were excluded from the study cohort if they were resection specimens performed after the administration of chemotherapy or radiotherapy. Inclusion criteria for case selection required available archival H&E slides of tumor for morphologic assessment and access to archival tissue blocks for immunohistochemical analysis. Cases with uniform morphology and without diverse architectural patterns were analyzed for morphologic assessment only and not included in biomarker analysis.

### Morphology and Architectural Assessment

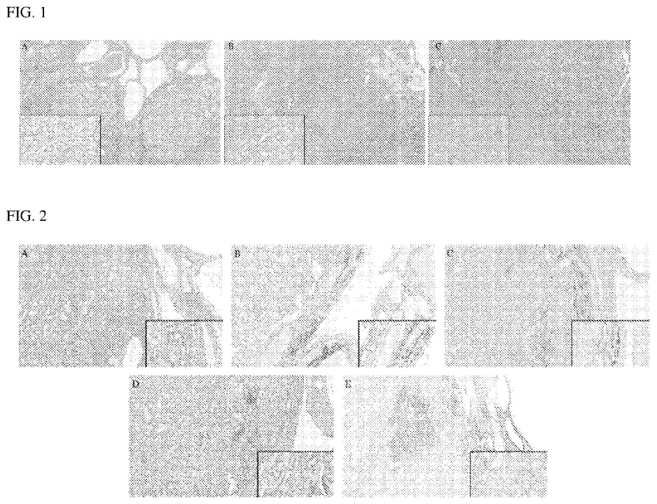

A morphologic review of all cases was performed on all H&E tumor sections taken for clinical diagnostic purposes. WHO/ISUP nucleolar grade was recorded as a relative percentage of each nucleolar grade for each tumor cross section individually, and then a combined percentage of each grade from all tumor slides for a single case was recorded and rounded to the nearest 5%. Each renal tumor was placed into a grade category that was determined based on the highest tumor grade identified. The presence or absence of distinct tumor nodules or defined differences in morphologic architecture and nucleolar grade was also recorded. These nodules were defined by abrupt juxtaposition of tumor clusters with distinctive architectural patterns and/or differences in WHO/ISUP grade. In addition to these features an architectural pattern assessment of the entire tumor was also performed. Architectural patterns recorded include nested, tubular/acinar, thick trabecular, alveolar, microcystic, bleeding follicles, solid, eosinophilic/granular, and papillary/pseudopapillary (examples of nodularity and some of the major architectural patterns are shown in FIG. 1). Architectural pattern was assessed similarly as nucleolar grade by first identifying a relative percentage for each cross section individually, and then calculating an overall percentage rounded to the nearest 5%.

Once a complete morphologic assessment was performed, formalin fixed, and paraffin embedded (FFPE) tissue block selection for immunohistochemical staining of biomarkers was performed. To interrogate intratumor heterogeneity, one representative tissue block was selected in cases that had less than 5 tissue blocks of tumor, and all other cases had two tissue blocks selected. Blocks were selected based on ability to demonstrate staining patterns in tumor areas with the highest variability of nucleolar grade (including highest-grade) and architectural patterns present in each tumor. Additionally, tissue blocks which showed areas of tumor that had abrupt morphologic transitions between architectural growth patterns or WHO/ISUP nucleolar grade were also preferred. These blocks had at minimum five consecutive 4 μm sections cut for immunohistochemical (IHC) staining. Based on the above-mentioned selection criteria, a total of 56 tumors from 77 cases were selected for biomarker interrogation; these included all cases with grade 3 or 4 tumor areas and select grade 2 tumors which exhibited architectural heterogeneity.

### Immunohistochemistry

IHC was performed on 4-micron formalin-fixed, paraffin-embedded tissue sections. The Ventana Benchmark XT staining platform with Discovery CCI and CC2 (Ventana cat #950-500 and 950-123) were used for antigen retrieval. The immune complexes were developed with either the ultra View or optiView Universal DAB (diaminobenzidine tetrahydrochloride) Detection Kit (Ventana catalogue no. 760-500 and 760-700). The details of the panel of primary antibodies utilized is as follows: carbonic anhydrase IX (CAIX; Novus Biologicals Ventana, rabbit monoclonal, catalogue no. NB100-417), ubiquitin C-terminal hydrolase L1 (UCHL1; Sigma, rabbit monoclonal, catalogue no. HPA005993), SET Domain Containing 2 (SETD2; Pro-Sci, rabbit monoclonal, catalogue no. 30-305), BRCA1 associated protein 1 (BAP1, Santa Cruz, mouse monoclonal, catalogue no. sc-28383), alpha methyacyl CoA racemase (AMACR/P504S, Zeta Corporation, rabbit monoclonal, catalogue no. z2001), and vimentin (mouse monoclonal, Ventana catalogue no. 790-2917).

### Biomarker Evaluation

Expression analysis of all 4 markers including BAP1, UCHL1, SETD2 and CAIX was performed for 56 cases with high-grade or appreciable architectural heterogeneity, utilizing full tissue sections. All tumor grades and areas of transformation were assessed. BAP1 IHC staining was assessed as presence or absence of nuclear expression and staining patterns were classified as homogenously retained, heterogeneously retained (defined as clearly defined nodules of both positive and negative staining), mixed pattern (defined as positive and negative staining cells without clearly defined nodules of differing expression), or homogenously lost; BAP1 expression in the background stromal/inflammatory cells was used as internal positive control. UCHL1 staining was assessed on presence or absence of cytoplasmic expression based on normal cytoplasmic expression in renal tubules and the promoter methylation loss that happens in low-grade tumors due to VHL alteration [13]. SETD2 staining was assessed as normal, weakened or lost nuclear expression. Internal control benign renal parenchyma was identified on at least one tissue block stained for each case, and this internal control issue was used as a reference for level of SETD2 expression. CAIX complete membranous staining was considered appropriate tumor expression (in non-necrotic areas). Correlation of abnormal staining pattern of any biomarker (identified as BAP1 loss, SETD2 weakened expression or loss, and UCHL1 expression) was correlated to the other biomarkers, CAIX staining pattern, and morphologic/architectural pattern.

### Results

Clinical Cohort Characteristics with Morphologic Variates

A total of 77 cases with the diagnosis of clear cell renal cell carcinoma were assessed for the purposes of this study. Tumors ranged in size from 1.5 cm to 20 cm in greatest dimension with only one tumor demonstrating multifocal lesions within the kidney. Additional features of these tumors including pathologic tumor stage are presented in Table 1. Twenty-one WHO/ISUP grade 4 tumors were identified, with 4 cases having sarcomatoid morphology and 16 cases having rhabdoid morphology. Three of these tumors had adrenal involvement at time of tumor resection, one with direct invasion and two with metastasis. The tumor with direct invasion of the adrenal gland also had lymphovascular invasion present. One case also had a positive ureteral margin. Twenty-nine cases were categorized as WHO/ISUP grade 3, and 27 cases were classified as WHO/ISUP grade 2. No cases were identified as WHO/ISUP grade 1. Lymph node involvement at the time of tumor resection was not identified in any case (8 cases with lymph nodes removed at time of surgery)

These cases were analyzed for the presence or absence of tumor nodularity as well as the morphologic patterns composing of all tumor sections used for clinical diagnosis. Nodularity was assessed at a scanning magnification of 20-50X (with a 2X or 5X objective lens). Nodules were defined by abrupt transitions of architectural pattern, histologic (nucleolar) grade or clearly defined clusters of tumor cells separated by thin fibrous bands or inflammatory cells. Architectural transitions most commonly were a low-grade pattern, such as the classical nested appearance of CCRCC, to higher-grade tumor areas with larger eosinophilic cells, papillary architecture, or solid sheets of smaller neoplastic cells. These nodules were present in the 56 cases that underwent biomarker analysis and the number of these defined nodular areas increased with size of tumor and tumor grade. Examples of this abrupt architectural transition with frequent variable grades can be seen in FIG. 1.

### CAIX IHC Expression and Correlates

Among all tumors, histologic grade was assessed and every tumor demonstrating grade 3 and/or 4 areas also had areas of lower-grade tumor present. Assessment of both low-grade areas and comparison to higher-grade areas was undertaken. Architecturally, the classic nested architecture was the most common pattern followed closely by bleeding follicles and microcystic patterns. Rarely, grade 2 tumors exhibited eosinophilic, alveolar, solid, or papillary/pseudopapillary areas. These patterns were not the predominant pattern identified in these low-grade tumors. In high-grade tumors, much of the grade 3 areas of the tumors showed eosinophilic, thick trabecular, alveolar, solid, or papillary/pseudopapillary patterns.

CAIX staining was performed in all cases that were WHO/ISUP grade 3 or higher. Fifteen of the high-grade tumors had areas of CAIX under-expression with only 3 cases (Case #'s 33, 36, and 46 in Table 2) not showing a correlation of loss of expression to any biomarker tested. Changes in the CAIX staining pattern were more common in the architectural patterns considered high-grade (eosinophilic, solid, papillary/pseudopapillary, alveolar and thick trabecular); and histologic grade in these areas was grade 3 or 4. Eosinophilic was the most common of these patterns to show weakened or loss of CAIX expression (11/15 cases). A detailed review of all IHC results for all high-grade tumors can be reviewed in Table 2 and a summary of biomarker phenotypes seen is represented in Table 3.

### BAP1 IHC Expression and Correlates

56 cases were stained with BAP1 IHC which included 20 WHO/ISUP grade 4 tumors, 27 grade 3 tumors, and 9 grade 2 tumors. In total, 19 (40%) cases showed homogenous nuclear retention of staining in the tissue blocks stained (in presence of appropriate internal tissue controls as described previously). Three cases (6%) within the grade 4 portion of the cohort homogenously stained negative for the presence of BAP1 protein (Case #'s 2, 10, and 13). This was agnostic of architectural pattern or histologic grade. Cytoplasmic staining of BAP1 protein was identified in three cases (case #'s 3,5, and 29) (6%) limited topographically to a nodular well-defined area of the tumor; These areas showed grade 3 histology. A later correlation analysis demonstrated positive expression of UCHL1 within the same spatial nodular area (FIG. 2). Additionally, 4 cases (#'s 4, 11, 20, and 26) (8%) showed areas with a mixed BAP1 staining pattern in high-grade regions.

Heterogenous staining patterns were seen in twenty (43%) high-grade cases (grade 3 or 4 tumors). In these heterogenous cases, the loss of BAP1 expression was found to occur in the high-grade areas of the tumor and many of these areas had eosinophilic, solid, or papillary/pseudopapillary architectural patterns. Clearly defined nodules of tumor that could be delineated by clear changes in architectural pattern that correlated with different expression of BAP1 protein was seen in 16 high-grade cases (Case #s 6-8, 10, 14, 15, 18-20, 23-25, 28-29, 35, 39 in Table 2). The remaining 4 high-grade cases, while having a heterogenous BAP1 pattern, this variable IHC pattern did not correlate well with architectural features; however, loss of staining was present in grade 3 and/or grade 4 cells. These tumors showed retention of BAP1 staining in the sarcomatoid areas and in two the staining of the sarcomatoid region was stronger than the remaining tumor areas.

A total of 9 low-grade tumors (grade 2) were stained for comparison to the high-grade tumors. Three cases exhibited focal loss of BAP1 staining which corresponded to areas that had borderline features between grade 2 and 3 nuclei. The BAP1 staining pattern matched a small nodular growth of cell clusters with grade 3-like regions where these areas showed nested pattern with larger cells than background tumor and have some cells within the cluster that exhibit grade 3 nuclei. The remaining six cases had retained nuclear BAP1 staining in all tumor cells.

Overall, nodular growth of tumor clusters was more prominent in the high-grade tumors. In total 80% of cases with a heterogenous BAP1 staining pattern showed that loss of nuclear BAP1 expression appeared within a distinctive nodule that had a morphological pattern different from surrounding background tumor cells. This defining morphological characteristics included larger cell size from background with an alveolar, eosinophilic, or papillary/pseudopapillary architecture.

Notably, in a recent study, immunohistochemistry showed loss of BAP1 expression in majority of tumors in addition to strong AMACR (p504S) expression with distinctive BAP1 retained regions showing patchy p504S [2]. This was interrogated by performing AMACR IHC on three representative cases of BAP1 loss higher-grade areas and found this association to be seen in 2/3 cases (one case exhibiting high p504s expression and other low) (Figure-1).

### UCHL1 IHC Expression and Correlates

A total of 28 (60%) high-grade tumors exhibited expression of UCHL1; expression was found most often in eosinophilic/rhabdoid (15 cases; 62%), papillary (4 cases; 17%), or sarcomatoid (2 cases; 8%) components of tumors assessed. Correlation of UCHL1 expression to BAP1 expression alterations was also performed. The three cases of cytoplasmic BAP1 expression (negative nuclear expression; case #s 2,5, and 29) also showed strong UCHL1 cytoplasmic expression. Of the 20 cases with heterogenous expression of BAP1, only 5 tumors had UCHL1 expression within the same nodule with loss of BAP1 expression. An additional 7 cases with heterogenous BAP1 expression also had UCHL1 staining in a different component of the tumor from the areas of BAP1 loss. The remaining 8 cases with heterogenous BAP1 expression did not show UCHL1 expression in any component of the tumor. No cases with a mixed BAP1 expression pattern showed correlation with UCHL1 expression. Of note, all cases that had UCHL1 staining exhibited positive cells with a high-grade histologic pattern.

In the grade 2 tumors stained with UCHL1, two cases had focal UCHL1 staining. These cases also had areas of tumor that showed loss of BAP1. One case had correlation of BAP1 and UCHL1 expression while the other case had altered expression patterns in different areas of the tumor. Both tumor areas with staining had borderline features between grade 2 and grade 3 features.

Correlation of UCHL1 expression to the morphologic characteristics of the tumors, those cases with UCHL1 expression showed expression most often when there was an eosinophilic nodule. The correlation to eosinophilia in cases which had UCHL1 expression was readily appreciable that individual cysts lined with eosinophilic cells was even seen in one case and a small cluster of less than 50 eosinophilic cells was highlighted in another case. In three cases (case #'s 2, 17 and 20) there was homogenous high expression across all the high-grade tumor areas and was not associated with any specific morphologic pattern or tumor nodule.

### SETD2 IHC Expression and Correlates

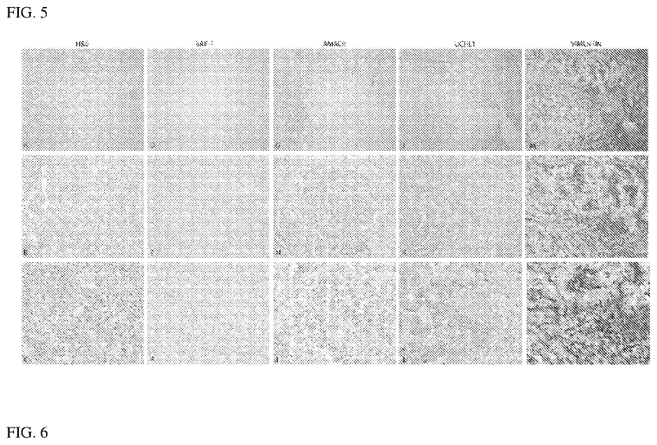

A total of 22 (47%) high-grade tumors had heterogenous staining for SETD2. Weakened and loss of expression staining patterns were identified based on the background normal kidney expression in each patient. Six cases (Case #s 1, 10, 26, 27, 37, 41) with heterogenous staining of SETD2 had areas of loss of expression that correlated with loss of BAP1 expression as well. No changes in expression of SETD2 was seen in 26 cases, of which 5 cases had no alterations in expression of all the biomarkers analyzed. An interesting finding in these cases included six cases that had SETD2 loss and weakened or decreased CAIX expression within the region of SETD2 loss (Case #s 3, 11, 19, 20, 26, and 41; cases 19 and 41 shown in FIG. 5). In the cases with correlated expression, the SETD2 loss of expression was seen in the high-grade component of the tumor only. However, there was one case (case #12) where loss of expression was seen in the low-grade component as well.

The 9 low-grade tumors that were stained for all biomarkers showed three cases with heterogenous loss of expression in SETD2. Two cases correlated to BAP1 loss without UCHL1 expression, and the third case only showed a focus of SETD2 loss in an area of papillary architecture without other biomarker alterations.

In terms of nodularity expression, ten cases exhibited clearly defined morphologic nodules with loss of SETD2 expression in the background normal expression. Two of these cases can be seen in FIGS. 2 & 3. In additional cases there was more widespread loss of expression of SETD2 that spanned multiple nodules of tumor morphology on a single slide.

### Combined Biomarker and Morphologic Annotations

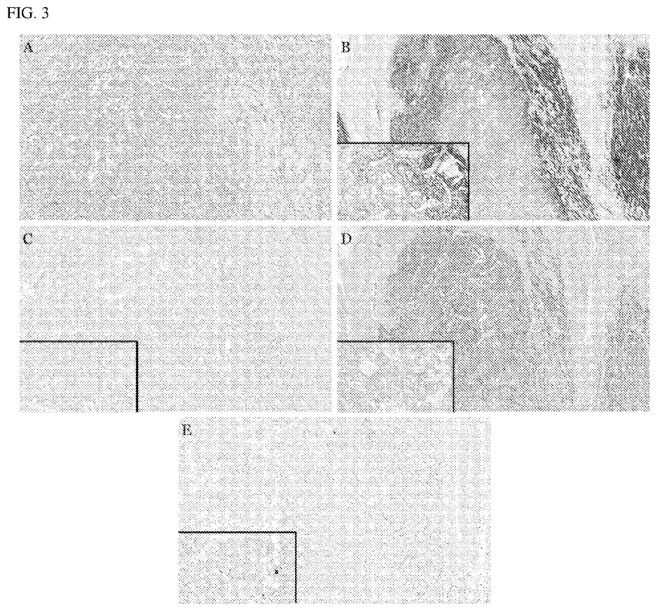

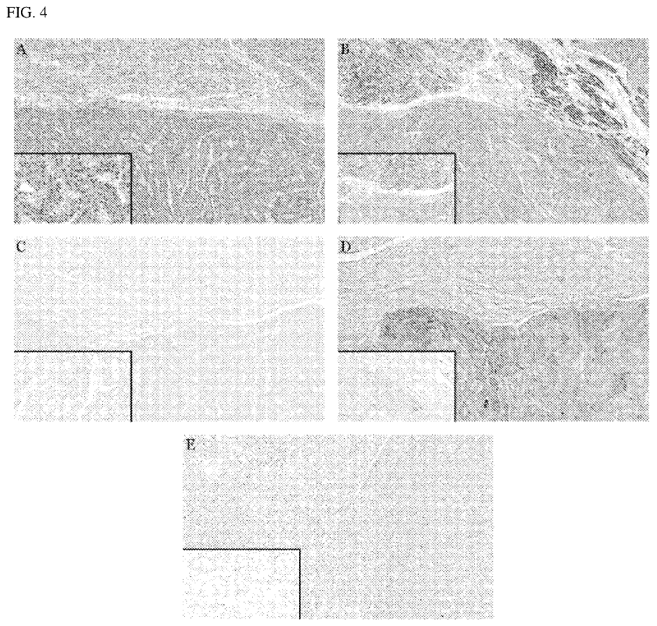

Analysis of the regions of all cases with altered biomarker expression and the correlation of these regions to nodularity and morphology was performed. In three cases (cases 20, 26 and 41), the distinctive morphologic nodular area of altered BAP1 expression correlated with UCHL1 expression and SETD2 loss as well as weakened CAIX expression (case #20 shown in FIG. 3 and case #26 shown in FIG. 4). These cases with all biomarkers altered displayed histologic grade 3 or 4 tumor cells. In other cases, (like case #3, seen in FIG. 2) all biomarkers showed altered expression, but these alterations were not all seen in the same location or within the same nodular region of tumor, but were still within high-grade histologic regions. In this example, cytoplasmic BAP1 alteration was seen in the same area as strong UCHL1 expression that showed a nested clear cell pattern, but a different region of high-grade tumor with eosinophilic morphology expressed SETD2 loss. Other variable alterations in biomarkers expressions noted within individual biomarker sections (presented above).

Sixteen tumors had correlation of BAP1 and/or SETD2 altered expression areas with changes in CAIX and/or UCHL1 expression in patterns altered from those seen in classical appearing low-grade tumor areas. These regions with altered biomarkers that show changes were nodules of tumor that had WHO/ISUP grade 3 or 4 nuclei that could be distinguished from the background lower grade tumor cells at low magnification. While other tumors had heterogeneity in biomarker expression, these changes did not correlate to specific spatial nodules of tumor with defined morphology or grade. In the heterogenous SETD2 expression cases, three cases showed correlation of UCHL1 expression to areas of SETD2 loss. Finally, as an extra validation step, to rule out artefactual factors such as tissue fixation, assay consistency, vimentin was used as a control in some select cases where heterogeneity for biomarker expression was seen. In all these cases there was a homogenous expression for vimentin thereby ruling out such extraneous elements (Figure-5).

Thus, overall, a notable subset of tumors was observed to exhibit a variable morphologic phenotype and with altered biomarker profiles.

TABLE 1
Summary of Cohort Clinical characteristics

| | | WHO/ISUP Grade | | |
|---|---|---|---|---|
| Clinical Characteristic | | 2 | 3 | 4 |
| No. Cases | | 27 | 29 | 21 |
| Specimen Laterality | Left | 13 | 13 | 13 |
| | Right | 14 | 16 | 8 |
| Tumor size (range in cm) | | 1.5-7.2 | 1.9-13.3 | 1.7-20 |
| Tumor focality | Unifocal | 27 | 29 | 20 |
| | Multifocal | 0 | 0 | 1 |
| No. Sarcomatoid features | | 0 | 0 | 4 |
| present | | | | |
| No. Rhabdoid features | | 0 | 0 | 13 |
| present | | | | |
| No. Tumor necrosis present | | 2 | 2 | 12 |
| No. Lymphovascular invasion | | 0 | 0 | 2 |
| No. Adrenal involvement | | 0 | 1 | 3 |
| No. Pathologic Stage | pT1a | 16 | 16 | 3 |
| | pT1b | 7 | 5 | 4 |
| | pT2a | 1 | 0 | 0 |
| | pT2b | 0 | 0 | 0 |
| | pT3a | 2 | 6 | 8 |
| | pT3b | 1 | 2 | 4 |
| | pT4 | 0 | 0 | 2 |

TABLE 2
High-Grade Tumor Biomarker Expression

| | Case | [symbol] | [symbol] | [symbol] | [symbol] |
|---|---|---|---|---|---|
| Grade 4 | 1 | [symbol] | [symbol] | [symbol] | [symbol] |
| | 2 | [symbol] | [symbol] | [symbol] | [symbol] |
| | 3 | [symbol] | [symbol] | [symbol] | [symbol] |
| | 4 | [symbol] | [symbol] | [symbol] | [symbol] |
| | 5 | [symbol] | [symbol] | [symbol] | [symbol] |
| | 6 | [symbol] | [symbol] | [symbol] | [symbol] |
| | 7 | [symbol] | [symbol] | [symbol] | [symbol] |
| | 8 | [symbol] | [symbol] | [symbol] | [symbol] |
| | 11 | [symbol] | [symbol] | [symbol] | [symbol] |
| | 12 | [symbol] | [symbol] | [symbol] | [symbol] |
| | 14 | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | 17 | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Grade 3 | 21 | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |

[symbol]

indicates data missing or illegible when filed

TABLE 3
Biomarker Patterns Observed in High Grade Tumors

| | | Number of | Number of |
|---|---|---|---|
| Immunohistochemical | Number of | Grade 3 | Grade 4 |
| Staining Pattern | Cases | Cases | Cases |
| CAIX loss of expression | 10 | 4 | 6 |
| Homogenous loss of BAP1 | 4 | 1 | 3 |
| Expression | | | |
| Mixed pattern BAP1 staining | 4 | 2 | 2 |
| Heterogenous loss of BAP1 | 23 | 12 | 11 |
| expression | | | |
| BAP1 retained | 16 | 12 | 4 |
| Heterogenous loss of SETD2 | 21 | 9 | 12 |
| expression | | | |
| Homogenous loss of SETD2 | 0 | 0 | 0 |
| expression | | | |
| UCHL-1 expression | 27 | 11 | 16 |
| UCHL-1 expression and BAP-1 | 11 | 3 | 8 |
| loss correlation | | | |
| UCHL-1 Expression correlates | 4 | 1 | 3 |
| with SETD2 loss | | | |

### Example 1 References

1. Surveillance, E., and End Results (SEER) Program, SEER*Stat Database: North American Association of Central Cancer Registries (NAACCR) Incidence Data-CiNA Analytic File, 1995-2016, for Expanded Races, Custom File With County, ACS Facts and Figures projection Project (Which Includes Data from CDC's National Program of Cancer Registries [NPCR], CCCR's Provincial and Territorial Registries, and the NCI's Surveillance, Epidemiology and End Results [SEER] Registries). North American Association of Central Cancer Registries, 2019.
2. Gallan, A. J., et al., BAP1-Mutated Clear Cell Renal Cell Carcinoma. Am J Clin Pathol, 2021. 155 (5): p. 718-728.
3. Bihr, S., et al., Expression and Mutation Patterns of PBRM1, BAP1 and SETD2 Mirror Specific Evolutionary Subtypes in Clear Cell Renal Cell Carcinoma. Neoplasia, 2019. 21 (2): p. 247-256.
4. da Costa, W. H., ct al., Prognostic impact of concomitant loss of PBRM1 and BAP1 protein expression in early stages of clear cell renal cell carcinoma. Urol Oncol, 2018. 36 (5): p. 243 e1-243 e8.
5. Cancer Genome Atlas Research, N., Comprehensive molecular characterization of clear cell renal cell carcinoma. Nature, 2013. 499 (7456): p. 43-9.
6. Eckel-Passow, J. E., et al., BAP1 and PBRM1 in metastatic clear cell renal cell carcinoma: tumor heterogeneity and concordance with paired primary tumor. BMC Urol, 2017. 17 (1): p. 19.
7. Miura, Y., et al., Loss of BAP1 protein expression in the first metastatic site predicts prognosis in patients with clear cell renal cell carcinoma. Urol Oncol, 2017. 35 (6): p. 386-391.
8. Gerlinger, M., et al., Intratumor heterogeneity and branched evolution revealed by multiregion sequencing. N Engl J Med, 2012. 366 (10): p. 883-892.
9. Singh, R. R., et al., Intratumoral morphologic and molecular heterogeneity of rhabdoid renal cell carcinoma: challenges for personalized therapy. Mod Pathol, 2015. 28 (9): p. 1225-35.
10. Joseph, R. W., et al., Loss of BAP1 protein expression is an independent marker of poor prognosis in patients with low-risk clear cell renal cell carcinoma. Cancer, 2014. 120 (7): p. 1059-67.
11. Mannan, R., et al., Integrated Clinico-Pathological and Proteogenomic Characterization of Clear Cell Renal Cell Carcinoma by the Clinical Proteomic Tumor Analysis Consortium (CPTAC). Lab Invest, 2022. 102 (Suppl 1): p. 583-732.
12. Li, Y., et al., Histopathologic and proteogenomic heterogeneity reveals features of clear cell renal cell carcinoma aggressiveness. Cancer Cell, 2022.
13. Seliger, B., et al., Epigenetic control of the ubiquitin carboxyl terminal hydrolase 1 in renal cell carcinoma. J Transl Med, 2009. 7: p. 90.
14. Seliger, B., et al., Ubiquitin COOH-terminal hydrolase 1: a biomarker of renal cell carcinoma associated with enhanced tumor cell proliferation and migration. Clin CancerRes, 2007. 13 (1): p. 27-37.
15. Rong, C., et al., Ubiquitin Carboxyl-Terminal Hydrolases and Human Malignancies: The Novel Prognostic and Therapeutic Implications for Head and Neck Cancer. Front Oncol, 2020. 10: p. 592501.
16. Mondal, M., et al, UCHL1 as a novel target in breast cancer: emerging insights from cell and chemical biology. Br J Cancer., 2022. 126 (1): 24-33
17. Vormittag-Nocito, E., et al., Morphologic and Molecular Profiling Decipher Intratumoral Heterogeneity in Clear Cell Renal Cell Carcinoma. Lab Invest, 2022. 102 (Suppl 1): p. 583-732.
18. Wagner, E J., et al., Understanding the language of Lys36 methylation at histone H3. Nature Rev Mol Cell Biol. 2012. 13 (2): 115-26.
19. Hakimi, A A., et al., Adverse outcomes in clear cell renal cell carcinoma with mutations of 3p21 epigenetic regulators BAP1 and SETD2: a report by MSKCC and the KIRC TCGA research network. Clin Cancer Res. 2013. 19 (12): 3259-67.
20. Liu, W., et al. Decreased expression of SETD2 predicts unfavorable prognosis in patients with nonmetastatic clear-cell renal cell carcinoma. Medicine. 2015. 94 (45): e2004.
21. Tiedemann, R L., et al. Dynamic reprogramming of DNA methylation in SETD2-deregulated renal cell carcinoma. Oncotarget. 2016. 7 (2): 1927.
22. Li, J., et al. Functional studies on primary tubular epithelial cells indicate a tumor suppressor role of SETD2 in clear cell renal cell carcinoma. Neoplasia. 2016. 18 (6): 339-46.
23. Cai, Q., et al., Ontological analyses reveal clinically-significant clear cell renal cell carcinoma subtypes with evolutionary trajectories into an aggressive type. EBioMedicine, 2020. 51:102526
24. Kapur, P., et al., Effects on survival of BAP1 and PBRM1 mutations in sporadic clear-cell renal-cell carcinoma: a retrospective analysis with independent validation. The Lancet Oncology, 2013. 14 (2): p. 159-167.
25. Okonska, A. and E. Felley-Bosco, BAP1 Missense Mutations in Cancer: Friend or Foe? Trends Cancer, 2019. 5 (11): p. 659-662.
26. Minardi, D., et al., Loss of nuclear BAP1 protein expression is a marker of poor prognosis in patients with clear cell renal cell carcinoma. Urol Oncol, 2016. 34 (8): p. 338 e11-8.
27. Moore, A. L., et al., Spatial Distribution of Private Gene Mutations in Clear Cell Renal Cell Carcinoma. Cancers (Basel), 2021. 13 (9).
28. Trpkov, K., et al., New developments in existing WHO entities and evolving molecular concepts: The Genitourinary Pathology Society (GUPS) update on renal neoplasia. Mod Pathol, 2021. 34 (7): p. 1392-1424.

### Example 2

### Methods

### Human Subjects

A total of 213 participants, with an age range of 30-90, were included in this study. This cohort contained males (n=149) and females (n=64) and reflects the gender distribution of clear cell renal cell carcinoma (ccRCC).83 Only histopathologically defined adult ccRCC tumors were only included in the analysis. Institutional review boards at each Tissue Source Site (TSS) reviewed protocols and consent documentation, in adherence to Clinical Proteomic Tumor Analysis Consortium (CPTAC) guidelines.

### Clinical Data Annotation

Clinical data were obtained from TSS and aggregated by the Biospecimen Core Resource (BCR, Van Andel Research Institute (Grand Rapids, MI)). Data forms were stored as Microsoft Excel files (.xls). Clinical data can be accessed and downloaded from the CPTAC Data Portal. Patients with any prior history of other malignancies within twelve months or any systemic treatment (chemotherapy, radiotherapy, of immune-related therapy) were excluded from this study. Demographics, histopathologic information, and treatment details were collected and summarized in Table 4. The characteristics of the CPTAC ccRCC cohort reflect the general incidence of ccRCC.83

### Cell Lines

The ccRCC cell line Caki-1 and a control cell line HK-2 were maintained in Dulbecco's Modified Eagle Medium/Nutrient Mixture F-12 (DMEM/F-12) culture medium (Gibco-11320033) supplemented with 10% FBS (Sigma, F-9665) and 1% Pen Strep (Gibco, 10,000 U/mL-15140122). 786-O cells were maintained in Gibco RPMI-1640 supplemented with 10% FBS. In addition, 769-P, A-498, and Caki-2 were used for in vitro experiments assessing the impact of select kinase inhibition.

### Sample Processing

The CPTAC Biospecimen Core Resource (BCR) at the Pathology and Biorepository Core of the Van Andel Research Institute in Grand Rapids, Michigan manufactured and distributed biospecimen kits to the Tissue Source Sites (TSS) located in the US, Europe, and Asia. Each kit contains a set of pre-manufactured labels for unique tracking of every specimen respective to TSS location, disease, and sample type, used to track the specimens through the BCR to the CPTAC proteomic and genomic characterization centers.

Tissue specimens averaging 200 mg were snap-frozen by the TSS within a 30 min cold ischemic time (CIT) (CIT average=13 min) and an adjacent segment was formalin-fixed paraffin embedded (FFPE) and H&E stained by the TSS for quality assessment to meet the CPTAC ccRCC requirements. Routinely, several tissue segments for each case were collected. Tissues were flash-frozen in liquid nitrogen (LN2) and then transferred to a liquid nitrogen freezer for storage until approval for shipment to the BCR.

Specimens were shipped using a cryoport that maintained an average temperature of under −140° C. to the BCR with a time and temperature tracker to monitor the shipment. Receipt of specimens at the BCR included a physical inspection and review of the time and temperature tracker data for specimen integrity, followed by barcode entry into a biospecimen tracking database. Specimens were again placed in LN2 storage until further processing. Acceptable ccRCC tumor tissue segments were determined by TSS pathologists based on the percent viable tumor nuclei (>60%), total cellularity (>50%), and necrosis (<50%). Segments received at the BCR were verified by BCR and Leidos Biomedical Research (LBR) pathologists and the percent of the total area of tumor in the segment was also documented. Additionally, disease-specific working group pathology experts reviewed the morphology to clarify or standardize specific disease classifications and correlation to the proteomic and genomic data.

Specimens selected for the discovery set were determined on the maximal percent in the pathology criteria and best weight. Specimens were pulled from the biorepository using an LN2 cryocart to maintain specimen integrity and then cryopulverized. The cryopulverized specimen was divided into aliquots for DNA (30 mg) and RNA (30 mg) isolation and proteomics (50 mg) for molecular characterization. Nucleic acids were isolated and stored at −80° C. until further processing and distribution; cryopulverized protein material was returned to the LN2 freezer until distribution. Shipment of the cryopulverized segments used cryoports for distribution to the proteomic characterization centers and shipment of the nucleic acids used dry ice shippers for distribution to the genomic characterization centers; a shipment manifest accompanied all distributions for the receipt and integrity inspection of the specimens at the destination.

### Comprehensive Schematic Histopathology Evaluation

A comprehensive evaluation of the hematoxylin and eosin (H&E) stained histopathologic samples was undertaken with a focus on the tumor epithelial component and the surrounding tumor microenvironmental alterations including the immune cell characterization. The overall grading of the tumor samples was based on the findings noted from the previous histopathologic patient reports which were re-confirmed. Broadly the epithelial cell assessment was done under three categories of recognition of nodular areas/distinct sudden transitional areas and sub-dividing the morphologic patterns and cytology under low-grade and high-grade parameters. Every histopathologic tissue section was annotated for recognized low-grade features (nested, tubular/acinar, microcystic, and bleeding follicles) and high-grade features (eosinophilic/granular, thick trabecular, alveolar, solid, papillary/pseudo-papillary, sarcomatoid, and rhabdoid). 26,29 Apart from the detailed spatial architecture and cytological assessment, tumor microenvironment evaluation was also performed detailing immune characterization (semi-quantitative scoring, type of infiltration-intratumoral, intratumoral septal, peri-tumoral and stromal, and immune subpopulation types) and the presence or absence of necrosis. In addition, specialized histopathologic annotations such as the presence of hyalinization, fibrotic response, extensive multi-nodularity, or histopathologic resemblance to other renal cell carcinoma subtypes were also noted. Thus, in each tumor sample instead of focusing on the higher grade or aggressive spatial topography, the whole tissue area was evaluated against the entire spectrum of morphological parameters as described above. These findings were recorded and tabulated. A semi-quantitative score for each tumor was rendered based on the presence (scored as 1) or absence (scored as 0) of the individual histologic parameters. This way a detailed assessment of histologic tumor heterogeneity was assessed (Table 4).

### Sample Processing for Genomic DNA and Total RNA Extraction

The study sampled a single site of the primary tumor from surgical resections, due to the internal requirement to process a minimum of 125 mg of tumor tissue and 50 mg of adjacent normal tissue. DNA and RNA were extracted from tumor and blood normal specimens in a co-isolation protocol using Qiagen's QIAsymphony DNA Mini Kit and QIAsymphony RNA Kit. Genomic DNA was also isolated from peripheral blood (3-5 mL) to serve as matched normal reference material. The QubitTM dsDNA BR Assay Kit was used with the Qubit® 2.0 Fluorometer to determine the concentration of dsDNA in an aqueous solution. Any sample that passed quality control and produced enough DNA yield to go through various genomic assays was sent for genomic characterization. RNA quality was quantified using both the NanoDrop 8000 and quality assessed using Agilent Bioanalyzer. A sample that passed RNA quality control and had a minimum RIN (RNA integrity number) score of 7 was subjected to RNA sequencing. Identity match for germline, normal adjacent tissue, and tumor tissue was assayed at the BCR using the Illumina Infinium QC array. This beadchip contains 15,949 markers designed to prioritize sample tracking, quality control, and stratification.

### Whole Exome Sequencing

### Library Construction

Library construction was performed as described in, 110 with the following modifications: initial genomic DNA input into shearing was reduced from 3 μg to 20-250 ng in 50 μL of solution. For adapter ligation, Illumina paired-end adapters were replaced with palindromic forked adapters, purchased from Integrated DNA Technologies, with unique dual-indexed molecular barcode sequences to facilitate downstream pooling. Kapa HyperPrep reagents in 96-reaction kit format were used for end repair/A-tailing, adapter ligation, and library enrichment PCR. In addition, during the post-enrichment SPRI cleanup, elution volume was reduced to 30 μL to maximize library concentration, and a vortexing step was added to maximize the amount of template eluted.

### In-Solution Hybrid Selection

After library construction, libraries were pooled into groups of up to 96 samples. Hybridization and capture were performed using the relevant components of Illumina's Nextera Exome Kit and following the manufacturer's suggested protocol, with the following exceptions. First, all libraries within a library construction plate were pooled prior to hybridization. Second, the Midi plate from Illumina's Nextera Exome Kit was replaced with a skirted PCR plate to facilitate automation. All hybridization and capture steps were automated on the Agilent Bravo liquid handling system.

### Preparation of Libraries for Cluster Amplification and Sequencing

After post-capture enrichment, library pools were quantified using qPCR (automated assay on the Agilent Bravo) using a kit purchased from KAPA Biosystems with probes specific to the ends of the adapters. Based on qPCR quantification, libraries were normalized to 2 nM.

### Cluster Amplification and Sequencing

Cluster amplification of DNA libraries was performed according to the manufacturer's protocol (Illumina) using exclusion amplification chemistry and flowcells. Flowcells were sequenced utilizing sequencing-by-synthesis chemistry. The flow cells were then analyzed using RTA v.2.7.3 or later. Each pool of whole-exome libraries was sequenced on paired 76 cycle runs with two 8 cycle index reads across the number of lanes needed to meet coverage for all libraries in the pool. Pooled libraries were run on HiSeq 4000 paired-end runs to achieve a minimum of 150x on target coverage per sample library. The raw Illumina sequence data were demultiplexed and converted to fastq files; adapter and low-quality sequences were trimmed. The raw reads were mapped to the hg38 human reference genome and the validated BAMs were used for downstream analysis and variant calling.

### PCR-Free Whole Genome Sequencing

### Preparation of Libraries for Cluster Amplification and Sequencing

An aliquot of genomic DNA (350 ng in 50 μL) was used as the input into DNA fragmentation (aka shearing). Shearing was performed acoustically using a Covaris focused-ultrasonicator, targeting 385 bp fragments. Following fragmentation, additional size selection was performed using a SPRI cleanup. Library preparation was performed using a commercially available kit provided by KAPA Biosystems (KAPA Hyper Prep without amplification module) and with palindromic forked adapters with unique 8-base index sequences embedded within the adapter (purchased from IDT). Following sample preparation, libraries were quantified using quantitative PCR (kit purchased from KAPA Biosystems), with probes specific to the ends of the adapters. This assay was automated using Agilent's Bravo liquid handling platform. Based on qPCR quantification, libraries were normalized to 1.7 nM and pooled into 24-plexes.

### Cluster Amplification and Sequencing (HiSeq X)

Sample pools were combined with HiSeq X Cluster Amp Reagents EPX1, EPX2, and EPX3 into single wells on a strip tube using the Hamilton Starlet Liquid Handling system. Cluster amplification of the templates was performed according to the manufacturer's protocol (Illumina) with the Illumina cBot. Flow cells were sequenced to a minimum of 15x on HiSeq X utilizing sequencing-by-synthesis kits to produce 151 bp paired-end reads. Output from Illumina software was processed by the Picard data processing pipeline to yield BAMs containing demultiplexed, aggregated, and aligned reads. All sample information tracking was performed by automated LIMS messaging.

### Illumina Infinium MethylationEPIC Beadchip Array

The MethylationEPIC array uses an 8-sample version of the Illumina Beadchip capturing >850,000 DNA methylation sites per sample. 250 ng of DNA was used for the bisulfite conversation using Infinium MethylationEPIC BeadChip Kit. The EPIC array includes sample plating, bisulfite conversion, and methylation array processing. After scanning, the data was processed through an automated genotype calling pipeline. Data generated consisted of raw idats and a sample sheet.

### RNA Sequencing

### Quality Assurance and Quality Control of RNA Analytes

All RNA analytes were assayed for RNA integrity, concentration, and fragment size. Samples for total RNA-seq were quantified on a TapeStation system (Agilent, Inc. Santa Clara, CA). Samples with RINs >8.0 were considered high quality.

### Total RNA-seq Library Construction

Total RNA-seq library construction was performed from the RNA samples using the TruSeq Stranded RNA Sample Preparation Kit and bar-coded with individual tags following the manufacturer's instructions (Illumina, Inc. San Diego, CA). Libraries were prepared on an Agilent Bravo Automated Liquid Handling System. Quality control was performed at every step and the libraries were quantified using the TapeStation system.

### Total RNA Sequencing

Indexed libraries were prepared and run on HiSeq 4000 paired-end 75 base pairs to generate a minimum of 120 million reads per sample library with a target of greater than 90% mapped reads. Typically, these were pools of four samples. The raw Illumina sequence data were demultiplexed and converted to FASTQ files, and adapter and low-quality sequences were quantified. Samples were then assessed for quality by mapping reads to the hg38 human genome reference, estimating the total number of reads that mapped, amount of RNA mapping to coding regions, amount of rRNA in sample, number of genes expressed, and relative expression of housekeeping genes. Samples passing this QA/QC were then clustered with other expression data from similar and distinct tumor types to confirm expected expression patterns. Atypical samples were then SNP typed from the RNA data to confirm the source analyte. FASTQ files of all reads were then uploaded to the GDC repository.

miRNA-seq Library Construction miRNA-seq library construction was performed from the RNA samples using the NEXTflex

Small RNA-Seq Kit (v3, PerkinElmer, Waltham, MA) and bar-coded with individual tags following the manufacturer's instructions. Libraries were prepared on Sciclone Liquid Handling Workstation Quality control was performed at every step, and the libraries were quantified using a TapeStation system and an Agilent Bioanalyzer using the Small RNA analysis kit. Pooled libraries were then size selected according to NEXTflex Kit specifications using a Pippin Prep system (Sage Science, Beverly, MA).

miRNA Sequencing

Indexed libraries were loaded on the Hiseq 4000 to generate a minimum of 10 million reads per library with a minimum of 90% reads mapped. The raw Illumina sequence data were demultiplexed and converted to FASTQ files for downstream analysis. Resultant data were analyzed using a variant of the small RNA quantification pipeline developed for TCGA.111 Samples were assessed for the number of miRNAs called, species diversity, and total abundance. Samples passing quality control were uploaded to the GDC repository.

### Single-nuclei RNA Library Preparation and Sequencing

About 20-30 mg of cryopulverized powder from ccRCC specimens was resuspended in Lysis buffer (10 mM Tris-HCl (pH 7.4); 10 mM NaCl; 3 mM MgCl₂; and 0.1% NP-40). This suspension was pipetted gently 6-8 times, incubated on ice for 30 seconds, and pipetted again 4-6 times. The lysate containing free nuclei was filtered through a 40 μm cell strainer. The filter was washed with 1 mL Wash and Resuspension buffer (1X PBS+2% BSA+0.2 U/uL RNase inhibitor) and the flow through was combined with the original filtrate. After 6-minute centrifugation at 500×g and 4° C., the nuclei pellet was resuspended in 500 μL of Wash and Resuspension buffer. After staining by DRAQ5, the nuclei were further purified by Fluorescence-Activated Cell Sorting (FACS). FACS-purified nuclei were centrifuged again and resuspended in a small volume (about 30 μL). After counting and microscopic inspection of nuclei quality, the nuclei preparation was diluted to about 1,000 nuclei/uL. About 20,000 nuclei were used for single-nuclei RNA sequencing (snRNAseq) by the 10X Chromium platform. The single nuclei were loaded onto a Chromium Chip B Single Cell Kit, 48 reactions (10x Genomics, PN-1000073) and processed through the Chromium Controller to generate GEMs (Gel Beads in Emulsion). Sequencing libraries were prepared with the Chromium Single Cell 3′ GEM, Library & Gel Bead Kit v3, 16 rxns (10x Genomics, PN-1000075) following the manufacturer's protocol. Sequencing was performed on an Illumina NovaSeq 6000 S4 flow cell. The libraries were pooled and sequenced using the XP workflow according to the manufacturer's protocol with a 28×8×98 bp sequencing recipe. The resulting sequencing files were available as FASTQs per sample after demultiplexing.

### MS Sample Processing and Data Collection

### Sample Processing for Protein Extraction and Tryptic Digestion

All samples for the current study were prospectively collected as described above and processed for mass spectrometric (MS) analysis at the PCC. Tissue lysis and downstream sample preparation for global proteomic and phosphoproteomic analysis were carried out as previously described. 13 Approximately 25-120 mg of each cryopulverized renal tumor tissues or NATs were homogenized separately in an appropriate volume of lysis buffer (8 M urea, 75 mM NaCl, 50 mM Tris, pH 8.0, 1 mM EDTA, 2 g/mL aprotinin, 10 g/mL leupeptin, 1 mM PMSF, 10 mM NaF, Phosphatase Inhibitor Cocktail 2 and Phosphatase Inhibitor Cocktail 3 [1:100 dilution], and 20 mM PUGNAc) by repeated vortexing. Lysates were clarified by centrifugation at 20,000×g for 10 min at 4° C., and protein concentrations were determined by BCA assay (Pierce). Lysates were diluted to a final concentration of 8 mg/ml with lysis buffer, and 800 g of protein was reduced with 5 mM dithiothreitol (DTT) for 1 h at 37° C. and subsequently alkylated with 10 mM iodoacetamide for 45 min at RT (room temperature) in the dark. Samples were diluted 1:3 with 50 mM Tris-HCl (pH 8.0) and subjected to proteolytic digestion with LysC (Wako Chemicals) at 1 mAU: 50 g enzyme-to-substrate ratio for 2 h at RT, followed by the addition of sequencing-grade modified trypsin (Promega) at a 1:50 enzyme-to-substrate ratio and overnight incubation at RT. The digested samples were then acidified with 50% trifluoroacetic acid (TFA, Sigma) to a pH value of approximately 2.0. Tryptic peptides were desalted on reversed-phase C18 SPE columns (Waters), followed by aliquoting 20 g of digested peptides for global proteomic analysis, dried in a Speed-Vac, and resuspended in 3% ACN/0.1% formic acid prior to ESI-LC-MS/MS analysis. The remaining sample was dried down in a Speed-Vac and utilized for phosphopeptide and intact glycopeptide enrichment.

### Enrichment of Phosphopeptides by Fe-IMAC.

A 450 g aliquot of digested peptide material was subjected to phosphopeptide enrichment using immobilized metal affinity chromatography (IMAC) as previously described. 112 In brief, Ni-NTA agarose beads were used to prepare Fe3+-NTA agarose beads, and 450 g of peptides were reconstituted in 80% ACN/0.1% trifluoroacetic acid and incubated with 10 L of the Fe3+-IMAC beads for 30 min. Samples were then centrifuged, and the supernatant containing unbound peptides was removed. The beads were washed twice and then transferred onto equilibrated C-18 Stage Tips with 80% ACN/0.1% trifluoroacetic acid. Tips were rinsed twice with 1% formic acid and eluted from the Fe3+-IMAC beads onto the C-18 Stage Tips with 70 L of 500 mM dibasic potassium phosphate, pH 7.0 a total of three times. C-18 Stage Tips were then washed twice with 1% formic acid, followed by elution of the phosphopeptides from the C-18 Stage Tips with 50% ACN/0.1% formic acid twice. Samples were dried down and resuspended in 3% ACN/0.1% formic acid prior to ESI-LC-MS/MS analysis.

Enrichment of Intact Glycopeptides by MAX Columns from Tryptic Peptides

The glycopeptides were enriched from 350 μg C18 cleaned up tryptic peptides using 30 mg MAX columns (Waters). 350 ug tryptic peptides were first dried down in SpeedVac and reconstituted in 50% ACN/0.1% TFA, then constituted to 95% ACN/1% TFA. MAX columns were sequentially conditioned with 1 ml 100% ACN 3 times, then 1 ml 100 mM triethylammonium acetate buffer 3 times and 1 ml 95% ACN/1% TFA 3 times. Tryptic peptides were conditioned to bind onto the MAX columns 2 times and then washed with 1 ml 95% ACN/1% TFA 3 times. Non-intact glycopeptides were eluted/washed off the MAX columns, while intact glycopeptides were bound onto the MAX column during the process. Intact glycopeptides were then eluted using 50% ACN/0.1% TFA, dried down, and reconstituted in 3% ACN/0.1% FA prior to ESI-LC-MS/MS analysis.

### ESI-LC-MS/MS for Global Proteome, Phosphoproteome, and Glycoproteome Using DIA-MS Analysis

Individual global proteome and phosphoproteome samples were analyzed using the same instrumentation and methodology; albeit with varied gradient settings. Individual glycoproteomic samples were analyzed using the same MS instrument and gradient settings as phosphoproteome, except the MS settings, which used the methodology as previously described. 113 Unlabeled, digested peptide material from individual tissue samples (ccRCC and NAT) was spiked with index Retention Time (iRT) peptides (Biognosys) and subjected to datain dependent acquisition (DIA) analysis. Peptides (˜0.8 g; ˜ 1 ug for glycopeptides) were separated on an Easy nLC 1200 UHPLC system (Thermo Scientific) on an in-house packed 20 cm×75 m diameter C18 column (1.9 m Reprosil-Pur C18-AQ beads (Dr. Maisch GmbH); Picofrit 10 m opening (New Objective). The column was heated to 50° C. using a column heater (Phoenix-ST). The flow rate was 0.200 μl/min with 0.1% formic acid and 3% acetonitrile in water (A) and 0.1% formic acid, 90% acetonitrile (B). For global proteomic characterization of ccRCC tumors and NATs, the peptides were separated using the following LC gradient: 0-3 min (2% B, isocratic), 3-103 min (7%-20% B, linear), 103-121 min (20-30% B, linear), 121-125 min (30-60% B, linear), 125-126 min (60-90% B, linear), 126-130 min (90% B, isocratic), 130-131 min (90-50% B, linear), 131-140 min (50% B, isocratic). For global proteomic characterization of samples annotated as intra-tumor heterogeneity segments, phosphoproteomic, and glycoproteomic characterization, the peptides were separated using the following LC gradient: 0-3 min (2% B, isocratic), 3-93 min (7%-25% B, linear), 93-121 min (25-30% B, linear), 121-125 min (30-60% B, linear), 125-126 min (60-90% B, linear), 126-130 min (90% B, isocratic), 130-131 min (90-50% B, linear), 131-140 min (50% B, isocratic). Samples were analyzed using the Thermo Fusion Lumos mass spectrometer (Thermo Scientific). For global and phosphoproteome, the DIA segment consisted of one MS1 scan (350-1650 m/z range, 120K resolution) followed by 30 MS2 scans (variable m/z range, 30K resolution) as described previously.114 Additional parameters were as follows: MS1: RF Lens-30%, AGC Target 4.0e5, Max IT-50 ms, charge state include-2-6; MS2: isolation width (m/z)-0.7, AGC Target-3.0e6, Max IT-120 ms. For glycoproteome, the DIA segment consisted of one MS1 scan (450-1650 m/z range, 120K resolution) followed by 50 MS2 scans (variable m/z range within 120-2000 m/z, 15K resolution) as described previously.113 Additional parameters were as follows: MS1: RF Lens-30%, AGC Target 3.0e6, Max IT-60 ms, charge state include-2-6; MS2: AGC Target-5.3e5, Max IT-44 ms.

### Kinase Inhibition Assessment in Renal Cell Carcinoma Cell Models

For in vitro experiments assessing the impact of select kinase inhibition on renal cancer cell models (786-0, 769-P, A-498, Caki-1, and Caki-2), the kinase inhibitors, Adaversotib, Everolimus, Sapanisterib, Gefitinib, and Trametinib were dissolved in dimethyl sulfoxide (DMSO) and subjected to sonication in a water bath at room temperature. Following an assessment of individual cell line growth rates to enable calculation of half maximal inhibitory concentration (IC50), cells were seeded in triplicate at concentrations of either 1,000 cells/well (786-0, 769-P, A-498, Caki-2) or 10,000 cells/well (Caki-1). Post-24 hour seeding, cells were subjected to kinase inhibitors at final concentrations of 1 nm, 10 nM, 50 nM, 100 nM, 500 nM, 1 mM, 10 mM, with non-treated cells and DMSO treated cells included as controls. Cell growth was measured on day 1 (kinase inhibitor treatment), day 4, and day 6 using the colorimetric CellTiter 96® Aqueous One Cell Proliferation Solution Assay (MTS) following the manufacturer's instructions. IC50 for each cell line in response to single kinase inhibitor exposure was determined by plotting inhibitor concentration against percent activity relative to DMSO-treated controls and calculating the x-intercept of the linear logarithmic trend line. For phosphoproteomic characterization of renal cell models treated with individual kinase inhibitors, six treatment conditions were devised—control, Adaversotib treatment, Everolimus treatment, Sapanisterib treatment, Gefitinib treatment, and Trametinib treatment—and cells were seeded at ˜5E6 cells/15 cm plate and allowed to reach ˜80% confluency. 30 minutes prior to kinase inhibitor treatment, fresh media was exchanged. Cells were then treated with kinase inhibitors at calculated IC50 values for 1.5 hours. Media was removed and cells were three times with a volume of ice-cold PBS. Cells were scraped using 1.5 mL of ice-cold PBS, transferred to Eppendorf tubes, and spun at 3,000×g for 5 minutes at 4° C. A volume of lysis buffer (8 M urea, 75 mM NaCl, 50 mM Tris, pH 8.0, 1 mM EDTA, 2 g/mL aprotinin, 10 g/mL leupeptin, 1 mM PMSF, 10 mM NaF, Phosphatase Inhibitor Cocktail 2 and Phosphatase Inhibitor Cocktail 3 [1:100 dilution], and 20 mM PUGNAc) was added and cells lysed. Subsequent sample preparation and ESI-LC-MS/MS analysis for global proteomic and phosphoproteomic characterization for DIA analysis were performed as described for tissue samples.

### Spectral Library Generation for DIA-MS Analysis of Intact Glycopeptides

For spectral library generation, an aliquot (5 g) of unlabeled glycopeptides from individual tissue samples (ccRCC and NAT) was pooled and subjected to bRPLC as previously described. 13 In brief, the desalted, pooled sample was reconstituted in 900 L of 20 mM ammonium formate (pH 10) and 2% acetonitrile (ACN) and loaded onto a 4.6 mm×250 mm RP Zorbax 300 A Extend-C18 column with 3.5 m size beads (Agilent). Peptides were separated at a flow-rate of 0.2 mL/min using an Agilent 1200 Series HPLC instrument via bHPLC with Solvent A (2% ACN, 5 mM ammonium formate, pH 10) and a non-linear gradient of Solvent B (90% ACN, 5 mM ammonium formate, pH 10) as follows: 0% Solvent B (7 min), 0% to 16% Solvent B (6 min), 16% to 40% Solvent B (60 min), 40% to 44% Solvent B (4 min), 44% to 60% Solvent B (5 min), then holding at 60% Solvent B for 14 min, 60% to 98% Solvent B (14 min). Collected fractions were concatenated into 12 fractions previously described 115 and dried down in a Speed-Vac. For glycoproteomic characterization, a 5% aliquot each of the 12 fractions was resuspended in 3% ACN, 0.1% formic acid, and was spiked with index Retention Time (iRT) peptides (Biognosys) prior to ESI-LC-MS/MS analysis. Data acquisition using the same instrumentation for DIA-based analyses was employed using the same corresponding LC gradient, with the following Thermo Fusion Lumos mass spectrometer (Thermo Scientific) parameters: MS1: resolution—60K, mass range—350 to 2000 m/z, RF Lens—30%, AGC Target 4.0e5, Max IT—50 ms, charge state include—2—6, dynamic exclusion—45 s, top 20 ions selected for MS2; MS2: resolution—15K, high-energy collision dissociation activation energy (HCD)—34, isolation width (m/z)—0.7, AGC Target—2.0e5, Max IT—105 ms.

### Metabolome Analysis of Tissue Samples

To extract metabolites, a solution consisting of 80% (vol/vol) mass spectrometry-grade methanol and 20% (vol/vol) mass spectrometry-grade water were used to extract the metabolites from the tissue samples as described previously.116-118 The metabolite samples then underwent speed vacuum processing to evaporate the methanol and lyophilization to remove the water. The dried metabolites were re-suspended in a solution consisting of 50% (vol/vol) acetonitrile and 50% (vol/vol) mass spectrometry-grade water before data acquisition. Data acquisition was performed using a Vanquish ultra-performance liquid chromatography (UPLC) system and a Thermo Scientific Q Exactive Plus Orbitrap Mass Spectrometer.

The samples were kept at 4° C. inside the Vanquish UPLC auto-sampler. The injection volume for each sample was 2 μL. A Discovery® HSF5 reverse phase HPLC column (Sigma) kept at 35° C. with a guard column was used for reverse-phase chromatography. The mobile aqueous phase was mass spectrometry-grade water containing 0.1% formic acid, while the mobile organic phase was acetonitrile containing 0.1% formic acid. Mass calibration was performed prior to data acquisition to ensure the sensitivity and accuracy of the system. The total run time for each sample was 15 minutes, for which 11 minutes was used for data acquisition. Full MS data were acquired to quantify the metabolites while Full MS/ddMS2 data were also acquired to identify the metabolites based on fragmentation matching.

### Immunohistochemistry Analysis

Immunohistochemistry (IHC) was performed on 4-micron formalin-fixed, paraffin-embedded (FFPE) tissue sections. The antibodies characterized include CA9 (Carbonic anhydrase IX) rabbit polyclonal primary antibody (Cat No. NB100-417, Novus Biologicals, Centennial, CO), BAP1 (BRCA1 associated protein 1) mouse monoclonal primary antibody (Cat No. sc-28382, Santa Cruz Biotechnology, Dallas, TX), UCHL1 (Ubiquitin C-terminal hydrolase 1) rabbit polyclonal primary antibody (Cat No. HPA005993, Sigma-Aldrich (Atlas), St. Louis, Mo), HYOU1 (Hypoxia up-regulated 1) rabbit polyclonal primary antibody (Cat No. HPA049296, Atlas Antibodies, Bromma, Sweden), IFI30 (Interferon gamma-inducible protein 30) rabbit polyclonal primary antibody (Cat No. HPA026650, Atlas Antibodies, Bromma, Sweden), CTSA (Cathepsin A) rabbit polyclonal primary antibody (HPA031068, Atlas Antibodies, Bromma, Sweden), GAL3ST1 (Galactose-3-O-sulfotransferase 1) rabbit polyclonal primary antibody (Cat No. HPA001220, Atlas Antibodies, Bromma, Sweden), KIF2A (Kinesin heavy chain member 2A) rabbit polyclonal primary antibody (Cat No. HPA004716, Atlas Antibodies, Bromma, Sweden), PLXDC2 (Plexin domain containing 2) rabbit polyclonal primary antibody (Cat No. HPA017268, Atlas Antibodies, Bromma, Sweden) and TGFBI (Transforming growth factor beta induced) rabbit polyclonal primary antibody (Cat No. HPA008612, Atlas Antibodies, Bromma, Sweden). IHC was carried out on the Benchmark XT automated slide staining system using the Ultra View Universal DAB detection kit for CA9 and UCHL1 and OptiView DAB detection kit for BAP1 (Cat No. 760-500 and 760-700 respectively, Roche-Ventana Medical Systems, Oro Valley, AZ). IHC for HYOU1, IFI30, CTSA, GAL3ST1, KIF2A, PLXDC2 and TGFBI was performed using an automated platform Dako Autostainer Link 48 and En Vision FLEX visualizing kit (cat. no. K800221-2; Dako, Agilent Technologies Inc., Carpinteria, CA). Appropriate known positive and negative control tissue were run in each assay batch.

A semi-quantitative product score was determined for BAP1 and UCHL1 where the presence and intensity of BAP1 nuclear and UCHL1 cytoplasmic/membranous staining were scored by the study pathologists. This product score represents the percentage of positive neoplastic cells and the staining intensity (none, 0; weak, 1; moderate, 2; strong, 3) which were recorded for each tumor as described previously. 119

### In Vitro Cell Line Drug Treatment and Growth Inhibition Assessment

The ccRCC cell line Caki-1 and a control cell line HK-2 were maintained in Dulbecco's Modified Eagle Medium/Nutrient Mixture F-12 (DMEM/F-12) culture medium (Gibco-11320033) supplemented with 10% FBS (Sigma, F-9665) and 1% Pen Strep (Gibco, 10,000 U/mL-15140122). All cell lines were seeded at 50,000 cells/well in duplicates in 24-well plates at day 0 and were treated with either UCHL1 inhibitor (CAS 668467-91-2-Calbiochem, Sigma Aldrich-662086-10 MG) or GLUL inhibitor (L-Methionine sulfoximine, Sigma Aldrich-M5379-500 MG) upon reaching 50-60% confluency at day 3 in culture. For both UCH-L1inhibitor or GLUL inhibitor treatment, the working concentrations were used at 1 μM, 5 μM, and 25 μM. Treatment was maintained in culture for a total of 7 days and growth inhibition assessment was performed using AlamarBlueTM Cell Viability Reagent (Invitrogen-DAL1025) at a ratio of 1:10 for 4 hours according to the manufacturer's protocol. Plots and IC50 concentrations were produced in Prism GraphPad (version 9.2.0) by plotting the percent growth inhibition on the y-axis and the Log (concentration) on the x-axis. The corresponding IC50 was extracted from the nonlinear regression curve fitting analysis using Prism GraphPad. Cells treated with only a growth medium without any drugs were used as negative controls.

786-O cells were maintained in Gibco RPMI-1640 supplemented with 10% FBS. CAS 668467-91-2 (UCHL-1 inhibitor) was purchased from Sigma-Aldrich (L4170) and its impact on cell viability was evaluated. Briefly, 2000 cells were seeded on white flat bottom 96 well plates and were treated with increasing concentrations of the inhibitor for a week. CellTiter-Glo Luminescent Cell Viability Assay (Promega) was used to assess cell viability and IC-50 was calculated using a graph pad. Impact of UCHL-1 inhibitor on cell morphology was evaluated using IncuCyte ZOOM assay. For western blot analysis, cell lysates were harvested from control and UCHL-1 treated 786-O cells. Following protein quantification, lysates were resolved in NuPAGE Bis-Tris Protein Gel (ThermoFisher Scientific), transferred on to nitrocellulose membranes, blocked with 5% milk, and incubated overnight with UCHL-1 antibody (HPA005993, Sigma-Aldrich). Following day, membranes were washed with TBST buffer, incubated with HRP-conjugated secondary antibodies, washed, and imaged using Odyssey Fc imager (LiCOR Biosciences).

### Genomic Data Analysis

### Harmonized Genome Alignment

WGS, WES, RNA-Seq sequence data were harmonized by NCI Genomic Data Commons (GDC) gdc.cancer.gov/about-data/gdc-data-harmonization, which included alignment to GDC's hg38 human reference genome (GRCh38.d1.vd1) and additional quality checks. All the downstream genomic processing was based on the GDC-aligned BAMs to ensure reproducibility.

### Somatic Mutation Calling

Somatic mutations were called by the Somatic wrapper pipeline v1.6 (github.com/dinglab/somaticwrapper), which includes four different callers, i.e., Strelka v.2,102 MUTECT v1.7,97 VarScan v.2.3.8,104 and Pindel v.0.2.598 from WES. Exonic SNVs called by any two callers among MUTECT v1.7, VarScan v.2.3.8, and Strelka v.2 and indels called by any two callers among VarScan v.2.3.8, Strelka v.2, and Pindel v.0.2.5 were kept. For the merged SNVs and indels, a 14X and 8X coverage cutoff was applied for tumor and normal, separately. SNVs and indels were filtered by a minimal variant allele frequency (VAF) of 0.05 in tumors and a maximal VAF of 0.02 in normal samples. Any SNV that was within 10 bp of an indel found in the same tumor sample was filtered. The rare mutations with VAF of [0.015, 0.05) in ccRCC driver genes were rescued based on the gene consensus list. 120

### DNP Calling

In step 12 of Somatic wrapper pipeline v1.6 (github.com/ding-lab/somaticwrapper), it combined adjacent SNVs into DNP by using COCOON (github.com/ding-lab/COCOONS): As input, COCOON takes a MAF file from standard variant calling pipeline. First, it extracts variants within a 2 bp window as DNP candidate sets. Next, suppose the corresponding BAM files used for variant calling are available. In that case, it extracts the reads (denoted as n_t) spanning all candidate DNP locations in each variant set, and then counts the number of reads with all the co-occurring variants (denoted as n_c) to calculate the co-occurrence rate (r_c=n_c/n_t); If r_c≥0.8, the nearby SNVs will be combined into DNP and it also updates annotation for the DNPs from the same codon based on the transcript and coordinates information in the MAF file.

### Mutational Signature Analysis

Non-negative matrix factorization algorithm (NMF) was used in deciphering mutation signatures in cancer somatic mutations stratified by 96 base substitutions in tri-nucleotide sequence contexts. To obtain a reliable signature profile, the Somatic wrapper pipeline was used to call mutations from WES data. Signature Analyzer exploited the Bayesian variant of the NMF algorithm and enabled an inference for the optimal number of signatures from the data itself at a balance between the data fidelity (likelihood) and the model complexity (regularization). 91 As decomposed into signatures, signatures are compared against known signatures derived from COSMIC,121 and cosine similarity is calculated to identify the best match (parameters: —cosmic cosmic3_exome—objective Poisson-n 200).

### Germline Variant Calling

Germline variant calling was performed using the Germline Wrapper v1.1 pipeline, which implements multiple tools for the detection of germline INDELs and SNVs. Germline SNVs were identified using VarScan v2.3.8 (with parameters:-min-var-freq 0.10,-p-value 0.10,-min-coverage 3,-strand-filter 1) operating on a mpileup stream produced by samtools v1.2 (with parameters:-q 1-Q 13) and GATK v4.0.0.0122 using its haplotype caller in single-sample mode with duplicate and unmapped reads removed and retaining calls with a minimum quality threshold of 10. All resulting variants were limited to the coding region of the full-length transcripts obtained from Ensembl release 95 plus additional two base pairs flanking each exon to cover splice donor/acceptor sites. Variants were required to have allelic depth≥5 reads for the alternative allele in both tumor and normal samples. Bam-readcount v0.8 was used for reference and alternative alleles quantification (with parameters:-q 10-b 15) in both normal and tumor samples. Additionally, all variants with >0.05% frequency in gnomAD v2.1123 and the 1000 Genomes Project were filtered. 124 To predict the pathogenicity of germline variants, each variant was annotated with Variant Effect Predictor (VEP) and processed using the CharGer pipeline with the parameters from a previous pan-cancer TCGA study. 89,125 Briefly, the CharGer pipeline considers pathogenic peptide changes from ClinVar, hotspot variants, minor allele frequency from ExAC, and several in silico analyses (such as Sift and PolyPhen). Each predicted pathogenic variant was then manually reviewed.

### Copy Number Variant Calling

Copy-number analysis was performed jointly leveraging both whole-genome sequencing (WGS) and whole-exome sequencing data of the tumor and germline DNA. To perform the analysis, CNVEX (github.com/mctp/cnvex), a comprehensive copy number analysis tool that has been used previously in ccRCC studies, was used.13,24 CNVEX uses whole-genome aligned reads to estimate coverage within fixed genomic intervals and whole-exome variant calls to compute Ballele frequencies (BAFs) at variant positions (called by Sentieon DNAscope algorithm). Coverages were computed in 10 kb bins, and the resulting log coverage ratios between tumor and normal samples were adjusted for GC bias using weighted LOESS smoothing across mappable and non-blacklisted genomic intervals within the GC range 0.3-0.7, with a span of 0.5 (the target and configuration files are provided with CNVEX). The adjusted log coverage ratios (LR) and BAFs were jointly segmented by a custom algorithm based on Circular Binary Segmentation (CBS). Alternative probabilistic algorithms were implemented in CNVEX, including algorithms based on recursive binary segmentation (RBS), as implemented in the R-package jointseg. 126 For the CBS-based algorithm, first LR and mirrored BAF were independently segmented using CBS (parameters alpha=0.01, trim=0.025) and all candidate breakpoints were collected. The resulting segmentation track was iteratively “pruned” by merging segments that had similar LR and BAFs, short lengths, were rich in blacklisted regions, and had a high coverage variation in coverage among whole cohort germline samples. For the RBS- and DP-based algorithms, jointbreak-points were “pruned” using a statistical model selection method (hal.inria.fr/inria-00071847). For the final set of CNV segments, the CBS-based results were used as they did not require specifying a prior number of expected segments (K) per chromosome arm, were robust to unequal variances between the LR and BAF tracks, and provided empirically the best fit to the underlying data. The resulting segmented copy-number profiles were then subject to the joint inference of tumor purity and ploidy and absolute copy number state, implemented in CNVEX, which is most similar to the mathematical formalism of ABSOLUTE127 and PureCN (bioconductor.org/packages/PureCN/). Briefly, the algorithm inputs the observed log-ratios (of 10 kb bins) and BAFs of individual SNPs. LRs and BAFs are assigned to their joint segments and their likelihood is determined given a particular purity, ploidy, absolute segment copy number, and the number of minor alleles. To identify candidate combinations with a high likelihood, a multi-step optimization procedure that includes grid-search (across purity-ploidy combinations), greedy optimization of absolute copy numbers, and maximum-likelihood inferences of minor allele counts was used. Following optimization, CNVEX ranks candidate solutions.

Because the copy-number inference problem can have multiple equally likely solutions, further biological insights are utilized to choose the most parsimonious result. The solutions have been reviewed by independent analysts following a set of guidelines. Solutions implying whole genome duplication must be supported by at least one large segment that cannot be explained by a low-ploidy solution, inferred purity must be consistent with the variant-allele-frequencies of somatic mutations, and large homozygous segments are not allowed. In parallel, BIC-seq 2, 128 a read-depth-based CNV calling algorithm, was used to detect somatic copy number variation (CNVs) from the WGS data of tumors. Briefly, BIC-seq2 divides genomic regions into disjoint bins and counts uniquely aligned reads in each bin. Then, it combines neighboring bins into genomic segments with similar copy numbers iteratively based on Bayesian Information Criteria (BIC), a statistical criterion measuring both the fitness and complexity of a statistical model. Paired-sample CNV calling that takes a pair of samples as input and detects genomic regions with different copy numbers between the two samples was used. A bin size of ˜100 bp and a lambda of 3 (a smoothing parameter for CNV segmentation) was used. Segments were called as copy gain or loss when their log 2 copy ratios were larger than 0.2 or smaller than-0.2, respectively (according to the BIC criteria).

### Structural Variant Calling

Structural variants (SVs) were called by Manta v1.6.096 from WGS tumor and normal paired BAMs. Manta was run on canonical chromosomes with the default record- and sample-level filters, retaining variants where sample site depth is less than 3x the median chromosome depth near one or both variant breakends, the somatic score is greater than 30, and for small variants (<1000 bases) in the normal sample, the fraction of reads with MAPQO around either breakend does not exceed 0.4. It is optimized for the analysis of somatic variation in tumor/normal sample pairs. The paired and split-read evidence were combined during the SV discovery and scoring to improve accuracy. The variants were prioritized by the number of spanning read pairs that strongly (Q30) support the variants (>5 as the high confidence level). Lastly, all the SV calls in the genes of interest were manually reviewed.

Instability (wGII Calculation)

To estimate the chromosomal instability, a modified version of the Genome Instability Index (GII) was used 129 to calculate GII scores for each sample as the portion of the autosome that has an absolute copy-number unequal to the weighted median absolute copy-number across the autosomal chromosomes. To account for the variation in chromosome size and avoid the overrepresentation of larger chromosomes in the CIN estimation, a modified version of GII called weighted Genome Instability Index (wGII) was used. 130 To generate wGII, the GII was calculated for each autosomal chromosome, then took the mean of all the GII scores for all 22 chromosomes.

### DNA Methylation Microarray Processing

Raw methylation idat files were downloaded from CPTAC DCC and GDC. Beta values of CpG loci were reported after functional normalization, quality check, common SNP filtering, and probe annotation using Li Ding Lab's methylation pipeline v1.1 github.com/dinglab/cptac_methylation.

### RNA Quantification and Analysis

### RNA Quantification

The gene-level read count, Fragments Per Kilobase of transcript per Million mapped reads (FPKM), and FPKM Upper Quartile (FPKM-UQ) values were obtained by following the GDC's RNA-Seq pipeline (Expression mRNA Pipeline) docs.gdc.cancer.gov/Data/Bioinformatics_Pipelines/Expression_mRNA_Pipeline/, except running the quantification tools in stranded mode. HTSeq v0.11.294 was used to calculate the gene-level stranded read count (parameters:-r pos-f bam-a 10-s reverse-t exon-i gene_id-m intersection-nonempty—nonunique=none) using GENCODE v22 (Ensembl v79) annotation downloaded from GDC (gencode.gene.info.v22.tsv). The read count was then converted to FPKM and FPKM-UQ using the same formula described in GDC's Expression mRNA Pipeline documentation.

miRNA Quantification

miRNA-Seq FASTQ files were downloaded from GDC. The mature miRNA and precursor miRNA expression are reported in TPM (Transcripts Per Million) after adapter trimming, quality check, alignment, annotation, reads counting using Li Ding Lab's miRNA pipeline github.com/ding-lab/CPTAC_miRNA. The mature miRNA expression was calculated irrespective of its gene of origin by summing the expression from its precursor miRNAs.

### RNA Fusion Detection

Three callers, STAR-Fusion v1.5.0,101 INTEGRATE v0.2.6,95 and EricScript v0.5.5,93 were used to call consensus fusion/chimeric events in the samples. Calls by each tool using tumor and normal RNA-Seq data were then merged into a single file and extensive filtering was done. As STARFusion has higher sensitivity, calls made by this tool with higher supporting evidence (defined by fusion fragments per million total reads, or FFPM>0.1) were required, or a given fusion must be reported by at least 2 callers. Fusions present in the panel of blacklisted or normal fusions, which included uncharacterized genes, immunoglobulin genes, mitochondrial genes, and others, as well as fusions from the same gene or paralog genes and fusions reported in TCGA normal samples, 131 GTEx tissues (reported in STAR-Fusion output), and non-cancer cell studies were removed. 132 Finally, normal fusions were removed from the tumor fusions to curate the final set.

snRNA-seq Quantification and Analysis
snRNA-seq Data Preprocessing

For each sample, the unfiltered feature-barcode matrix per sample were obtained by passing the demultiplexed FASTQs to Cell Ranger v3.1.0 ‘count’ command using default parameters, and a customized pre-mRNA GRCh38 genome reference was built to capture both exonic and intronic reads. The customized genome reference modified the transcript annotation from the 10x Genomics pre-built human genome reference 3.0.0 (GRCh38 and Ensembl 93). Seurat v3.1.2133,134 was used for all subsequent analyses. A Seurat object was constructed using the unfiltered feature-barcode matrix for each sample. A series of quality filters were applied to the data to remove those cell barcodes which fell into any one of these categories recommended by Seurat: too few total transcript counts (<300); possible debris with too few genes expressed (<200) and too few UMIs (<1,000); possibly more than one cell with too many genes expressed (>10,000) and too many UMIs (>10,000); possible dead cell or a sign of cellular stress and apoptosis with a too high proportion of mitochondrial gene expression over the total transcript counts (>10%).

Each sample was scaled and normalized using Seurat's ‘SCTransform’ function to correct for batch effects (with parameters: vars.to.regress=c (“nCount_RNA”, “percent.mito”), variable.features.n=3000). All samples were merged and the same scaling and normalization method was repeated. All cells in the merged Seurat object were then clustered and the top 30 PCA dimensions via Seurat's ‘FindNeighbors’ and ‘FindClusters’ (with parameters: resolution=0.5) functions. The resulting merged and normalized matrix was used for the subsequent analysis.

snRNA-seq Cell Type Annotation

Cell types were assigned to each cluster by manually reviewing the expression of marker genes. 135,136 For instance, the marker genes used were AIF1, CD68, LST1, IFITM2 (Macrophages). CD8A, CD8B, CD3E, CD3D, PRF1, GZMA, GZMB, GZMK, GZMH, CD4, IL7R, LTB, LDHB, CD69, FAS, KLRG1, CD28, DPP4 (CD4/CD8 T-cells); CD19, CD79A, CD79B, MS4A1, SDC1, IGHG1, IGHG3, IGH4 (B-cells/Plasma); EMCN, FLT1, PECAM1, KDR, PLVAP, PLVAP, TEK, VWF, ACTA2, ANGPT2, COL1A1, COL3A1, COL5A1, COL12A1, EMILIN1, LUM (Stroma).

snRNA-seq Analysis

Differentially expressed genes within each cell type were identified by the FindMarkers function comparing cells belonging to one subtype (immune subtype or multi-omic subtype) to the rest. Wilcoxon statistical test was used. log 2FC>0.25 and FDR <0.05 was used to filter DEGs.

### Trajectory-Based Analysis

The relationships between the tumor subclusters observed across the different segments of each of the four cases was observed by constructing their trajectories. Monocle-type analysis of ordering single cells in pseudotime placed the connections of multiple segments along the trajectory. snRNA-seq was imported into Monocle2.137 Parameters for the analysis were consistent with the tutorial (cole-trapnell-lab.github.io/monocle-release/docs/# constructing-single-celltrajectories), except that (1) cell type is set as the variable for differential expression text and (2) to select genes used for ordering, le-10 was set as the q value cutoff. The function “plot_cell_trajectory” was used to visualize subcluster projection in the trajectory.

### MS Data Interpretation

ccRCC Whole Proteome DIA Data (INI+EXP)

Data independent acquisition (DIA) proteomics technology was used to perform protein quantification across the combined set of 487 samples: 199 samples from the confirmatory ccRCC cohort (acquired as part of this work), 94 ITH samples (acquired as part of this work), and 194 discovery ccRCC study samples.13 In addition, 16 DDA files were used as part of the spectral library building step. The DDA files were obtained from fractionated peptide samples (8 fractions from the pooled confirmatory ccRCC sample, and 8 fractions from the pooled discovery ccRCC sample).

Raw mass spectrometry files were converted into mzML file format. FragPipe computational platform (version 15) with MSFragger (version 3.2), 138,139 Philosopher (version 3.4.13),140 and EasyPQP (version 0.1.9 doi: doi.org/10.1101/2021.03.08.434385) was used to build combined (DIA plus DDA) spectral libraries. DIA files were first processed using DIA-Umpire141 to extract the so-called pseudo-MS/MS spectra (3 mzML files for each input DIA file corresponding to MS/MS spectra assigned to precursors of different quality, indicated as Q1, Q2, and Q3 files). DDA mzML files and DIA-Umpire extracted DIA pseudo-MS/MS mzML files (using the highest quality, Q1, files only) were processed together through all subsequent stages of the spectral library building process. Peptide identification from MS/MS spectra was done using the MSFragger search engine against the CPTAC harmonized H. sapiens RefSeq protein sequence database13 (which included reversed protein sequences appended as decoys for subsequent false discovery rate, FDR, estimation). Both precursor and (initial) fragment mass tolerances were set to 20 ppm. Spectrum deisotoping, 142 mass calibration, and parameter optimization 139 were enabled. Enzyme specificity was set to ‘stricttrypsin’ (i.e. allowing cleavage before Proline). Up to two missed trypsin cleavages were allowed. Isotope error was set to 0/1/2. Peptide length was set from 7 to 50, and peptide mass was set from 500 to 5000 Da. Oxidation of methionine and acetylation of protein N-termini were set as variable modifications. Carbamidomethylation of Cysteine was set as a fixed modification. Maximum number of variable modifications per peptide was set to 3.

MSFragger search results (in pepXML format) were processed using the Philosopher toolkit.140 First, PeptideProphet143 (run with the high-mass accuracy binning and semi-parametric mixture modeling options) was run to compute the posterior probability of correct identification for each peptide to spectrum match (PSM). The resulting output files from PeptideProphet were processed together using ProteinProphet144 to perform protein inference (assemble peptides into proteins) and to create a combined file (protXML format) of high confidence proteins groups, encompassing both DDA and DIA-identified peptides. The minimum PeptideProphet probability for input to ProteinProphet was set to 0.9. The combined ProteinProphet file was further processed using Philosopher Filter command, which characterized each identified peptide as unique peptide to a particular protein (or protein group containing indistinguishable proteins) or assigned it as a razor peptide to a single protein (protein group) that had the most peptide evidence. Both unique and razor peptides were used for subsequent analysis. The data was filtered to 1% protein-level FDR using the picked FDR strategy. 145 The peptide, PSM, and ion-level reports were then generated and filtered using the 2D FDR approach (i.e. 1% protein FDR plus 1% PSM/ion/peptide-level FDR for each corresponding PSM.tsv, ion.tsv, and peptide.tsv files). 146 PSM.tsv files, filtered as described above, along with the spectral files (mzML files used as input to MSFragger) were used as input to EasyPQP for generation of the consensus spectrum library.

As an additional filter in EasyPQP, only peptides contained in the Philosopher-generated peptide.tsv report file were used, ensuring that the resulting spectral library was filtered to global 1% FDR at both protein and peptide level. EasyPQP was run with the ‘RT selection option’ set to ‘Automatic selection of a run as reference run’. Thus, peptide retention times (RT) in each run were non-linearly aligned (using loess method) by EasyPQP to a reference run (which was one of the DIA runs in the dataset showing the best average correlation coefficient against all other runs in the experiment). Only y and b fragments ions were considered, and the fragment ion annotation tolerance was set to 15 ppm. The final spectral library contained 178022 precursors representing 9245 proteins.

The spectral library described above was used for targeted extraction of precursor ion and protein intensities from the 487 DIA runs (samples) using DIA-NN (version 1.7.13) 146 as previously described. Protein inference in DIA-NN was disabled to use peptide-protein grouping as provided by the spectral library. The MS1 and MS2 tolerances and the RT extraction window were automatically determined for each run by the algorithm. Quantification mode was set to “Robust LC (high precision)”. The output was filtered at experiment-specific precursor Q-value <1%, global protein Q-value <1%, and run-specific protein Q-value <1%. Protein abundances were computed from the precursor ion intensities (summed to the unique gene symbol level) using the DIA-NN reimplementation of the MaxLFQ147 normalization method. The final table contained protein level quantification for 8363 genes.

ccRCC Phosphoproteomic Data (EXP)

Analysis of the phosphopeptide quantification data for the 199 samples from the confirmatory ccRCC cohort profiled using DIA was performed as described above, with the following changes. The spectral library was built from the 199 DIA runs supplemented with 9 DDA runs from the pooled fractionated phosphopeptide sample. All 3 sets of pseudo-MS/MS files extracted by DIAUmpire for each run (i.e., Q1, Q2, and Q3 mzML files) were used. MSFragger search parameters included an additional variable modification—phosphorylation on STY. Isotope error was set to 0/1. After PeptideProphet and before ProteinProphet, PTMProphet 148 was run to perform phosphosite localization, which was then propagated to the PSM.tsv reports by Philosopher. The resulting spectral library built with EasyPQP contained 7968 proteins and 121563 precursors (including non-phosphorylated proteins and peptides). When running DIA-NN, the PTM scoring option for phosphorylation was activated using the ‘—monitor-mod Unimod 21’ command. The precursor-level output table generated by DIA-NN was further processed using an R script available as part of the DIA-NN distribution to create a “sequence plus modification”-level report by summing precursor intensities based on the “Modified.Sequence” column. The data was filtered to global and run-specific precursor and protein Q-values <0.01. MaxLFQ methods were used to roll-up and normalize precursor intensities to the “sequence plus modification” level. The resulting table was then additionally processed to remove non-phosphorylated peptides and to mark which sites were localized with confidence by PTMProphet (localization probability 0.75 or higher) at the spectral library building step. The final table contained quantitative information for 71913 phosphorylated peptide forms, representing 26998 peptides from 6467 proteins (6262 unique gene symbols).

### Kinase Inhibitor Study, Proteomic and Phosphoproteomic Data

Analysis of the DIA data from the kinase inhibitor study (whole proteome and phosphopeptide enriched data) was performed as described above. For each data type, the libraries were built from the corresponding 30 DIA runs (6 treatments x 5 cell lines) supplemented with 8 or 9 fractionated DDA files for whole proteome and phosphopeptide-enriched samples, respectively. The resulting whole proteome spectral library contained 8882 proteins and 173932 precursors; the phosphopeptide enriched sample library contained 7841 proteins and 101491 precursors (including non-phosphorylated proteins and peptides). After DIA-NN quantification, the final quantification table for the phosphopeptide-enriched dataset contained quantification information for 46577 phosphorylated peptides forms, representing 22161 peptide sequences from 5154 proteins. The whole proteome dataset table contained quantification for 7654 genes.

### Quantification of Intact Glycopeptides

The DIA raw files of the intact glycopeptides were searched against the spectral library for the quantification of intact glycopeptides via Spectronaut (version 15.4, Biognosys). Mass tolerance of MS and MS/MS was set as dynamic with a correction factor of one. Source-specific iRT calibration was enabled with a local (non-linear) RT regression. All multi-channel interferences were excluded and the decoy method was set as “mutated”. The precursors were filtered by a Q value cutoff of 0.01 (which corresponds to an FDR of 1%). The quantity of a modified peptide was decided by summing the quantity of its precursors, whereas the quantity for a precursor was calculated by summing the area of its fragment ions at MS2 level. The reported quantification result was filtered as previously described 113. In brief, the filtering criteria consisted of following: the FWHM of XIC of the fragment ions <1 minute, the shape quality score for the XIC of the precursor transition groups >0.6, S/N ratio of the fragment ions >3, and cosine similarity between theoretical and measured isotopic patterns of precursors >0.9. The missing values were imputed using DreamAI (github.com/WangLab-MSSM/DreamAI), which was the tool used in the previous study for the imputation of phosphoproteomic data. Only glycopeptides with a missing rate less than 50% across all samples were imputed.

### Processing of Metabolomic Data

Acquired data were analyzed first using Thermo Scientific Compound Discoverer® software. The chromatographic peaks were integrated to obtain raw intensities of metabolites. Compounds with definite peaks and names in the software were selected. The data were then filtered based on the following criteria: m/z Cloud score greater than 60 (good fragmentation matching with compounds in the m/z Cloud database) or mass list match (mass lists include common pathways such as glycolysis, pentose phosphate pathway, hexosamine, and sialic acid pathway, purine and pyrimidine synthesis, and amino acid metabolism) and intensity >10000. Thermo Scientific TraceFinder® software was then used to quantify compounds in common pathways not found using Compound Discoverer® where the retention time (RT) was determined using Freestyle® software based on mass accuracy and fragmentation match. The data from Thermo Scientific Compound Discoverer® and TraceFinder® software were combined to generate the final list of compounds.

### Other Proteogenomic Analysis

### Differential Abundance Analysis

Global proteomic data and gene expression were used to perform pairwise differential analysis between groups of samples. A Wilcoxon rank-sum test was performed to determine the differential abundance of proteins and gene expression. At least four samples in both groups were required to have non-missing values, and the p-value was adjusted using the Benjamini-Hochberg procedure, and features were considered significant with an adjusted p-value <0.05. Proteomic features with at least a 2x fold increase in tumors were deemed to be tumor-associated markers.

These markers were the DEGs/DEPs captured by the “level 1” DE analysis on the cohort level using bulk proteogenomic data. To select the top feature-associated marker candidates, DE analysis was performed utilizing the bulk proteogenomic data in the intratumoral heterogeneity (ITH) cohorts (e.g., given cases with multiple segments) on the case level as “level 2”; snRNAseq on the segment level as “level 3”, specifically, among the tumor cell population; and last, snRNA-seq on the tumoral-cluster level as “level 4” with the resolution to identify specific tumor subpopulations.

### Tumor Microenvironment Inference

The ESTIMATE scores reflecting the overall immune and stromal infiltration were calculated by the R package ESTIMATE113 using the normalized RNA expression data (FPKM-UQ).

### Cell Type Enrichment Using Gene Expression

The abundance of each cell type was inferred by the xCell web tool, 105 which performed the cell type enrichment analysis from gene expression data for 64 immune and stromal cell types (default xCell signature). xCell is a gene signatures-based method learned from thousands of pure cell types from various sources. The FPKM-UQ expression matrix was used as the input of xCell. xCell generated an immune score per sample that integrates the enrichment scores of B cells, CD4+Tcells, CD8+ T-cells, DC, eosinophils, macrophages, monocytes, mast cells, neutrophils, and NK cells; a micro-environment score which was the sum of the immune score and stroma score. Besides, CIBERSORTx106 was applied to compute immune cell fractions from bulk gene expression data.

### Immune Clustering Using Cell Type Enrichment Scores

Immune subtypes of each of the four cancer types were generated based on the consensus Clustering 90 of the cell type enrichment scores by xCell. Among the 64 cell types tested in xCell, the cell types that were significant in at least 10% of the samples (xCell enrichment p<0.05, which filtered out the cell types not typical in kidneys) were selected. Consensus immune clustering was performed based on the z-score normalized xCell enrichment scores. The consensus clustering was determined by the R package ConsensusClusterPlus (parameters: reps=2000, pItem=0.9, pFeature=0.9, clusterAlg= “kmdist”, distance= “spearman”).

### Survival Analysis

The R package “survival” was used to perform survival analysis. The Kaplan-Meier curve of overall survival was used to compare the prognosis among subtypes (function survfit). Log-rank test (from the R package survminer) was used to test the differential survival outcomes between categorical variables. The standard multivariate Cox-proportional hazard modeling was applied to estimate the hazard ratio among subtypes (function coxph). Age, gender, histopathologic subtype, and BAP1 mutation status, as the covariates, were included in the model.

### Panoptes-Based Multi-Resolution Neural Network Imaging Models

The Panoptes-based multi-resolution neural network imaging models were trained with digitized H&E stained histopathologic slide images. Due to the size and the multi-resolution data structure of the whole slide images, they were cut into 299×299 pixel tiles at 10x, 5x, and 2.5x equivalent magnification of the scanned whole slide images. 10x, 5x, and 2.5x tiles covering the same regions were then grouped into tilesets and were treated as 1 sample following the Panoptes sample preparation protocol. 33 The samples were split into training, validation, and testing set at 70:15:15 ratio at the per-patient level for BAP1 mutation prediction task, and per-slide level for immune and methylation subtype prediction tasks. The models were trained with a batch size of 24, the initial learning rate of 0.0001, the dropout rate of 0.5, and Adam optimizer with early stop criteria when the validation loss did not decrease for at least 10000 iterations and the state at which the lowest validation loss was achieved were recorded to be the final model for testing. 4 Panoptes architectures were trained simultaneously into models and the best performing models were selected based on various statistical metrics, particularly AUROC. The activations of the second-to-the-last layer of the test set were extracted and dimensionally reduced and plotted with tSNE for feature visualization. Example tiles were highlighted and sent to pathologists for a secondary review. Selected whole slide cases from the test set were fed into the trained model and per-tile level predictions were aggregated into heatmap layers to overlay onto the original slides for feature visualization and localization.

Ancestry Prediction Using SNPs from 1000 Genomes Project

A reference panel of genotypes and a clustering based on principal components was used to identify likely ancestry. 107,765 coding SNPs with a minor allele frequency >0.02 were selected from the final phase release of the 1000 Genomes Project. 149 From this set of loci, the depth and allele counts of each sample in the cohort was measured using bam-readcount v0.8.0. Genotypes were then called for each sample based on the following criteria: 0/0 if reference count ≥8 and alternate count <4; 0/1 if reference count ≥4 and alternate count ≥4; 1/1 if reference count <4 and alternate count ≥8; and./. (missing) otherwise. After excluding markers with missingness >5%, 70,968 markers were kept for analysis. PCA was performed on the 1000 Genomes samples to identify the top 20 principal components. The cohort was projected onto the 20-dimensional space representing the 1000 Genomes data. A random forest classifier was trained with the 1000 Genomes dataset using these 20 principal components. The 1000 Genomes dataset was split 80/20 for training and validation respectively. On the validation dataset, the classifier achieved 99.6% accuracy. The fitted classifier was used to predict the likely ancestry of the cohort.

### MSI Prediction

MSI scores were calculated by MSIsensor (github.com/ding-lab/msisensor) and interpreted as the percentage of microsatellite sites (with deep enough sequencing coverage) that have a lesion. Samples with an MSIscore >3.5 are classified as “MSI-High” and the rest are classified as “MSS.” An intermediate class with 1.0<=score <=3.5 can be defined as “MSI-Low.”

### Unsupervised Multi-Omic Clustering Using NMF

Non-negative matrix factorization (NMF)-based multi-omic clustering using protein abundance, RNA transcript abundance, and log ratios of gene copy number variants (CNV) was used.

### Balancing Contribution of Data Types

To mitigate the impact of a potential bias towards a particular data type in the multi-omic clustering (e.g. vastly different number of genomic and proteomic features), the following filtering approach was applied: Data matrices were concatenated and all rows containing missing values were removed. The resulting multi-omic data matrix was then standardized by z-scoring of the rows followed by z-scoring of columns. Principal component analysis (PCA) was applied to the resulting standardized multi-omic data matrix. The PCA-derived factors matrix was used to determine the number of principal components (PCs) cumulatively explaining 90% of the variance in the standardized multi-omic data matrix (PCs90). The PCA-derived loadings matrix was used to calculate the relative contribution of each feature to each PCs90, equivalent to the squared cosine described in (Abdi and Williams wires.onlinelibrary.wiley.com/doi/10.1002/wics.101), and the relative, cumulative contributions of each feature across all PCs90 was subsequently derived. The resulting vector of relative contributions of each feature (i.e. vector sums up to 1) was then used to balance the contribution of the different data types using the following procedure:
1. For each data type sum up the contributions of all features; this determines the overall contribution of each data type, which ideally should be equal across the data types within a given tolerance, i.e.: sumome ˜1/(No. data types)
2. Remove the feature with the lowest contribution that belongs to the data type with the largest overall contribution
3. Recalculate the overall contributions of each data type and repeat steps 1-2 until the deviation is within the specified tolerance (tol=0.01).

### Non-Negative Transformation

The data matrix of z-scores was converted to a non-negative input matrix required by NMF as follows:
1. Create one data matrix with all negative numbers zeroed.
2. Create another data matrix with all positive numbers zeroed and the signs of all negative numbers removed.
3. Concatenate both matrices resulting in a data matrix twice as large as the original, but containing only positive values and zeros and hence appropriate for NMF.

### Non-Negative Matrix Factorization

Given a factorization rank k, where k is the number of clusters, NMF decomposes a p x n data matrix V (p-number of features; n-number of samples) into two matrices W and H such that multiplication of W and H approximates V. Matrix His a k×n matrix whose entries represent weights for each sample (1 to n) to contribute to each cluster (1 to k), whereas matrix W is a p×k matrix representing weights for each feature (1 to p) to contribute to each cluster (1 to k). Matrix H was used to assign samples to clusters by choosing the row (i.e. cluster) with the maximum score in each column of H.

### Determination of Factorization Rank

To determine the optimal factorization rank k (number of clusters) for the multi-omic data matrix, a range of clusters between k=2 and 8 was tested. For each value of the k matrix, V was subjected to NMF using 50 iterations with random initialization of W and H. To determine the optimal factorization rank two metrics for each value of k were calculated: 1) cophenetic correlation coefficient measuring how well the intrinsic structure of the data is recapitulated after clustering and 2) the dispersion coefficient of the consensus matrix as defined in150 measuring the reproducibility of the clustering across the 50 iterations. The optimal kopt is defined as kopt=max (dispK\hat{ }(1-cophK) for cluster numbers between k=3 and 8. Having determined the optimal factorization rank k, to achieve robust factorization of the multiomic data matrix V, the NMF procedure described above was repeated using 500 iterations with random initializations of W and H. Due to the non-negative transformation applied to the z-scored data matrix as described above, matrix W of feature weights contained two separate weights for positive and negative z-scores of each feature, respectively. To revert the non-negative transformation and to derive a single signed weight for each feature, each row in matrix W was normalized by dividing by the sum of feature weights in each row, aggregated both weights per feature and cluster by keeping the maximal normalized weight and multiplication with the sign of the z-score in the initial data matrix. Thus, the resulting transformed version of matrix Wsigned contained signed cluster weights for each feature in the input matrix.

### Cluster Membership

For each sample, a cluster membership score was calculated as the maximal fractional score of the corresponding column in matrix H. The score indicates how representative a sample is to each cluster and was used to define the “cluster core”, a set of samples most representative for a given cluster. Core samples were required to have a minimal membership score difference between all pairs of clusters to be greater than 1/k, where k is the total number of clusters. The entire workflow described above has been implemented as a module for PANOPLY151 (github.com/broadinstitute/PANOPLY) which runs on Broad's Cloud platform Terra (app.terra.bio/).

### Unsupervised Clustering of DNA Methylation

Methylation subtypes were segregated based on the top 8,000 most variable probes using kmeans consensus clustering as previously described. 152 Underperforming probes were removed, 153 and then the samples with more than 30% missing values. Remaining missing values were imputed using the mean of the corresponding probe value. Clustering was performed 1000 times using the ConsensusClusterPlus R package (parameters: maxK=10 reps=1000 pItem=0.8 pFeature=1 clusterAlg= “km” distance= “euclidean”). K=6 was chosen based on the delta area plot of consensus CDF.

### Determination of Stemness Score

Stemness scores were calculated as previously described. 154 Firstly, MoonlightR 155 was used to query, download, and preprocess the pluripotent stem cell samples (ESC and iPSC) from the Progenitor Cell Biology Consortium (PCBC) dataset.156,157 Secondly, to calculate the stemness scores based on mRNA expression, a predictive model was bult using one-class logistic regression (OCLR) 158 on Progenitor Cell Biology Consortium (PCBC) dataset. For mRNA expression-based signatures, to ensure compatibility with the cohort, the Ensembl IDs were mapped to Human Genome Organization (HUGO) gene names and any genes that had no such mapping were dropped. The resulting training matrix contained 12,945 mRNA expression values measured across all available PCBC samples. To calculate the mRNA-based sternness index (mRNASi), FPKM-UQ mRNA expression values were used for all CPTAC ccRCC tumors. The TCGAanalyze_Stemness function from the R package TCGAbiolinks was used159 following the previously described workflow, 160 with “stemSig” argument set to PCBC stemSig.

### Mutation Impact on RNA, Proteome, Phosphoproteome, and Metabolome

A set of interacting proteins (e.g. kinase/phosphatase-substrate or complex partners) was aggregated from OmniPath (downloaded on 2018 Mar. 29),84 DEPOD (downloaded on 2018 Mar. 29), 161 CORUM (downloaded on 2018 Jun. 29), 162 Signor2 (downloaded on 2018 Oct. 29), 163 and Reactome (downloaded on 2018 Nov. 1).164 Analyses were focused on ccRCC SMGs previously reported in the literature. 120

For each interacting protein pair, samples were split with and without mutations in partner A and expression levels (RNA, protein, and phosphosites) were compared both in cis (partner A) and in trans (partner B), calculating a median difference in expression and testing for significance with the Wilcoxon rank-sum test, with the Benjamini-Hochberg multiple test correction. For mutational impact analysis on metabolomes, all possible pairs between SMGs and metabolites were tested.

### Kinase-substrate Pairs Regression Analysis

For each kinase-substrate protein pair supported by previous experimental evidence (OmniPath, NetworKIN, DEPOD, and SIGNOR), the associations between all sufficiently detected phosphosites on the substrate and the kinase were tested. For a kinase-substrate pair to be tested, both kinase protein/phosphoprotein expression and phosphosite phosphorylation needed to be observed in at least 20 samples in the respective datasets and the overlapped dataset. The linear regression model was applied using Im function in R to test for the relation between kinase and substrate phosphosite. For the i-th trial for kinase phosphosite abundance in the cis associations, kinase phosphosite abundance Ai depends on kinase protein expression Si and error Ei, Ai=M1Si+B+Ei

For the i-th trial for kinase phosphosite abundance in the trans associations, substrate phosphosite abundance Ai depends on kinase phosphosite expression Ki substrate protein expression Si and error Ei, Ai=M1Si+M2Ki+B+Ei where the regression slope M coefficients are determined by least-square calculation. Bs are y-axis intercepts. The resulting p-values were adjusted for multiple testing using the Benjamini-Hochberg procedure.

### Phosphoproteomic and Glycoproteomic Subtyping

Phosphopeptides with CVs in the >25% quartile were analyzed by CancerSubtypes 165 for consensus clustering of tumor subtypes. The same procedure was carried out for glyco subtyping using intact glycopeptides as well. Specifically, 80% of the original sample pool was randomly subsampled without replacement and partitioned into four major clusters (phospho) and three major clusters (glyco) using hierarchical clustering, which was repeated 2000 times. The consensus-clustered samples were overlaid with other features (e.g., grade, stage) and other omics subtypes (e.g., methylation subtype, histopathologic subtype). Phosphopeptides and intact glycopeptides were grouped into four and three clusters using K-means clustering in ComplexHeatmap, 166 respectively. The predictive models of phospho- and glyco-signatures were built using caret doi.org/10.18637/jss.v028.i05) and ROC curves were generated using pROC.167 KEGG pathway enrichment analysis as performed via WebGestalt. 168 PTMSEA was utilized to find signatures (pathways and kinases) of the phospho subtypes. Differential analysis between a phospho subtype to the remaining phospho subtypes (P1 vs Others, P2 vs Others etc.) as well as the pairwise comparison between phospho subtypes (P1 vs P2, P1 vs P3 etc.) on phosphosite level was conducted by calculating median log 2 fold change and obtaining pvalue from Wilcoxon rank-sum test. Next, each differentially expressed phosphosites in one phospho subtype was examined relative to the remaining phospho subtypes (p≤0.05 and fold change ≥1.5) to ensure that it was also differentially expressed (p≤0.05 and fold change ≥1.5) in the particular subtype from the pairwise comparison (at least compared to two out of three other phospho subtypes) in order to generate a list of phosphosites for PTM-SEA input. To obtain a single enrichment score from PTM-SEA and adequately account for variance in phosphosite abundance across subtypes, the differential analysis results from one subtype vs the remaining was used to calculate signed (according to the fold change between one subtype and the remaining), log-transformed p-value from Wilcoxon Rank Sum Test as input to PTM-SEA. Only pathways and kinases significantly enriched (FDR<0.05) in at least one of the subtypes were plotted. The differential analysis between a glyco subtype to the remaining glyco subtypes was conducted by calculating median log 2 fold change and using Wilcoxon rank-sum test (p-value was adjusted using Benjamini Hochberg method). The significance threshold was set as FDR <0.05. The setting in PTM-SEA is as follows.
sample.norm.type: rank
weight: 1
statistic: area.under.RES
output.score.type NES
nperm: 5000
min.overlap: 5
correl.type: rank

### Metabolome Analysis

Metabolome data were used to perform pairwise differential analysis between groups of samples. A Wilcoxon rank-sum test was performed to determine the differential abundance of metabolites. At least four samples in both groups were required to have non-missing values and the p-value was adjusted using the Benjamini-Hochberg procedure. The metabolite annotations were based on HMDB (hmdb.ca/), MetaboAnalyst (www.metaboanalyst.ca/), and KEGG (www.genome.jp/kegg/).

### Interactive Data Visualization and Exploration

A ProTrack web portal 169 was developed for interactive visualization and exploration of this data set. The ProTrack web app consists of two main views: a sample dashboard and an interactive heatmap. The sample dashboard visualizes the distribution of the cohorts along clinical, demographic, and molecular variables. The graphs can be reordered and hidden or shown according to user preference. The graphs can also be used to create custom cohorts, as users can filter samples into a custom cohort by toggling demographic features on and off. The filtered cohort can optionally be used to generate an interactive heatmap. On the heatmap view, users input a query list of genes of interest. A multi-omic heatmap is then generated for those genes, including protein, RNA, phosphoprotein, and glycoprotein data tracks when available. Additionally, using the interactive legend, users can add or remove top tracks to include immune subtype classification tracks, mutation information, chromosomal gains or losses, and clinical or demographic data such as BMI, hypertension, vital status. To facilitate the visualization of trends of interest, users can select any track and sort the entire heatmap along that axis. The underlying ordered data can then be downloaded as an Excel table and the heatmap can be exported as an image file. The ProTrack application is available at ccrcc-conf.cptac-data-view.org.

### Results

Overview of study design, cohort, and data types

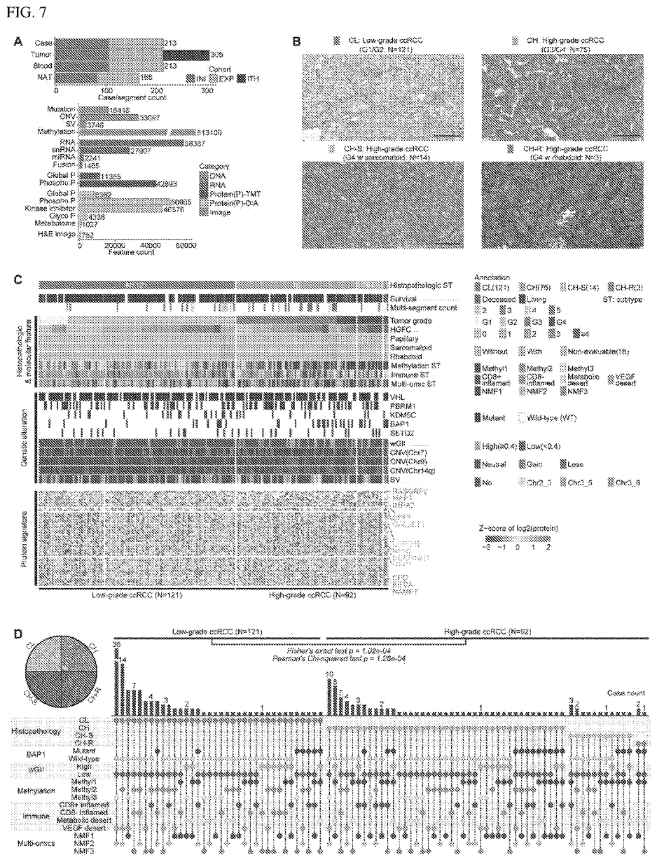

CPTAC previously characterized 103 treatment-naïve ccRCC cases using Tandem Mass Tagging (TMT)-based global proteomics and phosphoproteomics platforms (Clark et al., 2019). This study increased the cohort size to 213 cases, with 40 cases being selected for multiple-segment profiling of an additional 92 segments to evaluate tumor evolution and ITH. The final analyzed dataset contained 305 tumor samples, 165 paired NATs, and 213 blood normal samples among 16 different data types from the initial (INI), expanded (EXP), and ITH cohorts (FIG. 7A). Samples were genomically and epigenetically characterized as before (Clark et al., 2019), while DIA-based proteomic analysis was used to profile all samples for the global proteome and the newly added 110 cases for phosphoproteome and glycoproteome (FIG. 7A). In addition, 106 selected cases were analyzed by metabolome analysis, and 15 tumor specimens from 7 cases had single-nuclei RNA-seq (snRNA-seq) to investigate both the tumor-intrinsic cell populations and the TME (FIG. 7A). In parallel, a comprehensive histopathologic evaluation was performed based upon 21 parameters (Methods) to define low- and high-grade features, spatial architecture, and TME. Molecular profiles and histopathologic annotations were integrated to characterize distinct histological features, understand molecular mechanisms that drive ccRCC, and provide a reference for selecting effective therapy.

ccRCC histopathologic heterogeneity and its molecular underpinnings

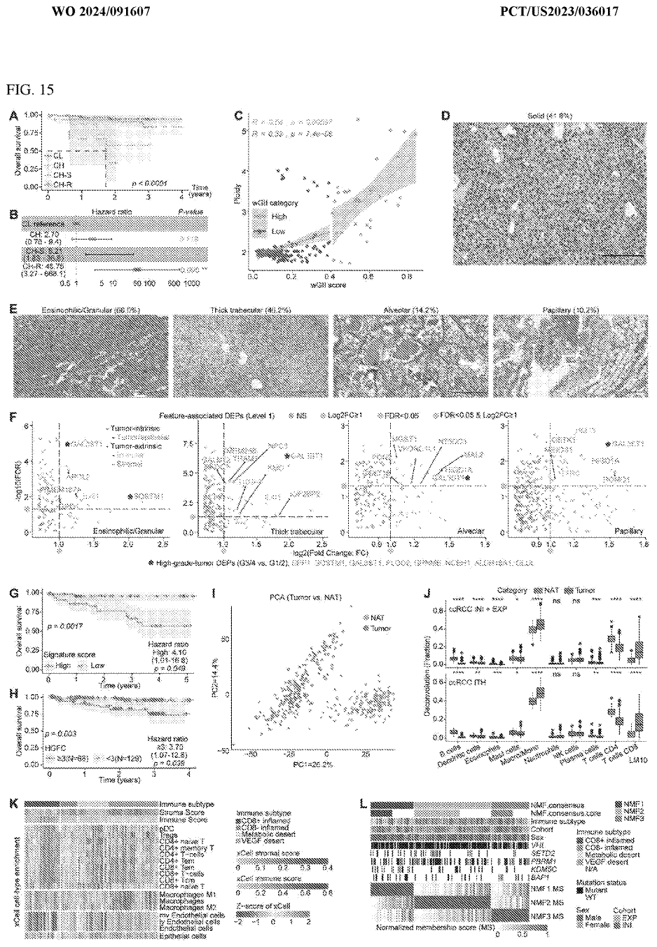

ccRCC tissues display extensive histopathologic heterogeneity within the tumor epithelia manifesting as differences in nuclear/nucleolar features that form the basis of clinical Fuhrman grading (Fuhrman et al., 1982; Novara et al., 2007). In parallel, heterogeneity is observed in tumor architecture (pattern), cytology, and changes in the microenvironment (Cai et al., 2020). High Fuhrman grade tumors are associated with an elevated risk of recurrence post-surgery and warrant more frequent post-surgical surveillance. Differences in architecture/cytological patterns have also recently been linked to aggressive disease (Cai et al., 2020; Verine et al., 2018). The underlying molecular changes associated with histopathologic heterogeneity are not fully understood. Hence, in the first approach based on tumor grade and the presence of sarcomatoid or rhabdoid histologic features (GSR), the 213 ccRCC cases were classified into four histopathologic subtypes that were used to guide integrative multi-omic analysis (FIG. 7B). Low-grade ccRCC (CL) tumors (G1/G2: N=121) and high-grade ccRCC (CH) tumors (G3 or G4: N=92) represented the remaining tumor cases. Among the CH group, 14 exhibited sarcomatoid features (CH-S), and 3 showed rhabdoid features (CH-R) that were linked to the distinct morphological pattern as shown in the H&E images (FIG. 7B). Overall, CHR and CH-S were associated with worse prognosis compared to CL (FIGS. 15A-B). Differential expression (DE) analysis identified tumor markers associated with each of the four major histopathologic subtypes. Notably, LRRC59 and SERPINH1 were highly expressed in CH-S tumors. KIF2A, a kinesin family (KIF) member that has been reported to be aberrantly expressed and correlated with patient survival (Li et al., 2019a), had significantly increased expression in CH-R (FIG. 7C). Methylation subtype Methyl1 was significantly enriched in high grade tumors while VEGF immune desert was associated with CL (respective p=2.15e-10; 1.69e-10) (FIG. 7C). Although limited in number, all 3 CH-R tumors demonstrated BAP1 mutations and chr14 loss in addition to VHL mutation. High-grade tumors had significant enrichment of high weighted Genome Instability Index (wGII) scores (score>0.4, p=0.0023) in addition to enrichment for loss of chr9, 14p, and 14q (respective p=0.00058; 0.0019; 6.36e-7) (FIG. 7C). Following the initial clonal chr3p loss and acquisition of 3p driver gene mutations, a subset of ccRCC underwent whole-genome duplication (WGD), resulting in tetraploidy. Following WGD, a significant subset of these tumors acquired several additional copy number changes (gains/losses) at an increased rate, resulting in genomic instability (GI). Distinguishing patient subsets with high GI may have clinical and therapeutic implications. The GI quantified here by wGII score correlated (R=0.54) with ploidy in the wGII high group (FIG. 15C).

In the second approach, the information obtained by systematic histopathologic review of 197 tumors (with available H&E slides) contained 21 morphological parameters (Methods). To identify underlying molecular changes associated with histopathologic heterogeneity, 7 high-grade morphologic features were systematically assessed for eosinophilic/granular change, thick trabeculae, alveolar, solid, papillary/pseudopapillary patterns, and rhabdoid or sarcomatoid cytology (FIGS. 7B, 15D-15E), and quantified as High-Grade Feature Count (HGFC) per tumor based on the presence of identifiable high-grade features. High-grade features contributing to considerable histologic heterogeneity within tumors, were specifically enriched among CH-S and CH-R tumors shown in the histopathologic annotation block of the heatmap (FIG. 7C), with clinical implications (Cai et al., 2020; Verine et al., 2018).

In addition to identifying sarcomatoid and rhabdoid feature-associated events (FIG. 15F, the differentially expressed proteins (DEPs) of other high-grade features was investigated by comparing corresponding tumors to controls (tumors without any of the above-mentioned 7 high-grade features). The papillary/pseudo-papillary feature was captured in 10.2% of the tumors with associated upregulation of HIGDIA and ROMO1 (FIGS. 15E-15F). Some markers were not specific to a certain high-grade feature but generally overlapped with the high-grade-tumor DEPs (G3/4 tumors vs. G1/2 tumors). The top altered proteins included SQSTM1, GAL3ST1, and PLOD2 (FIG. 15F). Protein abundances for LRRC59, RPN2, and SERPINH1, the top sarcomatoid-associated-markers as DEPs, were converted into an integrative signature score that may serve as a prognostic indicator (e.g., high expression correlating with worse prognosis) (FIGS. 7C, 15G). The group with a high signature score carries a statistically significant higher hazard ratio of 4.1 with a p of 0.049 adjusting by age, sarcomatoid feature status, tumor stage, and immune subtype in the Cox proportional hazards (Cox) models. Considering the status of all 7 high-grade features, an HGFC was determined (range from 0 to 7) for each tumor and evaluated for its prognostic value. Among the 197 tumors with evaluable H&E images and annotations, 68 (34.5%) presented an HGFC >3 that was associated with a worse prognosis (p=0.003) (FIG. 15H). By adjusting for other covariates (histopathologic subtype, age, sex, BAP1 mutation) in the Cox model, the hazard ratio of this group was 3.7 (p=0.039) compared with the group of HGFC (<3), as indicated in FIG. 15H.

In conjunction with evaluating histopathologic features, detailed proteogenomic characterizations were performed and associations between the omic layers and each of the seven high-grade histopathologic features mentioned above were evaluated. For example, methylation subtype, immune subtype, and BAP7 mutation showed strong associations with the sarcomatoid phenotype. The tumors presented distinct features compared to NATs, as revealed by immune cell-type deconvolution analysis (FIGS. 15I-J). Abundances of macrophages and CD8+ T cells were significantly higher in tumors, while CD4+ T cells were enriched in NATs, a consistent feature across the ccRCC cohorts (FIG. 15J). Among the 305 ccRCC specimens (FIG. 15K), four distinct immune subtypes (CD8+inflamed with high immune infiltration; CD8-inflamed with high fibroblast signature; metabolic desert with high epithelial signature; and VEGF desert with high endothelial signature), which were largely consistent with the four previously reported immune subtypes (Clark et al., 2019) were detected. Tumors in the CD8+inflamed group may be more likely to respond to immunotherapy than immune-desert tumors (metabolic desert, VEGF desert) (FIG. 15K). Based on the clinical annotation, 19 patients received adjuvant postoperative immunological therapy. Four were classified into the CD8+inflamed subtype. This immune subtyping approach provided an additional resolution to immune-inflamed and immune-desert tumors (Braun et al., 2020), identifying two distinct immune-desert subtypes, and shared some similarities with the unsupervised transcriptomic subtypes previously reported (Motzer et al., 2020a). By utilizing multi-omic data (e.g., CNV, gene expression, and global protein abundance), three major multi-omic subtypes, NMF1, NMF2, and NMF3 associated with metabolic desert, VEGF desert, and CD8-inflamed tumors, respectively were identified (FIG. 15L). These correlated with other molecular and clinical features such as wGII high and high-grade tumors that were enriched in NMF1. Moreover, a cluster membership score was calculated for each sample that defined the “cluster core”, a set of samples most representative of a given cluster (Methods). Among the core samples in the three subtypes, overall survival differed significantly (p=0.038) as NMF1 was associated with a worse prognosis, and NMF1, compared with NMF3, carried a higher hazard ratio of 9.98 (p=0.059) adjusting by age, sex, and tumor grade in the Cox model. In a comprehensive exploration of phenotype-genotype association, details of histopathologic heterogeneity were integrated in multi-omic analysis. Using this approach, clinical and molecular features associated with high-risk disease, including Fuhrman grade, HGFC, genome instability (underexplored in the current literature), and novel proteomic markers were identified. UCHL1 protein expression was investigated as a prognostic biomarker associated with poor survival, BAP1 mutation, high wGII, and specific DNA methylation subtype. Detailed characterization of UCHL1 is presented in the DNA methylation section below. In summary, the results revealed a higher level of intertumoral heterogeneity in high-grade tumors compared with low-grade tumors (p=1.02e-04) (FIG. 7D).

ccRCC Proteogenomic and TME ITH Revealed by Comprehensive Multi-Segment Integrative Analysis

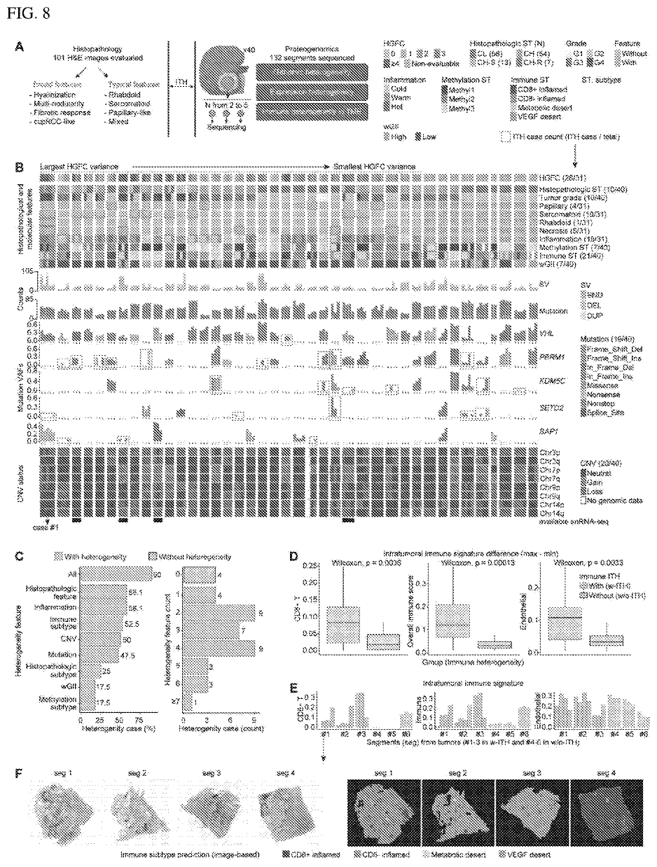

To understand ITH in ccRCC, spatial proteogenomic patterns were analyzed using 132 distinct samples obtained from 40 ccRCC cases. Multi-segment (2-5 segments from distinct regions of tumor tissue from a given case) multi-omic profiling and integrative analysis was performed with various histopathologic features (FIG. 8A). Following the schema described in the previous section, GSR and HGFC parameters were determined for each segment from the corresponding H&E images (N=101) upon pathology review. Briefly, each segment was scored against pre-decided low (4 parameters) and high grade (7 parameters) histopathologic features, including identifying areas of transition between phenotypes, broad histopathologic features relatively prevalent in a subset of tumors (e.g., hyalinization and multi-nodularity), and some unique features in selected cases (FIG. 8A). The second part of the ITH workflow generated comprehensive proteogenomic molecular profiles that captured genomic and expression heterogeneity from bulk proteogenomic data. snRNA-seq added details at the single cell resolution on immune heterogeneity in the TME (FIG. 8A). Using integrative analysis, the association between histopathologic features and molecular profiles was explored for a deeper understanding of ccRCC ITH (FIG. 8A).

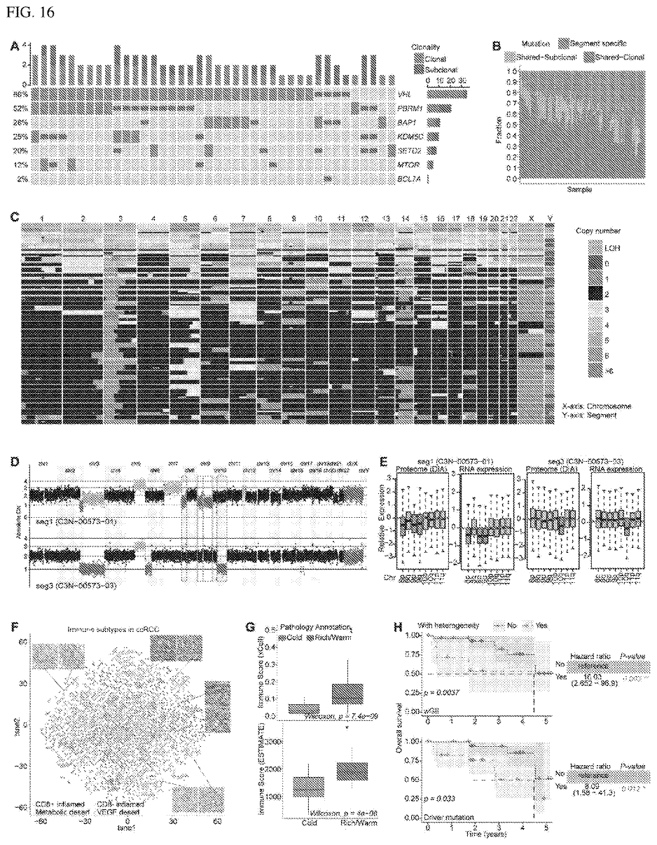

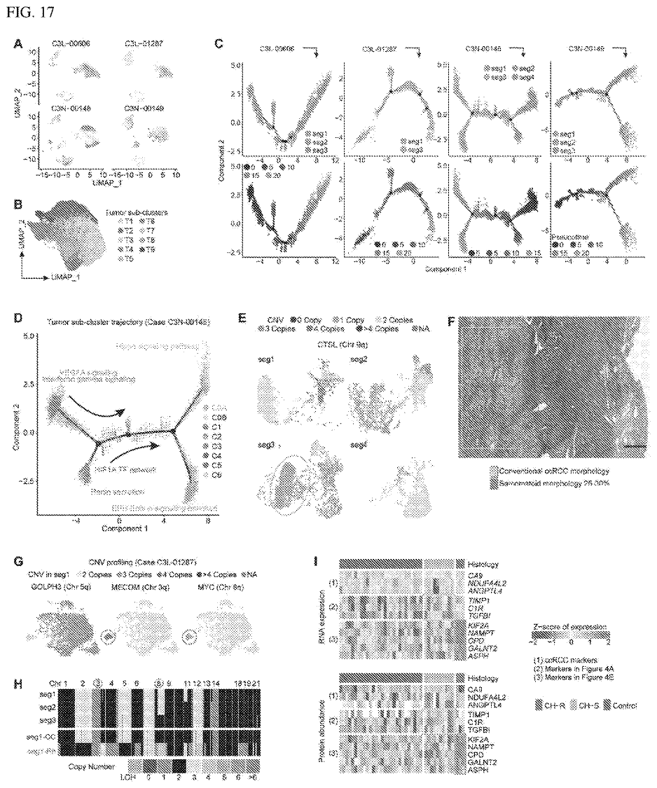

To investigate ITH in ccRCC somatic aberrations and its proteomic impact, the cases were sorted according to the variances of HGFC (FIG. 8B). Features enclosed with red rectangles highlight the heterogeneity observed across segments at various levels (FIG. 8B). ITH at istopathologic and genomic levels was more prevalent in a subset of cases, as represented by case #1 (FIGS. 8B-8C). Among the five segments profiled from this case, two lacked sarcomatoid or rhabdoid features, placing them into a different histopathologic subtype (CH vs. CH-R), yet one contained a SETD2 missense mutation. This contrasts with the VHL and BAP1 mutations common to all case #1 segments. Other strong evidence of ITH noted in this case includes additional somatic aberration differences where 2 segments showed distinct patterns such as high wGII, Methyl1, metabolic desert, high structural variation (SV) counts, copy number variation (CNV) gain events in chr7, and CNV loss in chr9p (FIG. 8B). Overall, 90% (36/40) of the cases presented heterogeneity in at least 1 of the 8 heterogeneity features and more than half showed immune or histologic feature heterogeneity (FIG. 8C). Among ccRCC driver genes, the vast majority of somatic mutations in VHL were clonal events, while subclonal events were more frequent in PBRM1 (FIG. 16A). The fractions of segment-specific, shared subclonal, and shared-clonal events varied across tumors or segments of a given tumor (FIG. 16B). Additionally, CNV heterogeneities that indicated the varied tumor subpopulations (FIG. 16C) and, as demonstrated in FIGS. 16D-16E, will contribute to significant variation in the proteo-transcriptomic expression milieu in the tumor epithelia were detected.

Using data-driven approaches and histopathologic review, the immune heterogeneity level of each case was classified across its respective multiple segments (FIG. 8B). By comparing signature distributions (e.g., CD8+ T, endothelial cell, and overall immune score) between groups with (w-ITH) and without (w/o-ITH) intratumoral immune heterogeneity, the signature difference (max-min values among segments of each case) tended to be higher in the w-ITH group (p<0.05) (FIG. 8D), and 6 representative tumors (3 in w-ITH and 3 in w/o-ITH) were presented in FIG. 8E. Overall, the w-ITH group showed a high level of immune ITH. Evidence for heterogeneity in immune presentation may be a feature signaling response to immunotherapy. More importantly, heterogeneity in the immune landscape may lead to treatment failure or inappropriate therapy choices. Panoptes-based multi-resolution neural network models were trained to predict immune subtypes based on H&E images (Hong et al., 2021). Prediction and feature localization of a case with high immune ITH from the test set were highlighted in FIG. 8F. As an immune prediction tool, Panoptes showed high consistency of immune subtype prediction based on H&E images and transcriptomic immune subtyping (FIG. 8F). Tiles with similar histopathologic features related to immune subtypes were clustered together from the prediction (FIG. 16F). Furthermore, the immune characterization was evaluated to confirm the consistency between the histopathologic review and data driven delineation of the immune signature (FIG. 16G). Heterogeneities in wGII status or mutations in ccRCC driver genes were associated with worse prognosis with hazard ratios of 16.03 (p=0.003) and 8.09 (p=0.012) (FIG. 16H), respectively, after adjusting by age, sex, and tumor grade in the Cox model. ITH analysis showed that regional histologic and proteogenomic variations within a patient's tumor were common in ccRCC. Regional variations in somatic driver clonality plus numerous chromosomal CNV induced ITH in the proteotranscriptomic milieu which may play a significant role in shaping the observed regional TME heterogeneity. ITH and how some histologic features frequently associated with aggressive disease, such as sarcomatoid differentiation and rhabdoid features, can be characterized by snRNA-seq was investigated.

### Single-Cell Analysis Identified ITH, Sarcomatoid and Rhabdoid Expression Signatures

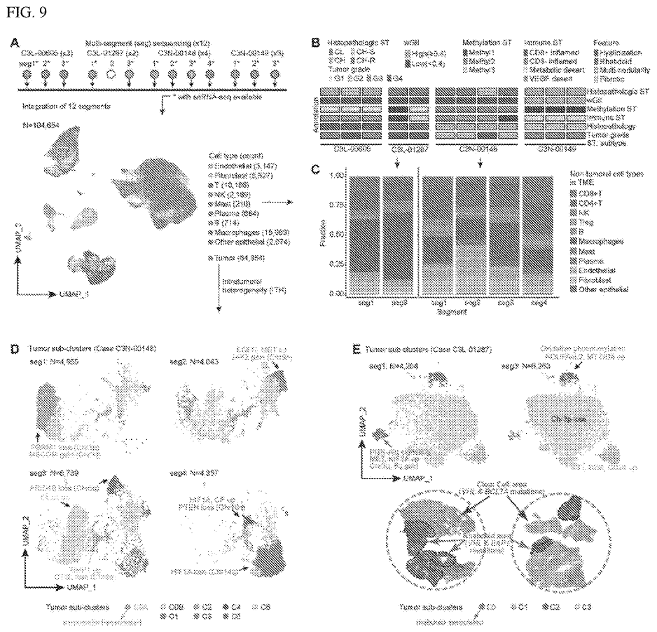

Twelve regions were selected from 4 cases in the ITH cohort for multi-segment snRNA-seq. Sample selection was based on the presence of certain features, including rhabdoid, sarcomatoid, multinodularity, and hyalinization (FIGS. 9A-B). Among the 104,654 nuclei sequenced, 62% were tumor nuclei, and they formed a main tumor cluster that contained case-specific subclusters. The remaining TME nuclei made of T, NK, B, macrophages, fibroblasts, and endothelial cells, formed cell-type-specific clusters (FIGS. 9A). Collectively, these data represented the cellular ITH observed in both the tumor and TME compartments (FIGS. 9A-C, 17A-B). The cell-type fractions characterized in snRNA-seq agreed well with the molecular and pathologic annotations. For example, case C3N-00149 presented a higher abundance of fibroblasts, which demonstrated correlative morphologic fibrotic features (FIGS. 9A-B, 17A) and was distinct from the other three cases. Comparing the TME of four segments of case C3N-00148, CD8+ T cells were significantly enriched in segment 4 (seg 4) with adjusting for the proportion of tumor, which was the only segment classified as CD8+inflamed with a higher immune infiltration level (FIGS. 9B-C).

The tumor population (64,854 nuclei) contained tumor-intrinsic markers associated with certain features and the corresponding enriched pathways at the case level. snRNA-seq from C3N-00148 presented an ideal opportunity to understand ITH and sarcomatoid differentiation, as this poor-prognosis feature was variably distributed across segments in this case. Trajectory analysis shown in FIG. 17C of C3N-00148 revealed enrichment differences of segments in distinct branches and predicted a later evolution of tumor subpopulations in seg3 labeled as C0 with high expression of GLUL as a high-grade-tumor DEP (FIG. 15F), chr9q loss, and enriched Hippo signaling pathway corresponding to the trajectory branches (FIGS. 9D, 17D-E). chr9q (Figure S3E) loss is associated with sarcomatoid changes in RCC (Ito et al., 2016). Two subpopulations (e.g., C0A, COB) were captured in C0 as C0A, in addition, showed unique expression signatures (FIGS. 9D). In agreement with the histopathologic review, the sarcomatoid and fibroblastic proliferations were mainly observed in seg3 (˜25-30%), while the other tumor segments had little or focal fibroblastic proliferation mainly in high-grade areas (<10%) (FIGS. 17F).

Similarly, snRNA-seq was examined from C3N-01287, as this case presented rhabdoid phenotype, another poor prognosis ccRCC histologic feature. The C3N-01287 tumor contained juxtaposed regions with clear cell and rhabdoid morphology, and the snRNA-seq captured both tumor compartments as distinct cell clusters (FIG. 9E). To annotate tumor subclusters further, inferred copy number results from snRNA-seq and CNV derived from microdissected rhabdoid and clear cell regions were compared using whole-exome sequencing (FIGS. 9E, 17G-H). This integrated approach revealed that the rhabdoid cell cluster/region contained BAP1 mutation, chr3q and 8q copy gains, and enrichment of PI3K-AKT and Rho GTPase signaling labeled as C0. In contrast, the clear cell cluster/region contained BCL7A mutation and chr2 and 5 gains, while VHL mutation was common to both regions (FIGS. 9E, 17G-H). Comparatively, chr5q gain is more typical in areas with clear cell istomorphology (Perrino et al., 2015); 8q gain is enriched in renal medullary carcinoma (FIGS. 17G-H) (Msaouel et al., 2020).

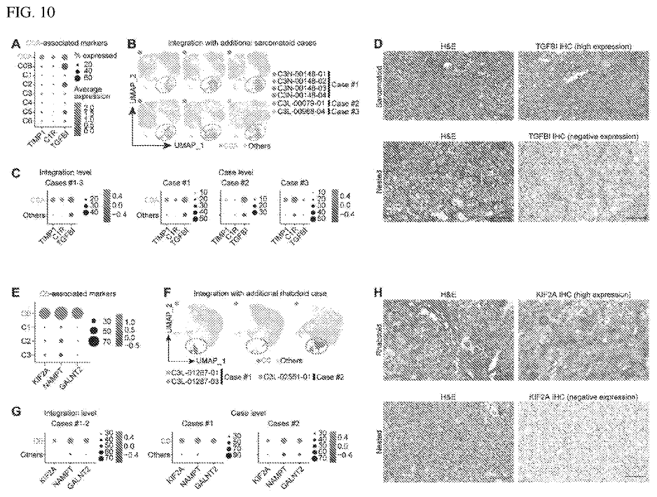

The representative genomic alterations and marker expressions were used to render additional evidence for the feature-associated subcluster annotation. C0A in C3N-00148 showed significantly higher expressions in TIMP1, C1R, and TGFBI (FIGS. 10A). The increased expression of TIMPs in RCC was reported to be correlated with sarcomatoid RCCs (Kallakury et al., 2001). To further validate the refined tumor-subpopulation assignments and identify sarcomatoid-associated markers, two additional sarcomatoid cases were sequenced for snRNA-seq integration (FIG. 10B). As C0A overlapped with all sarcomatoid cases, TIMP1, CIR, and TGFBI were highly expressed in C0A at both integration and case levels (FIG. 10C). Two representative cases with high and diffuse staining intensity of TGFBI noted in sarcomatoid area with the absence of staining in conventional nested clear cell area (FIG. 10D). As for the subcluster with rhabdoid features in C3N-01287, it presented higher expression profiles of KIF2A, NAMPT, and GALNT2 (FIG. 10E) and confirmed by another independent case with rhabdoid features (FIGS. 10F-G). Similarly, high TGFBI corresponded to strong staining intensity in sarcomatoid area rather than nested clear cell area (FIG. 10H). Furthermore, most of these markers presented consistent patterns in bulk RNA expression and global protein abundance, such as high KIF2A in rhabdoid cases compared with controls without any high-grade features based on the systematic histopathologic annotation (FIG. 9I).

Methylation Subtype Associated with BAP1 Mutations and Poor Survival

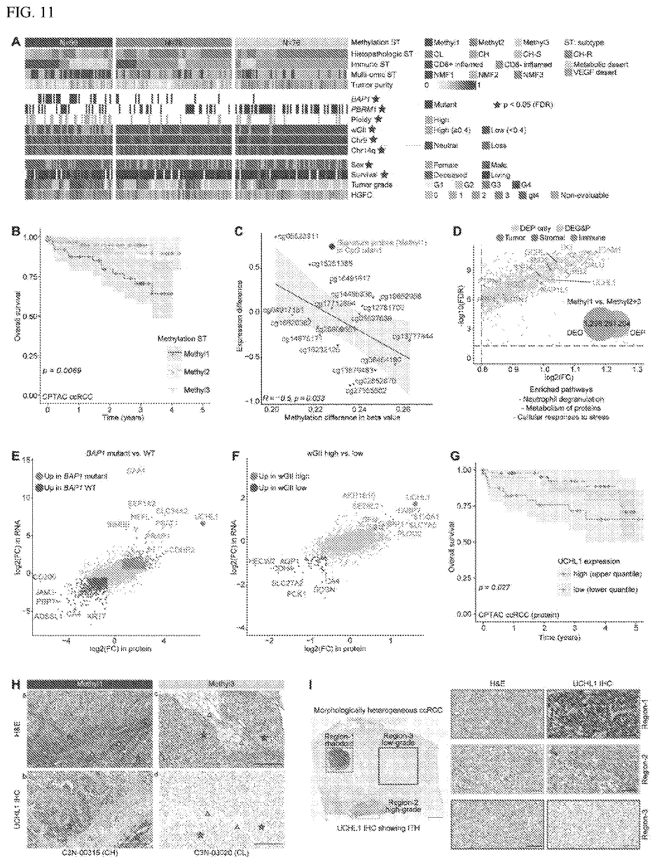

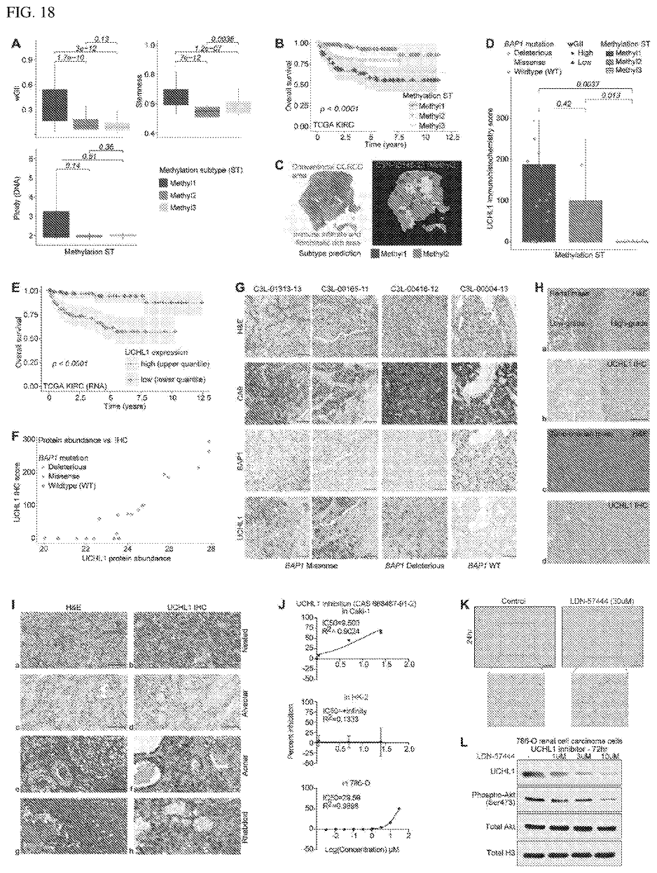

Dysregulation of the epigenetic DNA methylation marks is considered an early event in carcinogenesis (Evelonn et al., 2016; Evelonn et al., 2019; Lasseigne and Brooks, 2018; Malouf et al., 2016) and is of particular significance in RCC. Previous pan-RCC genomic studies have noted a strong association between increased DNA methylation and worse prognosis in ccRCC and more so in papillary RCC (Ricketts et al., 2018). In this regard, proteomic characterization and identification of specific prognostic markers to distinguish this patient subset can now be explored with the extended cohort. To address this unmet clinical need, the tumor samples were first classified into distinct methylation subtypes, examined the role of DNA methylation in ccRCC disease etiology and progression, and defined signatures associated with each of the four histopathologic subtypes. Among the 8,000 most variable CpG sites (probes) that distinguished tumors from NATs, the signature probes and related genes associated with histopathologic subtypes were identified (Methods). For instance, it was noticed that seven probes in the RNF39 CpG island were hypermethylated in CH-S(FDR<0.05 & beta value difference >0.1 & in CpG) as a part of an altered methylation profile. Three methylation subtypes (Methyl1-3) were detected in both CPTAC ccRCC and TCGA KIRC cohorts by applying the consensus clustering on the 8000 probes (FIG. 11A). Methyl1 was significantly associated with samples containing higher tumor grades, higher stemness score, and worse prognosis, as well as metabolic desert followed by CD8+ inflamed. Interestingly, several molecular features that are significantly associated with Methyl1 include high ploidy, high wGII, loss of chr9, 14p, 14q, and mutations of BAP1 (FIGS. 11A-B, 18A-B). Panoptes-based models were trained to predict methylation subtypes based on H&E images (FIG. 18C). The best-performing model achieved a macro-averaged multi-class per-slide area under the receiver-operating characteristic (ROC) curve of 0.836 (95% CI: 0.830-0.841) on the test set. The prediction with the histopathologic annotations was tested. For example, in case C3N-00148, classified into Methyl2, heterogeneous features such that the immune-infiltrate, fibroblastic-rich area was predicted as Methyl3, while the majority of conventional ccRCC areas with marked trabecular change were labeled with Methyl2 were observed (FIG. 18C).

As Methyl1 was significantly associated with worse disease prognosis (FIG. 11B), the differentially methylated (DM) probes were captured in both CPTAC ccRCC and TCGA KIRC cohorts (Cancer Genome Atlas Research, 2013; Ricketts et al., 2018) and prioritized as signature probes if (1) common DM probes were significant in both cohorts; (2) beta value differences were >0.2; (3) probes were located in CpG island followed by shelf and shore regions; and (4) corresponding genes identified as tumor-intrinsic were more highly expressed in tumor/epithelial cells than in immune or stromal cells. In total, 235 common significant DM probes were found in the two cohorts corresponding to 198 genes showing an overall negative correlation (R=−0.5, p=0.033) with their corresponding gene expressions (FIG. 11C). The top signature probes for Methyl1, especially those in CpG islands, include cg04917181 (TSPYL5), cg05523911 (TCHH), cg14875171 (NRXNI), cg16232126 (SLC5A7), and cg25809561 (MYO1D) (FIG. 11C).

To learn the characteristics of each methylation subtype, DE analysis was conducted on both RNA level (differentially expressed genes: DEGs) and protein abundance (DEPs). The Methyl1 subtype showed significant upregulation of 251 species as both DEGs and DEPs, including UCHL1. In addition, 204 markers were significantly up only at the protein level as DEPs contributing to the pathways including cellular responses to stress (FIG. 11D). Methyl3, being enriched with VEGF desert, PBRM1 somatic mutations, and high tumor purity, carried 60 markers as both DEGs and DEPs and 116 additional DEPs contributing to the pathways including glycolysis/gluconeogenesis.

UCHL1, in addition to being enriched in Methyl1, was significantly associated with BAP1 mutants and the wGII-high category based on RNA expression, protein abundance, quantified UCHL1 immunohistochemistry (IHC) score, and IHC staining (FIGS. 11E-F, 18D). UCHL1, a deubiquitinase, could serve as a prognostic marker of ccRCC whose high expression status is associated with worse prognosis in both the CPTAC ccRCC and TCGA KIRC cohorts (FIG. 11G, 18E). 32 representative cases were examined by a panel of IHC markers (UCHL1, BAP1, and CA9) to validate UCHL1 associations with BAP1 mutated and Methyl1 subgroups. IHC-based UCHL1 proteome expression assessment showed a high correlation between quantified UCHL1 protein abundance and UCHL1 IHC score, where BAP1 mutants frequently displayed higher levels of UCHL1 (FIGS. 18F-G). BAP1 IHC is currently used in the clinic as a diagnostic marker to evaluate BAP1 protein loss. In this context, all 14 BAP1 deleterious mutant cases tested showed loss of BAP1 staining, and 12 of these cases were positive for UCHL1. Among the 7 BAP1 missense mutant cases examined, only 3 showed the absence of BAP1, and these 3 were positive for UCHL1. Of the 4 missense mutants that displayed BAP1 positivity, only one was UCHL1 positive (FIG. 18D). The ccRCC clinical diagnostic marker CA9 was positive in all the ccRCC cases evaluated (FIG. 18G). When these data were analyzed for methylation subtypes, 68.7% (11/16) of Methyl1 showed UCHL1 positivity and was significantly different from the Methyl3 group. In addition, UCHL1 staining of a matched RCC primary (renal mass) and metastatic RCC (ovarian tubular mass) tumor from a patient with pathogenic germline BAP1 mutation also showed strong UCHL1 positivity (FIG. 18H). Thus, UCHL1 positivity was associated with BAP1 mutation, wGII high, worse survival, and Methyl1 (FIGS. 11E-H, 18D-H) in a collective manner, making it an important prognostic marker. Examination of an independent RCC primary tumor cohort (n=16) of patients who subsequently developed metastatic RCC disease, indicated that 68% (11/16 cases) of the primary tumors showed strong UCHL1 positivity. This was a dramatic increase compared to the 10-15% cases with UCHL1 expression noted in unenriched RCC primary tumor cohorts (CPTAC and TCGA). Several different histopathologic features were observed in these tumors (FIG. 18I). Finally, UCHL1 staining was characterized topographically in one of the 16 independent clinically aggressive cases which showed morphological heterogeneity, where the rhabdoid nodule and high-grade tumor showed strong and moderate UCHL1 staining, respectively, while the staining was negative in the low-grade clear cell area (FIG. 11I). Hence, it was also possible to demonstrate alignment of UCHL1 expression with ITH. Panoptes-based models were trained to classify BAP1 mutated and WT samples. Overall, the cluster of BAP1 mutated tiles showed higher grade aggressive-looking phenotypes, while the BAP1 WT tiles contained predominantly low-grade tumor components, such as acinar and tubular with areas of hemorrhage and hemosiderin-laden macrophages and hyalinization. Encouraged by the availability of UCHL1 small molecule inhibitor (CAS 668467-91-2, also known as LDN-57444) and studies on its targetability from triple negative breast and neuroendocrine lung cancer models (Shimada et al., 2020), cell viability assays were performed in RCC cell line models. Renal cancer cell lines Caki-1 and 786-0 showed dose-dependent inhibition of cell viability with CAS 668467-91-2, while the normal kidney HK-2 cell line was resistant to the treatment (FIG. 18J; Methods). CAS 668467-91-2 treatment in 786-0 renal cancer cells resulted in altered morphology being elongated and stressed (FIG. 18K). Western blot analysis in 786-O cells demonstrated that UCHL1 inhibition suppressed activation of the Akt signaling pathway in a dose-dependent manner (FIG. 18L).

Key Phosphorylation Signaling Pathways and Kinase-Substrate Interactions in ccRCC

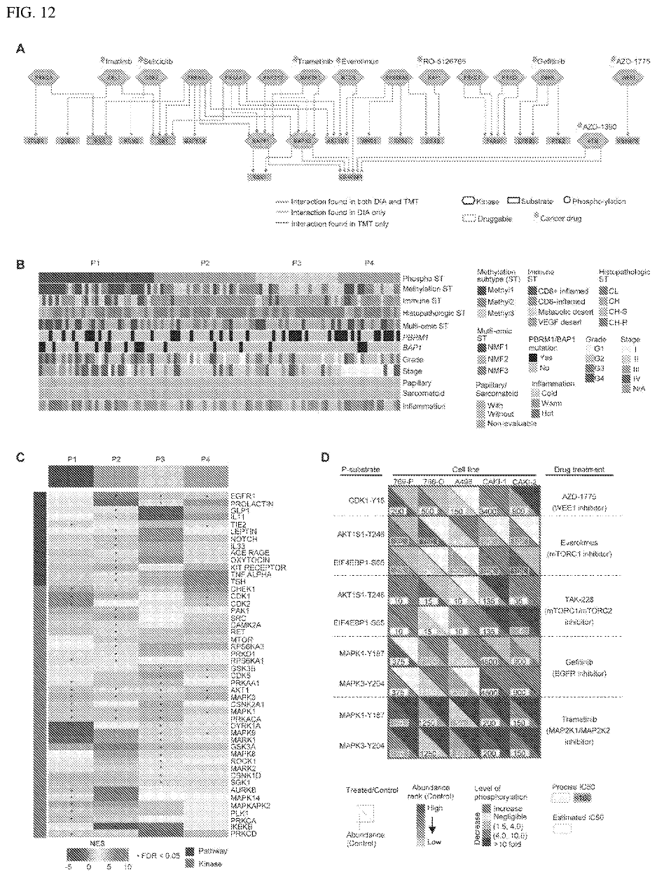

To identify key phosphorylation signaling pathways in ccRCC, altered phosphosignaling networks were investigated based on the association of kinase-substrate (K-S) pairs. The phosphoproteomic data were obtained using two independent methodologies. The dataset comprised DIA-MS analysis of 110 newly added cases and quantitative TMT-based profiling from the initial 103 cases (Clark et al., 2019) (FIG. 12A, 19A). The K-S pairs with the highest phospho-substrate abundance between tumors and NATs from DIA-MS and TMT datasets are shown in FIG. 12A. Approximately 80% of K-S pairs identified from both DIA or TMT-based analysis provided good cross-verification. These K-S pairs included signaling networks involving EGFR, MEK, ERK, and WEE1. Furthermore, it was found that PRKCZ phosphorylation was positively associated with phosphorylation of PARD3, both involved in the Rap1 signaling pathway. Another positive association noted between phosphorylation of RPS6KA3 and RPS6 was of interest as they both were members of the mTOR signaling pathway (FIG. 12A).

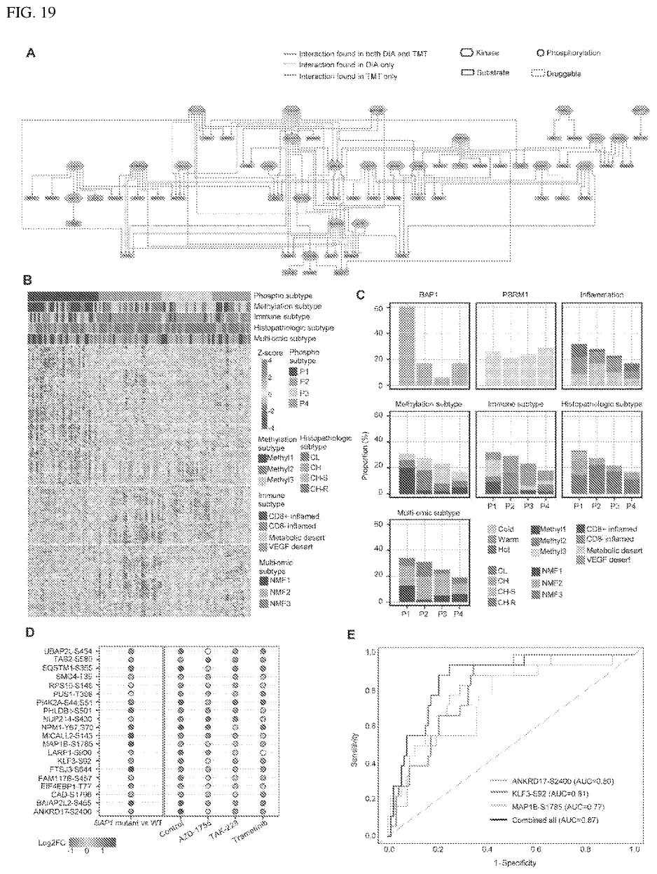

To examine ccRCC inter-tumor phosphoproteomic heterogeneity, an unbiased phosphoproteomic grouping of 110 ccRCC tumors was constructed using the phosphorylation events with coefficient of variation (CV) in >25% quartile. Four major ccRCC phosphoproteomic subtypes emerged from the analysis, which were annotated as P1 to P4 (FIGS. 12B, 19B). Among these subtypes, tumors in P1 had higher grades and stages and were enriched in BAP1 mutation, Methyl1, CD8+ inflamed, and metabolic desert. P2 and P3 had lower grade tumors, with a higher percentage of tumors classified as Methyl2 and VEGF desert, respectively (FIGS. 12B, 19C). P4 showed a more mixed profile. PTM-SEA (Krug et al., 2019) analysis of the tumor phosphoproteomics revealed distinct signatures for the phospho subtypes (FIG. 12C). MAPK14 and its direct downstream kinase, MAPKAPK2, were significantly enriched in P1. MAPK14 and downstream pathways are activated in response to various stresses and inflammation; moreover, MAPK14 activates MAPKAPK2, which is involved in regulating several biological processes, including apoptosis and cell cycle; the role of MAPK14 and MAPKAPK2 in cancer cell survival has been reported in the literature (Koul et al., 2013; Martinez-Limon et al., 2020). On the other hand, activities of some kinases in P2, such as mTOR, SRC, and RPS6KA3, were inferred from the changes of phosphosite abundance. P2 tumors showed phosphosite-driven activation of the leptin pathway, and leptin is associated with ccRCC progression and poor clinical outcome (Fan et al., 2021). The P3 subtype was associated with the EGFR pathway and kinases involved in pathways such as VEGF/angiogenesis signaling (e.g., ROCK1, MAPK3) and focal adhesion (e.g., MAPK9, GSK3B). ROCK may be a target for P3 tumors since P3 is enriched with VEGF-desert samples, and ROCK inhibitors can reduce VEGF-induced angiogenesis (Chen et al., 2014; Liu et al., 2018). Furthermore, P1 and P4 showed enrichment in the TIE2 pathway, whose activity is associated with the activation of MAPK14, ERK1/2, and PI3K/AKT pathways (Kim et al., 2016; Makinde and Agrawal, 2008).

Previous work (Clark et al., 2019) paired case-matched ccRCC tumors and NATs to examine the differentially-expressed K-S pairs. Elevated levels were found in the majority of ccRCC tumors for K-S pairs, such as cell cycle regulator WEE1 and ERK signaling. Similar results were found in the expanded cohort containing 110 new cases. The current study investigates the functional impacts of select kinases using inhibitors focusing on a panel of six K-S pairs prioritized previously (FIG. 12D). Using the DIA-MS approach, the phosphoproteome of 5 RCC cell lines treated with inhibitors targeting MAPK, EGFR, mTOR signaling, and WEE1 was characterized. Results were largely concordant with the predicted mechanisms of action of each kinase. Variations were observed in the inhibitory effects among the cell lines based on the level of phosphorylation of the downstream targeted substrates. Among the five inhibitors, the WEE1 inhibitor (AZD-1775), dual mTOR complex inhibitor (TAK-228), and MEK inhibitor (Trametinib) showed better responses. The WEE1 inhibitor reduced CDK1 phosphorylation levels in all five cell lines, with the highest reduction observed in CAKI-2 relative to the others. TAK-228 reduced phosphorylation of the mTOR complex component, AKT1S1, and its downstream phospho-substrate target, EIF4EBP1, while the MEK inhibitor reduced phosphorylation of both MAPK1 and MAPK3. In contrast, everolimus (mTORC1 inhibitor) and gefitinib (EGFR inhibitor) showed minimal impact on their signaling-related phosphorylation events.

Among the five RCC cell lines, 769-P contains BAP1 and VHL mutations. A broader examination of the expression levels of various phospho-substrates between 769-P cells and the remaining cell lines in comparison to the EXP cohort (FIG. 19D) identified phospho-substrates associated with various biological functions, such as cell cycle (e.g., ANKRD17, SMC4) and DNA binding (e.g., KLF3) that showed distinct expression profiles in the clinical cohort and drug-treated cell lines (FIG. 19D). ANKRD17 is a known interactor of BAP1 protein identified by previous MS studies (Baas et al., 2021). ROC analysis of the phospho-substrates shown in FIG. 19E showed that ANKRD17-S2400, KLF3-S92, and MAP1B-S1785 demonstrated the ability to distinguish BAP1 mutation and wild-type, with the area under the curve (AUC) of 0.80, 0.81, and 0.77, respectively. The AUC was further improved to 0.87 when combining the three phospho-substrates (FIG. 19E). The phosphoproteomic analysis identified multiple signaling pathways activated in tumors and revealed four major phosphoproteomic groups in ccRCC linked to unique K-S pairs. A subsequent kinase inhibition study and ROC analysis indicated targets, especially targets involving MAPK signaling. The MEK inhibitor showed the best performance in reducing phosphorylation of downstream phospho substrates and inducing death at a low IC50 compared to other inhibitors. Taken together, the current results indicated the possibility of expanding treatment options beyond the current FDA-approved therapies targeting VEGF and mTOR (Khetani et al., 2020).

Alteration of Protein Glycosylation Specific to ccRCC and High-Grade ccRCC

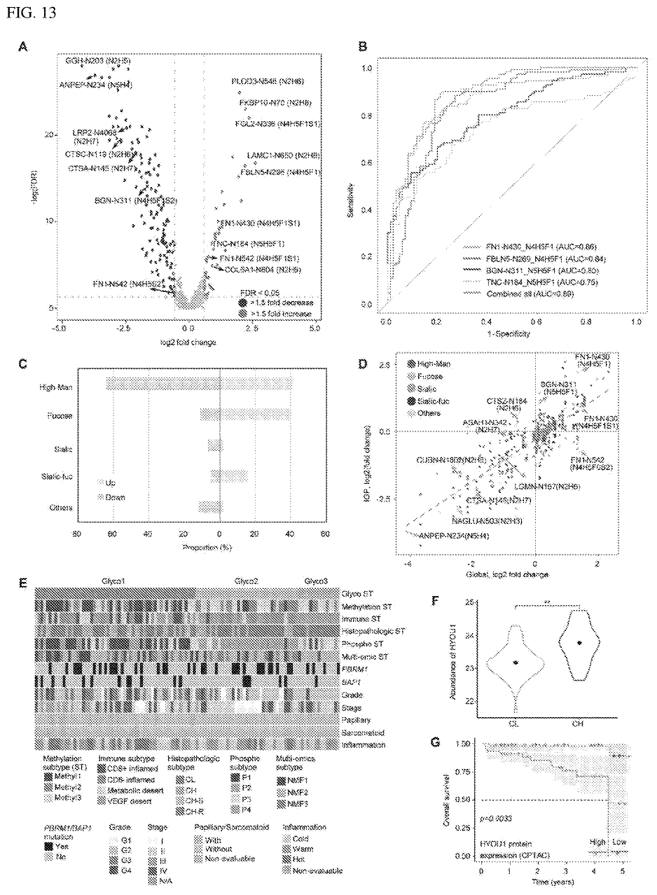

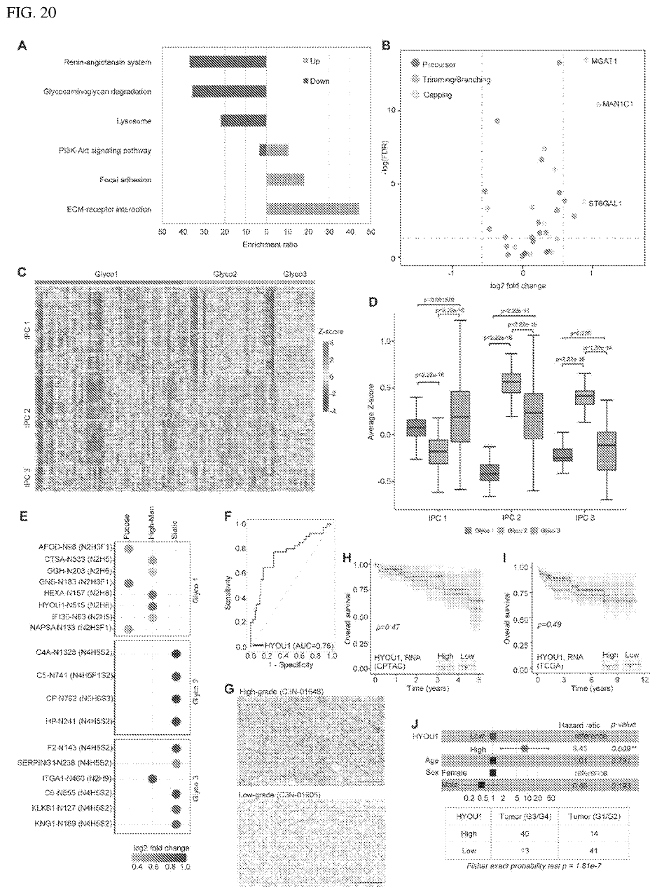

Glycosylation of cell surface receptors is involved in cell signaling, and aberrant glycosylation is associated with cancers (Meany and Chan, 2011; Xu et al., 2021). Glycoproteomic analysis of ccRCC tumors and NATs identified 51 upregulated and 131 downregulated intact glycopeptides with >1.5-fold change (FDR<0.05) in tumors relative to NATs (FIG. 13A). The differentially expressed glycopeptides were used as glyco-signatures to investigate the discrimination power of these signatures for separating tumor from non-tumor samples. As shown in FIG. 13B, four glyco-signatures from four glycoproteins (FN1, FBLN5, BGN, and TNC) demonstrated an ability to differentiate tumor and non-tumor tissues with the AUCs ranging from0.75 to 0.86. Combining the four signatures into a multi-signature panel using logistic regression, the AUC increased to 0.89. KEGG pathway enrichment analysis using significantly altered intact glycopeptides in tumors based on their corresponding genes via WebGestalt (Liao et al., 2019) (FIG. 20A) revealed that ECM-receptor interaction, focal adhesion, and PI3K-Akt signaling pathways were enriched from the glycoproteins of positively-regulated intact glycopeptides. On the other hand, renin-angiotensin system, glycosaminoglycan degradation, and lysosome pathways were enriched from negatively-regulated intact glycopeptides.

According to the monosaccharide composition of the identified glycopeptides, five glycan types were investigated: glycans containing oligomannose (High-Man) only, sialic acid containing glycans (Sialic), glycans containing sialic acid and fucose (Sialic-fuc), fucosylated glycans only (Fucose), and other glycans (Others). As shown in FIG. 13C, Fucose or Sialic-fuc glycans were enriched for the upregulated glycopeptides, whereas most of the downregulated glycopeptides were High-Man, Sialic, or other glycans (FIG. 13C). Furthermore, analysis of the glycoproteins in both protein expression and glycosylation levels in tumors showed that glycopeptide abundance was regulated in both protein level and glycosylation by different glycans when the glycoproteins were analyzed in global and glycopeptide levels in tumors and NATs (FIG. 13D). The alteration of intact glycopeptides was positively correlated to the global protein expression of the corresponding glycoproteins. However, heterogeneities were noted in intact glycopeptide abundances from the same proteins due to different glycan types. Glycans that modify glycoproteins are regulated by glycan biosynthesis enzymes. The altered glycosylation enzymes find use as treatment targets. The levels of glycosylation enzymes were evaluated by comparing tumor to non-tumor tissues at the protein level, revealing upregulated glycosylation enzymes, including MAN1C1, MGAT1, and ST6GAL1, in tumors relative to NATs (FIG. 20B). MAN1C1 and MGAT1 regulate the synthesis of complex glycans, while ST6GAL1 is responsible for transferring sialic acid from CMP-sialic acid to galactose-containing acceptor substrates.

The ccRCC intertumoral heterogeneity based on glycoproteomics was also investigated. Three major ccRCC glycoproteomic subtypes emerged from the analysis (Glyco 1-3, FIG. 13E) with three intact glycopeptide clusters (IPC 1-3, FIG. 20C). Among the three glycoproteomic subtypes, tumors in Glyco 1 were associated with higher grade, BAP1 mutation, Methyl1 subtype, CD8+ inflamed (FIG. 13E), and IPC 1 compared to the other glyco subtypes (FIG. 20D). The significantly upregulated intact glycopeptides in Glyco 1 were mostly occupied by High-Man and Fucose type glycans (FIG. 20E), and there were glycopeptides from glycoproteins (e.g., HYOU1) with influence on metastasis of various cancers (Li et al., 2019b). The comparison between CL and CH tumors indicated that HYOU1 was elevated in CH tumors and may serve as a prognostic marker with an AUC of 0.76 (FIGS. 13F, 20F). By carrying out immunohistochemistry evaluation of HYOU1 expression, higher HYOU1 expression was observed. in high-grade ccRCC compared to low-grade tumors where the strongest signal came from immune cells (FIG. 20G). The association between HYOU1 expression and survival was examined using CPTAC ccRCC and TCGA KIRC cohorts. HYOU1 abundance may serve as a prognostic indicator only at the protein level in the CPTAC cohort but not at the RNA level in both cohorts (FIGS. 13G, 20H-I). HYOU1 protein abundance also showed a significant association with high-grade (G3/G4) tumors (p=1.81e-7, FIG. 20J). Furthermore, Glyco 2 had an association mainly with IPC 2 (FIG. 20D). The significantly upregulated intact glycopeptides in Glyco 2 were occupied by sialylated glycans (FIG. 20E). Since Glyco 2 and 3 were dominated by low-grade and immune-desert tumors, targeted therapy against sialylated glycans provides an alternative approach for Glyco 2 and 3 subtypes.

Metabolic Signatures of High-Grade ccRCC and Low-Grade ccRCC

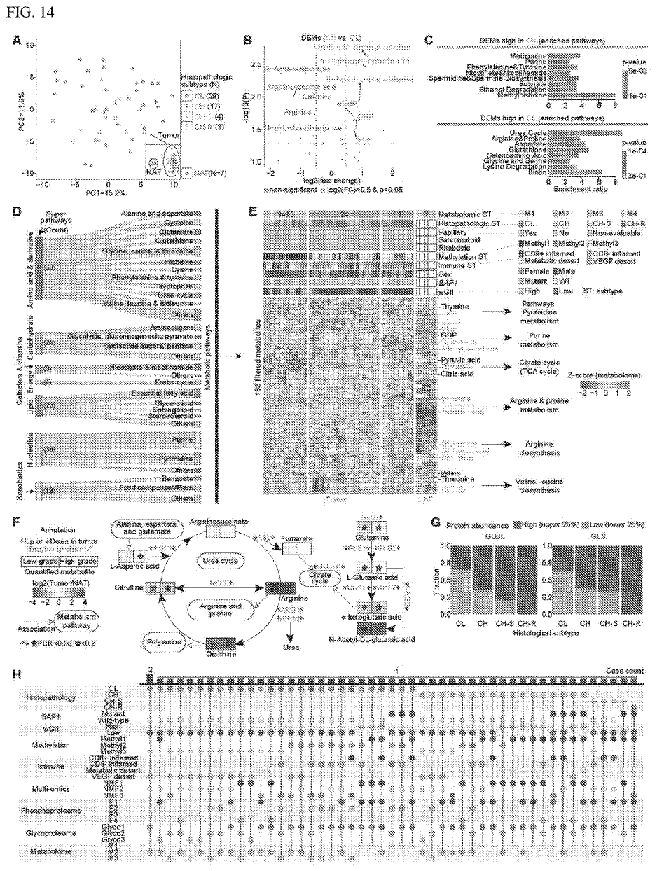

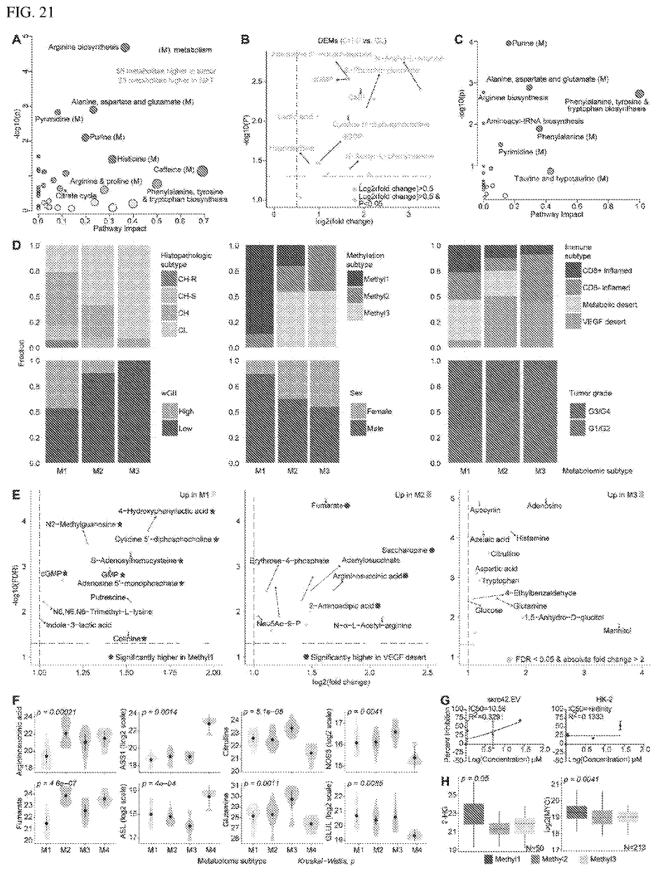

Reprogrammed tumor metabolism is a hallmark of cancers, manifested through alterations in metabolite abundances and composition (Hakimi et al., 2016; Linehan et al., 2019). Mutation of genes associated with kidney cancer, such as VHL in ccRCC and FLCN, TFE3, FH, or SDHB in other RCCs, dysregulates the tumor's responses to changes in oxygen, iron, nutrient, or energy levels, thereby ascribing kidney cancer as a metabolic disease (Linehan et al., 2019). In this multiomic study, 250 metabolites with high confidence from 50 ccRCCs and 7 NATs were quantified. The metabolites detected showed good coverage of various metabolic pathways (Methods). PCA analysis found definitive separation between tumors and NATs, and distribution among the 50 tumors by histopathologic subtypes (FIG. 14A). 55 metabolites with significantly higher (FC>2 and FDR<0.05) in abundance were detected in tumors that contributed to arginine biosynthesis, alanine, aspartate and glutamate metabolism, pyrimidine metabolism, and purine metabolism, while 35 were reduced in tumors compared to NATs (FIG. 21A). Further, CH and CL tumors differed dramatically in their metabolic profiles (FIGS. 14A-C). Arginine is known to be used in the biosynthesis of proteins (Wu and Morris, 1998) and it was low in tumors compared with NATs; it was differentially expressed among tumors, being significantly high in CL (FIG. 14B). The top 10 enriched pathways also displayed distinct patterns between CH and CL tumors (FIG. 14C). CL was used as a control group to capture the metabolic signature associated with CH-S(N=4 with the sarcomatoid feature) (FIGS. 21B-C). Differentially expressed metabolites (DEMs) such as GMP, N-acetyl-L-phenylalanine, and dGMP are high in CH, whereas N-acetyl-L-tyrosine, inosine, and hypoxanthine were elevated in CH-S tumors (FIG. 21B). To identify distinct high- and low-grade subsets associated with molecular and histological features, four well-defined metabolomic subtypes (M1-M4) shown in FIGS. 14D-E were defined. M4 represented the 7 NATs, while the other three subtypes delineated the tumors. Specifically, M1 was significantly enriched with high-grade histopathologic subtypes (CH, CH-S, and CH-R), Methyl1, BAP1 mutants, wGII-high status, a mostly mutual exclusivity from the VEGF desert, and female patients (FIGS. 14E, 21D). The three metabolomic subtypes related to tumors with similar features using the validation set were consistently observed. DEMs were investigated among the three metabolomic subtypes. As Methyl1 was significantly enriched in M1, a considerable overlap of M1-associated and Methyl-associated metabolites such as 4-Hydroxyphenyllactic acid was found (FIG. 21E). Using a combination of metabolomic, genomic, transcriptomic, and proteomic analyses, the expression of metabolites and their enzymes, and their associations with pathways, molecular and histopathologic features, and clinical information was connected. Dramatic changes in arginine and proline metabolism, including arginine biosynthesis and urea cycle for both metabolites and related enzymes were detected (FIG. 14F). Glutamine, a-ketoglutaric acid, ornithine, and citrulline were significantly high in tumors, while L-glutamic acid, N-acetyl-DL-glutamic acid, and argininosuccinic acid were higher in NATs (FIG. 14F). Correspondingly, homologous trends were revealed between metabolites and their enzymes, such as elevated GLUL and reduced ASS1 in tumors (FIGS. 14F, 21F). This agreed well with the previous study showing that argininosuccinate synthase 1 (ASS1), was strongly repressed in ccRCCs compared with nontumorous kidney tissues, and re-expression of ASS1 in ccRCC xenograft models reduced tumor growth (Khare et al., 2021; Ochocki et al., 2018; Wettersten et al., 2017). Glutamine synthetase (GLUL) catalyzed the synthesis of glutamine from glutamate and ammonia (Yang et al., 2014). Higher fractions of GLUL-high and GLS-high samples were seen in the higher-grade tumors (CH, CH—S, CH-R) (FIG. 8G). Given that inhibition of glutaminolysis in combination with other therapies can improve cancer treatment, GLUL may serve as a therapeutic target (Shen et al., 2021). By performing inhibitor treatment (L-Methionine sulfoximine) and a cell viability assay on GLUL, skrc42.EV responded to the anti-GLUL treatment, while the normal kidney HK-2 cell line was not sensitive to the treatment (FIG. 21G). Moreover, tumors among the 3 metabolomic subtypes (M1-3) displayed a strong heterogeneity. Argininosuccinic acid and Fumarate were significantly elevated in M2, whereas Citrulline and Glutamine were high in M3 (FIG. 21F), showing that the inhibition of glutaminolysis in combination with other therapies may be more effective for patients in a certain metabolomic subtype (FIG. 21F). MYC-driven accumulation of 2-hydroxyglutarate (2-HG) was reported to be associated with breast cancer prognosis (Terunuma et al., 2014), and 2-HG and MYC expression were significantly increased in Methyl1, with a worse prognosis (FIG. 21H). Based on the comprehensive characterization depicting all available omics layers, it was found that 48 of 50 tumors presented unique characterization profiles (FIG. 8H). Such integrative histological and proteogenomic profiling helps understand the strong intertumoral heterogeneity in ccRCC.

### Example 2 References

1. Hsieh, J. J., Purdue, M. P., Signoretti, S., Swanton, C., Albiges, L., Schmidinger, M., Heng, D. Y., Larkin, J., and Ficarra, V. (2017). Renal cell carcinoma. Nat Rev Dis Primers 3, 17009. 10.1038/nrdp.2017.9.
2. Siegel, R. L., Miller, K. D., Fuchs, H. E., and Jemal, A. (2021). Cancer Statistics, 2021. CA Cancer J Clin 71, 7-33. 10.3322/caac.21654.
3. Motzer, R. J., Jonasch, E., Boyle, S., Carlo, M. I., Manley, B., Agarwal, N., Alva, A., Beckermann, K., Choueiri, T. K., Costello, B. A., et al. (2020). NCCN Guidelines Insights: Kidney Cancer, Version 1.2021. J Natl Compr Canc Netw 18, 1160-1170. 10.6004/jnccn.2020.0043.
4. Blanco, A. I., Teh, B. S., and Amato, R. J. (2011). Role of radiation therapy in the management of renal cell cancer. Cancers (Basel) 3, 4010-4023. 10.3390/cancers3044010.
5. Diamond, E., Molina, A. M., Carbonaro, M., Akhtar, N. H., Giannakakou, P., Tagawa, S. T., and Nanus, D. M. (2015). Cytotoxic chemotherapy in the treatment of advanced renal cell carcinoma in the era of targeted therapy. Crit Rev Oncol Hematol 96, 518-526. 10.1016/j.critrevonc.2015.08.007.
6. Hsieh, J. J., Le, V. H., Oyama, T., Ricketts, C. J., Ho, T. H., and Cheng, E. H. (2018). Chromosome 3p Loss-Orchestrated VHL, HIF, and Epigenetic Deregulation in Clear Cell Renal Cell Carcinoma. J Clin Oncol, JCO2018792549. 10.1200/JCO.2018.79.2549.
7. Atkins, M. B., and Tannir, N. M. (2018). Current and emerging therapies for first-line treatment of metastatic clear cell renal cell carcinoma. Cancer Treat Rev 70, 127-137. 10.1016/j.ctrv.2018.07.009.
8. Choueiri, T. K., and Motzer, R. J. (2017). Systemic Therapy for Metastatic Renal-Cell Carcinoma. N Engl J Med 376, 354-366. 10.1056/NEJMra1601333.
9. Powles, T., Plimack, E. R., Soulieres, D., Waddell, T., Stus, V., Gafanov, R., Nosov, D., Pouliot, F., Melichar, B., Vynnychenko, I., et al. (2020). Pembrolizumab plus axitinib versus sunitinib monotherapy as first-line treatment of advanced renal cell carcinoma (KEYNOTE-426): extended follow-up from a randomised, open-label, phase 3 trial. Lancet Oncol 21, 1563-1573. 10.1016/S1470-2045 (20) 30436-8.
10. Sanchez, D. J., and Simon, M. C. (2018). Genetic and metabolic hallmarks of clear cell renal cell carcinoma. Biochim Biophys Acta Rev Cancer 1870, 23-31. 10.1016/j.bbcan.2018.06.003.
11. Sato, Y., Yoshizato, T., Shiraishi, Y., Maekawa, S., Okuno, Y., Kamura, T., Shimamura, T., Sato-Otsubo, A., Nagae, G., Suzuki, H., et al. (2013). Integrated molecular analysis of clear-cell renal cell carcinoma. Nat Genet 45, 860-867. 10.1038/ng.2699.
12. Mitchell, T. J., Turajlic, S., Rowan, A., Nicol, D., Farmery, J. H. R., O'Brien, T., Martincorena, I., Tarpey, P., Angelopoulos, N., Yates, L. R., et al. (2018). Timing the Landmark Events in the Evolution of Clear Cell Renal Cell Cancer: TRACERx Renal. Cell 173, 611-623 e617. 10.1016/j.cell.2018.02.020.
13. Clark, D. J., Dhanasekaran, S. M., Petralia, F., Pan, J., Song, X., Hu, Y., da Veiga Leprevost, F., Reva, B., Lih, T. M., Chang, H. Y., et al. (2019). Integrated Proteogenomic Characterization of Clear Cell Renal Cell Carcinoma. Cell 179, 964-983 e931. 10.1016/j.cell.2019.10.007.
14. Cancer Genome Atlas Research, N., Ley, T. J., Miller, C., Ding, L., Raphael, B. J., Mungall, A. J., Robertson, A., Hoadley, K., Triche, T. J., Jr., Laird, P. W., et al. (2013). Genomic and epigenomic landscapes of adult de novo acute myeloid leukemia. N Engl J Med 368, 2059-2074. 10.1056/NEJMoa1301689.
15. Hakimi, A. A., Ostrovnaya, I., Reva, B., Schultz, N., Chen, Y. B., Gonen, M., Liu, H., Takeda, S., Voss, M. H., Tickoo, S. K., et al. (2013). Adverse outcomes in clear cell renal cell carcinoma with mutations of 3p21 epigenetic regulators BAP1 and SETD2: a report by MSKCC and the KIRC TCGA research network. Clin Cancer Res 19, 3259-3267. 10.1158/1078-0432.CCR-12-3886.
16. Kapur, P., Pena-Llopis, S., Christie, A., Zhrebker, L., Pavia-Jimenez, A., Rathmell, W. K., Xie, X. J., and Brugarolas, J. (2013). Effects on survival of BAP1 and PBRM1 mutations in sporadic clear-cell renal-cell carcinoma: a retrospective analysis with independent validation. Lancet Oncol 14, 159-167. 10.1016/S1470-2045 (12) 70584-3.
17. Ricketts, C. J., De Cubas, A. A., Fan, H., Smith, C. C., Lang, M., Reznik, E., Bowlby, R., Gibb, E. A., Akbani, R., Beroukhim, R., et al. (2018). The Cancer Genome Atlas Comprehensive Molecular Characterization of Renal Cell Carcinoma. Cell Rep 23, 313-326 e315. 10.1016/j.celrep.2018.03.075.
18. Hakimi, A. A., Reznik, E., Lee, C. H., Creighton, C. J., Brannon, A. R., Luna, A., Aksoy, B. A., Liu, E. M., Shen, R., Lee, W., et al. (2016). An Integrated Metabolic Atlas of Clear Cell Renal Cell Carcinoma. Cancer Cell 29, 104-116. 10.1016/j.ccell.2015.12.004.
19. Senbabaoglu, Y., Gejman, R. S., Winer, A. G., Liu, M., Van Allen, E. M., de Velasco, G., Miao, D., Ostrovnaya, I., Drill, E., Luna, A., et al. (2016). Tumor immune microenvironment characterization in clear cell renal cell carcinoma identifies prognostic and immunotherapeutically relevant messenger RNA signatures. Genome Biol 17, 231. 10.1186/s13059-016-1092-z.
20. Rooney, M. S., Shukla, S. A., Wu, C. J., Getz, G., and Hacohen, N. (2015). Molecular and genetic properties of tumors associated with local immune cytolytic activity. Cell 160, 48-61. 10.1016/j.cell.2014.12.033.
21. Gerlinger, M., Rowan, A. J., Horswell, S., Math, M., Larkin, J., Endesfelder, D., Gronroos, E., Martinez, P., Matthews, N., Stewart, A., et al. (2012). Intratumor heterogeneity and branched evolution revealed by multiregion sequencing. N Engl J Med 366, 883-892. 10.1056/NEJMoa1113205.
22. Gerlinger, M., Horswell, S., Larkin, J., Rowan, A. J., Salm, M. P., Varela, I., Fisher, R., McGranahan, N., Matthews, N., Santos, C. R., et al. (2014). Genomic architecture and evolution of clear cell renal cell carcinomas defined by multiregion sequencing. Nat Genet 46, 225-233. 10.1038/ng.2891.
23. Turajlic, S., Xu, H., Litchfield, K., Rowan, A., Horswell, S., Chambers, T., O'Brien, T., Lopez, J. I., Watkins, T.B.K., Nicol, D., et al. (2018). Deterministic Evolutionary Trajectories Influence Primary Tumor Growth: TRACERx Renal. Cell 173, 595-610 e511. 10.1016/j.cell.2018.03.043.
24. Zhang, Y., Narayanan, S. P., Mannan, R., Raskind, G., Wang, X., Vats, P., Su, F., Hosseini, N., Cao, X., Kumar-Sinha, C., et al. (2021). Single-cell analyses of renal cell cancers reveal insights into tumor microenvironment, cell of origin, and therapy response. Proc Natl Acad Sci USA 118. 10.1073/pnas.2103240118.
25. Hu, J., Chen, Z., Bao, L., Zhou, L., Hou, Y., Liu, L., Xiong, M., Zhang, Y., Wang, B., Tao, Z., and Chen, K. (2020). Single-Cell Transcriptome Analysis Reveals Intratumoral Heterogeneity in ccRCC, which Results in Different Clinical Outcomes. Mol Ther 28, 1658-1672. 10.1016/j.ymthe.2020.04.023.
26. Cai, Q., Christie, A., Rajaram, S., Zhou, Q., Araj, E., Chintalapati, S., Cadeddu, J., Margulis, V., Pedrosa, I., Rakheja, D., et al. (2020). Ontological analyses reveal clinically-significant clear cell renal cell carcinoma subtypes with convergent evolutionary trajectories into an aggressive type. EBioMedicine 51, 102526. 10.1016/j.ebiom.2019.10.052.
27. Novara, G., Martignoni, G., Artibani, W., and Ficarra, V. (2007). Grading systems in renal cell carcinoma. J Urol 177, 430-436. 10.1016/j.juro.2006.09.034.
28. Fuhrman, S. A., Lasky, L. C., and Limas, C. (1982). Prognostic significance of morphologic parameters in renal cell carcinoma. Am J Surg Pathol 6, 655-663. 10.1097/00000478-198210000-00007.
29. Verine, J., Colin, D., Nheb, M., Prapotnich, D., Ploussard, G., Cathelineau, X., Desgrandchamps, F., Mongiat-Artus, P., and Feugeas, J. P. (2018). Architectural Patterns are a Relevant Morphologic Grading System for Clear Cell Renal Cell Carcinoma Prognosis Assessment: Comparisons With WHO/ISUP Grade and Integrated Staging Systems. Am J Surg Pathol 42, 423-441. 10.1097/PAS.0000000000001025.
30. Li, X., Shu, K., Wang, Z., and Ding, D. (2019). Prognostic significance of KIF2A and KIF20A expression in human cancer: A systematic review and meta-analysis. Medicine (Baltimore) 98, e18040. 10.1097/MD.0000000000018040.
31. Braun, D. A., Hou, Y., Bakouny, Z., Ficial, M., Sant′ Angelo, M., Forman, J., Ross-Macdonald, P., Berger, A. C., Jegede, O. A., Elagina, L., et al. (2020). Interplay of somatic alterations and immune infiltration modulates response to PD-1 blockade in advanced clear cell renal cell carcinoma. Nat Med 26, 909-918. 10.1038/s41591-020-0839-y.
32. Motzer, R. J., Banchereau, R., Hamidi, H., Powles, T., McDermott, D., Atkins, M. B., Escudier, B., Liu, L. F., Leng, N., Abbas, A. R., et al. (2020). Molecular Subsets in Renal Cancer Determine Outcome to Checkpoint and Angiogenesis Blockade. Cancer Cell 38, 803-817 e804. 10.1016/j.ccell.2020.10.011.
33. Hong, R., Liu, W., DeLair, D., Razavian, N., and Fenyo, D. (2021). Predicting endometrial cancer subtypes and molecular features from histopathology images using multi-resolution deep learning models. Cell Rep Med 2, 100400. 10.1016/j.xcrm.2021.100400.
34. Ito, T., Pei, J., Dulaimi, E., Menges, C., Abbosh, P. H., Smaldone, M. C., Chen, D. Y., Greenberg, R. E., Kutikov, A., Viterbo, R., et al. (2016). Genomic Copy Number Alterations in Renal Cell Carcinoma with Sarcomatoid Features. J Urol 195, 852-858. 10.1016/j.juro.2015.10.180.
35. Kallakury, B. V., Karikehalli, S., Haholu, A., Sheehan, C. E., Azumi, N., and Ross, J. S. (2001). Increased expression of matrix metalloproteinases 2 and 9 and tissue inhibitors of metalloproteinases 1 and 2 correlate with poor prognostic variables in renal cell carcinoma. Clin Cancer Res 7, 3113-3119.
36. Lasseigne, B. N., and Brooks, J. D. (2018). The Role of DNA Methylation in Renal Cell Carcinoma. Mol Diagn Ther 22, 431-442. 10.1007/s40291-018-0337-9.
37. Evelonn, E. A., Degerman, S., Kohn, L., Landfors, M., Ljungberg, B., and Roos, G. (2016). DNA methylation status defines clinicopathological parameters including survival for patients with clear cell renal cell carcinoma (ccRCC). Tumour Biol 37, 10219-10228. 10.1007/s13277-016-4893-5.
38. Evelonn, E. A., Landfors, M., Haider, Z., Kohn, L., Ljungberg, B., Roos, G., and Degerman, S. (2019). DNA methylation associates with survival in non-metastatic clear cell renal cell carcinoma. BMC Cancer 19, 65. 10.1186/s12885-019-5291-3.
39. Malouf, G. G., Su, X., Zhang, J., Creighton, C. J., Ho, T. H., Lu, Y., Raynal, N.J., Karam, J. A., Tamboli, P., Allanick, F., et al. (2016). DNA Methylation Signature Reveals Cell Ontogeny of Renal Cell Carcinomas. Clin Cancer Res 22, 6236-6246. 10.1158/1078-0432.CCR-15-1217.
40. Bell, H. N., Kumar-Sinha, C., Mannan, R., Zakalik, D., Zhang, Y., Mehra, R., Jagtap, D., Dhanasekaran, S. M., and Vaishampayan, U. (2022). Pathogenic ATM and BAP1 germline mutations in a case of early-onset, familial sarcomatoid renal cancer. Cold Spring Harb Mol Case Stud 8. 10.1101/mcs.a006203.
41. Shimada, Y., Kudo, Y., Maehara, S., Matsubayashi, J., Otaki, Y., Kajiwara, N., Ohira, T., Minna, J. D., and Ikeda, N. (2020). Ubiquitin C-terminal hydrolase-L1 has prognostic relevance and is a therapeutic target for high-grade neuroendocrine lung cancers. Cancer Sci 111, 610-620. 10.1111/cas.14284.
42. Krug, K., Mertins, P., Zhang, B., Hornbeck, P., Raju, R., Ahmad, R., Szucs, M., Mundt, F., Forestier, D., Jane-Valbuena, J., et al. (2019). A Curated Resource for Phosphositespecific Signature Analysis. Mol Cell Proteomics 18, 576-593. 10.1074/mcp.TIR118.000943. 43. Koul, H. K., Pal, M., and Koul, S. (2013). Role of p38 MAP Kinase Signal Transduction in Solid Tumors. Genes Cancer 4, 342-359. 10.1177/1947601913507951.
44. Martinez-Limon, A., Joaquin, M., Caballero, M., Posas, F., and de Nadal, E. (2020). The p38 Pathway: From Biology to Cancer Therapy. Int J Mol Sci 21. 10.3390/ijms21061913.
45. Fan, W. L., Yeh, Y. M., Liu, T. T., Lin, W. M., Yang, T. Y., Lee, C. W., and Lin, T. C. (2021). Leptin Is Associated with Poor Clinical Outcomes and Promotes Clear Cell Renal Cell Carcinoma Progression. Biomolecules 11. 10.3390/biom11030431.
46. Liu, J., Wada, Y., Katsura, M., Tozawa, H., Erwin, N., Kapron, C. M., Bao, G., and Liu, J. (2018). Rho-Associated Coiled-Coil Kinase (ROCK) in Molecular Regulation of Angiogenesis. Theranostics 8, 6053-6069. 10.7150/thno.30305.
47. Chen, W., Mao, K., Liu, Z., and Dinh-Xuan, A. T. (2014). The role of the RhoA/Rho kinase pathway in angiogenesis and its potential value in prostate cancer (Review). Oncol Lett 8, 1907-1911. 10.3892/ol.2014.2471.
48. Makinde, T., and Agrawal, D. K. (2008). Intra and extravascular transmembrane signalling of angiopoietin-1-Tie2 receptor in health and disease. J Cell Mol Med 12, 810-828. 10.1111/j.1582-4934.2008.00254.x.
49. Kim, M., Allen, B., Korhonen, E. A., Nitschke, M., Yang, H. W., Baluk, P., Saharinen, P., Alitalo, K., Daly, C., Thurston, G., and McDonald, D. M. (2016). Opposing actions of angiopoietin-2 on Tie2 signaling and FOXO1 activation. J Clin Invest 126, 3511-3525. 10.1172/JCI84871.
50. Baas, R., F, J.v.d.W., Bleijerveld, O. B., van Attikum, H., and Sixma, T. K. (2021). Proteomic analysis identifies novel binding partners of BAP1. PLOS One 16, e0257688. 10.1371/journal.pone.0257688.
51. Khetani, V. V., Portal, D. E., Shah, M. R., Mayer, T., and Singer, E. A. (2020). Combination drug regimens for metastatic clear cell renal cell carcinoma. World J Clin Oncol 11, 541-562. 10.5306/wjco.v11.i8.541.
52. Xu, Y., Wang, Y., Hoti, N., Clark, D. J., Chen, S. Y., and Zhang, H. (2021). The next “sweet” spot for pancreatic ductal adenocarcinoma: Glycoprotein for early detection. Mass Spectrom Rev, e21748. 10.1002/mas.21748.
53. Meany, D. L., and Chan, D. W. (2011). Aberrant glycosylation associated with enzymes as cancer biomarkers. Clin Proteomics 8, 7. 10.1186/1559-0275-8-7.
54. Li, X., Zhang, N. X., Ye, H. Y., Song, P. P., Chang, W., Chen, L., Wang, Z., Zhang, L., and Wang, N,N. (2019). HYOU1 promotes cell growth and metastasis via activating PI3K/AKT signaling in epithelial ovarian cancer and predicts poor prognosis. Eur Rev Med Pharmacol Sci 23, 4126-4135. 10.26355/eurrev_201901_17914.
55. Linehan, W. M., Schmidt, L. S., Crooks, D. R., Wei, D., Srinivasan, R., Lang, M., and Ricketts, C. J. (2019). The Metabolic Basis of Kidney Cancer. Cancer Discov 9, 1006-1021. 10.1158/2159-8290.CD-18-1354.
56. Wu, G., and Morris, S. M., Jr. (1998). Arginine metabolism: nitric oxide and beyond. Biochem J 336 (Pt 1), 1-17. 10.1042/bj3360001.
57. Ochocki, J. D., Khare, S., Hess, M., Ackerman, D., Qiu, B., Daisak, J. I., Worth, A. J., Lin, N., Lee, P., Xie, H., et al. (2018). Arginase 2 Suppresses Renal Carcinoma Progression via Biosynthetic Cofactor Pyridoxal Phosphate Depletion and Increased Polyamine Toxicity. Cell Metab 27, 1263-1280 e1266. 10.1016/j.cmet.2018.04.009.
58. Wettersten, H. I., Aboud, O. A., Lara, P. N., Jr., and Weiss, R. H. (2017). Metabolic reprogramming in clear cell renal cell carcinoma. Nat Rev Nephrol 13, 410-419. 10.1038/nrneph.2017.59.
59. Khare, S., Kim, L. C., Lobel, G., Doulias, P. T., Ischiropoulos, H., Nissim, I., Keith, B., and Simon, M. C. (2021). ASS1 and ASL suppress growth in clear cell renal cell carcinoma via altered nitrogen metabolism. Cancer Metab 9, 40. 10.1186/s40170-021-00271-8.
60. Shen, Y. A., Chen, C. L., Huang, Y. H., Evans, E. E., Cheng, C. C., Chuang, Y. J., Zhang, C., and Le, A. (2021). Inhibition of glutaminolysis in combination with other therapies to improve cancer treatment. Curr Opin Chem Biol 62, 64-81. 10.1016/j.cbpa.2021.01.006.
61. Terunuma, A., Putluri, N., Mishra, P., Mathe, E. A., Dorsey, T. H., Yi, M., Wallace, T. A., Issaq, H. J., Zhou, M., Killian, J. K., et al. (2014). MYC-driven accumulation of 2-hydroxyglutarate is associated with breast cancer prognosis. J Clin Invest 124, 398-412. 10.1172/JCI71180.
62. Yang, B., Xia, H., Xu, C., Lu, M., Zhang, S., Wang, G., and Ma, L. (2020). Impact of sarcomatoid differentiation and rhabdoid differentiation on prognosis for renal cell carcinoma with vena caval tumour thrombus treated surgically. BMC Urol 20, 14. 10.1186/s12894-020-0584-z.
63. Li, L., Shen, C., Nakamura, E., Ando, K., Signoretti, S., Beroukhim, R., Cowley, G. S., Lizotte, P., Liberzon, E., Bair, S., et al. (2013). SQSTM1 is a pathogenic target of 5q copy number gains in kidney cancer. Cancer Cell 24, 738-750. 10.1016/j.ccr.2013.10.025.
64. Porubsky, S., Nientiedt, M., Kriegmair, M. C., Siemoneit, J. H., Sandhoff, R., Jennemann, R., Borgmann, H., Gaiser, T., Weis, C. A., Erben, P., et al. (2021). The prognostic value of galactosylceramide-sulfotransferase (Gal3ST1) in human renal cell carcinoma. Sci Rep 11, 10926. 10.1038/s41598-021-90381-6.
65. Baba, M., Furuya, M., Motoshima, T., Lang, M., Funasaki, S., Ma, W., Sun, H. W., Hasumi, H., Huang, Y., Kato, I., et al. (2019). TFE3 Xp11.2 Translocation Renal Cell Carcinoma Mouse Model Reveals Novel Therapeutic Targets and Identifies GPNMB as a Diagnostic Marker for Human Disease. Mol Cancer Res 17, 1613-1626. 10.1158/1541-7786.MCR-18-1235.
66. Zhai, J. P., Liu, Z. H., Wang, H. D., Huang, G. L., and Man, L. B. (2022). GPNMB overexpression is associated with extensive bone metastasis and poor prognosis in renal cell carcinoma. Oncol Lett 23, 36. 10.3892/ol.2021.13154.
67. Ott, P. A., Hamid, O., Pavlick, A. C., Kluger, H., Kim, K. B., Boasberg, P. D., Simantov, R., Crowley, E., Green, J. A., Hawthorne, T., et al. (2014). Phase I/II study of the antibodydrug conjugate glembatumumab vedotin in patients with advanced melanoma. J Clin Oncol 32, 3659-3666. 10.1200/JCO.2013.54.8115.
68. Tray, N., Adams, S., and Esteva, F. J. (2018). Antibody-drug conjugates in triple negative breast cancer. Future Oncol 14, 2651-2661. 10.2217/fon-2018-0131.
69. Koneru, R., and Hotte, S. J. (2009). Role of cytokine therapy for renal cell carcinoma in the era of targeted agents. Curr Oncol 16 Suppl 1, S40-44. 10.3747/co.v1610.417.
70. Bakouny, Z., Braun, D. A., Shukla, S. A., Pan, W., Gao, X., Hou, Y., Flaifel, A., Tang, S., Bosma-Moody, A., He, M. X., et al. (2021). Integrative molecular characterization of sarcomatoid and rhabdoid renal cell carcinoma. Nat Commun 12, 808. 10.1038/s41467-021-21068-9.
71. Chen, W., Hill, H., Christie, A., Kim, M. S., Holloman, E., Pavia-Jimenez, A., Homayoun, F., Ma, Y., Patel, N., Yell, P., et al. (2016). Targeting renal cell carcinoma with a HIF-2 antagonist. Nature 539, 112-117. 10.1038/nature19796.
72. Zhang, H., Liu, T., Zhang, Z., Payne, S. H., Zhang, B., McDermott, J. E., Zhou, J. Y., Petyuk, V. A., Chen, L., Ray, D., et al. (2016). Integrated Proteogenomic Characterization of Human High-Grade Serous Ovarian Cancer. Cell 166, 755-765. 10.1016/j.cell.2016.05.069.
73. Jonasch, E., Donskov, F., Iliopoulos, O., Rathmell, W. K., Narayan, V. K., Maughan, B. L., Oudard, S., Else, T., Maranchie, J. K., Welsh, S. J., et al. (2021). Belzutifan for Renal Cell Carcinoma in von Hippel-Lindau Disease. N Engl J Med 385, 2036-2046. 10.1056/NEJMoa2103425.
74. Chen, X., Pan, X., Zhang, W., Guo, H., Cheng, S., He, Q., Yang, B., and Ding, L. (2020). Epigenetic strategies synergize with PD-L1/PD-1 targeted cancer immunotherapies to enhance antitumor responses. Acta Pharm Sin B 10, 723-733. 10.1016/j.apsb.2019.09.006.
75. Diaz-Montero, C. M., Mao, F. J., Barnard, J., Parker, Y., Zamanian-Daryoush, M., Pink, J. J., Finke, J. H., Rini, B. I., and Lindner, D. J. (2016). MEK inhibition abrogates sunitinib resistance in a renal cell carcinoma patient-derived xenograft model. Br J Cancer 115, 920-928. 10.1038/bjc.2016.263.
76. Chauhan, A., Semwal, D. K., Mishra, S. P., Goyal, S., Marathe, R., and Semwal, R. B. (2016). Combination of mTOR and MAPK Inhibitors-A Potential Way to Treat Renal Cell Carcinoma. Med Sci (Basel) 4. 10.3390/medsci4040016.
77. Voss, M. H., Gordon, M. S., Mita, M., Rini, B., Makker, V., Macarulla, T., Smith, D. C., Cervantes, A., Puzanov, I., Pili, R., et al. (2020). Phase 1 study of mTORC1/2 inhibitor sapanisertib (TAK-228) in advanced solid tumours, with an expansion phase in renal, endometrial or bladder cancer. Br J Cancer 123, 1590-1598. 10.1038/s41416-020-01041-x.
78. Hsieh, C. C., Shyr, Y. M., Liao, W. Y., Chen, T. H., Wang, S. E., Lu, P. C., Lin, P. Y., Chen, Y. B., Mao, W. Y., Han, H. Y., et al. (2017). Elevation of beta-galactoside alpha2,6-sialyltransferase 1 in a fructoseresponsive manner promotes pancreatic cancer metastasis. Oncotarget 8, 7691-7709. 10.18632/oncotarget.13845.
79. Suzuki, O. (2019). Glycosylation in lymphoma: Biology and glycotherapy. Pathol Int 69, 441-449. 10.1111/pin.12834.
80. Vajaria, B. N., Patel, K. R., Begum, R., and Patel, P. S. (2016). Sialylation: an Avenue to Target Cancer Cells. Pathol Oncol Res 22, 443-447. 10.1007/s12253-015-0033-6.
81. Garnham, R., Scott, E., Livermore, K. E., and Munkley, J. (2019). ST6GAL1: A key player in cancer. Oncol Lett 18, 983-989. 10.3892/ol.2019.10458.
82. Chakraborty, S., Balan, M., Sabarwal, A., Choueiri, T. K., and Pal, S. (2021). Metabolic reprogramming in renal cancer: Events of a metabolic disease. Biochim Biophys Acta Rev Cancer 1876, 188559. 10.1016/j.bbcan.2021.188559.
83. Cancer Genome Atlas Research, N. (2013). Comprehensive molecular characterization of clear cell renal cell carcinoma. Nature 499, 43-49. 10.1038/nature12222.
84. Turei, D., Korcsmaros, T., and Saez-Rodriguez, J. (2016). OmniPath: guidelines and gateway for literature-curated signaling pathway resources. Nat Methods 13, 966-967. 10.1038/nmeth.4077.
85. Linding, R., Jensen, L. J., Pasculescu, A., Olhovsky, M., Colwill, K., Bork, P., Yaffe, M. B., and Pawson, T. (2008). NetworKIN: a resource for exploring cellular phosphorylation networks. Nucleic Acids Res 36, D695-699. 10.1093/nar/gkm902.
86. Damle, N. P., and Kohn, M. (2019). The human DEPhosphorylation Database DEPOD: 2019 update. Database (Oxford) 2019. 10.1093/database/baz133.
87. Licata, L., Lo Surdo, P., Iannuccelli, M., Palma, A., Micarelli, E., Perfetto, L., Peluso, D., Calderone, A., Castagnoli, L., and Cesareni, G. (2020). SIGNOR 2.0, the SIGnaling Network Open Resource 2.0:2019 update. Nucleic Acids Res 48, D504-D510. 10.1093/nar/gkz949.
88. Li, H., and Durbin, R. (2009). Fast and accurate short read alignment with Burrows-Wheeler transform. Bioinformatics 25, 1754-1760. 10.1093/bioinformatics/btp324.
89. Scott, A. D., Huang, K. L., Weerasinghe, A., Mashl, R. J., Gao, Q., Martins Rodrigues, F., Wyczalkowski, M. A., and Ding, L. (2019). CharGer: clinical Characterization of Germline variants. Bioinformatics 35, 865-867. 10.1093/bioinformatics/bty649.
90. Wilkerson, M. D., and Hayes, D. N. (2010). ConsensusClusterPlus: a class discovery tool with confidence assessments and item tracking. Bioinformatics 26, 1572-1573. 10.1093/bioinformatics/btq170.
91. Alexandrov, L. B., Kim, J., Haradhvala, N.J., Huang, M. N., Tian Ng, A. W., Wu, Y., Boot, A., Covington, K. R., Gordenin, D. A., Bergstrom, E. N., et al. (2020). The repertoire of mutational signatures in human cancer. Nature 578, 94-101. 10.1038/s41586-020-1943-3.
92. Sun, S. Q., Mashl, R. J., Sengupta, S., Scott, A. D., Wang, W., Batra, P., Wang, L. B., Wyczalkowski, M. A., and Ding, L. (2018). Database of evidence for precision oncology portal. Bioinformatics 34, 4315-4317. 10.1093/bioinformatics/bty531.
93. Benelli, M., Pescucci, C., Marseglia, G., Severgnini, M., Torricelli, F., and Magi, A. (2012). Discovering chimeric transcripts in paired-end RNA-seq data by using EricScript. Bioinformatics 28, 3232-3239. 10.1093/bioinformatics/bts617.
94. Anders, S., Pyl, P. T., and Huber, W. (2015). HTSeq—a Python framework to work with high-throughput sequencing data. Bioinformatics 31, 166-169. 10.1093/bioinformatics/btu638.
95. Zhang, J., White, N. M., Schmidt, H. K., Fulton, R. S., Tomlinson, C., Warren, W. C., Wilson, R. K., and Maher, C. A. (2016). INTEGRATE: gene fusion discovery using whole genome and transcriptome data. Genome Res 26, 108-118. 10.1101/gr.186114.114.
96. Chen, X., Schulz-Trieglaff, O., Shaw, R., Barnes, B., Schlesinger, F., Kallberg, M., Cox, A. J., Kruglyak, S., and Saunders, C. T. (2016). Manta: rapid detection of structural variants and indels for germline and cancer sequencing applications. Bioinformatics 32, 1220-1222. 10.1093/bioinformatics/btv710.
97. Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nat Biotechnol 31, 213-219. 10.1038/nbt.2514.
98. Ye, K., Schulz, M. H., Long, Q., Apweiler, R., and Ning, Z. (2009). Pindel: a pattern growth approach to detect break points of large deletions and medium sized insertions from paired-end short reads. Bioinformatics 25, 2865-2871. 10.1093/bioinformatics/btp394.
99. Polpitiya, A. D., Qian, W. J., Jaitly, N., Petyuk, V. A., Adkins, J. N., Camp, D. G., 2nd, Anderson, G. A., and Smith, R. D. (2008). DAnTE: a statistical tool for quantitative analysis of-omics data. Bioinformatics 24, 1556-1558. 10.1093/bioinformatics/btn217.
100. Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079. 10.1093/bioinformatics/btp352.
101. Haas, B. J., Dobin, A., Li, B., Stransky, N., Pochet, N., and Regev, A. (2019). Accuracy assessment of fusion transcript detection via read-mapping and de novo fusion transcript assembly-based methods. Genome Biol 20, 213. 10.1186/s13059-019-1842-9.
102. Kim, S., Scheffler, K., Halpern, A. L., Bekritsky, M. A., Noh, E., Kallberg, M., Chen, X., Kim, Y., Beyter, D., Krusche, P., and Saunders, C. T. (2018). Strelka2: fast and accurate calling of germline and somatic variants. Nat Methods 15, 591-594. 10.1038/s41592-018-0051-x.
103. Conway, J. R., Lex, A., and Gehlenborg, N. (2017). UpSetR: an R package for the visualization of intersecting sets and their properties. Bioinformatics 33, 2938-2940. 10.1093/bioinformatics/btx364.
104. Koboldt, D. C., Zhang, Q., Larson, D. E., Shen, D., Mclellan, M. D., Lin, L., Miller, C. A., Mardis, E. R., Ding, L., and Wilson, R. K. (2012). VarScan 2: somatic mutation and copy number alteration discovery in cancer by exome sequencing. Genome Res 22, 568-576. 10.1101/gr.129684.111.
105. Aran, D., Hu, Z., and Butte, A. J. (2017). xCell: digitally portraying the tissue cellular heterogeneity landscape. Genome Biol 18, 220. 10.1186/s13059-017-1349-1.
106. Newman, A. M., Steen, C. B., Liu, C. L., Gentles, A. J., Chaudhuri, A. A., Scherer, F., Khodadoust, M. S., Esfahani, M. S., Luca, B. A., Steiner, D., et al. (2019). Determining cell type abundance and expression from bulk tissues with digital cytometry. Nat Biotechnol 37, 773-782. 10.1038/s41587-019-0114-2.
107. Pang, Z., Chong, J., Zhou, G., de Lima Morais, D. A., Chang, L., Barrette, M., Gauthier, C., Jacques, P. E., Li, S., and Xia, J. (2021). MetaboAnalyst 5.0: narrowing the gap between raw spectra and functional insights. Nucleic Acids Res 49, W388-W396. 10.1093/nar/gkab382.
108. Vasaikar, S. V., Straub, P., Wang, J., and Zhang, B. (2018). LinkedOmics: analyzing multi-omics data within and across 32 cancer types. Nucleic Acids Res 46, D956-D963. 10.1093/nar/gkx1090.
109. Clark, K., Vendt, B., Smith, K., Freymann, J., Kirby, J., Koppel, P., Moore, S., Phillips, S., Maffitt, D., Pringle, M., et al. (2013). The Cancer Imaging Archive (TCIA): maintaining and operating a public information repository. J Digit Imaging 26, 1045-1057. 10.1007/s10278-013-9622-7.
110. Fisher, S., Barry, A., Abreu, J., Minie, B., Nolan, J., Delorey, T. M., Young, G., Fennell, T. J., Allen, A., Ambrogio, L., et al. (2011). A scalable, fully automated process for construction of sequence-ready human exome targeted capture libraries. Genome Biol 12, R1. 10.1186/gb-2011-12-1-r1.
111. Chu, A., Robertson, G., Brooks, D., Mungall, A. J., Birol, I., Coope, R., Ma, Y., Jones, S., and Marra, M. A. (2016). Large-scale profiling of microRNAs for The Cancer Genome Atlas. Nucleic Acids Res 44, e3. 10.1093/nar/gkv808.
112. Mertins, P., Qiao, J. W., Patel, J., Udeshi, N. D., Clauser, K. R., Mani, D. R., Burgess, M. W., Gillette, M. A., Jaffe, J. D., and Carr, S. A. (2013). Integrated proteomic analysis of post-translational modifications by serial enrichment. Nat Methods 10, 634-637. 10.1038/nmeth.2518.
113. Dong, M., Lih, T. M., Ao, M., Hu, Y., Chen, S. Y., Eguez, R. V., and Zhang, H. (2021). Data-Independent Acquisition-Based Mass Spectrometry (DIA-MS) for Quantitative Analysis of Intact N-Linked Glycopeptides. Anal Chem 93, 13774-13782. 10.1021/acs.analchem.1c01659.
114. Cho, K. C., Clark, D. J., Schnaubelt, M., Teo, G. C., Leprevost, F.D.V., Bocik, W., Boja, E. S., Hiltke, T., Nesvizhskii, A. I., and Zhang, H. (2020). Deep Proteomics Using Two Dimensional Data Independent Acquisition Mass Spectrometry. Anal Chem 92, 4217-4225. 10.1021/acs.analchem.9b04418.
115. Mertins, P., Tang, L. C., Krug, K., Clark, D. J., Gritsenko, M. A., Chen, L., Clauser, K. R., Clauss, T. R., Shah, P., Gillette, M. A., et al. (2018). Reproducible workflow for multiplexed deep-scale proteome and phosphoproteome analysis of tumor tissues by liquid chromatography-mass spectrometry. Nat Protoc 13, 1632-1661. 10.1038/s41596-018-0006-9.
116. Nguyen, T., Kirsch, B. J., Asaka, R., Nabi, K., Quinones, A., Tan, J., Antonio, M. J., Camelo, F., Li, T., Nguyen, S., et al. (2019). Uncovering the Role of N-Acetyl-Aspartyl-Glutamate as a Glutamate Reservoir in Cancer. Cell Rep 27, 491-501 e496. 10.1016/j.celrep.2019.03.036.
117. Elgogary, A., Xu, Q., Poore, B., Alt, J., Zimmermann, S. C., Zhao, L., Fu, J., Chen, B., Xia, S., Liu, Y., et al. (2016). Combination therapy with BPTES nanoparticles and metformin targets the metabolic heterogeneity of pancreatic cancer. Proc Natl Acad Sci USA 113, E5328-5336. 10.1073/pnas.1611406113.
118. Udupa, S., Nguyen, S., Hoang, G., Nguyen, T., Quinones, A., Pham, K., Asaka, R., Nguyen, K., Zhang, C., Elgogary, A., et al. (2019). Upregulation of the Glutaminase II Pathway Contributes to Glutamate Production upon Glutaminase 1 Inhibition in Pancreatic Cancer. Proteomics 19, e1800451. 10.1002/pmic.201800451.
119. Skala, S. L., Wang, X., Zhang, Y., Mannan, R., Wang, L., Narayanan, S. P., Vats, P., Su, F., Chen, J., Cao, X., et al. (2020). Next-generation RNA Sequencing-based Biomarker Characterization of Chromophobe Renal Cell Carcinoma and Related Oncocytic Neoplasms. Eur Urol 78, 63-74. 10.1016/j.eururo.2020.03.003.
120. Bailey, M. H., Tokheim, C., Porta-Pardo, E., Sengupta, S., Bertrand, D., Weerasinghe, A., Colaprico, A., Wendl, M. C., Kim, J., Reardon, B., et al. (2018). Comprehensive Characterization of Cancer Driver Genes and Mutations. Cell 173, 371-385 e318. 10.1016/j.cell.2018.02.060. 121. Tate, J. G., Bamford, S., Jubb, H. C., Sondka, Z., Beare, D. M., Bindal, N., Boutselakis, H., Cole, C. G., Creatore, C., Dawson, E., et al. (2019). COSMIC: the Catalogue of Somatic Mutations in Cancer. Nucleic Acids Res 47, D941-D947. 10.1093/nar/gkyl015.
122. McKenna, A., Hanna, M., Banks, E., Sivachenko, A., Cibulskis, K., Kernytsky, A., Garimella, K., Altshuler, D., Gabriel, S., Daly, M., and DePristo, MA. (2010). The Genome Analysis Toolkit: a MapReduce framework for analyzing next-generation DNA sequencing data. Genome Res 20, 1297-1303. 10.1101/gr.107524.110.
123. Karczewski, K. J., Francioli, L. C., Tiao, G., Cummings, B. B., Alfoldi, J., Wang, Q., Collins, R. L., Laricchia, K. M., Ganna, A., Birnbaum, D. P., et al. (2020). The mutational constraint spectrum quantified from variation in 141,456 humans. Nature 581, 434-443. 10.1038/s41586-020-2308-7. 124. Genomes Project, C., Auton, A., Brooks, L. D., Durbin, R. M., Garrison, E. P., Kang, H. M., Korbel, J. O., Marchini, J. L., McCarthy, S., McVean, G. A., and Abecasis, G. R. (2015). A global reference for human genetic variation. Nature 526, 68-74. 10.1038/nature15393.
125. Huang, K. L., Mashl, R. J., Wu, Y., Ritter, D. I., Wang, J., Oh, C., Paczkowska, M., Reynolds, S., Wyczalkowski, M. A., Oak, N., et al. (2018). Pathogenic Germline Variants in 10,389 Adult Cancers. Cell 173, 355-370 e314. 10.1016/j.cell.2018.03.039.
126. Pierre-Jean, M., Rigaill, G., and Neuvial, P. (2015). Performance evaluation of DNA copy number segmentation methods. Brief Bioinform 16, 600-615. 10.1093/bib/bbu026.
127. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat Biotechnol 30, 413-421. 10.1038/nbt.2203.
128. Xi, R., Lee, S., Xia, Y., Kim, T. M., and Park, P. J. (2016). Copy number analysis of whole-genome data using BIC-seq2 and its application to detection of cancer susceptibility variants. Nucleic Acids Res 44, 6274-6286. 10.1093/nar/gkw491.
129. Chin, S. F., Teschendorff, A. E., Marioni, J. C., Wang, Y., Barbosa-Morais, N. L., Thorne, N. P., Costa, J. L., Pinder, S. E., van de Wiel, M. A., Green, A. R., et al. (2007). Highresolution aCGH and expression profiling identifies a novel genomic subtype of ER negative breast cancer. Genome Biol 8, R215. 10.1186/gb-2007-8-10-r215.
130. Burrell, R. A., McClelland, S. E., Endesfelder, D., Groth, P., Weller, M. C., Shaikh, N., Domingo, E., Kanu, N., Dewhurst, S. M., Gronroos, E., et al. (2013). Replication stress links structural and numerical cancer chromosomal instability. Nature 494, 492-496. 10.1038/nature11935.
131. Gao, Q., Liang, W. W., Foltz, S. M., Mutharasu, G., Jayasinghe, R. G., Cao, S., Liao, W. W., Reynolds, S. M., Wyczalkowski, M. A., Yao, L., et al. (2018). Driver Fusions and Their Implications in the Development and Treatment of Human Cancers. Cell Rep 23, 227-238 e223. 10.1016/j.celrep.2018.03.050.
132. Babiceanu, M., Qin, F., Xie, Z., Jia, Y., Lopez, K., Janus, N., Facemire, L., Kumar, S., Pang, Y., Qi, Y., et al. (2016). Recurrent chimeric fusion RNAs in non-cancer tissues and cells. Nucleic Acids Res 44, 2859-2872. 10.1093/nar/gkw032.
133. Butler, A., Hoffman, P., Smibert, P., Papalexi, E., and Satija, R. (2018). Integrating single-cell transcriptomic data across different conditions, technologies, and species. Nat Biotechnol 36, 411-420. 10.1038/nbt.4096.
134. Hafemeister, C., and Satija, R. (2019). Normalization and variance stabilization of singlecell RNA-seq data using regularized negative binomial regression. Genome Biol 20, 296. 10.1186/s13059-019-1874-1.
135. Young, M. D., Mitchell, T. J., Custers, L., Margaritis, T., Morales-Rodriguez, F., Kwakwa, K., Khabirova, E., Kildisiute, G., Oliver, T.R.W., de Krijger, R. R., et al. (2021). Single cell derived mRNA signals across human kidney tumors. Nat Commun 12, 3896. 10.1038/s41467-021-23949-5.
136. Borcherding, N., Vishwakarma, A., Voigt, A. P., Bellizzi, A., Kaplan, J., Nepple, K., Salem, A. K., Jenkins, R. W., Zakharia, Y., and Zhang, W. (2021). Mapping the immune environment in clear cell renal carcinoma by single-cell genomics. Commun Biol 4, 122. 10.1038/s42003-020-01625-6.
137. Qiu, X., Mao, Q., Tang, Y., Wang, L., Chawla, R., Pliner, H. A., and Trapnell, C. (2017). Reversed graph embedding resolves complex single-cell trajectories. Nat Methods 14, 979-982. 10.1038/nmeth.4402.
138. Kong, A. T., Leprevost, F. V., Avtonomov, D. M., Mellacheruvu, D., and Nesvizhskii, A.I. (2017). MSFragger: ultrafast and comprehensive peptide identification in mass spectrometry-based proteomics. Nat Methods 14, 513-520. 10.1038/nmeth.4256.
139. Yu, F., Teo, G. C., Kong, A. T., Haynes, S. E., Avtonomov, D. M., Geiszler, D. J., and Nesvizhskii, A.I. (2020). Identification of modified peptides using localization-aware open search. Nat Commun 11, 4065. 10.1038/s41467-020-17921-y.
140. da Veiga Leprevost, F., Haynes, S. E., Avtonomov, D. M., Chang, H. Y., Shanmugam, A. K., Mellacheruvu, D., Kong, A. T., and Nesvizhskii, A. I. (2020). Philosopher: a versatile toolkit for shotgun proteomics data analysis. Nat Methods 17, 869-870. 10.1038/s41592-020-0912-y.
141. Tsou, C. C., Avtonomov, D., Larsen, B., Tucholska, M., Choi, H., Gingras, A. C., and Nesvizhskii, A.I. (2015). DIA-Umpire: comprehensive computational framework for data independent acquisition proteomics. Nat Methods 12, 258-264, 257 p following 264. 10.1038/nmeth.3255.
142. Teo, G. C., Polasky, D. A., Yu, F., and Nesvizhskii, A.I. (2021). Fast Deisotoping Algorithm and Its Implementation in the MSFragger Search Engine. J Proteome Res 20, 498-505. 10.1021/acs.jproteome.0c00544.
143. Keller, A., Nesvizhskii, A.I., Kolker, E., and Aebersold, R. (2002). Empirical statistical model to estimate the accuracy of peptide identifications made by MS/MS and database search. Anal Chem 74, 5383-5392. 10.1021/ac025747h.
144. Nesvizhskii, A.I., Keller, A., Kolker, E., and Aebersold, R. (2003). A statistical model for identifying proteins by tandem mass spectrometry. Anal Chem 75, 4646-4658. 10.1021/ac0341261. 145. Savitski, M. M., Wilhelm, M., Hahne, H., Kuster, B., and Bantscheff, M. (2015). A Scalable Approach for Protein False Discovery Rate Estimation in Large Proteomic Data Sets. Mol Cell Proteomics 14, 2394-2404. 10.1074/mcp.M114.046995.
146. Demichev, V., Messner, C. B., Vernardis, S. I., Lilley, K. S., and Ralser, M. (2020). DIANN: neural networks and interference correction enable deep proteome coverage in high throughput. Nat Methods 17, 41-44. 10.1038/s41592-019-0638-x.
147. Cox, J., Hein, M. Y., Luber, C. A., Paron, I., Nagaraj, N., and Mann, M. (2014). Accurate proteome-wide label-free quantification by delayed normalization and maximal peptide ratio extraction, termed MaxLFQ. Mol Cell Proteomics 13, 2513-2526. 10.1074/mcp.M113.031591.
148. Shteynberg, D. D., Deutsch, E. W., Campbell, D. S., Hoopmann, M. R., Kusebauch, U., Lee, D., Mendoza, L., Midha, M. K., Sun, Z., Whetton, A. D., and Moritz, R. L. (2019). PTMProphet: Fast and Accurate Mass Modification Localization for the Trans-Proteomic Pipeline. J Proteome Res 18, 4262-4272. 10.1021/acs.jproteome.9b00205.
149. Genomes Project, C., Abecasis, G. R., Altshuler, D., Auton, A., Brooks, L. D., Durbin, R. M., Gibbs, R. A., Hurles, M. E., and McVean, G. A. (2010). A map of human genome variation from population-scale sequencing. Nature 467, 1061-1073. 10.1038/nature09534.
150. Kim, H., and Park, H. (2007). Sparse non-negative matrix factorizations via alternating non-negativity-constrained least squares for microarray data analysis. Bioinformatics 23, 1495-1502. 10.1093/bioinformatics/btm134.
151. Mani, D. R., Maynard, M., Kothadia, R., Krug, K., Christianson, K. E., Heiman, D., Clauser, K. R., Birger, C., Getz, G., and Carr, S. A. (2021). PANOPLY: a cloud-based platform for automated and reproducible proteogenomic data analysis. Nat Methods 18, 580-582. 10.1038/s41592-021-01176-6.
152. Sturm, D., Witt, H., Hovestadt, V., Khuong-Quang, D. A., Jones, D. T., Konermann, C., Pfaff, E., Tonjes, M., Sill, M., Bender, S., et al. (2012). Hotspot mutations in H3F3A and IDH1 define distinct epigenetic and biological subgroups of glioblastoma. Cancer Cell 22, 425-437. 10.1016/j.ccr.2012.08.024.
153. Zhou, W., Laird, P. W., and Shen, H. (2017). Comprehensive characterization, annotation and innovative use of Infinium DNA methylation BeadChip probes. Nucleic Acids Res 45, e22. 10.1093/nar/gkw967.
154. Malta, T. M., Sokolov, A., Gentles, A. J., Burzykowski, T., Poisson, L., Weinstein, J. N., Kaminska, B., Huelsken, J., Omberg, L., Gevaert, O., et al. (2018). Machine Learning Identifies Stemness Features Associated with Oncogenic Dedifferentiation. Cell 173, 338-354 e315. 10.1016/j.cell.2018.03.034.
155. Colaprico, A., Olsen, C., Bailey, M. H., Odom, G. J., Terkelsen, T., Silva, T. C., Olsen, A. V., Cantini, L., Zinovyev, A., Barillot, E., et al. (2020). Interpreting pathways to discover cancer driver genes with Moonlight. Nat Commun 11, 69. 10.1038/s41467-019-13803-0.
156. Daily, K., Ho Sui, S. J., Schriml, L. M., Dexheimer, P. J., Salomonis, N., Schroll, R., Bush, S., Keddache, M., Mayhew, C., Lotia, S., et al. (2017). Molecular, phenotypic, and sample-associated data to describe pluripotent stem cell lines and derivatives. Sci Data 4, 170030. 10.1038/sdata.2017.30.
157. Salomonis, N., Dexheimer, P. J., Omberg, L., Schroll, R., Bush, S., Huo, J., Schriml, L., Ho Sui, S., Keddache, M., Mayhew, C., et al. (2016). Integrated Genomic Analysis of Diverse Induced Pluripotent Stem Cells from the Progenitor Cell Biology Consortium. Stem Cell Reports 7, 110-125. 10.1016/j.stemcr.2016.05.006.
158. Sokolov, A., Paull, E. O., and Stuart, J.M. (2016). One-Class Detection of Cell States in Tumor Subtypes. Pac Symp Biocomput 21, 405-416.
159. Colaprico, A., Silva, T. C., Olsen, C., Garofano, L., Cava, C., Garolini, D., Sabedot, T. S., Malta, T. M., Pagnotta, S. M., Castiglioni, I., et al. (2016). TCGAbiolinks: an R/Bioconductor package for integrative analysis of TCGA data. Nucleic Acids Res 44, e71. 10.1093/nar/gkv1507.
160. Silva, T. C., Colaprico, A., Olsen, C., D'Angelo, F., Bontempi, G., Ceccarelli, M., and Noushmehr, H. (2016). TCGA Workflow: Analyze cancer genomics and epigenomics data using Bioconductor packages. F1000Res 5, 1542. 10.12688/f1000research.8923.2.
161. Duan, G., Li, X., and Kohn, M. (2015). The human DEPhosphorylation database DEPOD: a 2015 update. Nucleic Acids Res 43, D531-535. 10.1093/nar/gku1009.
162. Ruepp, A., Waegele, B., Lechner, M., Brauner, B., Dunger-Kaltenbach, I., Fobo, G., Frishman, G., Montrone, C., and Mewes, H. W. (2010). CORUM: the comprehensive resource of mammalian protein complexes—2009. Nucleic Acids Res 38, D497-501. 10.1093/nar/gkp914.
163. Perfetto, L., Briganti, L., Calderone, A., Cerquone Perpetuini, A., Iannuccelli, M., Langone, F., Licata, L., Marinkovic, M., Mattioni, A., Pavlidou, T., et al. (2016). SIGNOR: a database of causal relationships between biological entities. Nucleic Acids Res 44, D548-554. 10.1093/nar/gkv1048.
164. Jassal, B., Matthews, L., Viteri, G., Gong, C., Lorente, P., Fabregat, A., Sidiropoulos, K., Cook, J., Gillespie, M., Haw, R., et al. (2020). The reactome pathway knowledgebase. Nucleic Acids Res 48, D498-D503. 10.1093/nar/gkz1031.
165. Xu, T., Le, T. D., Liu, L., Su, N., Wang, R., Sun, B., Colaprico, A., Bontempi, G., and Li, J. (2017). CancerSubtypes: an R/Bioconductor package for molecular cancer subtype identification, validation and visualization. Bioinformatics 33, 3131-3133. 10.1093/bioinformatics/btx378.
166. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.
167. Robin, X., Turck, N., Hainard, A., Tiberti, N., Lisacek, F., Sanchez, J. C., and Muller, M. (2011). pROC: an open-source package for R and S+ to analyze and compare ROC curves. BMC Bioinformatics 12, 77. 10.1186/1471-2105-12-77.
168. Liao, Y., Wang, J., Jaehnig, E. J., Shi, Z., and Zhang, B. (2019). WebGestalt 2019: gene set analysis toolkit with revamped UIs and APIs. Nucleic Acids Res 47, W199-W205. 10.1093/nar/gkz401.
169. Calinawan, A. P., Song, X., Ji, J., Dhanasekaran, S. M., Petralia, F., Wang, P., and Reva, B. (2020). ProTrack: An Interactive Multi-Omics Data Browser for Proteogenomic Studies. Proteomics 20, e1900359. 10.1002/pmic.201900359.

### Example 3

### Methods

### Sample Processing

The CPTAC Biospecimen Core Resource (BCR) at the Pathology and Biorepository Core of the Van Andel Research Institute in Grand Rapids, Michigan manufactured and distributed biospecimen kits to the Tissue Source Sites (TSS) located in the US, and Europe. Each kit contains a set of pre-manufactured labels for unique tracking of every specimen respective to TSS location, disease, and sample type, used to track the specimens through the BCR to the CPTAC proteomic and genomic characterization centers. Tissue specimens averaging 200 mg were snap-frozen by the TSS within a 30 min cold ischemic time (CIT) (CIT average=15 min) and an adjacent segment was formalin-fixed paraffin embedded (FFPE) and H&E stained by the TSS for quality assessment to meet the CPTA tissue requirements. Routinely, several tissue segments for each case were collected. Tissues were flash-frozen in liquid nitrogen (LN2) and then transferred to a liquid nitrogen freezer for storage until approval for shipment to the BCR. Specimens were shipped using a cryoport that maintained an average temperature of under −140° C. to the BCR with a time and temperature tracker to monitor the shipment. Receipt of specimens at the BCR included a physical inspection and review of the time and temperature tracker data for specimen integrity, followed by barcode entry into a biospecimen tracking database.

Specimens were again placed in LN2 storage until further processing. Acceptable non-ccRCC tumor tissue segments were determined by TSS pathologists based on the percent viable tumor nuclei (>80%), total cellularity (>50%), and necrosis (<20%). Segments received at the BCR were verified by BCR and Leidos Biomedical Research (LBR) pathologists and the percent of the total area of tumor in the segment was also documented. Additionally, disease-specific working group pathology experts reviewed the morphology to clarify or standardize specific disease classifications and correlation to the proteomic and genomic data. The cryopulverized specimen was divided into aliquots for DNA (30 mg) and RNA (30 mg) isolation and proteomics (50 mg) for molecular characterization. Nucleic acids were isolated and stored at −80° C. until further processing and distribution; cryopulverized protein material was returned to the LN2 freezer until distribution. Shipment of the cryopulverized segments used cryoports for distribution to the proteomic characterization centers and shipment of the nucleic acids used dry ice shippers for distribution to the genomic characterization centers; a shipment manifest accompanied all distributions for the receipt and integrity inspection of the specimens at the destination.

### Sample Cohort Details

In this study, proteogenomics profiling of 194 tumor and NAT samples from the discovery cohort (110 tumors profiled with proteomics and RNA-seq, 84 NATs profiled with proteomics and 73 NATs profiled with RNA-seq), 4 samples from confirmatory (2 tumors and 2 NATs profiled with both proteomics and RNA-seq) and 56 samples from non-ccRCC cohorts (39 tumors profiled with proteins and RNA-seq, 17 NATs profiled with proteomics and 14 NATs profiled with RNA-seq) was performed. Within the 110 tumor samples from the discovery ccRCC cohort35, 103 were confirmed ccRCC 7 were non-ccRCC.

Across all three cohorts, 103 ccRCC tumor samples (all from the discovery cohort) and 48 non-ccRCC tumor samples (7 samples from the discovery cohort, 2 samples from the confirmatory cohort, 39 from the non-ccRCC cohort) were profiled. Within the 48 non-ccRCC samples, 15 ROs (3 RO type 1, 8 RO type 2, 4 RO variant), 13 papillary RCC (pRCC, 8 pRCC type 1, 5 other pRCC), 3 chromophobe RCC (chRCC), 2 angiomyolipoma (AML), 2 eosinophilic solid and cystic RCC (ESCRCC), 1 Birt-Hogg-Dube syndrome-associated renal cell carcinoma (BHD), 1 mixed epithelial and stromal tumor of the kidney (MEST), 1 MTOR mutated RCC, 1 translocation RCC (TRCC), 8 unclassified or other RCC (unRCC/other), and 1 plasmacytoid urothelial carcinoma (PUC), which is not renal cell carcinoma hence excluded in downstream analysis were observed. The following three samples were excluded from all downstream analysis: 2 NAT samples (C3N-00314-N and C3N-01524-N) that were found to be contaminated with tumor tissue and 1 plasmacytoid urothelial carcinoma (PUC) sample (C3L-02212) which is not renal cell carcinoma.

### Immunohistochemistry (IHC)

Immunohistochemistry (IHC) was performed on 4-micron formalin-fixed, paraffin-embedded (FFPE) tissue sections. The Ventana Benchmark XT staining platform with Discovery CCI and CC2 (Ventana cat #950-500 and 950-123) were used for antigen retrieval. The immune complexes were developed with either the ultra View or optiView Universal DAB (diaminobenzidine tetrahydrochloride) Detection Kit (Ventana cat #760-500 and cat #760-700). he details of the panel of primary antibodies utilized is as follows: polymeric immunoglobulin receptor (PIGR/Anti-SC; Santa Cruz, mouse monoclonal, catalog no. SC-374343), cyclin D1 (CCND1; Cell Marque, rabbit monoclonal, catalog no. 241R-18), transmembrane glycoprotein NMB (GPNMB, R&D systems, goat polyclonal, catalog no. AF2550), microtubule-associated protein RP/EB family member-3 (MAPRE3, Atlas antibodies, rabbit polyclonal, catalog no. HPA009263), and forkhead boxIl (FOXI1, Origene antibodies, mouse monoclonal, catalog no. TA800146). Brown pigmentation within the subcellular component (cytoplasmic and or membranous for PIGR, GPNMB, MAPRE3 and nuclear for FOXI1 and CCND1) were taken as positive expressions. For PIGR the presence and intensity of cytoplasmic staining were scored where the percentage of PIGR positive neoplastic cells and the staining intensity (none, 0; weak, 1; moderate, 2; strong, 3) were recorded for each tumor as described previously 12. Appropriate positive and negative control tissue were run in each assay batch.

### RNA In Situ Hybridization (RNA-ISH)

RNA-ISH was performed using the RNAscope 2.5 HD Brown kit (Advanced Cell Diagnostics, Newark, CA) and target probes against PIGR (472681 Hs-PIGR targeting NM_002644.3, 2-903nt), PYCR1 (509259 Hs-PYCR1 targeting NM_001282281.1, 64-1770nt), and SOSTDC1 469929 Hs-SOSTDC1 targeting NM_015464.2, 2-938nt) according to the manufacturer's instructions. RNA quality was evaluated in each case utilizing a positive and a negative control probe against human housekeeping gene Peptidylprolyl Isomerase B (PPIB) (313901 for manual and 313909 for Ventana automated system) and bacillus bacterial gene DapB (310043 for manual and 312039 for Ventana automated system) respectively. The assay was run according to the protocol previously described 10,13.

Stained slides were evaluated under a light microscope at ×100 and ×200 magnification for RNA-ISH signals in neoplastic cells by multiple study investigators. Each RNA molecule in this assay's result is represented as a punctate brown dot. The expression level was evaluated according to the RNAscope scoring criteria: score 0=no staining or &lt; 1 dot per 10 cells; score 1=1-3 dots per cell, score 2=4-9 dots per cell, and no or very few dot clusters; score 3=10-15 dots per cell and <10% dots in clusters; score 4=>15 dots per cell and >10% dots in clusters. The H-score was calculated for each examined tissue section as the sum of the percentage of cells with score 0-4 [(A %× 0)+ (B %× 1)+ (C %×2)+ (D %×3)+ (E %×4), A+B+C+D+E=100], using previously published scoring criteria 10,13.

### Cell Viability, Cell Migration and Drug Treatment Experiments

The cell viability was determined by CellTiter-Glo assays (Promega). Cells were seeded in 96-well plates (3000 cells per well) in respective culture medium. After 24 hrs incubation, a serial dilution of compounds was added into plates with six replications for each dose. After 120 hrs incubation, the CellTiter-Glo assays were performed based on the manufacturer's instruction to analyze cell proliferation rates. The bioluminescence signal was acquired by the Infinite M1000 Pro plate reader (Tecan). The data were analyzed by GraphPad Prism software (GraphPad Software).

siRNA Mediated Knockdown

The ON-TARGETplus SMARTpool siRNA targeting PYCR1 mRNA was purchased from Dharmacon horizon (cat. no.L-012349-00-0005). The cells were plated on 6-well plates (5×10⁵ cells/well) and cultured overnight. Then the siRNA was transfected into cells by using Lipofectamine™ RNAiMAX Transfection Reagent from Thermofisher. After transfection, the cells were cultured for 48 hrs and then harvested for western blot or CTG analysis. The on-target effect was confirmed by western blot analysis.

### Compound Treatments and Western Blot

The cells were plated on 6-well plates (5x10⁵ cells/well) and cultured overnight. The cells were treated with compounds for 72 hrs and then the cell lysates were prepared by RIPA buffers (ThermoFisher Scientific) with complete™ protease inhibitor cocktail tablets (Sigma-Aldrich). The equal amount of protein was resolved in NuPAGE 4 to 12%, Bis-Tris Protein Gel (ThermoFisher Scientific) and blotted with primary antibodies overnight in 4° C. After incubation with HRP-conjugated secondary antibodies, the membranes were imaged by an Odyssey CLx Imager (LiCOR Biosciences).

### Genomic and Transcriptomic Sample Preparation and Data Acquisition

### Sample Processing for Genomic DNA and Total RNA Extraction

This study sampled a single site of the primary tumor from surgical resections, due to the internal requirement to process a minimum of 125 mg of tumor issue and 50 mg of adjacent normal tissue. DNA and RNA were extracted from tumor and blood normal specimens in a co-isolation protocol using Qiagen's QIAsymphony DNA Mini Kit and QIAsymphony RNA Kit. Genomic DNA was also isolated from peripheral blood (3-5 mL) to serve as matched normal reference material. The Qubit™ dsDNA BR Assay Kit was used with the Qubit® 2.0 Fluorometer to determine the concentration of dsDNA in an aqueous solution. Any sample that passed quality control and produced enough DNA yield to go through various genomic assays was sent for genomic characterization. RNA quality was quantified using both the NanoDrop 8000 and quality assessed using Agilent Bioanalyzer. A sample that passed RNA quality control and had a minimum RIN (RNA integrity number) score of 7 was subjected to RNA sequencing. Identity match for germline, normal adjacent tissue, and tumor tissue was assayed at the BCR using the Illumina Infinium QC array. This beadchip contains 15,949 markers designed to prioritize sample tracking, quality control, and stratification.

### PCR-Free Whole Genome Sequencing (WGS)

### Preparation of Libraries for Cluster Amplification and Sequencing

An aliquot of genomic DNA (350 ng in 50 μL) was used as the input into DNA fragmentation (aka shearing). Shearing was performed acoustically using a Covaris focused-ultrasonicator, targeting 385 bp fragments. Following fragmentation, additional size selection was performed using a SPRI cleanup. Library preparation was performed using a commercially available kit provided by KAPA Biosystems (KAPA Hyper Prep without amplification module) and with palindromic forked adapters with unique 8-base index sequences embedded within the adapter (purchased from IDT). Following sample preparation, libraries were quantified using quantitative PCR (kit purchased from KAPA Biosystems), with probes specific to the ends of the adapters. This assay was automated using Agilent's Bravo liquid handling platform. Based on qPCR quantification, libraries were normalized to 1.7 nM and pooled into 24-plexes.

### Cluster Amplification and Sequencing (HiSeq X)

Sample pools were combined with HiSeq X Cluster Amp Reagents EPX1, EPX2, and EPX3 into single wells on a strip tube using the Hamilton Starlet Liquid Handling system. Cluster amplification of the templates was performed according to the manufacturer's protocol (Illumina) with the Illumina cBot. Flow cells were sequenced to a minimum of 15x on HiSeq X utilizing sequencing-by-synthesis kits to produce 151 bp paired-end reads. Output from Illumina software was processed by the Picard data processing pipeline to yield BAMs containing demultiplexed, aggregated, aligned reads. All sample information tracking was performed by automated LIMS messaging.

### Whole Exome Sequencing (WES)

### Library Construction

Library construction was performed as described in180, with the following modifications: initial genomic DNA input into shearing was reduced from 3 μg to 20-250 ng in 50 μL of solution. For adapter ligation, Illumina paired-end adapters were replaced with palindromic forked adapters, purchased from Integrated DNA Technologies, with unique dual-indexed molecular barcode sequences to facilitate downstream pooling. Kapa HyperPrep reagents in 96-reaction kit format were used for end repair/A-tailing, adapter ligation, and library enrichment PCR. In addition, during the post-enrichment SPRI cleanup, elution volume was reduced to 30 μL to maximize library concentration, and a vortexing step was added to maximize the amount of template eluted.

### In-Solution Hybrid Selection

After library construction, libraries were pooled into groups of up to 96 samples. Hybridization and capture were performed using the relevant components of Illumina's Nextera Exome Kit and following the manufacturer's suggested protocol, with the following exceptions. First, all libraries within a library construction plate were pooled prior to hybridization. Second, the Midi plate from Illumina's Nextera Exome Kit was replaced with a skirted PCR plate to facilitate automation. All hybridization and capture steps were automated on the Agilent Bravo liquid handling system.

### Preparation of Libraries for Cluster Amplification and Sequencing

After post-capture enrichment, library pools were quantified using qPCR (automated assay on the Agilent Bravo) using a kit purchased from KAPA Biosystems with probes specific to the ends of the adapters. Based on qPCR quantification, libraries were normalized to 2 nM.

### Cluster Amplification and Sequencing

Cluster amplification of DNA libraries was performed according to the manufacturer's protocol (Illumina) using exclusion amplification chemistry and flowcells. Flowcells were sequenced utilizing sequencing-by-synthesis chemistry. The flow cells were then analyzed using RTA v.2.7.3 or later. Each pool of whole-exome libraries was sequenced on paired 76 cycle runs with two 8 cycle index reads across the number of lanes needed to meet coverage for all libraries in the pool. Pooled libraries were run on HiSeq 4000 paired end runs to achieve a minimum of 150x on target coverage per sample library. The raw Illumina sequence data were demultiplexed and converted to fastq files; adapter and low-quality sequences were trimmed. The raw reads were mapped to the hg38 human reference genome and the validated BAMs were used for downstream analysis and variant calling.

### RNA Sequencing

### Quality Assurance and Quality Control of RNA Analytes

All RNA analytes were assayed for RNA integrity, concentration, and fragment size. Samples for total RNA-seq were quantified on a TapeStation system (Agilent, Inc. Santa Clara, CA). Samples with RINs >8.0 were considered high quality.

### Total RNA-Seq Library Construction

Total RNA-seq library construction was performed from the RNA samples using the TruSeq Stranded RNA Sample Preparation Kit and bar-coded with individual tags following the manufacturer's instructions (Illumina, Inc. San Diego, CA). Libraries were prepared on an Agilent Bravo Automated Liquid Handling System. Quality control was performed at every step and the libraries were quantified using the TapeStation system.

### Total RNA Sequencing

Indexed libraries were prepared and run on HiSeq 4000 paired-end 75 base pairs to generate a minimum of 120 million reads per sample library with a target of greater than 90% mapped reads. Typically, these were pools of four samples. The raw Illumina sequence data were demultiplexed and converted to FASTQ files, and adapter and low-quality sequences were quantified. Samples were then assessed for quality by mapping reads to the hg38 human genome reference, estimating the total number of reads that mapped, amount of RNA mapping to coding regions, amount of rRNA in sample, number of genes expressed, and relative expression of housekeeping genes. Samples passing this QA/QC were then clustered with other expression data from similar and distinct tumor types to confirm expected expression patterns. Atypical samples were then SNP typed from the RNA data to confirm the source analyte. FASTQ files of all reads were then uploaded to the GDC repository.

### Single-Nuclei RNA Library Preparation and Sequencing

About 20-30 mg of cryopulverized powder from ccRCC specimens was resuspended in Lysis buffer (10 mM Tris-HCl (pH 7.4); 10 mM NaCl; 3 mM MgCl2; and 0.1% NP-40). This suspension was pipetted gently 6-8 times, incubated on ice for 30 seconds, and pipetted again 4-6 times. The lysate containing free nuclei was filtered through a 40 μm cell strainer. The filter was washed with 1 mL Wash and Resuspension buffer (1X PBS+2% BSA+0.2 U/uL RNase inhibitor) and the flow through was combined with the original filtrate. After 6-minute centrifugation at 500×g and 4° C., the nuclei pellet was resuspended in 500 μL of Wash and Resuspension buffer. After staining by DRAQ5, the nuclei were further purified by Fluorescence-Activated Cell Sorting (FACS). FACS-purified nuclei were centrifuged again and resuspended in a small volume (about 30 μL). After counting and microscopic inspection of nuclei quality, the nuclei preparation was diluted to about 1,000 nuclei/uL.

About 20,000 nuclei were used for single-nuclei RNA sequencing (snRNA seq) by the 10X Chromium platform. The single nuclei were loaded onto a Chromium Chip B Single Cell Kit, 48 rxns (10x Genomics, PN-1000073), and processed through the Chromium Controller to generate GEMs (Gel Beads in Emulsion). Sequencing libraries were then prepared with the Chromium Single Cell 3′ GEM, Library & Gel Bead Kit v3, 16 rxns (10x Genomics, PN 1000075) following the manufacturer's protocol. Sequencing was performed on an Illumina NovaSeq 6000 S4 flow cell. The libraries were pooled and sequenced using the XP workflow according to the manufacturer's protocol with a 28×8×98 bp sequencing recipe. The resulting sequencing files were available as FASTQs per sample after demultiplexing.

Illumina Infinium methylationEPIC Beadchip Array

The MethylationEPIC array uses an 8-sample version of the Illumina Beadchip capturing >850,000 DNA methylation sites per sample. 250 ng of DNA was used for the bisulfite conversation using Infinium MethylationEPIC BeadChip Kit. The EPIC array includes sample plating, bisulfite conversion, and methylation array processing. After scanning, the data was processed through an automated genotype calling pipeline. Data generated consisted of raw idats and a sample sheet.

### Proteomic Sample Preparation and Data Acquisition

### Sample Processing for Protein Extraction and Tryptic Digestion

All samples for the current study were prospectively collected as described above and processed for mass spectrometric (MS) analysis at Johns Hopkins University. Tissue lysis and downstream sample preparation for global proteomic, phosphoproteomic and glycoproteomic analysis were carried out as previously described36,63,181. Each of cryopulverized renal tumor tissues or NATs were homogenized separately in an appropriate volume of lysis buffer (8 M urea, 75 mM NaCl, 50 mM Tris, pH 8.0, 1 mM EDTA, 2 μg/mL aprotinin, 10 μg/mL leupeptin, 1 mM PMSF, 10 mM NaF, Phosphatase Inhibitor Cocktail 2 and Phosphatase Inhibitor Cocktail 3 [1:100 dilution], and 20 μM PUGNAc) by repeated vortexing.

Proteins in the lysates were clarified by centrifugation at 20,000×g for 10 min at 4C, and protein concentrations were determined by BCA assay (Pierce). The proteins were diluted to a final concentration of 8 mg/mL with a lysis buffer for downstream reduction, alkylation and digestion. 1.2 mg of protein was reduced with 5 mM dithiothreitol (DTT) for 1 h at 37 C and subsequently alkylated with 10 mM iodoacetamide for 45 min at RT (room temperature) in the dark. Samples were then diluted by 1:4 with 50 mM Tris-HCl (pH 8.0) and subjected to proteolytic digestion with LysC (Wako Chemicals, at 1:50 enzyme-to-substrate weight ratio for 2 h incubation at RT) followed by the addition of sequencing-grade modified trypsin (Promega, at a 1:50 enzyme-to-substrate weight ratio for overnight incubation at RT). The digested samples were then acidified with 50% formic acid (FA, Fisher Chemicals) to pH<3. Tryptic peptides were desalted on reversed-phase C18 SPE columns (Waters) and dried using a Speed-Vac (Thermo Scientific).

### TMT Labeling of Peptides

Tandem-mass-tag (TMT) quantitation utilizes reporter ion intensities to determine protein abundance and facilitate quantitative proteomic analysis 182. The samples from the discovery cohort were labeled with TMT-10plex as described 63, while the samples from the non-ccRCC cohort were labeled with TMT-11plex reagents (Thermo Fisher Scientific). 70 non-ccRCC samples were co-randomized to 7 TMT 11-plex sets. The sample-to-TMT channel mapping is available in PDC portal (proteomic.datacommons.cancer.gov/). 300ug desalted peptides from each non-ccRCC and NAT sample were dissolved in 120 μL of 100 mM HEPES, pH 8.5 solution. 5 mg TMT reagent was dissolved in 500 μL of anhydrous acetonitrile, and 45 μL of each TMT reagent was added to the corresponding aliquot of peptides. After 1 h incubation at RT, the reaction was quenched by incubation with 5% hydroxylamine at RT for 15 min. The reference sample used in the ccRCC discovery cohort study was included in all TMT 11-plexes as a reference channel s described63, labeled with the TMT-131 reagent. Following labeling, peptides were mixed according to the sample-to-TMT channel mapping, concentrated and desalted on reversed-phase C18 SPE columns (Waters), and dried using a Speed-Vac (Thermo Scientific).

### Peptide Fractionation by Basic Reversed-Phase Liquid Chromatography

To reduce the likelihood of peptides co-isolating and co-fragmenting in these highly complex samples, extensive, high-resolution fractionation via basic reversed-phase liquid chromatography (bRPLC) was utilized. The desalted and dried peptides from each TMT set were reconstituted in 900 mL of 5 mM ammonium formate (pH 10) and 2% acetonitrile (ACN) and loaded onto a 4.6 mm×250 mm RP Zorbax 300 A Extend-C18 column with 3.5 μm size beads (Agilent). Peptides were separated at a flow-rate of 1 mL/min using an Agilent 1200 Series HPLC instrument with Solvent A (2% ACN, 5 mM ammonium formate, pH 10) and a non-linear gradient of Solvent B (90% ACN, 5 mM ammonium formate, pH 10) as follows: 0% Solvent B (7 min), 0%-16% Solvent B (6 min), 16% to 40% Solvent B (60 min), 40% to 44% Solvent B (4 min), 44% to 60% Solvent B (5 min), and holding at 60% Solvent B for 14 min. Collected fractions were concatenated into 24 fractions by combining four fractions that are 24 fractions apart as described previously36; a 5% aliquot of each of the 24 fractions was used for global proteomic analysis, dried in a Speed-Vac, and resuspended in 3% ACN/0.1% formic acid prior to ESI-LC-MS/MS analysis. The remaining sample was utilized for phosphopeptide enrichment.

### Enrichment of Phosphopeptides by Fe-IMAC

The remaining 95% of the sample was further concatenated into 12 fractions before being subjected to phosphopeptide enrichment using immobilized metal affinity chromatography (IMAC) as previously described36. In brief, Ni-NTA agarose beads (Qiagen) were conditioned and incubated with 10 mM FeC13 to prepare Fe3+-NTA agarose beads. Dried peptides from each fraction were reconstituted in 80% ACN/0.1% trifluoroacetic acid and incubated with 10 μL of the Fe3+-IMAC beads for 30 min. Samples were then centrifuged at 1000*g for 1 min to collect the beads coupled with phophopeptides, and the supernatant containing unbound peptides was removed for the subsequent glycopeptides enrichment (Cao, PDA paper, cell, 2021). The beads were resuspended with 80% ACN/0.1% trifluoroacetic acidand then transferred onto equilibrated C-18 Stage Tips. Tips were washed twice with 80% ACN/0.1% trifluoroacetic acid followed by 1% formic acid.

The flowthroughs were collected and combined with the supernatants for subsequent glycopeptides enrichments. Phosphopeptides were eluted from the Fe3+-IMAC beads onto the C-18 Stage Tips with 70 μL of 500 mM dibasic potassium phosphate, pH 7.0 three times. C-18 Stage Tips were then washed twice with 1% formic acid to remove salts, followed by elution of the phosphopeptides from the C-18 Stage Tips with 50% ACN/0.1% formic acid twice. Eluted phosphopeptides were dried down and resuspended in 3% ACN/0.1% formic acid prior to ESI-LC-MS/MS analysis.

### Enrichment of Intact Glycopeptides by MAX Columns

All unbound peptides from phosphopeptide enrichment were desalted on reversed phase C18 SPE column (Waters). The glycopeptides were enriched with OASIS MAX solid-phase extraction (Waters). The MAX cartridge was conditioned with 3×1 mL ACN, then 3×1 mL of 100 mM triethylammonium acetate buffer, followed by 3×1 mL of water, and finally 3×1 mL of 95% ACN (1% TFA). The peptides were loaded twice. The cartridge was washed with 4×1 mL of 95% ACN (1% TFA) to remove non-glycosylated peptides. The glycopeptide fraction was eluted with 50% ACN (0.1% TFA), dried down, and reconstituted in 3% ACN, 0.1% FA prior to ESI-LC-MS/MS analysis.

### ESI-LC-MS/MS for Global Proteome, Phosphoproteome, and Glycoproteome Analysis

The TMT-labeled global proteome, phosphoproteome, and glycoproteome fractions were analyzed using Orbitrap Fusion Lumos mass spectrometer (Thermo Scientific). Approximately 0.8 μg of peptides were separated on an in-house packed 28 cm×75 mm diameter C18 column (1.9 mm Reprosil-Pur C18-AQ beads (Dr. Maisch GmbH); Picofrit 10 mm opening (New Objective)) lined up with an Easy nLC 1200 UHPLC system (Thermo Scientific). The column was heated to 50° C. using a column heater (Phoenix-ST). The flow rate was set at 200 nl/min. Buffer A and B were 3% ACN (0.1% FA) and 90% ACN (0.1% FA), respectively. The peptides were separated with a 6%-30% B gradient in 84 min. Peptides were eluted from the column and nanosprayed directly into the mass spectrometer. The mass spectrometer was operated in a data-dependent mode.

Parameters for global proteomic and phosphoproteomic samples were set as follows: MS1 resolution—60,000, mass range—350 to 1800 m/z, RF Lens—30%, AGC Target—4.0e5, Max injection time—50 ms, charge state include—2-6, dynamic exclusion—45 s. The cycle time was set to 2 s, and within this 2 s the most abundant ions per scan were selected for MS/MS in the orbitrap. MS2 resolution 50,000, high-energy collision dissociation activation energy (HCD)—34, isolation width (m/z)—0.7, AGC Target—2.0e5, Max injection time—100 ms. Parameters for glycoproteomic samples were set as follows: MS1 resolution—60,000, mass range—500 to 2000 m/z, RF Lens—30%, AGC Target—5.0e5, Max injection time—50 ms, charge state include—2-6, dynamic exclusion—45 s. The cycle time was set to 2 s, and within this 2 s the most abundant ions per scan were selected for MS/MS in the orbitrap. MS2 resolution—50,000, high-energy collision dissociation activation energy (HCD)—35, isolation width (m/z)—0.7, AGC Target—1.0e5, Max injection time—100 ms.

### Metabolomic Acquisition

To extract metabolites, a solution consisting of 80% (vol/vol) mass spectrometry-grade methanol and 20% (vol/vol) mass spectrometry-grade water were used to extract the metabolites from the tissue samples as described previously 183-185. The metabolite samples then underwent speed vacuum processing to evaporate the methanol and lyophilization to remove the water. The dried metabolites were re-suspended in a solution consisting of 50% (vol/vol) acetonitrile and 50% (vol/vol) mass spectrometry-grade water before data acquisition. Data acquisition was performed using a Vanquish ultra-performance liquid chromatography (UPLC) system and a Thermo Scientific Q Exactive Plus Orbitrap Mass Spectrometer.

The samples were kept at 4° C. inside the Vanquish UPLC auto-sampler. The injection volume for each sample was 2 μL. A Discovery® HSF5 reverse phase HPLC column (Sigma) kept at 35° C. with a guard column was used for reverse-phase chromatography. The mobile aqueous phase was mass spectrometry-grade water containing 0.1% formic acid, while the mobile organic phase was acetonitrile containing 0.1% formic acid. Mass calibration was performed prior to data acquisition to ensure the sensitivity and accuracy of the system. The total run time for each sample was 15 minutes, for which 11 minutes was used for data acquisition. Full MS data were acquired to quantify the metabolites while Full MS/ddMS2 data were also acquired to identify the metabolites based on fragmentation matching.

### Genomic Data Processing

### Somatic Mutation Calling

WES reads were aligned FASTQ files to the GRCh38 references, including alternate haplotypes. Variant calling was performed using VarDict (germline & somatic) and Strelka2 (somatic). Variant callers were run with default settings, but custom filters were applied. Strelka was used to generate the primary somatic call-set. Variants called by Strelka had to be either (FILTER== “PASS”) or meet the following threshold criteria: allele frequency in the tumor >0.05, allele frequency in the normal <0.01, at least five variant reads, depth in normal >50, Somatic Evidence Score (EVS)>90th percentile of overall EVS distribution. These calls were supplemented by variants called confidently (FILTER== “PASS” and manual review) by VarDict in genes recurrently mutated in ccRCC: VHL, PBRM1, BAP1, SETD2, KDM5C, PTEN, MTOR, TP53, PIK3CA, ARIDIA, STAG2, KDM6A, KMT2C, KMT2D. This strategy improved sensitivity in ccRCC-mutated genes without sacrificing the accuracy of variant calls genome wide.

### Gene Fusion

RNAseq data was further supplied to call gene fusions using CRISP, CODAC TPO pipeline previously described 186,187.

### Copy Number Estimation

Copy-number analysis was performed jointly leveraging both whole-genome sequencing (WGS) and whole-exome sequencing data of the tumor and germline DNA. To perform the analysis, CNVEX (github.com/mctp/cnvex), a comprehensive copy number analysis tool that has been used previously25,35, was utilized. CNVEX uses whole-genome aligned reads to estimate coverage within fixed genomic intervals and whole exome variant calls to compute B-allele frequencies (BAFs) at variant positions (called by Sentieon DNAscope algorithm). Coverages were computed in 10 kb bins, and the resulting log coverage ratios between tumor and normal samples were adjusted for GC bias using weighted LOESS smoothing across mappable and non-blacklisted genomic intervals within the GC range 0.3-0.7, with a span of 0.5 (the target and configuration files are provided with CNVEX). The adjusted log coverage ratios (LR) and BAFs were jointly segmented by a custom algorithm based on Circular Binary Segmentation (CBS). Alternative probabilistic algorithms were implemented in CNVEX, including algorithms based on recursive binary segmentation (RBS), as implemented in the R-package jointseg188. For the CBS-based algorithm, first LR and mirrored BAF were independently segmented using CBS (parameters alpha=0.01, trim=0.025) and all candidate breakpoints were collected. The resulting segmentation track was iteratively “pruned” by merging segments that had similar LR and BAFs, short lengths, were rich in blacklisted regions, and had a high coverage variation in coverage among whole cohort germline samples. For the RBS- and DP-based algorithms, joint-break-points were “pruned” using a statistical model selection method (hal.inria.fr/inria-00071847). For the final set of CNV segments, the CBS-based results were used as they did not require specifying a prior number of expected segments (K) per chromosome arm, were robust to unequal variances between the LR and BAF tracks, and provided empirically the best fit to the underlying data. The resulting segmented copy-number profiles were then subject to the joint inference of tumor purity and ploidy and absolute copy number state, implemented in CNVEX, which is most similar to the mathematical formalism of ABSOLUTE189 and PureCN190 (bioconductor.org/packages/PureCN/).

Briefly, the algorithm inputs the observed log-ratios (of 10 kb bins) and BAFs of individual SNPs. LRs and BAFs are assigned to their joint segments and their likelihood is determined given a particular purity, ploidy, absolute segment copy number, and the number of minor alleles. To identify candidate combinations with a high likelihood, a multi-step optimization procedure that includes grid-search (across purity-ploidy combinations), greedy optimization of absolute copy numbers, and maximum-likelihood inferences of minor allele counts was used. Following optimization, CNVEX ranks candidate solutions. Because the copy-number inference problem can have multiple equally likely solutions, further biological insights are necessary to choose the most parsimonious result. The solutions have been reviewed by independent analysts following a set of guidelines. Solutions implying whole genome duplication must be supported by at least one large segment that cannot be explained by a low-ploidy solution, inferred purity must be consistent with the variant-allele-frequencies of somatic mutations, and large homozygous segments are not allowed.

In parallel, BIC-seq2191, a read-depth-based CNV calling algorithm, was used to detect somatic copy number variation (CNVs) from the WGS data of tumors. Briefly, BIC-seq2 divides genomic regions into disjoint bins and counts uniquely aligned reads in each bin. Then, it combines neighboring bins into genomic segments with similar copy numbers iteratively based on Bayesian Information Criteria (BIC), a statistical criterion measuring both the fitness and complexity of a statistical model. Paired-sample CNV calling that takes a pair of samples as input and detects genomic regions with different copy numbers between the two samples was used with a bin size of ˜100 bp and a lambda of 3 (a smoothing parameter for CNV segmentation). Segments were called as copy gain or loss when their log 2 copy ratios were larger than 0.2 or smaller than-0.2, respectively (according to the BIC-seq publication).

### Transcriptomic Data Processing

### RNAseq

Transcriptomic data were analyzed as described previously 186, using the Clinical RNA-seq Pipeline (CRISP) (github.com/mcieslik-mctp/crisp-build) of TPO. Briefly, raw sequencing data were trimmed, merged using BBMap, and aligned to GRCh38 using STAR. The resulting BAM files were analyzed for expression using feature counts against a transcriptomic reference based on Gencode 34. The resulting gene-level counts for protein-coding genes were transformed into FPKMs using edgeR 192.

snRNA-seq

Read alignment and quantification were conducted with Cell Ranger (v3.1.0) and pre-mRNA reference genome created based on 10X pre-built reference genome (GRCh38). Specifically, for each sample, the unfiltered feature-barcode matrix per sample was obtained by passing the demultiplexed FASTQs to Cell Ranger v3.1.0 ‘count’ command using default parameters, and a customized pre-mRNA GRCh38 genome reference was built to capture both exonic and intronic reads. The customized genome reference modified the transcript annotation from the 10x Genomics pre-built human genome reference 3.0.0 (GRCh38 and Ensembl 93). Starting with unfiltered count matrix, non-empty barcodes were identified with DropletUtils 193,194, correction for potential background RNA contamination was performed with SoupX 195. Cells with outlier numbers of total UMIs/genes and mitochondrial gene fraction were identified using scatter and discarded. For total UMI/genes, values were 3 median-absolute-deviations or MADs higher or lower from median were considered outliers; for mitochondrial fractions, values were 3 MADs higher than median were considered outliers. Subsequently, mitochondrial genes were removed from the entire count matrix as they probably represented contamination from cytoplasm during nuclei preparation.

### Proteomic and Metabolic Data Processing

### Identification and Quantification of Global Proteome and Phosphoproteome

Raw mass spectrometry files were converted into open mzML format using the msconvert utility of the Proteowizard software suite, and analyzed using FragPipe computational platform (fragpipe.nesvilab.org) using the TMT11-bridge workflow where the common ccRCC pool sample was used as bridge to link the two cohort. (the 11th channel was removed later in the discovery cohort). MS/MS spectra were searched using the database search tool MSFragger v3.4169 against a harmonized Homo sapiens GENCODE34 protein sequence database appended with an equal number of decoy sequences. Whole cell lysate MS/MS spectra were searched using a precursor-ion mass tolerance of 20 ppm and allowing C12/C13 isotope errors-1/0/1/2/3. Mass calibration and parameter optimization were enabled. Cysteine carbamidomethylation (+57.0215) and lysine TMT labeling (+229.1629) were specified as fixed modifications, and methionine oxidation (+15.9949), N-terminal protein acetylation (+42.0106), and TMT labeling of peptide N terminus and serine residues were specified as variable modifications.

For the analysis of phosphopeptide enriched data, the set of variable modifications also included phosphorylation (+79.9663) of serine, threonine, and tyrosine residues. The search was restricted to tryptic peptides, allowing up to two missed cleavage sites. Peptide to spectrum matches (PSMs) were further processed using Percolator196 to compute the posterior error probability, which was then converted to posterior probability of correct identification for each PSM. The resulting files from Percolator were converted to pep.xml format, and with the phosphopeptide-enriched data set, pep.xml files were additionally processed using PTMProphet197 to localize the phosphorylation sites. The resulting files were then processed together to assemble peptides into proteins (protein inference) using ProteinProphet198 run via the Philosopher toolkit v4.0.1168 to create a combined set of high confidence protein groups. The combined prot.xml file and the individual PSM lists for each TMT experiment were further processed using the Philosopher filter command as follows.

Each peptide was assigned either as a unique peptide to a particular protein group or assigned as a razor peptide to a single protein group that had the most peptide evidence. The protein groups assembled by Percolator were filtered to 1% protein-level False Discovery Rate (FDR) using the target-decoy strategy and the best peptide approach (allowing both unique and razor peptides). The PSM lists were filtered using a sequential FDR strategy, keeping only those PSMs that passed 1% PSM-level FDR filter and mapped to proteins that also passed the global 1% protein-level FDR filter. In addition, for all PSMs corresponding to a TMT-labeled peptide, reporter ion intensities were extracted from the MS/MS scans (using 0.002 Da window) using Philosopher and the precursor ion purity scores were calculated using the intensity of the sequenced precursor ion and that of other interfering ions observed in MS1 data (within a 0.7 Da isolation window). The PSM output files were further processed using TMT-Integrator v3.2.0 to generate summary reports at the gene level and modification site level. TMT-Integrator171 (github.com/Nesvilab/TMT-Integrator) used as input the PSM tables generated by the Philosopher pipeline as described above and created integrated reports with quantification across all samples. First, PSMs were filtered to remove all entries that did not pass at least one of the quality filters, such as PSMs with (a) no TMT label; (b) precursor-ion purity less than 50%; (c) summed reporter ion intensity (across all channels) in the lower 5% percentile of all PSMs in the corresponding PSM.tsv file (2.5% for phosphopeptide enriched data); (d) peptides without phosphorylation (for phosphopeptide enriched data). In the case of redundant PSMs (i.e., multiple PSMs in the same MS run sample corresponding to the same peptide ion), only the single PSM with the highest summed TMT intensity was retained for subsequent analysis. Both unique and razor peptides were used for quantification, while PSMs mapping to common external contaminant proteins (that were included in the searched protein sequence database) were excluded.

Next, for each PSM the intensity in each TMT channel was converted into a log 2-based ratio to the reference channel. The PSMs were grouped to the gene level, and the gene ratios were computed as the median of the corresponding PSM ratios after outlier removal. Ratios were then converted back to absolute intensity in each sample by using the reference gene intensity estimated, using the sum of all MS2 reporter ions from all corresponding PSMs. To generate peptide-level and site-level tables, additional post-processing was applied to generate all non-conflicting phosphosite configurations using a strategy similar to that described in Huang et al.199. In doing so, confidently localized sites were defined as sites with PTMProphet localization probability of 0.75 or higher. The same peptide sequences but with different site configurations, i.e., different site localization configurations or peptides with unlocalized sites, were retained as separate entries in the site-level tables. In the peptide-level tables, different site-level configurations were combined into a single peptide-level index, grouping PSMs with all site configurations together if they corresponded to the same peptide sequence. The tutorial describing all steps of the analysis, including specific input parameter files, command-line option, and all software tools necessary to replicate the results are available at github.com/Nesvilab.

### Quantification of Intact Glycopeptides and Glycosite Localization

Raw files of the glyco-enriched samples and phospho-enriched samples were searched for N-linked glycopeptides via MSFragger96 (version 3.3) and Philosopher168 (version 4.0). Parameters were as described for whole proteome search, except as follows. C12/C13 isotope errors of 0/1/2 were allowed, and methionine oxidation (+15.99491) was the only specified variable modification for glyco-enriched samples. Phosphorylation of serine, threonine, and tyrosine (+79.96633) was specified for phospho-enriched samples. “Nglycan” search mode was used, restricting glycosylation sites to the consensus sequon N—X-S/T, where X is any residue other than proline. A customized human N-glycan database which contained 252 compositions181. Diagnostic ion filtering for glycopeptide spectra was enabled with a minimum intensity threshold of 10% and the following list of oxonium ions considered: 204.086646, 186.076086, 168.065526, 366.139466, 144.0656, 138.055, 512.197375, 292.1026925, 274.0921325, 657.2349, 243.026426, 405.079246, 485.045576, 308.09761. Glycan Y ions of 203.07937 and 406.15874 were included in search, along with a remainder mass of 203.07937 on peptide b and y ions (“b˜/y˜” ions). PSMs were further processed with PeptideProphet, using the extended mass model with a mass width of 4000, as described in Polasky et al 96. Protein inference, FDR filtering, and reporter ion intensity extraction were accomplished as in the whole proteome search.

Glycan assignment and glycan-specific FDR filtering was subsequently performed in PTM-Shepherd as previously described 97. Briefly, possible glycan compositions given the observed delta mass recorded by MSFragger were scored using the glycan fragment ions observed in the spectrum and filtered to 1% FDR by comparison to spectrum-based decoy glycans. Default settings were used except for consideration of a single ammonium adduct on possible glycan compositions. PTM-Shepherd assigned glycan compositions and confidence scores were written back to the PSM tables. The PSM output files were then processed with TMT-Integrator v3.1.2 to generate summary reports at the gene, protein, peptide, site, and “multi-mass” levels from glycopeptide spectra. Multi-mass refers to the combination of glycan and site, i.e., each distinct glycan identified at a given site generates a separate entry. The PSM filtering and summarization process was the same as for whole proteome searches, with the exception of restricting the PSMs considered to those of glycopeptides and using the MSFragger-reported localization of the glycosite within identified peptides rather than PTMProphet.

### Identification and Quantification of Metabolomic Data

Acquired data were analyzed first using Thermo Scientific Compound Discoverer® software. The chromatographic peaks were integrated to obtain raw intensities of metabolites. Compounds with definite peaks and names in the software were selected. The data were then filtered based on the following criteria: m/z Cloud score greater than 60 (good fragmentation matching with compounds in the m/z Cloud database) or mass list match (mass lists include common pathways such as glycolysis, pentose phosphate pathway, hexosamine, and sialic acid pathway, purine and pyrimidine synthesis, and amino acid metabolism) and intensity >10000. Thermo Scientific TraceFinder® software was then used to quantify compounds in common pathways not found using Compound Discoverer® where the retention time (RT) was determined using Freestyle® software based on mass accuracy and fragmentation match. The data from Thermo Scientific Compound Discoverer® and TraceFinder® software were combined to generate the final list of compounds.

### Downstream Statistical Data Analysis

### Proteomic Data Normalization and Imputation

With the output from FragPipe, TMT-Integrator reports, contaminated genes and samples are filtered out, ENSEMBL IDs are mapped to gene symbols, and duplicated genes and samples are removed by using the average quantification. For global proteomics and phosphoproteomics data, data imputation was performed to support some downstream analysis. Genes with missing values more than 50% are filtered out. Separately in the discovery cohort and non-ccRCC cohort, the batch effect caused by potential uneven TMT plexing with Combat after imputing missing values in the remaining genes with KNN was removed. The two datasets are then joined together using normal samples as control. Imputed values were replaced with NA and run DreamAI imputation with default parameter setting.

### PTM Proteomics Data Normalization

With TMT-Integrator's ratio reports on global proteome and PTM (phosphorylation and glycosylation (protein level)) datasets, a linear regression model was built with all the samples' global protein ratio as predictor and their respective PTM ratio data as response. After the model is fitted, the residual values are taken as normalized PTM intensity.

### Principal Component Analysis

PCA was performed on 150 tumor samples including (103 ccRCC, 15 RO, 13 pRCC, 3 chRCC, 2 AML, 2 ESCRCC, 1 BHD, 1 MEST, 1 MTOR mutated RCC, 1 TRCC, 8 unclassified) and 101 normal adjacent (NAT) samples to illustrate the gene expression (RNAseq, 89 NAT), global proteomic, phosphoproteomic, glycoproteomic difference between tumor and NAT samples. Due to sample availability, in metabolomics data analysis, only 28 tumors (8 RO, 8 pRCC, 2 AML, 1 chRCC, 1 ESCRCC, 1 BHD, 1 MEST, 1 MTOR mutated RCC and 5 unRCC) and 7 NATs went through PCA. R function prcomp was employed to calculate loadings on each principal component. R function fviz from library “factoextra” was employed to visualize the results in 2D, ellipse of subtype groups were added with specifying parameter addEllipses=T and ellipse.level=0.5

Tumor versus Normal and Between-Subtypes Differential Expression/Proteomic Analysis

TMT-based global proteomics data were used to perform differential proteome analysis between tumor and normal samples, as well as between different subtypes. R package limma173 was used to fit a linear regression model between sample groups for proteomics data in log 2 scale. Tumor purity adjustment is achieved differently in different types of comparisons. In tumor subtype comparisons, tumor purity is added as a co-variable in the regression model. In tumor versus normal comparisons, tumor purity is the only variable in the regression model, as tumor purity for normal tissues is zero. After model fitting, the regression coefficient is the fold change in log 2 scale between comparison groups (mean difference between two groups) and the p value and q value associated with the moderated t statistic (p.mod and q.mod) calculated with the ebayes function are the resultant significance measurement.

### Nonnegative Matrix Factorization (NMF) Clustering and Heatmap Analysis

Filtered RNA (TPM) data based on raw read counts, imputed global proteomics ratio data, and imputed phosphoproteomics ratio data were supplied to SignatureAnalyzer (github.com/getzlab/SignatureAnalyzer) to perform automatic relevance determination (ARD) NMF clustering44. Clustering results with immune deconvolution result, mutation information, copy number variations are visualized with heatmaps through R library ComplexHeatmap200 (FIG. 1A).

### Immune Deconvolution

To estimate the fraction of different cell types in the tissue microenvironment, a multi-omic based deconvolution integrating global proteomic and RNAseq data was performed via BayesDebulk 178. Only samples with both gene expression and global proteomic measurements were considered. Protein abundance was imputed as described above before being inputted to BayesDebulk. To perform the deconvolution, BayesDeBulk requires a list of cell-type specific markers for each cell type. For immune cells, such list was derived from the LM22 signature matrix 201 in a similar fashion as in Petralia et al178. For this analysis, an aggregated version of the LM22 signature matrix was utilized. Specifically, the LM22 values mapping to different types of CD4 T Cells (e.g., Memory T Cells, Naïve T Cells) were averaged to create a gene signature for CD4 T Cells. The same strategy was utilized for Dendritic cells, Natural Killers cells, Mast Cells and B Cells. For each pair of cell types, a marker was considered to be upregulated in the first cell type compared to the other cell type if the corresponding value of the LM22 matrix for the first cell type was greater than 1,000 and 5 times the value of the other cell type. For Endothelial-PLVAP, Endothelial-ACKR1, Pericytes and vSMC, marker signatures from a previous ccRCC single-cell RNAseq study25 were used. To derive these signatures, differential expression between different single-cell clusters was performed and only markers significant at 10% FDR and a log fold change greater than 1 were considered as cell-type specific markers.

Finally, Macrophage A and Macrophage B signatures from Zhang et al25 were considered. As common markers for Macrophage A and B, C1QA, C1QB, C1QC, MS4A6A, LYZ, TYROBP, FCGR2A, FCERIG, AIF1, CD14, CD68 were used; as markers specific of Macrophages A CXCL8, CXCL2, CCL4, CCL3, CCL4L2, CXCL3, CCL3L3, CCL20, NFKB1, ILIB were used; while for Macrophage B the following cell-type specific markers were considered: CTSL, LGMN, ASAHI, LIPA, CTSD, LAMP1. BayesDeBulk was estimated via 10,000 Monte Carlo Markov Chain (MCMC) iterations. Cell-type fractions were estimated as the mean across MCMC iterations after discarding a burn-in of 1,000 iterations. Once estimated, cell-type fractions for each patient were standardized to sum to the total fraction of immune/stromal cells in the tissue microenvironment. The total fraction of immune/stromal cells was computed as one minus the tissue purity inferred from gene expression data. The estimation of the purity was performed via TSNet202.

### Immune/Methylation Subtype Clustering

Immune deconvolution results and methylation data were subjected to a consensus clustering algorithm to identify subtypes within tumors. Percentages of immune cells calculated by BayesDebulk were used for immune subtyping. For methylation subtyping, beta values from the methylation array harmonization workflow based on SeSAMe203 were downloaded from CPTAC DCC and GDC. To avoid methylation probes of ccRCC from overrepresentation, top 4000 most variable probes with less than 50% missing values were selected independently for ccRCC-Discovery cohort and non-ccRCC cohort. Selected probes were then combined with remaining missing values imputed by the mean of the corresponding probe. Consensus clustering was performed via CancerSubtypes 172 with following parameters: maxK=10, reps=1000, pItem=0.8, pFeature=1, clusterAlg= “km”, distance= “euclidean”. Numbers of clusters were chosen based on the delta area plot of the consensus CDF.

High versus Low wGII Groups Determination

To measure overall instability, a published measure of Weighted Genome Instability Index (wGII) was used45. WGII was measured as the proportion of each chromosome which has a different copy number compared to the baseline copy number of the sample. Then the average of scores for each chromosome was calculated, weighted by the length of the chromosome such that each chromosome has the same contribution to the overall instability score.

To validate the results, wGII were also calculated for TCGA kidney cohorts (KIRC, KIRP, KICH). All the required information regarding the segmentation, absolute copy numbers and purity values were acquired through TCGA PanCanAtlas GDC portal20. The cutoff of high or low wGII grouping was determined by finding the cutoff that minimizes the p-value of survival differences of the two groups using a cox-regression modeling (cutoff=0.32), using the TCGA-KIRP cohort. In practice, to make sure each group has enough samples, a cutoff of 0.3, which yields a more balanced populated grouping, but still a significant p-value was used.

### Dimension Reduction in Single Nucleus RNAseq and Cell Type Assignment

Following data processing all the sample libraries passed various QC measures including floating RNA contamination that was estimated by SOUPX median 6% (range 1.2 to 17%). Data from 79,673 nuclei from 8 samples (median 10,592) were used in integrative analysis with previously published snRNAseq data from benign kidney samples. Cell type annotations were rendered by examining biomarker expression patterns identified based on previous single cell RNA-seq and single nucleus RNA-seq data25,62.

Downstream analyses were performed with Seurat204 v4.1. Filtered count matrix was normalized to 10000 UMI per cell and natural-log transformed, top 2000 highly variable genes (HVGs) were identified by modeling the mean-variance relationship, PCA was then performed on scaled and centered matrix (including HVGs only); finally cells were projected into a 2-D map with UMAP205 using the first 30 PCs. Clusters were identified using the Louvain clustering algorithm (resolution=0.5) on a shared nearest neighbor graph. Expression of known tumor markers [FOXI1 and LINC01187 for ChRCC and oncocytoma12, TRIM63 for TRCC10, ITGB8 for pRCC206, PAX8 for ESCRCC207, PECAM1 and ENG for endothelial cells, ACTA2 and RGS5 for SMC, PTPRC for immune cells, FABP4 and PLN1 for adipocytes] were used to annotate cell clusters. Since AMLs consist of blood vessels, smooth muscle cells, and adipocytes, cells expressing markers of SMC, endothelial cells and adipocytes were considered “Tumor” for AML libraries. Annotations of non-tumor cells were complemented by prediction using a published snRNA-seq data set of human normal kidney62 as reference. Re-clustering of all tumor cells with resolution=0.2 was done for each sample to identify tumor subclusters. Cell cycle phase was assigned based on scoring of expression of G2/M and S phase markers 208.

To visualize clustering patterns of cell types from all RCC subtypes, a subset of 2000 cells of each library were randomly selected and pooled together. Downstream analyses from normalization to dimension reduction (UMAP) followed the same procedure as for individual libraries. To show similarity and dissimilarity among tumor cells of different subtypes, tumor cells of all libraries except AMLs were pooled and PCA was performed on top 500 highly variable genes.

### Cell-of-Origin Prediction and Bulk Data Projection

Putative cell-of-origin of each profiled RCC subtype was inferred following previously published procedure 25. A random forest model was trained with a snRNA-seq dataset of normal kidney epithelial cells (only HVGs were used; 300 cells were randomly selected for overrepresented clusters to minimize bias due to unbalanced sample sizes). The model was then applied to snRNA-seq data of RCC tumor cells to predict their closest normal cell types (putative COO). In addition, prediction based on bulk RNA-seq data of ccRCC and rare RCCs of this study was performed by first applying rank-based inverse normal transformation. Random forest classifier was then built on transformed sn data of normal kidney epithelial cells; transformed bulk data of RCCs were then used to predict putative COO.

### Proteogenomic Signature of RCC Subtypes

To identify subtype-specific markers, differentially expression (DE) analysis was performed for each of the rare RCC subtypes profiled by RNA-seq and proteomics. For RNA-seq, the input data is voom-transformed data with associated precision weights 209. For proteomic data, the input was normalized and log 2 transformed data. Input data was fit into linear models with limma 173 and contrasts were constructed to compare each subtype with the average of all other subtypes. Top 100 upregulated genes ranked by p value were selected for each subtype (for RNA, additional filter of logFC>2, adjusted p value <0.01 were applied) as the signature gene set. To visualize expression of the gene sets as “metagenes”, z-score was calculated for each gene of each gene set and the average was used to make the heatmap.

### Gene Set/Pathway Enrichment Analysis

To visualize the pathway enriched across different RCC subtypes, differential expression analysis was first performed to identify differential expressed RNAs and proteins, respectively. Then, gene set enrichment analysis was performed to identify enriched concepts. GSEA for RNA and proteomics data were performed through the ClusterProfiler R package. The annotation of concepts are fetched from REATCOME 210, MSigDB 71 and KEGG 211.

### Phosphorylation Site Level Enrichment Analysis

Based on the results of differential expression analysis with phosphorylation sites intensity data between each tumor subtype and normal samples, phosphosite-specific signature enrichment analysis174 (PTM-SEA) was performed to identify dysregulated phosphorylation-driven pathways. To adequately account for both magnitude and variance of measured phosphosite abundance, t.mod, moderated t statistic resulting from the ebayes function as ranking for PTM-SEA was used. The PTM signature database (PTMsigDB) v1.9.0downloaded from/prot-shiny-vm.broadinstitute.org: 3838/ptmsigdb-app/was quieried using the Uniprot ID plus residue location as identifier. The functions of PTM-SEA available on GitHub (github.com/broadinstitute/ssGSEA2.0) within R were called. The following parameters were used to run PTM-SEA; weight: 1, statistic: “area.under.RES”, output.score.type: “NES”, nperm: 1000, min.overlap: 5, correl.type: “z.score”

The sign of the normalized enrichment score (NES) calculated for each signature corresponds to the sign of the tumor-normal log fold change. P-values for each signature were derived from 1,000 random permutations and further adjusted for multiple hypothesis testing using the method proposed by Benjamini & and Hochberg (Benjamini and Hochberg, 1995). Due to limited sample size, signatures with a lenient FDR threshold 0.2 were considered to be differential. Pathway signatures that are significant in at least one of the 9 comparisons are displayed in the bubble plot. Kinase signatures are plotted in pseudo volcano plots in high vs low wGII non-ccRCC tumor comparison and chromosome 7 gain vs no gain non-ccRCC (pRCC, TRCC and ESCRCC) comparison.

Metabolic Pathway Enrichment Analysis with Metabolites and Enzymes

Upregulated and downregulated metabolites and proteins (q value <0.05, absolute value of log 2 fold change >1) in ccRCC, pRCC type1, AML, RO type2 combined with RO variants compared to normal samples are sent to IMPaLA175 (impala.molgen.mpg.de/) for pathway over-representation analysis separately. KEGG ID is the metabolite identifier and gene symbol is the protein identifier. HumanCyc metabolic pathways212 (humancyc.org/) from the analysis results were further investigated.

### Transcription Factor Regulon Identification

pySCENIC (pyscenic.readthedocs.io/en/latest/) and R package SCENIC (version 1.1.2) were used for transcription factor regulon identification. Input of this analysis are raw bulk RNA-Seq data and the raw proteome data. An in-house constructed pipeline via combining GRNBoost2 from pySCENIC algorithms and RSCENIC algorithms with default parameters was used. To predict the regulons, human v9 motif collection, as well as both hg38_refseq-r80_10kb_up_and_down_tss.mc9nr.feather and hg38_refseq-r80_500 bp_u-p_and_100 bp_down_tss.mc9nr.feather databases from the cisTarget (resources.aertslab.org/cistarget/databases/homo_sapiens/hg38/refseq_r80/mc9nr/gene_based/) were used. The resulting AUC scores matrix was used for downstream analysis (Heatmap and RSS plot)

### Kinase-Substrate Co-Regulation Analysis

Kinase and residue level substrate relationships were collected from OmniPath (OmniPath: Intra-& intercellular signaling knowledge (omnipathdb.org)) using R package OmnipathR for comprehensive coverage. Keeping only “phosphorylation” modifications, one obtains 40,122 kinase-substrate pairs. After mapping the proteome data to kinases and the phosphoproteome data to substrates (with site level resolution), 9,932 kinase-substrate pairs were identified for joint differential analysis. Two-dimensional Z score vectors were derived for both kinase (k) and substrate(s):

$$Z = < Zk , Zs >$$

Using ImFit and cBayes function from the limma package, kinase protein abundance/phosphorylation site intensity data was separately regressed against sample grouping (low wGII or high wGII) with tumor purity adjusted as a covariate. The resultant t statistic is assigned as the Z score for kinase and substrate respectively. The distribution of all Z scores derived from all the kinase-substrate pairs f was modeled as a mixture of pairs which are up-or down-regulated between conditions (e.g. high or low wGII) and pairs which are non-differentially regulated. Assuming the Z scores of the differentially regulated proteins follow an empirical distribution f1 and the Z scores of the non-differentially regulated proteins follow an empirical null distribution f0, one can write the observed distribution for Z score as

$$f ( Z ) = p 0 f 0 ( Z ) + p 1 f 1 ( Z ) ,$$

where p0,p1 are the proportion of the non-differentially and differentially regulated pairs in the data. p0+p1=1. To estimate the distribution of the non-differentially regulated pairs, the null distribution of Z scores by is simulated by randomly permuting the measurements of the two conditions for 200 times. Following this mixture deconvolution, the posterior probability of being differentially regulated for each protein is calculated with the following equation:

$$p 1 ( Z ) = 1 - p 0 f 0 ( Z ) / f ( Z )$$

pois estimated by taking the ratio of densities at:

$$Z 00 = < Zk = 0 , Zs = 0 > ,$$

$$p 0 = f ( Z 00 ) / f 0 ( Z 00 ) .$$

The local false discovery rate is computed as:

$$fdr ( Z ) = p 0 f 0 ( Z ) / f ( Z ) .$$

Throughout this modeling, only the proteins and phosphosites with missing data in fewer than 6 samples were used for construction of reliable Z score. The R implementation of KSA2D can be found in www.github.com/ginnyintifa/KSA2D.

pRCC MTSCC Specific Marker Validation with External Data

Protein expression of a pRCC and MTSCC cohort was fetched from the previous publication34. Differential expression analysis was performed in the same way as described in the Differential Expression Analysis section. One outliner MTSCC sample was removed due to absence of canonical copy number loss events of chr1 chr6, and chr9.

### Survival Analysis

The R package “survival” was used to perform survival analysis. The Kaplan-Meier curve of overall survival was used to compare the prognosis among subtypes (function survfit). The standard multivariate Cox-proportional hazard modeling (function coxph) was applied to calculate hazard ratio between categories of interest (e.g., high or low expression of certain gene, methylation groups, wGII groups). Age, gender, race, tumor stage and tumor purity were adjusted as the model covariates. Log-rank test was used to test the differential survival outcomes between categorical variables.

Concordance Index calculation in prognostic marker selection

To calculate concordance index (GI) for each gene, samples were divided into 5 folds with the same distribution of event and censored cases. 4 folds are assigned as training data to fit a survival model (function coxph), then the fitted model was used to calculate survival probability for the rest sample in the left over 1-fold (function predictSurvProb). Hence CI is calculated using the median survival time with the function “Cindex” in these test samples. This process was repeated 30 times with a random split of training and test for each run, and the median of 30 runs is taken as the final CI.

### Results

### Specimens, Multi-Omics Data Types, and Protrack Web Browser

Understanding the shared and unique proteogenomic aberrations between ccRCC and non-ccRCC will inform disease diagnosis, prognosis, and therapy. Proteogenomic multi-omics data was examined from 151 kidney tumors including 103 ccRCC from Clark et al 34. 48 non-ccRCC tumors, and 101 benign adjacent kidney tissue (NAT) samples (79 from ccRCC and 22 non-ccRCC patients) (FIG. 29A). The 48 non-ccRCC patients represent various histologic subtypes of which 41 patients were newly profiled and seven were from a previous study 34. Integrated proteogenomics analysis was carried out on eight different multi-omics data types (genomic, transcriptomic, proteomic, metabolomic (selected samples) and post-translational modifications (PTMs-phosphorylation and glycosylation) generated from a common sample aliquot 35 (FIG. 29A). Whole genome sequencing (WGS), whole exome sequencing (WES), DNA methylation profiling, and RNA sequencing (RNA-seq) was available for all 151 tumor samples, while RNA-seq data was available for 89 out of 101 NATs (ccRCC n=71; non-ccRCC, n=18) (FIG. 29A). Single nuclei RNAseq (snRNAseq) was performed for eight non-ccRCC patients.

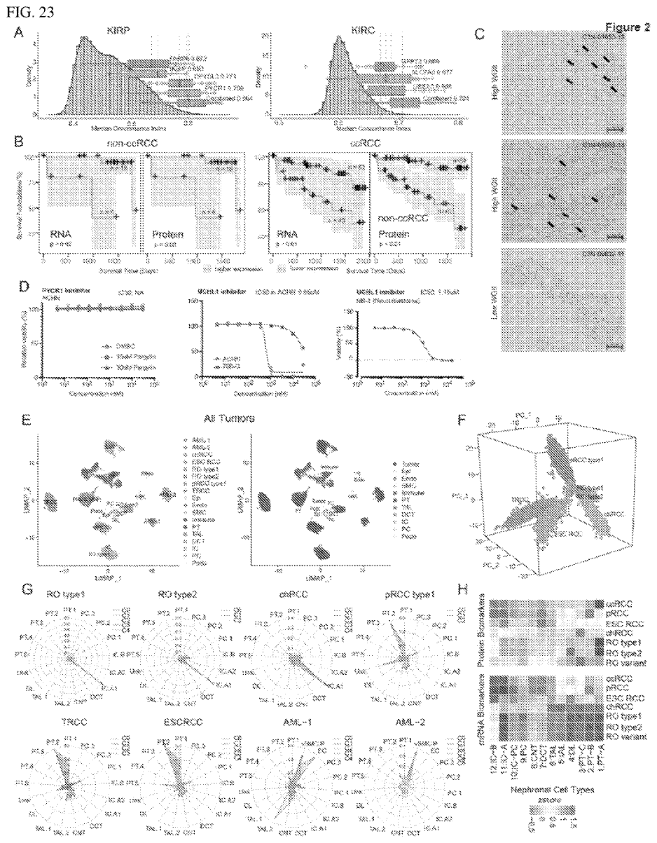

Tumor classification based on histological subtyping was refined further by signature molecular aberrations such as copy number variation (CNV) patterns, somatic/germline mutations, marker gene expression, and gene fusions (FIG. 23A) 36. The final analysis cohort composition included 103 clear cell RCC (ccRCC), 15 renal oncocytomas (RO, 3 type-1, 8 type-2, and 4 variant), 13 papillary RCC (pRCC, 8 pRCC type-1, and 5 other pRCC), 3 chromophobe RCC (chRCC), 2 angiomyolipoma (AML), 2 eosinophilic solid and cystic RCC (ESCRCC), 1 Birt-Hogg-Dube syndrome-associated renal cell carcinoma (BHD), 1 mixed epithelial and stromal tumor of the kidney (MEST), 1 MTOR mutated RCC, 1 translocation RCC (TRCC), and 8 unclassified/other RCC (unRCC) (FIG. 23A). Based on molecular assessment, a plasmacytoid urothelial carcinoma (PUC) and 2 NAT samples which were contaminated by tumor tissues were excluded from further analysis (Methods).

The multi-omics data in a queryable format, various QC analyses, and clinical parameters have been added to the existing ProTrack web application (ccrcc-conf.cptac-data-view.org, FIG. 29B) which serves as a public resource to enable processed data navigation, visualization, and download 37. The demographic and clinical composition are largely comparable between ccRCC and non-ccRCC cohorts, except for a higher proportion of female patients in non-ccRCC (p-value=0.036) (FIG. 29B). A total of 12,299 proteins, 9,396 phosphorylated proteins, and 1,035 glycoproteins, of which 9,528 proteins, 6,465 phosphorylated proteins were identified, and 639 glycoproteins were quantified in more than half of all samples. Principal component analysis (PCA) on global proteome, phosphoproteome, and glycoproteome data on the RCC types (ccRCC, pRCC, RO) showed clear separation in 2-dimensional space (FIG. 29C).

### Subtype-Specific and Common Proteogenomic Signatures in Kidney Tumors

Proteogenomic multi-omic analysis revealed both unique and shared molecular features across disease subtypes. Non-ccRCC tumors contained distinct subtype-specific recurrent genomic aberrations that were vastly different from ccRCC where near universal chr3p loss, frequent chr14q loss, VHL, BAP, SETD2, and PBRM1 mutations were noted (FIG. 23A). Supporting previous classification of renal RO molecular subtypes 28, RO type-1 was enriched with CCND1 gene rearrangement with a diploid genome and was mutually exclusive with the RO type-2 subtype, which was typified by one copy loss of chromosome 1. Heterogenous RO cases lacking the above stated molecular characteristics that were collectively grouped under “RO variant”, a subgroup that is not fully characterized, were also identified 28. TP53 mutations and the signature chromosomal losses in chRCC, chr7/17 gain (8/8 cases) and MET mutations (3/8 cases) in pRCC, TSC gene mutations in ESC-RCC (2/2 cases) and AML tumors (2/2 cases), and the TFE3 gene fusion in the TRCC case were notable subtype-specific events in non-ccRCC. However, even among the apparent subtype-specific genomic aberrations, there was an underlying common theme, where bi-allelic loss of tumor suppressor genes were the most recurrent driver events in kidney malignancies, barring some outliers, such as type-1 pRCC, TRCC, and other rarer instances.

Regarding shared molecular features, Gene Set Enrichment Analysis (GSEA) of RCC subtype-specific differentially expressed genes and proteins revealed several pathway similarities and differences between ccRCC and non-ccRCC (FIG. 23B). For instance, immune/inflammatory response concepts, including allograft_rejection, inflammatory_response, Interferon_alpha, and interferon gamma pathways, were significantly upregulated, especially at the protein levels, in both pRCC and ccRCC. In contrast, pathways such as glycolysis, hypoxia, and EMT were significantly enriched in the ccRCC proteome but showed a negative enrichment trend in pRCC and RO. Interestingly, oxidative phosphorylation was down in ccRCC and pRCC as expected but showed significant positive enrichment in RO (FIGS. 23A and 23B) 28, 32, 34, 38.

Immune deconvolution analysis was performed based on both RNA and protein data (Methods, FIG. 23A) to study the immune infiltration status of kidney tumors. Clustering analysis revealed seven different immune clusters, including the previous four ccRCC clusters 34, one myeloid-high non-ccRCC cluster with the majority of pRCC samples, one immune-absent non-ccRCC cluster with all of the oncocytic tumors, and one myeloid-lymphoid high non-ccRCC cluster (FIG. 23A). Overall, the extent of immune infiltration was lower in non-ccRCC than in ccRCC (FIG. 23C). Interestingly, myeloid-lymphoid high non-ccRCC tumors showed high immune infiltration and high whole genome instability index (wGII) (FIG. 23A). In both ccRCC and non-ccRCC, 10-15% of CPTAC and TCGA patients showed higher ploidy and CNV burden (FIG. 23D, 29D, 29E), as scored by wGII. This disease molecular subset was associated with poor survival in both TCGA and CPTAC ccRCC and non-ccRCC datasets (FIG. 29F).

### Proteogenomic Impact of Genome Instability

Genome instability (GI), a recognized cancer hallmark, includes CNV burden, tumor mutational burden (TMB), and microsatellite instability and is variably associated with several clinical/prognostic features in solid tumors 39, 40. GI and intratumor mutational heterogeneity in ccRCC were associated with poor prognosis based on multi-region genomic profiling of 38 ccRCC patients in the landmark TRACERx 41, 42 program, but these studies lacked DNA methylation data. Meanwhile, TCGA studies 21, 29 which profiled 894 patients representing three major RCC subtypes (KIRC [n=503], KIRP [n=285], KICH [n=78]) and miscellaneous tumors (n=28) uncovered the important association between DNA methylation patterns and survival. However, the copy number-based GI assessment in the TCGA study was less robust as they utilized focal CNVs from only 39 regions 29. The ccRCC proteogenomic study (See above Examples) revealed association between poor survival and combination of molecular features, including DNA hypermethylation (DNA-Methyl1), BAP1 mutations, and GI in treatment naive patients. Hence, to comprehensively evaluate the overlap between samples with GI and DNA methylation patterns and to study the proteogenomic impact of GI, integrative analysis with both CPTAC and TCGA ccRCC and non-ccRCC datasets was performed.

In an unsupervised approach, RNA, protein, and phosphosite expression data was collectively analyzed using automatic relevance determination nonnegative matrix factorization (ARD-NMF) clustering 43 to identify multi-omics clusters that might capture subtype-specific and common molecular events. Among the six ARD-NMF clusters, ccRCC samples were found in ARD-NMF1 and 5 (FIG. 23A). The smaller ARD-NMF-1 is associated with DNA hypermethylated Methyl1 group, higher grade ccRCC and worse prognosis, while the larger ARD-NMF-5 ccRCC cluster is enriched (p<0.05, chi-squared test) with low grade ccRCC tumors. Other major NMF clusters were largely tumor type-specific as most pRCC were clustered in ARD-NMF-0 and oncocytic tumors (RO, chRCC) under ARD-NMF-3, while ARD-NMF-2 and ARD-NMF-4 contained all the NATs. Next, as previous studies 21, 29 showed an association between DNA hypermethylation subgroups and worse survival, consensus clustering was performed with DNA methylation data and five different methylation clusters were identified. Methyl3 and Methyl5 were largely subtype-specific and contained ccRCC and all oncocytic tumors, respectively. Methyl1 was enriched with ccRCC samples with high wGII, BAP1 mutants, and a subset of non-ccRCC samples with high wGII and high ploidy mostly from the unRCC/other category (FIG. 23E).

To identify patients with GI, WES/WGS was used for CPTAC samples and the WES data from the three TCGA kidney cancer (KIRC, KIRP, and KICH) cohorts were reprocessed to determine absolute CNV, genome ploidy, and instability values (wGII score) 44 (Methods). Chen et al. 29 in their TCGA pan-RCC analysis identified distinct molecular RCC subtypes such as “CCe-3” and “P.CIMP-e” that were significantly associated with poor survival among ccRCC and pRCC, respectively. The absolute CNV analysis revealed a higher fraction of cases with high wGII among TCGA ccRCC CCe-3 and in pRCC e-CIMP molecular subgroups29 (FIG. 29D). In contrast, Chen et al 29 called more CCe-1 samples as unstable using only 39 focal copy number data. In this regard, dimensionality reduction analysis collectively on TCGA and CPTAC RNA-seq data also showed that CPTAC ccRCC and non-ccRCC samples containing high wGII overlapped with TCGA ccRCC CCe-3 and non-ccRCC P.CIMP-e samples, respectively (FIG. 23F). KICH in general had a uniformly higher wGII score reflective of the recurrent chromosomal losses considered an aberration signature of this disease and served as a good internal control. A subset of KICH/KIRP/KIRC cases that were later re-classified as mixed tumors by the TCGA pan-RCC studies, as they lacked signature chromosomal losses but contained MTOR mutations and diploid genomes, had the lowest wGII scores, as expected (FIGS. 29D). High wGII cases in the TCGA cohorts irrespective of the molecular subtypes were associated with poor survival (FIGS. 29D, 29F).

mRNA and protein differential expression analysis was performed (Methods Section) between high vs low wGII in CPTAC non-ccRCC samples (FIG. 23G) and results were compared with similar analysis in TCGA KIRP RNA data. Differentially expressed genes (DEGs) associated with high wGII in the CPTAC non-ccRCC cohort highly overlapped with high wGII TCGA KIRP, and similar results were noted for ccRCC tumors. Simultaneously, each gene was screened in both KIRP and KIRC cohorts to assess whether their mRNA expression levels were predictive of survival outcome. One important observation was that genes showing stronger association with poor survival were enriched in the high wGII feature sets (FIG. 29G). It was observed that PYCR1, FABP6, MAP1B, DPYSL3, TOP2A, RRM2, CSRP2, IKBIP, STK26, and STEAP3, among others, were significantly upregulated at the protein and RNA levels in high wGII non-ccRCC cases 45, 46 (FIG. 23G, 1H). PYCR1, FABP6, TOP2A, RRM2, and STEAP3 were also upregulated in high wGII ccRCC samples. Hallmark pathways associated with high wGII cases revealed increased cell cycle/proliferation concepts (enrichment of E2F targets, G2-M checkpoint), differential enrichment of immune and inflammation related concepts, EMT, hypoxia, and glycolysis were also observed and provided further insights into the biology of this disease subset (FIG. 29H). TOP2A, MYBL2, and STEAP3 are part of the cell cycle, while PYCR1 is from the cellular response to stimulus concept (FIG. 23H). The mitochondrial enzyme PYCR1 (Pyrroline-5-carboxylate reductase 1) catalyzes the last step in proline biosynthesis, and its importance in cancer cell survival in oxygen limiting conditions and to promotion of cancer invasion and progression has been noted in multiple cancer types. In summary, though the overlap of DEGs and DEPs associated with high wGII between ccRCC and non-ccRCC is minimal (14 genes with RNA expression level evidence), higher similarity was observed at the overall pathway level processes between the two cancer types that are active in high wGII tumors (FIGS. 29H and G). Further, it was demonstrated that the hyper-methylated group, immune-enriched group, and high wGII group are all associated with worse survival in the pan-RCC setting (FIG. 29F and 29I).

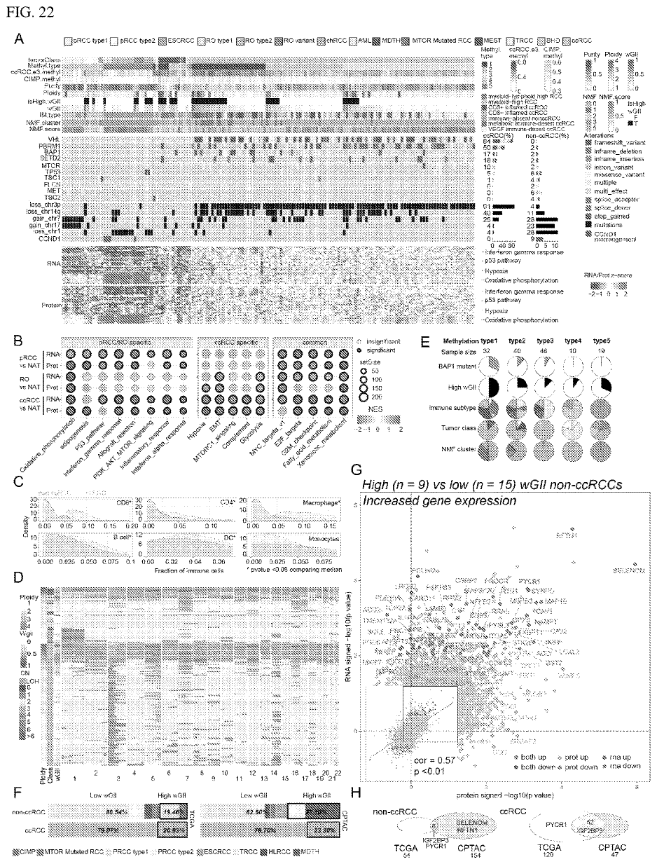

mRNA and protein differential expression analysis between was compared between high (n=9) vs low (n=15) wGII non-ccRCC samples from pRCCs, TRCC, ESCRCC, MTOR mutated and MDTH categories. Two facets of gene expression data correlate well in terms of differential expression significance (FIG. 22G). A collection of prominent wGII markers, comprising RFTN1, SELENOM, PYCR1, MAPIB, CAV1, CAV2, NAMPT, NNMT, GPX8, FKBP11, IKBIP, LOXL2 and BST2, was concordantly identified. It is noteworthy that these significantly higher expressed genes are not necessarily cell cycle-associated genes. For instance, the mitochondrial proline biosynthetic pathway enzyme PYCR1 (Pyrroline-5-carboxylate reductase 1) is known to support cancer cell survival under oxygen-deprived conditions and cancer invasion and progression across multiple cancer types. RNA-ISH staining validates PYCR1 upregulation in high wGII samples (FIG. 22F). NAMPT and NNMT are involved directly and indirectly in NAD+ (Nicotinamide Adenine Dinucleotide) biosynthesis which has a critical role in energy production, DNA repair and regulation of gene expression. The expression of IKBIP (I kappa B kinase interacting protein) has been linked to unfavorable prognosis in GBM patients, potentially attributed to its inhibition of CDK4 ubiquitination and degradation. LOXL2 (Lysyl oxidase homolog 2), has been implicated in driving tumor progression and metastasis possibly through activation of EMT. IGF2BP3 shows significantly higher mRNA expression in non-ccRCC high wGII samples (FIG. 22G), and upregulation trend in protein expression which was validated by IHC results (FIG. 22). Importantly, in both CPTAC and TCGA, non-ccRCC and ccRCC cohort RNAseq data, high vs low wGII differential expression (DE) analysis noted high IGF2BP3 RNA expression in high wGII samples (FIG. 22H). The RNA binding protein and N6-methyladenosine reader IGF2BP3 has not been previously associated with high wGII.

In summary, though the overlap of DEGs and DEPs associated with high wGII between ccRCC and non-ccRCC is minimal (14 genes with RNA expression level evidence), Hallmark pathways associated with high wGII cases implicate increased cell cycle/proliferation concepts (enrichment of E2F targets, G2-M checkpoint). Differential enrichment of immune and inflammation related concepts, EMT, hypoxia, and glycolysis were also observed and provided further insights into the biology of this disease subset (FIG. 29H). Furthermore, higher similarity at the overall pathway level processes was observed between the two cancer types that are active in high wGII tumors (FIG. 29H). It is worth noting that MDTH non-ccRCC tumors are largely genome unstable (6/7). They also tend to be hyper methylated (4/7) and immune infiltrated (6/7)

### Identification of Non-ccRCC Prognostic Markers

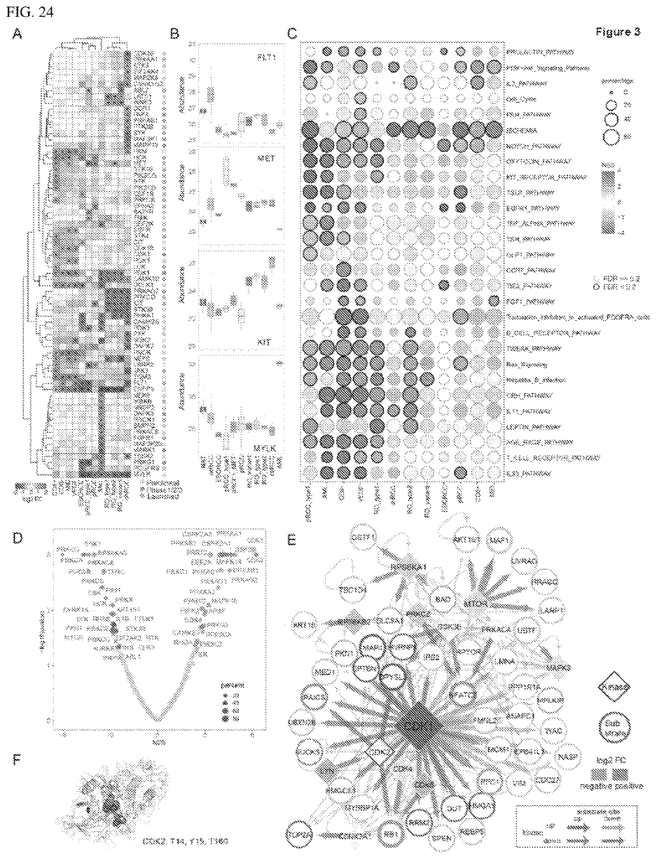

To obtain the most prognostic genes for the non-ccRCC cohort, survival prediction was performed using RNA expression of genes in the KIRP cohort. The prediction power of each gene was evaluated using a concordance index with patients' age and tumor purity adjustment (CI) (Methods). 998 genes tested in both survival predictability and wGII DE analysis show median CI larger than 0.659 (90% percentile of all CI). Among these genes, five of them were identified as high WGII features with both KIRP RNA and CPTAC non-ccRCC RNA data, at the same time, there are increased protein levels in high wGII group. Four genes were selected as being highly prognostic and highly associated with genome instability. These four genes were PYCR1, DPYSL3, IKBIP, and FABP6, with median CI at 0.799, 0.771, 0.693, and 0.672, respectively. The four genes combined yielded an even higher median CI at 0.804 (FIG. 24A). A similar approach was applied to KIRC data for prognostic marker comparison. The distribution of CI of all genes' RNA expression in KIRC was centered above 0.6 and with less variation, though the maximum predictability was smaller than that of KIRP (FIG. 24A). Three markers were nominated for KIRC: UBE2C, SLC7A5, and GFPT2 with median CI at 0.686, 0.677, and 0.669, respectively; the three gene combined panel yielded median CI at 0.704. These prognostic marker panels were next validated in this RCC dataset. Setting cutoff values of mean protein or RNA expression of the panel genes and grouping the ccRCC or non-ccRCC cohort into higher expression and lower expression arms, it was validated that the two arms exhibit significantly different survival outcomes (FIG. 24B).

FABP6 is highly expressed in the ileum and is an intracellular transporter of bile acids in ileal epithelial cells, which helps catalyze and metabolize cholesterol. Studies also show FABPs are important in cell proliferation 47, and FABP6 is proposed as a prognostic marker for colorectal cancer 47,48. Similarly, IKBIP expression was found to be associated with poor prognosis in GBM patients, possibly through inhibiting ubiquitination and degradation of CDK449. DPYSL3 (CRMP) is a pancreatic cancer prognostic marker which is known to be involved in proliferation, apoptosis, differentiation, and invasion in several cancers 50, 51. PYCR1 has been previously shown to be associated with prognosis in papillary RCC 52 and plays a role in ECM modulation by cancer-associated fibroblasts in breast cancer 53. PYCR1 RNA differential expression was validated in GI high versus low non-ccRCC tumors by RNA-ISH (FIG. 23C). However PYCR1 small molecular inhibitors pargyline and compound 454 treatments did not affect ACHN1cell viability. The UCHL1 gene that was associated with wGII in ccRCC was also upregulated in high wGII KIRP samples (FIG. 30A). ACHN1 cells when treated with UCHL1 inhibitor showed a dramatic decrease in cell viability of IC50 nM which is comparable to the effect in neuroblastoma cell line NB1 (IC50 1.6 μM) showing that the UCHL1 protein is also a target in non-ccRCC (FIG. 23D). UCHL1 expression was identified to be marker of good prognosis in neuroblastoma.

Non-ccRCC snRNAseq Cell Atlases Reveal Transcriptomic Heterogeneity Among Tumor Subclusters and Low Immune Infiltration

Intratumoral genomic aberration heterogeneity, frequently encountered in ccRCC, has been associated with a worse prognosis 55. In addition, intratumor morphologic heterogeneity is also common, but the underlying reason for this phenomenon has not been fully elucidated. Single cell profiling allows exploration of the genotype-phenotype association between molecular and cellular processes; however, most of the available data is from ccRCC samples. To explore this association in non-ccRCCs, eight rare kidney tumor samples were subjected to snRNA-seq and were jointly analyzed with three ccRCC samples sequenced in the companion study (Above Examples) to delineate the cell type-specific transcriptomes of tumor and microenvironment compartments. A total of 79,673 single nuclei transcriptomes (median 10,592 nuclei per sample) from rare kidney tumors were obtained. Cell type composition of the samples were deduced following cell type annotations based on known marker expression.

Dimensionality reduction UMAP analysis of the downsampled (2000 nuclei per sample) non-ccRCC samples showed tumor microenvironment cell types including immune, endothelial, and stromal cells clustered by cell type irrespective of the patient of origin (FIGS. 23E and 30B) while the tumor epithelia formed patient-specific clusters. The underlying transcriptional similarity between tumor clusters determined their relative positions where the two AML tumor clusters were closer to each other in PCA space (FIG. 23F). Likewise, ROs and chRCC were closer in space, and the papillary spectrum tumors, pRCC type-1, ESCRCC, and TRCC were more distinctly positioned. Most non-ccRCC samples had higher tumor cell fractions, implying higher tumor content and lower immune infiltration 21 as compared to ccRCC (FIG. 30B, 30C and 30D) except for the two AML cases with higher immune fraction (FIGS. 30B and 30C). Between the two AMLs, one had high macrophage numbers while the other had a high T-cell fraction, even though both cases are defined by the loss of function bi-allelic driver mutations in TSC genes, implying that other tumor intrinsic factors may dictate immune infiltration patterns.

Multiple tumor subclusters in a given sample due to transcriptomic heterogeneity helped capture gene signatures of the constituent tumor cell types (FIG. 30D). In renal AML for instance, the tumor compartment comprises an admixture of cells that are histologically and molecularly similar to vascular (angio-), smooth muscle (myo-), and fat (lipo-) lineages 56. It is hypothesized that trans differentiation from a common cell of origin gives rise to these distinct tumor cell types in a given tumor 57. In the AML snRNAseq data, the major tumor cell cluster showed a myoepithelial/smooth muscle gene expression pattern. However, among the two rare tumor clusters, one contained an endothelial-like gene expression pattern and the other showed an adipocyte-like signature (FIG. 30D), thereby capturing for the first-time gene signatures of the constituent tumor cell types of AML.

Similarly, in ESCRCC, another kidney tumor also containing somatic bi-allelic loss of TSC genes as the key driver aberration, several tumor clusters were observed (FIG. 30E). Following the above logic, it is contemplated that the distinct tumor clusters in the ESCRCC sample might represent different tumor epithelial morphologic features. ESCRCC tumor epithelia appears in solid and cystic growth patterns composed of cells with abundant eosinophilic cytoplasm. Among ROs, type-1 tumor intriguingly showed multiple tumor subclusters as compared to type-2 which had a single tumor cluster. Specifically, one of the RO type-1 tumor subclusters can be entirely associated with the S phase of the cell cycle indicating higher proliferation rates in these tumors defined by CCND1 gene rearrangement (FIG. 30F). In certain ccRCC tumors, subclusters are associated with variation in copy number heterogeneity or other genomic aberration 24; however a similar phenomenon cannot explain the tumor subclusters in RO type-1, ESCRCC, and AML as these tumors have a diploid genome and very few subclonal mutations (FIG. 22D). Among other tumors analyzed, all tumor clusters from a given sample showed significant reduction in mRNA expression from index chromosomes with clonal loss such as chr7 and 17 gain in pRCC, chr1 loss in type-2 RO etc (FIG. 30G).

Single cell resolution transcriptome data from the tumor cell clusters can be employed to predict tumor cell of origin. To explore this, integrative analysis was performed between publicly available benign human kidney snRNA-seq data 58 and the various RCC subtype tumor snRNAseq data generated here, using a previously described methodology 24 (FIG. 23G). Briefly, a random forest model trained on benign nephronal epithelial cell types was serially employed to identify the similarity between the tumor clusters in each of the RCC subtypes to the benign nephronal epithelium (FIG. 23G, Methods). TRCC, ESCRCC, and pRCC showed highest probability to the PT2 proximal tubule population, a rare cell type that is equivalent to the PT-B population (designated from single cell RNAseq data) that was previously demonstrated to contain stem-like marker gene expression 24 (FIG. 23H). In contrast, the ROs and chRCC consistently showed highest probability to the intercalated-A (IC-A) population, indicating a distal nephron origin for these tumor types (FIG. 23H). Among the AML tumor compartment, maximum similarity was noted with the mesenchymal vSMC cells, and one subcluster also showed similarity to endothelial cells (FIG. 23H). Similar cell of origin probabilities were obtained when bulk tumor RNAseq data was analyzed with single cell data from benign kidney tissues (FIG. 30H). Finally, to bridge the single cell and single nuclei RNAseq-based predictions, it was demonstrated that the PT2 and cluster 29 populations of PT cells published by Lake et al 58 were equivalent to the previously identified PT_B and PT_C rare stem-like populations, and PT2/PT_B cells was nominated as the cell of origin for several RCC subtypes (FIG. 301). Here, for the first time evidence from single nuclei RNAseq data that IC-A cells are also the potential cell of origin for the oncocytomas is provided.

Identification of potential RCC cells of origin enabled an investigation of the fraction of biomarkers that showed shared expression between tumor epithelia and the corresponding cell of origin. Towards this, the top 100 proteogenomic markers that were differentially expressed in each of the RCC subtypes was identified (FIG. 30J) and their enrichment among normal cell types of the nephron (FIG. 23H). The data showed that at least a subset of these shared proteogenomic events between the tumor and putative cell of origin could be attributed to lineage-specific marker expression retained in the tumors. Select examples of these lineage-specific markers were identified and validated in subsequent sections including MAPRE3 in RO and PIGR in papillary RCC.

### Phosphoproteomic Signatures of RCC Subtypes and GI Tumors

Whole proteome profiling coupled with phosphoproteome quantification enables collective activity assessments of kinases and their phosphorylation substrates. This approach allows exploration of vital cell signaling pathways dysregulated in the tumor and its microenvironment. Using protein abundance data from 150 RCC tumors and 101 NATs, differentially expressed kinases were identified in each major RCC subtype and normal renal tissue (FIG. 24A). Prior observations including vascular endothelial growth factor (VEGF) receptor FLT1 enrichment in ccRCC 59, receptor tyrosine kinases MET and KIT (commonly referred as CD117) in pRCC and chRCC/RO, respectively, and serine threonine kinase MYLK in AML (FIGS. 24A and 24B) were verified. It was also discovered differential expression of CDK18, NEK6, and PNCK in ccRCC, while DAPK2, MAPK13, MAP3K1, SYK, DDR1, EIF2AK4, PAK4, and PTK2B were chRCC-specific. LATS1, PRKCD, PRKAG2, and STK39 were common between RO and chRCC. Therapeutic intervention of some of these kinases are a viable option as many are currently being evaluated in clinical and preclinical settings (FIG. 24A).

In order to identify phosphorylation changes that are associated with the subtype enriched kinase expression noted above, the corresponding phospho data comparisons were performed and select examples of phosphosite changes noted across the RCC subtypes (FIG. 31A) were highlighted. Phosphorylation sites T507 and S645 in the delta type of protein kinase C (PRKCD) show significantly elevated intensity in RO and chRCC compared to pRCC and ccRCCs (FIG. 31A). Phosphorylation of these two sites act as a priming step that allows the catalytic maturation of the protein kinase60,61. PRKCD phosphorylation is a component of the phospholipase C (PLC)-PKC signaling system, which is a part of leptin stimulation 62, and the leptin pathway is significantly enriched in RO (FIG. 24C and FIG. 31A). Directly comparing phosphorylation activity between RO and pRCC type1 (FIG. 31B), higher phosphorylation was observed on activating sites of the leptin pathway, such as those on PRKCD, STAT3, MAPKs, SRC, and BAD. At the same time, lower phosphorylation intensity in inhibiting sites on FOXO3 and GSK3A/B were observed. Leptin signals through the JAK-STAT axis and stimulates phosphorylation of IRS1/2, thereby resulting in regulation of the PI3K-AKT pathway 63 (FIG. 24C and FIG. 31A). Similar to the leptin pathway, the KIT receptor pathway was also activated in RO subtypes but depleted in malignant tumors such as ccRCC and pRCC, evidenced by up-regulation in ACACB, BAD, MAPK1, STAT1, STAT5B, and SRC. IL2 pathway was found to be uniquely up-regulated in RO type2, with high phosphorylation intensity in BAD, STAT1, and ACACB (FIG. 31A). Except for IL2 signaling, other immune-related pathways including IL33, TSLP, and T cell and B cell receptor were generally highly phosphorylated in multiple immune subtypes of ccRCC as well as in pRCC, but not in ROs. This observation agreed with the single nuclei RNA-seq findings where higher immune content in ccRCC and pRCC tumors was seen (FIG. 30C).

Next, the phosphorylation changes in GI non-ccRCCs were explored by comparing wGII high versus low samples. Focusing on 54 significant kinase-substrate co-regulation (fdr <0.05, abs (kinase log 2 fc)>0.05, abs (substrate site log 2 fc)>0.5), cyclin dependent kinases (CDK1, CDK2) were mostly enriched with up-regulated signaling events (FIGS. 24D and 24E). CDK1 promotes G2/M transition in the cell cycle and replicative DNA synthesis, while CDK2 has a role in G1/S transition, the initiation of DNA synthesis, and the regulation of S phase exit 64. Hence, their activities are imperative to genomic stability 65, 66. Many significantly up-regulated CDK1 substrates are E2F targets such as RRM2, MCM4, DUT, RFC1, PAICS, NASP, and HMGA1, which regulate DNA replication and chromosomal replication 67. T356 on RB1 is among the many potential CDK2 and CDK4/6 substrates 68, 69 (FIGS. 24D and 24E) and predictive of survival in HPV-negative squamous cell carcinoma of the head and neck 70. Hyperphosphorylation of this site inhibits its binding with E2F, thereby allowing E2F nuclear localization and subsequent transcriptional regulation 71. Phosphorylated RB1 also promotes apoptosis in response to replication stress and DNA damage 72. CDK2 can also be phosphorylated by other kinases such as LYN, a proto-oncogene, at Y15 73. CLUMPS-PTM analysis, a modified tool based on previously published CLUMPS, is used to identify mutation clusters in protein 3-D structure 74. Here, this analysis revealed that three phosphorylation sites (T14, Y15, and T160) formed a phosphorylation hotspot on CDK2 in the 3D space (FIG. 24F). Y15 and T160 are known to have opposing roles in CDK2 function, with Y15 being inhibitory and T160 activating the kinase. Their up-regulation was also observed in ovarian high-grade serous cancer 75, where they noted this seemingly counterintuitive Y15 hyperphosphorylation could be inhibiting just a subpopulation of CDK2 even if the overall CDK2 activity is increased 76. In addition, increased phosphorylation of CDK2 on Y15 has been associated with cell cycle exit in response to replication stress 77, 78. All of these observations again delineate the links between abnormal cell cycle transitions, increased proliferation, replication stress, and genomic instability, which is increasingly recognized as a hallmark of cancer 79, 80.

On the other hand, MTOR kinase and its substrates such as LARP1, UVRAG, and MAF1 showed decreased abundance and phosphorylation, respectively (FIGS. 24D and 24E), and this finding has clinical significance. Phosphorylation of LARP1 by MTOR dissociates it from binding with 5′UTRs of ribosome protein mRNAs whereas non-phosphorylated LARP1 interacts with ribosome protein mRNAs and inhibits their translation 81. Similarly, as a direct substrate of MTOR, dephosphorylation of MAF1 represses RNA polymerase III transcription 82. MTOR can also inhibit later stages of autophagy by phosphorylating UVRAG 83. Similar to the findings described herein, MTOR and CDK1 abundance were observed to be negatively correlated in autophagy in a previous study 84. MTOR is also actively involved in growth factor signaling pathways. In conjunction with the protein level enrichment analysis results (FIG. 29H), upregulation of CDK1 was associated with high proliferation, and, at the same time, down-regulation of MTOR was associated with decreased fatty acid metabolism and oxidative phosphorylation. However, comparing high and low wGII ccRCC tumors indicated attenuated CDK1 increase and no change in MTOR (FIG. 31C). MTOR inhibitor everolimus and VEGF receptor FLT1 inhibitor sunitinib have been evaluated in metastatic RCC, where sunitinib showed greater therapeutic value 85. Thus, it is contemplated that genome unstable non-ccRCC tumors, which are more likely to metastasize, are also likely to have downregulated MTOR pathway activity which might explain the relatively poor response noted with everolimus as compared to sunitinib.

### RCC Glycoproteomics Data Reflects Tumor Immune Infiltration and Angiogenesis

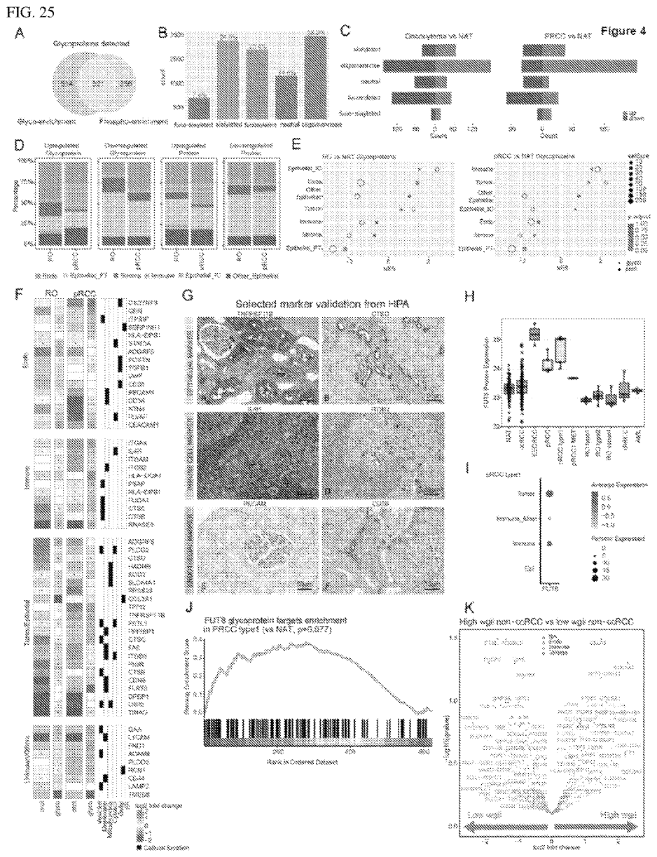

Protein glycosylation patterns linked with cancer development and progression 86,87 can be monitored by tumor glycoproteomics profiling 88 that allows exploration of RCC biology, biomarkers, and therapeutic target discovery 89. Cell surface proteins in endothelial and immune cells are known to be heavily glycosylated 90, and aberrant glycosylation of the lymphocyte marker PTPRC (CD45) can regulate tumor microenvironment (TME) function 90. To explore RCC glycobiology and its implications on TMEs, the two different glycosylation datatypes generated for this cohort were analyzed. First, a total of 56 rare RCC samples were enriched for N-glycopeptides using mixed ion exchange (MAX) enrichment 91 and analyzed by the MSFragger-Glyco search pipeline 92,93. Second, recent studies found phosphorylation enrichment via immobilized metal affinity chromatography (IMAC) technology also co-enriched a substantial number of glycopeptides, particularly sialoglycopeptides 94. Thus, by analyzing phosphorylation-enriched experiments of rare RCC samples and ccRCC 34, it was possible to gain insight into the glycoproteomics landscape in a pan-RCC setting. Given the overall better enrichment of intact glycopeptides (IGPs), observations in glyco-enriched data were validated in the phospho-enriched dataset. Several aspects were investigated, including glycan type abundance and differential expression analysis between RCC subtypes and finally deconvoluted glycoprotein expression patterns using markers identified from kidney single cell RNAseq (scRNA-seq) data 24. The N-linked glycoproteomics pipeline identified 12,503 IGPs with glycans (glycoforms) from 1,035 glycoproteins in glyco-enriched samples and 29,850 glycoforms from 1,591 glycoproteins in the phospho-enriched samples, respectively, with an overlap of 521 glycoproteins (FIG. 25A).

Based on the glycans' monosaccharide composition, IGPs were classified into five categories 95, namely oligomannose, sialylated, fucosylated, fuco-sialylated, and neutral moieties. Identified IGPs were mainly attached to oligomannose glycans, followed by sialylated glycans, in glyco-enriched samples (FIG. 25B). In the phospho-enriched samples, IGPs were largely sialylated (FIG. 32A) 94. Glycopeptides attached with oligomannose glycans accounted for a large number of the differential expression (tumor versus normal) events in both RO and pRCC glyco-enriched samples (FIG. 25C). Differential glycopeptide abundance positively correlated to corresponding protein abundance changes, but discordant events were also noted (FIGS. 32B and 32C). Integration with kidney scRNAseq (Methods) data revealed that a significant fraction of the dysregulated glycoproteins was contributed by the TME, both in ccRCC and pRCC 24 (FIG. 25D, 25E, 32D and 32E), and the contribution from immune compartment was higher in pRCC (30%) and ccRCC (30%) compared to RO (5%) (FIG. 25D, 25E, 32D, 32E). Only RO samples showed a higher fraction of upregulated markers of intercalated cells, a cell type proposed as the cell of origin for RO.

Similar trends were seen in the phospho-enriched samples for RO and pRCC, and significant differences between ccRCC immune subtypes were also observed (FIG. 32E). As differential glycosylation of key targets have been associated with altered immune and endothelial cell functions 90, select glycoprotein markers (both up and downregulated) contributed by TME cell types (FIGS. 25F and 32F) were investigated. Specifically, RO showed the upregulation of IGPs of known marker PLCG2 96, and ADGRF5 from epithelial/tumor, VWF, POSTN, and STAT5 from endothelial, and CTSD, PTGS2, and SOD2 from the immune compartments. On the other hand, pRCC showed upregulation of TFPI2, FSTL1, FAS, and PIGR in the epithelial/tumor, C1QTNF3 and GRN in the endothelial, and ITGAX, HLA-DQA1, IL411, and CTSC in the immune compartments. In addition to the above examples, other IGPs from proteins not assigned to a specific cell type by scRNAseq were present, such as cancer stem cell marker CD44 97 and ENPP3 in ccRCC, and CD44 in pRCC also showed differential glycosylation. Protein expression patterns noted across cell types by immunohistochemistry in the Human Protein Atlas 98 rendered additional support (FIG. 25G).

Glycosylation enzyme expression patterns assessed from global protein and RNA levels across pan-RCC samples might explain glycan aberrations (FIG. 32G). Particularly, glycotransferases including MGAT1 and FUT11 were high and glycohydrolases GLB1, FUCA1, FUCA2, HEXA, and HEXB were low in ccRCC versus NATs and other RCC subtypes. RO, on the other hand, showed upregulated expression of MAN2A1, ST3GAL4, and ALG10, while pRCC showed higher expression of FUT8 and B3GALT5 (FIG. 32G). The RNA and protein levels of glycosylation enzymes were similar, supporting the rationale for using RNAseq data to predict glycosylation pathway changes 99. Further analysis on N-glycan processing pathways identified FUT8 as one of the key upregulated changes in pRCC (FIG. 25H), which was also observed in TCGA dataset (FIG. 32H) and was largely expressed in tumor cells (FIG. 251). FUT8, a glycosylation enzyme known for its core-fucosylation function, is upregulated in multiple cancers 100 and is considered a driver of melanoma metastasis 101. Using the N-glycoproteomics profiling result, it was confirmed that the putative glycoprotein targets of FUT8 101,102 had an overall upregulated glycosylation pattern (FIGS. 25J and 32I). One of the intriguing targets was MET, a driver oncogene receptor tyrosine kinase in pRCC type-1 103,104 and a crucial regulator of EMT 105. A previous study showed that c-MET (the protein encoded by MET) is functionally regulated by N-glycosylation 106, 107 and core fucosylation can potentiate its ligand binding ability 108. Loss of the FUT8 gene in the HepG2 cell line resulted in the attenuated responses of hepatocyte growth factor 109, which supports regulation of MET by FUT8 a potential tumorigenesis mechanism. Upregulation of c-MET glycosylation was observed in only type 1 pRCC samples (FIG. 32J). In addition to MET, several glycoproteins including CTSC and LGALS3BP that were previously described as biomarkers in different cancers 30, 110 were also upregulated. The upregulation of MET glycosylation led to further site-specific analysis, and differential expression analysis showed that the MET_785 site had increased glycosylation (FIG. 32K). This site is largely core-fucosylated 106.

Finally, the glycosylation patterns of non-ccRCC samples with high wGII versus low wGII were compared to understand the glycobiology associated with genome instability. High wGII samples were enriched with immune markers such as GZMA (cytotoxic T Cells), FCGR1A, PTPRC (lymphocyte), and CD163 (macrophage), endothelial markers such as POSTN, ITRIP, ANO6, CD74, CD14, and STAB, stromal markers such as FBN, FBLN2, ITGA5, and COL1A1, and other markers MERTK and FH (FIG. 25K). This supports increased tumor microenvironment cell involvement in high wGII samples. GZMA is mainly expressed by cytotoxic T cells of the immune system and is proposed to promote colorectal cancer development 111. MERTK is a receptor tyrosine kinase aberrantly expressed in several malignancies and a novel target for cancer therapeutics 112. GSEA also revealed glycoproteins upregulated in high wGII samples involved in EMT hallmark, which was not shown in global proteomics and transcriptomics, highlighting the benefit of including glycoproteomics data to complement results from other data types.

### Metabolomic Profiling of Major RCC Subtypes Delineates Tumor Growth Dynamics

The kidney establishes numerous gradients that regulate and respond to oxygen, glucose, urea, and other nutrients. Its functional unit, the nephron, maintains nutrition and oxygen levels in the cortex and depleted levels in the medulla to faithfully navigate through various metabolic pathways 113. RCC is found to exhibit a diverse array of metabolic defects and perturbations driven by genomic alterations; thus, RCC is seen as a metabolic disease 114. For example, VHL, a major mediator of oxygen sensing, is mutated commonly in ccRCC, resulting in accumulation of hypoxia inducible factor (HIF) family members, leading to glucose uptake and glycolytic metabolism 38. Germline or somatic mutation of FH and decreased FH expression were found in type 2 pRCC tumors 103, which is a driver mutation in TCA cycle seen in highly aggressive disease 115. FH-deficient RCC is dependent on glucose for ATP production needed for rapid proliferation 116, 117. One common metabolic feature of RCCs is the invocation of aerobic glycolysis, the Warburg phenomenon, which is dependent on the pentose phosphate shunt and decreased oxidative phosphorylation, and is associated with high grade, high stage, and low survival tumors 118.

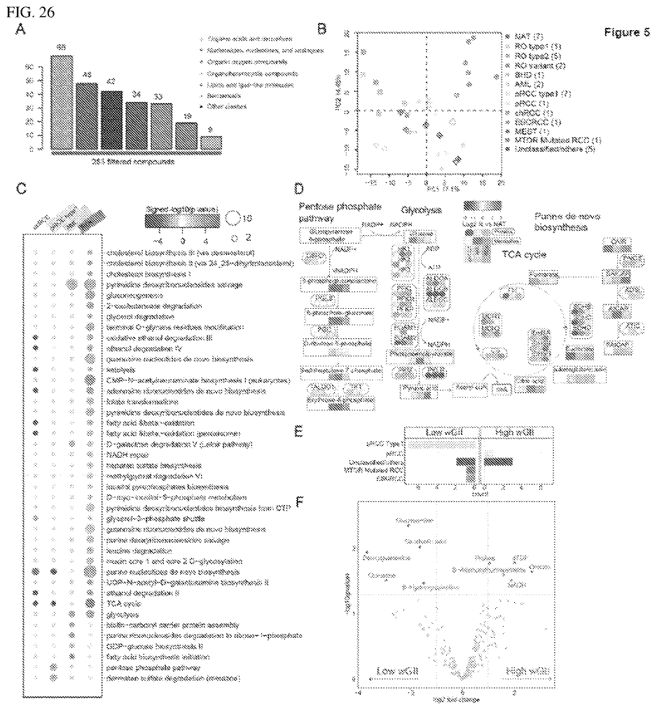

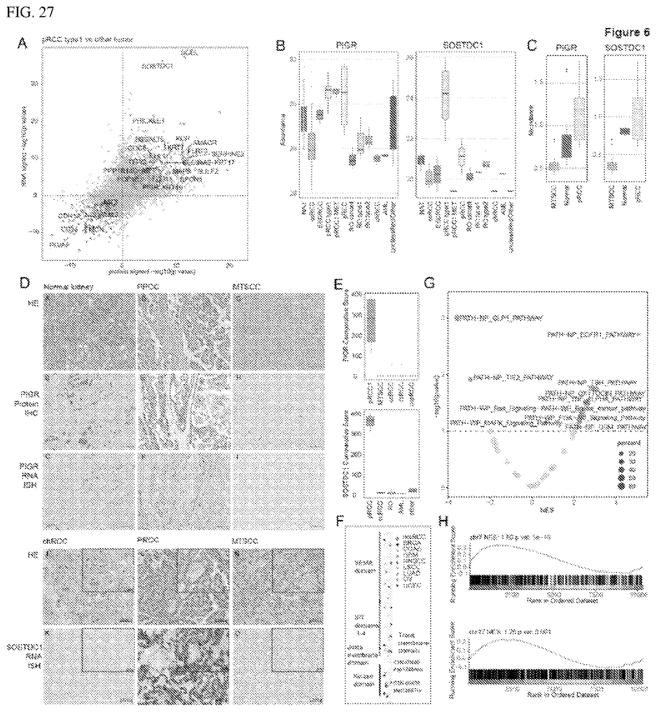

Metabolomics profiling informs on the metabolic reprogramming associated with kidney tumorigenesis 119. Here, 253 metabolites were profiled across 28 tumors (excluding the PUC tumor) and seven normals within the non-ccRCC cohort (FIG. 29A). Various metabolites quantified are intermediates in several metabolic pathways and can broadly be grouped under organic acids and derivatives (68), nucleosides, nucleotides and analogues (48), organic oxygen compounds (42), and other compounds such as organoheterocyclic compounds, lipids, and benzenoids, together covering major metabolic pathways (FIG. 26A and FIG. 33A). Two-dimensional visualization of the abundance of these compounds via PCA showed clear separation between sample classes (FIG. 26B), indicating differential metabolomic characteristics across tumor subtypes. 65, 136, and 97 compounds were identified in pRCC type1, AML, and ROs, respectively, showing significantly higher abundance compared to NATs (>=2 fold change and q value <=0.05) at log 2 scaled fold change cutoff 1 and q value cutoff 0.05 (FIG. 33B). Combining with differentially expressed metabolic enzymes, joint pathway analysis was used to pinpoint metabolic pathways that were potentially perturbed across RCC subtypes. In accordance with differences in tumor cells of origins, ccRCC and pRCC type1 tumors shared some common pathway enrichment as compared to chRCC and ROs (FIG. 26C). Purine nucleotides de-novo biosynthesis and TCA cycle were depleted in both ccRCC and pRCC type1 tumors, but were enriched in a number of up-regulated components in ROs. Pentose phosphate pathway and dermatan sulfate degradation were potentially up-regulated in pRCC type1 but not in other tumor types. Pyrimidine deoxyribonucleosides salvage pathway and glycolysis were active in both AML and ROs. High levels of ACACA, ACACB enzymes, and phosphoric acid in AML indicate increased activity in fatty acid biosynthesis (FIG. 33C, 33D).

A number of enzymes in the pentose phosphate pathway were highly expressed in pRCC type1 tumors (FIG. 26D, FIGS. 33C and 33D). In the oxidative branch, highly expressed G6PD converts glucopyranose 6-phosphate to 6-phosphoglyconolactone. In the non-oxidative branch, highly expressed TALDO1 and TKT work together transferring sedoheptulose-7-phosphate to erythrose-4-phosphate. This process is known to be accelerated in tumors to meet the increased need of ribonucleotides generation in rapidly proliferating cancer cells 96. However, in renal ROs, these enzymes are not differentially expressed, which may present as a progression barrier in these largely benign tumors for the progression of RO 96. Pyruvic acid, a product of glycolysis, was particularly highly accumulated in ROs (FIG. 26D, FIG. 33C, and 33D), which can be converted to Acetyl-CoA and utilized in the TCA cycle. In the TCA cycle, low levels of various enzymes such SDH (SDHB, SDHC, SDHD) and FH are observed in pRCC, resulting in low conversion from succinate to fumarate to malate, indicating impaired oxidative phosphorylation 118. On the contrary 120, FH, together with other enzymes such as IDH3 (IDH3A, IDH3B, IDH3G), and CS are up-regulated in ROs. A possible explanation for this observation is that the TCA cycle takes place in mitochondria 119, and 121 has a large number of mitochondria, though defective 120. In addition, high abundance of mitochondrially located proteins were also observed in ROs in previous literature 31. SAICAR, an oncometabolite in the purine de-novo biosynthesis pathway that supports the growth of cancer cells in a nutrient-limited medium 123, was highly abundant in ROs (FIG. 26D, FIG. 33C, and 33D). Combining the evidence in elevated abundance of its upstream and downstream compounds and enzymes, it was concluded that the accumulation of SAICAR is a result of high conversion from CAIR under high PAICS level and low conversion to AICAR under low ADSL level.

Finally, tumors with available metabolomic data including four high wGII versus 11 low wGII non-ccRCC samples were compared to identify five up-regulated and five down-regulated compounds in the high-wGII group (FIGS. 26E and 26F). Significantly up-regulated proline and NADH, coupled with high PYCR1 expression (FIG. 29G), indicated higher proline biosynthesis, which might support cancer cell proliferation and survival in oxygen-limiting conditions 124. High S-Adenosyl-L-homocysteine (SAH) is associated with homocysteine production and is known as an inhibitor of S-adenosyl-L-methionine-dependent methylation 125. Moreover, increased levels of orotate and dTDP may imply higher activity in pyrimidine metabolism 126 in high-wGII samples. On the other hand, three compounds, saccharic acid, glucosamine, and 8-hydroxyquinoline, of which the derivatives are known to have anticancer effects, were abundantly expressed in genome-stable samples. The salt form of saccharic acid has potent antiproliferative properties in vivo 127; glucosamine exhibits its antitumor role through the inhibition of epidermal growth factor-induced proliferation and cell cycle progression 128; the anticancer activity of 8-hydroxyquinoline relies on complex formation with redox active copper and iron ions 129.

### Papillary RCC Biomarkers and Proteogenomics of Activating MET Mutations

Malignant papillary renal cell carcinomas (pRCC) 103 are histomorphologically and genetically heterogeneous diseases that account for 15% of all RCCs. They are broadly classified into type-1 and 2, and chromosome 7/17 copy gains are the most recurrent genomic aberration in type-1 tumors followed by activating mutations in the MET gene. In type-2 disease which takes a more aggressive clinical course, genomic aberrations are more heterogeneous and largely associated with NRF2 pathway activation. Similar to oncocytic tumors discussed above, diagnostic challenges in biopsy samples coupled with limitations of the current clinical immunohistochemistry panel for pRCC can be alleviated by discovery of tumor type-specific biomarkers. Current pRCC markers lack tumor specificity and the added challenge of intratumoral variation in staining encountered at times which ranges from patchy to uniform and strong to weak regions within a given specimen. Current diagnosis partly relies on cytokeratins (that are not specific to any tumor type) such as KRT7 staining (similar to oncocytic tumors discussed above) where immunohistochemically type-1 tumors have stronger KRT7 and markers such as AMACR, whereas type-2 shows loss of KRT7 3. A less common largely benign tumor, namely the mucinous tubular and spindle cell carcinoma (MTSCC) may show overlapping morphology with type 1 papillary RCCs, particularly in limited biopsy samples 130. In order to aid in the discovery of cancer-specific biomarkers that can distinguish type-1 pRCC from MTSCC, a series of differential expression analysis using the CPTAC RNA and protein data was performed on an independent publicly available pRCC and MTSCC proteomics dataset t 33.

First, differential expression analysis of CPTAC pRCC type1 (n=8) vs the other samples identified pRCC type1 specific up-(n=176, log 2 fc >1, q value <0.05) and downregulated (n=108, log 2 fc<−1, q value <0.05) genes that were significant with both RNA seq and proteomics data (FIG. 27A). Proteins such as polymeric immunoglobulin receptor (PIGR) and sclerostin domain containing 1 (SOSTDC1) were specifically upregulated in pRCC compared to other kidney tumors (FIG. 27B and FIG. 34A) and were further validated. PIGR is a transmembrane protein involved in transcytosis of polymeric immunoglobulins from basolateral to apical surface of epithelial cells, while secreted glycoprotein SOSTDC1 is a bone morphogenetic protein antagonist 131. The MTSCC and pRCC proteome from Xu et al. 33 was compared to identify proteins specifically upregulated in pRCC. Interestingly, PIGR and SOSTDC1 were highly upregulated in protein expression in pRCC compared to MTSCC (FIG. 27C) and were validated by immunohistochemistry and RNA-ISH methodologies (FIGS. 27D and 27E). PIGR and SOSTDC1 RNA-ISH assay done on the tumor tissues was seen as brown dots (each dot corresponding to mRNA transcript), and clusters seen within the cytoplasm and nuclear compartment were especially enriched in pRCC-1. Minimal to absence of PIGR signal was seen (FIG. 27E). PIGR was evaluated as an IHC marker where the protein expression was seen predominantly in the membranous to cytoplasmic compartment as homogeneously strong to moderate expression within the pRCC-1 tumor cells. The adjoining benign kidney parenchyma showed a sub-population of tubular epithelium also expressing PIGR. For MTSCC, the PIGR expression was either minimal or patchy to absent within the tumor tissues when compared to pRCC-1.

The most recurrent genomic aberrations in type-1 pRCC include activating MET gene kinase domain mutations and whole chromosome 7 copy gains; hence, the proteogenomic impact of these recurrent events in pRCC was examined. Among the type-1 pRCC samples, two had hotspot somatic activating kinase domain mutations in the MET gene, namely Asp1246Asn and Met1268Thr (FIG. 27F), and 11 samples had chr 7 copy gain. Hence, the impact of this driver aberration in the protein and phosphoprotein data in a two-group analysis was performed by comparing the index MET mutant samples with MET wildtype cases. Differential expression analysis revealed several up-regulated phosphor serine/threonine and phosphotyrosine events, included several known MET substrates such as Y689 of GAB1 and Y427 of SHC1 (FIG. 34B). This is consistent with the knowledge that MET kinase domain missense mutations activate its kinase activity. In addition to known MET substrates, performing enrichment analysis with PTM-SEA, several signaling pathways were enriched with up-regulated phosphorylation sites, such as EGFR, PI3K-AKT, and MAPK (FIG. 27G). The intracellular signaling cascades activated by MET include the PI3K-AKT, RAC1-cell division control protein CDC42, RAP1, and RAS-MAPK pathways. An intricate network of cross-signalling involving the MET-EGFR has been shown recently to have major implications for therapy 132. One recent study has demonstrated cooperative signaling between MET and EGFR during kidney development 133.

Large whole chromosomal gains and losses with fewer focal events are signature features of kidney tumors which is unlike other solid tumors such as lung whose CNV landscape is dominated by focal events. Broad impact of whole chromosomal CNVs on select tumors, such as the recurrent losses of chrs 1,4,6,9,14,15 and 22 observed in MTSCC tumors on mRNA 9 and protein abundances 33 have been examined. However a more systematic analysis of this dosage effect remains underexplored in kidney tumors, especially in terms of protein and phosphoprotein changes where the latter can serve as a functional readout. In ccRCC, chr14 and 9p loss noted in a subset of patients is associated with poor survival 59. Chromosome 7 gain is noted in a subset of ccRCC largely mutually exclusive with chr 14 loss events. In comparison, chr7 gain is a recurrent signature event in type1 pRCC tumors. It remains to be studied if chr7 gain in ccRCC and non-ccRCC has similar proteogenomic impact. Hence, for a systematic analysis of dosage effect in ccRCC and non-ccRCC, gene expression comparison was performed between chr7 gain with no gain tumors. CNV alteration had a significant impact on the transcript/protein abundance encoded by the affected chromosome where losses and gains were always associated with downregulation and upregulation, respectively, in RNA and protein abundance (FIG. 34C). One of the example genes is SOSTDC1, which is located on chr 7 and shows increase in both levels. While most genes followed that pattern, there were instances where the opposite was seen. For instance, chr 7 gene AOC1 was found to be down-regulated in both RNA and protein expression in ccRCC tumors which have chromosome 7 gain (FIG. 34E). In addition, Chr7 gain in non-ccRCC was linked to increased chr17 gene expression, which is not seen in ccRCC (FIG. 27H, 34D). It was next examined how the CNV chr7 gain event impacts ccRCC and non-ccRCC pRCC tumors at a pathway level. EMT, angiogenesis, KRAS signaling up were found to be up-regulated, whereas adipogenesis and fatty acid metabolism were down in both ccRCC and non-ccRCC. Myogenesis-associated genes were up in ccRCC and down in non-ccRCC chr7 gain samples. On the contrary, immune-related pathways such as IL6_JAK_STAT3 signaling, interferon alpha and gamma response, and allograft rejection were down in ccRCC but up in non-ccrCC chr7 gain samples (FIG. 34F). Finally, to examine the impact of chr 7 copy gain in phosphorylation data, phosphorylation activities were compared between papillary lineage tumors with and without Chr7 gain. It is clear that up-regulated phosphorylation activities were substrates enriched in a number of Chr7 kinases, such as HIPK2, CDK13, MET, CDK6, and BRAF. (FIG. 34G)

### Proteogenomic Analysis of Oncocytic Tumors Discovers Differences in Gene Regulatory Networks and New Differential Diagnosis Biomarkers

Discovering protein biomarkers to aid differential diagnosis is one of the aims of this study. In this realm, distinguishing chRCC from RO has clinical significance as RO patients can avoid morbidity and economic burden related to a surgical intervention and could be monitored periodically and undergo planned surgery at a later time. Existing challenges include similarities in histological and immunohistochemical readout between the two tumors which often pose diagnostic dilemmas during both needle biopsy samples and whole tissue section evaluations 19. Molecular analysis using immunohistochemical (IHC) markers for chRCC, such as KRT7, CD117 (c-kit), epithelial mesenchymal antigen, and parvalbumin (PVALB) are commonly used in the clinic by pathologists 21, 134. RO diagnosis is assisted by an IHC stain of cytokeratin 7, CD117 (c-kit), S100A1 135, and kidney-specific cadherins 136; however, limitations exist due to instances of patchy staining and pattern overlap with chRCC 21, 134, 135. In clinical diagnostic criteria, patchy KRT7 expression usually supports RO, while strong uniform staining is usually supportive of chRCC. Also, despite concerted attempts by various researchers, medical imaging tools, such as CT-Scan or MRI, do not conclusively differentiate these tumors due to their similarity in appearance 20. Hence, the three chRCC and 15 ROs profiled in this study and publicly available proteomics data from RO and chRCC samples 96, 137 were examined to address this clinical need.

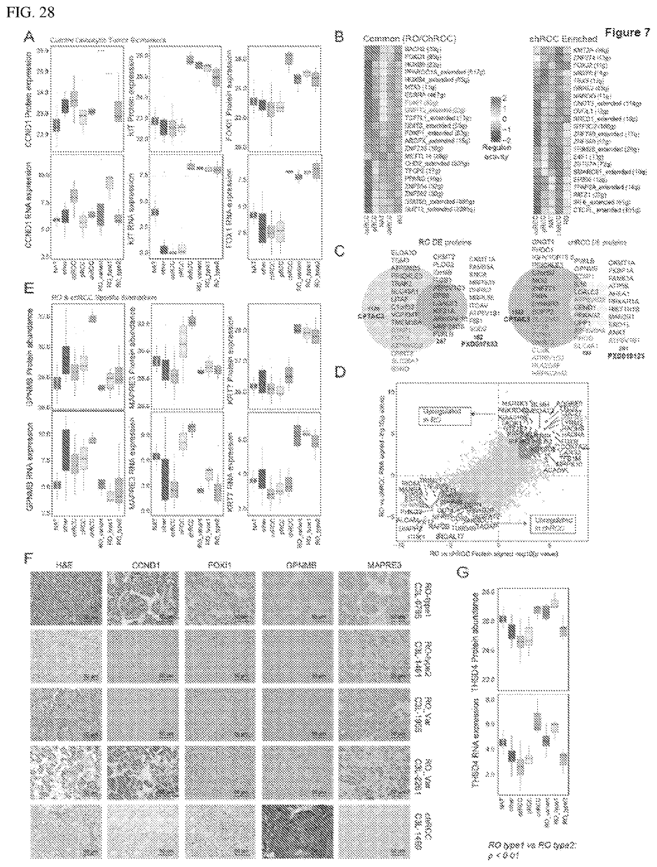

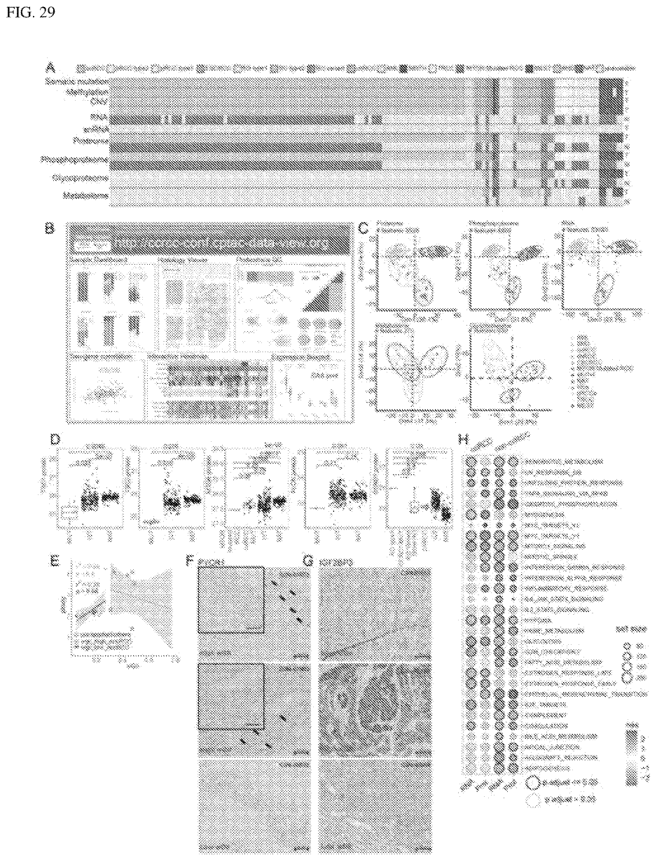

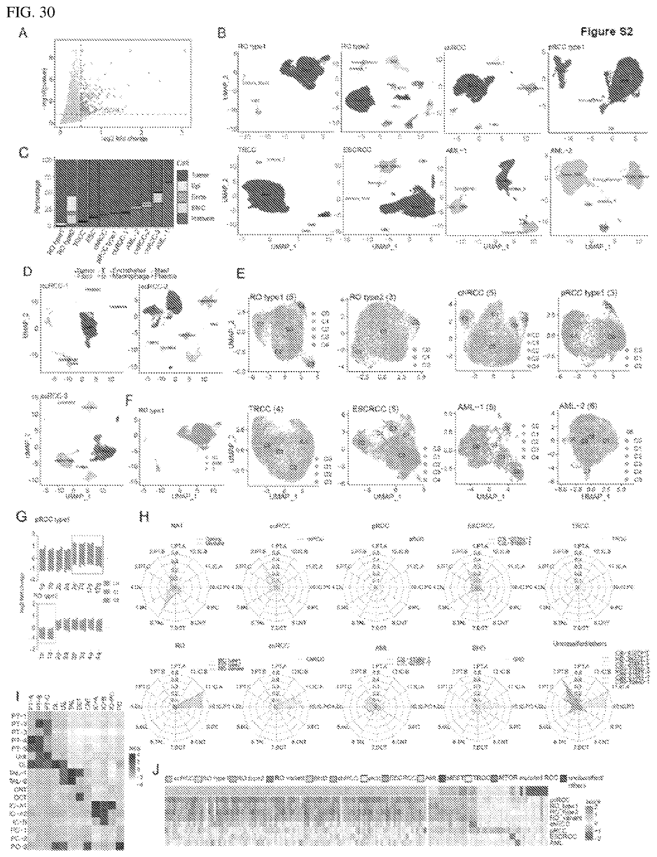

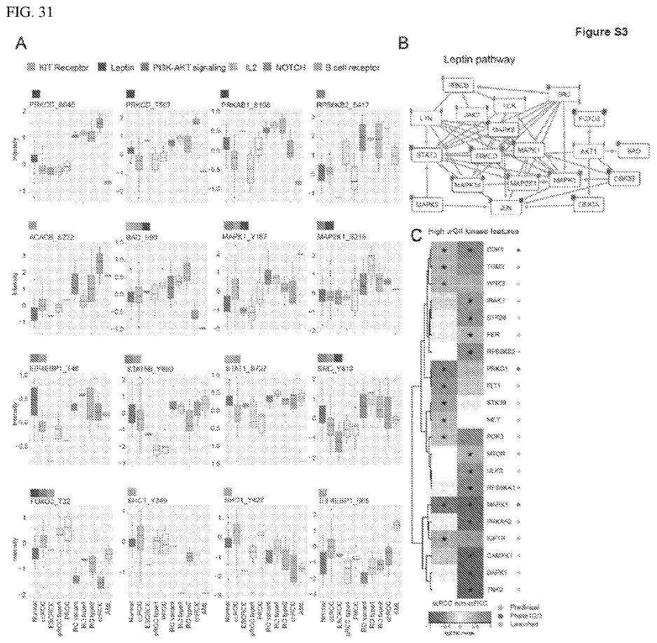

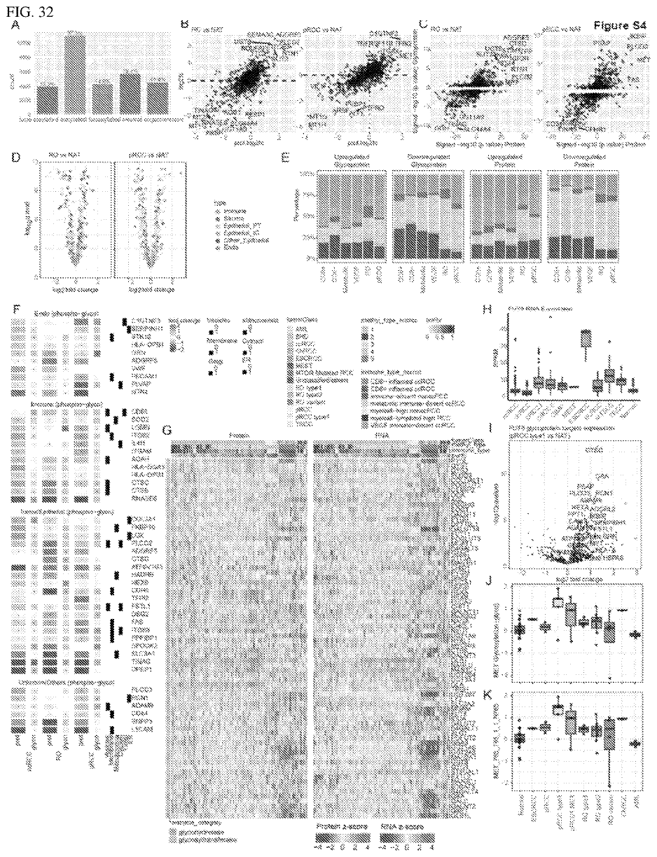

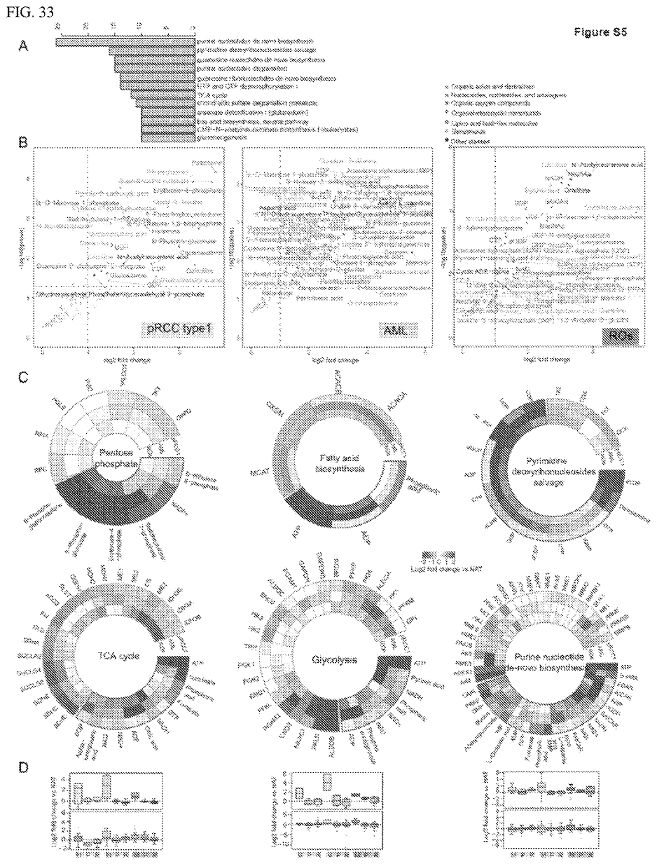

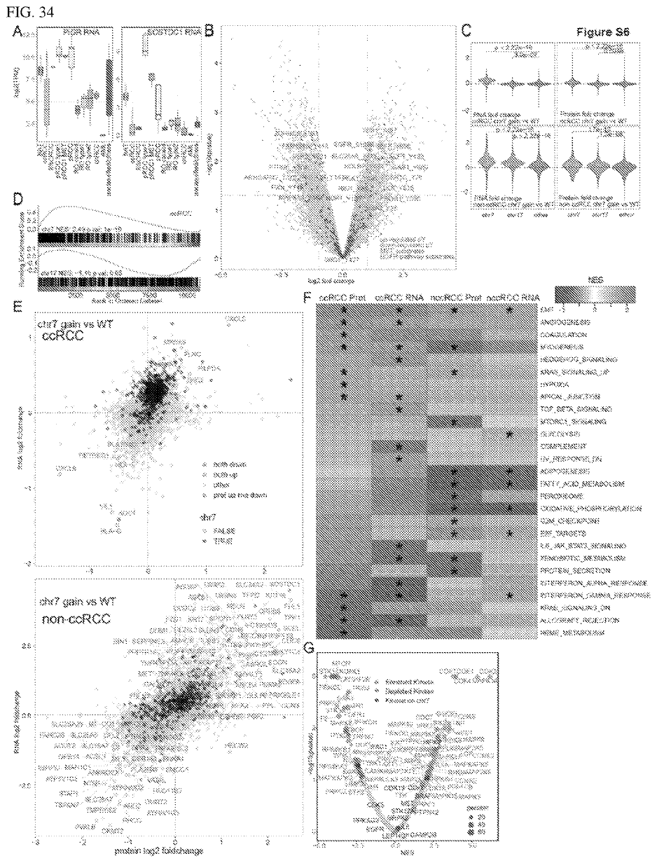

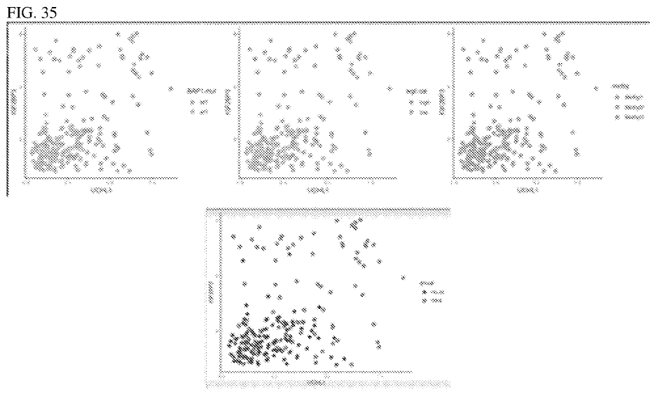

All three chRCC cases in the cohort had recurrent chromosomal losses 1, 2, 6, 10, 13, 17 and TP53 loss of function mutations. Among the ROs, three broad molecular subtypes, with RO subtype1 (n=3) containing the signature CCND1 gene rearrangements were identified 28 (FIG. 22A) and resulting overexpression of this driver gene was observed both at RNA and protein levels (FIG. 28A). All RO type2 (n=8) cases showed the signature chromosome1 copy loss. The remaining samples showed vast heterogeneity in genetic aberration and were placed in the less appreciated and emerging third RO molecular subtype (n=4) (FIG. 22A). KRT7 was differentially expressed, while other markers such as KIT and FOXI1, did not distinguish between ROs and chRCC (FIG. 28A).

Proteogenomic analyses at the gene regulatory network (GRN) and mRNA/proteomic levels revealed differences in transcriptional modules and marker expression between these two tumors. The SCENIC tool, 138 which examines the coexpression of a given transcription factor and its cognate target genes, was used to characterize transcriptional modules (regulons) differences among the different kidney tumor and benign tissues (FIG. 28B). FOXI1 is an important transcription factor that plays a key role in the nephronal intercalated cells and in tumors that arise from them, including chRCC and Ros 12,139. Regulons shared between chRCC and RO include the lineage-specific transcription factors FOXI1 and DMRT2, the latter being transcriptionally regulated by FOXI1. The CPTAC proteomic dataset had a better coverage of FOXI1 and DMRT2 where both of these transcription factors and most of their gene targets (such as ATPVOD2, HEPACAM2, and DMRT2 etc) showed differential expression in tumor versus normal comparisons, as expected. This is in stark contrast to two publicly available datasets, likely due to low protein coverage (FIG. 28C) 96, 137. SCENIC analysis also identified several regulons that were enriched in chRCC but not in RO, such as ZBTB7A, SMARCB1, E4F1, and FOXJ2, among others. These regulons were not previously associated with this disease, and these new findings shed light on major transcriptional programs active in chRCC. DEPs and DEGs were identified by performing detailed analysis in RNA and protein datasets for RO vs normal and chRCC vs normal (FIG. 28D) and candidates that were specific to the two tumors were identified such as microtubule associated protein RP/EB family member 3 (MAPRE3, specific to RO) and glycoprotein nonmetastatic melanoma protein B (GPNMB upregulated in chRCC) (FIG. 28E). Upregulation of KRT7 in chRCC served as a positive control (FIG. 28D). Using immunohistochemistry, the specificity of these biomarkers was next confirmed and validated (FIG. 28F). CCND1 protein was overexpressed only in the gene fusion positive ROs, while the newly identified MAPRE3 expression was noted in all RO subtypes. In stark contrast, GPNMB was solely expressed in chRCC and not in RO. FOXI1 showed nuclear staining in both chRCC and ROs. While CCND1 and FOXI1 were localized in the nuclei, GPNMB showed a homogeneous and moderate/strong expression within the cytoplasmic compartment of the chRCC tumor cells, and MAPRE3 protein expression was noted as predominantly in the membranous compartment of the RO tumor tissues.

Additionally, by integrating RO markers from the snRNAseq and global proteomics data, upregulation of THSD4, also known as ADAMTSL6, was identified in RO type 1 but decreased in type 2 when compared to NAT (FIG. 28G). This observation underlines the additional molecular differences that exist therein, and future validation of this marker might enable rapid distinction between RO types 1 and 2. THSD4 facilitates ECM assembly in various tissues 140, and it's reported to directly bind TGF-β and attenuate TGF-β signaling 129. Recent studies identified THSD4 as a potential GATA3 target in tumorigenesis130, as well as its regulatory role in tumor cellular dormancy 131. The molecular mechanism of THSD4 in regulating cancer metastasis was previously validated in colorectal cancer 132. In summary, the proteogenomic analysis of oncocytic tumors identified differentially expressed GRNs and proteogenomic biomarkers that inform on disease biology and provide tools to advance clinical diagnosis.

### Example 3 References

1. Moch, H., Amin, M. B., Berney, D. M., Compérat, E. M., Gill, A. J., Hartmann, A., Menon, S., Raspollini, M. R., Rubin, M. A., Srigley, J. R., et al. (2022). The 2022 World Health Organization Classification of Tumours of the Urinary System and Male Genital Organs-Part A: Renal, Penile, and Testicular Tumours. Eur. Urol. 82, 458-468. 10.1016/j.eururo.2022.06.016.
2. Moch, H., Cubilla, A. L., Humphrey, P. A., Reuter, V. E., and Ulbright, T. M. (2016). The 2016 WHO Classification of Tumours of the Urinary System and Male Genital Organs-Part A: Renal, Penile, and Testicular Tumours. Eur. Urol. 70, 93-105. 10.1016/j.eururo.2016.02.029.
3. Warren, A. Y., and Harrison, D. (2018). WHO/ISUP classification, grading and pathological staging of renal cell carcinoma: standards and controversies. World J. Urol. 36, 1913-1926. 10.1007/s00345-018-2447-8.
4. Wobker, S. E., Matoso, A., Pratilas, C. A., Mangray, S., Zheng, G., Lin, M.-T., Debeljak, M., Epstein, J. I., and Argani, P. (2019). Metanephric Adenoma-Epithelial Wilms Tumor Overlap Lesions: An Analysis of BRAF Status. Am. J. Surg. Pathol. 43, 1157-1169. 10.1097/PAS.0000000000001240.
5. Sirintrapun, S. J., Geisinger, K. R., Cimic, A., Snow, A., Hagenkord, J., Monzon, F., Legendre, B. L., Ghazalpour, A., Bender, R. P., and Gatalica, Z. (2014). Oncocytoma-like renal tumor with transformation toward high-grade oncocytic carcinoma: a unique case with morphologic, immunohistochemical, and genomic characterization. Medicine (Baltimore) 93, e81. 10.1097/MD.0000000000000081.
6. Kim, M., Joo, J. W., Lee, S. J., Cho, Y. A., Park, C. K., and Cho, N. H. (2020). Comprehensive Immunoprofiles of Renal Cell Carcinoma Subtypes. Cancers 12, E602. 10.3390/cancers12030602.
7. Guo, G., Gui, Y., Gao, S., Tang, A., Hu, X., Huang, Y., Jia, W., Li, Z., He, M., Sun, L., et al. (2011). Frequent mutations of genes encoding ubiquitin-mediated proteolysis pathway components in clear cell renal cell carcinoma. Nat. Genet. 44, 17-19. 10.1038/ng.1014.
8. Hsieh, J. J., Le, V., Cao, D., Cheng, E. H., and Creighton, C. J. (2018). Genomic classifications of renal cell carcinoma: a critical step towards the future application of personalized kidney cancer care with pan-omics precision. J. Pathol. 244, 525-537. 10.1002/path.5022.
9. Mehra, R., Vats, P., Cieslik, M., Cao, X., Su, F., Shukla, S., Udager, A. M., Wang, R., Pan, J., Kasaian, K., et al. (2016). Biallelic Alteration and Dysregulation of the Hippo Pathway in Mucinous Tubular and Spindle Cell Carcinoma of the Kidney. Cancer Discov. 6, 1258-1266. 10.1158/2159-8290.CD-16-0267.
10. Wang, X.-M., Zhang, Y., Mannan, R., Skala, S. L., Rangaswamy, R., Chinnaiyan, A., Su, F., Cao, X., Zelenka-Wang, S., McMurry, L., et al. (2021). TRIM63 is a sensitive and specific biomarker for MIT family aberration-associated renal cell carcinoma. Mod. Pathol. Off. J. U. S. Can. Acad. Pathol. Inc 34, 1596-1607. 10.1038/s41379-021-00803-z.
11. Baba, M., Furuya, M., Motoshima, T., Lang, M., Funasaki, S., Ma, W., Sun, H.-W., Hasumi, H., Huang, Y., Kato, I., et al. (2019). TFE3 Xp11.2 Translocation Renal Cell Carcinoma Mouse Model Reveals Novel Therapeutic Targets and Identifies GPNMB as a Diagnostic Marker for Human Disease. Mol. Cancer Res. MCR 17, 1613-1626. 10.1158/1541-7786.MCR-18-1235.
12. Skala, S. L., Wang, X., Zhang, Y., Mannan, R., Wang, L., Narayanan, S. P., Vats, P., Su, F., Chen, J., Cao, X., et al. (2020). Next-generation RNA Sequencing-based Biomarker Characterization of Chromophobe Renal Cell Carcinoma and Related Oncocytic Neoplasms. Eur. Urol. 78, 63-74. 10.1016/j.eururo.2020.03.003.
13. Wang, L., Zhang, Y., Chen, Y.-B., Skala, S. L., Al-Ahmadie, H. A., Wang, X., Cao, X., Veeneman, B. A., Chen, J., Cieślik, M., et al. (2018). VSTM2A Overexpression Is a Sensitive and Specific Biomarker for Mucinous Tubular and Spindle Cell Carcinoma (MTSCC) of the Kidney. Am. J. Surg. Pathol. 42, 1571-1584. 10.1097/PAS.0000000000001150.
14. Akgul, M., and Williamson, S. R. (2022). How New Developments Impact Diagnosis in Existing Renal Neoplasms. Surg. Pathol. Clin. 15, 695-711. 10.1016/j.path.2022.07.005.
15. Andrici, J., Gill, A. J., and Hornick, J. L. (2018). Next generation immunohistochemistry: Emerging substitutes to genetic testing? Semin. Diagn. Pathol. 35, 161-169. 10.1053/j.semdp.2017.05.004.
16. Li, Y., Lih, T.-S.M., Dhanasekaran, S. M., and Mannan, R. (2023). Histopathologic and proteogenomic heterogeneity reveals features of clear cell renal cell carcinoma aggressiveness. Cancer Cell 41.
17. Sato, Y., Yoshizato, T., Shiraishi, Y., Maekawa, S., Okuno, Y., Kamura, T., Shimamura, T., Sato-Otsubo, A., Nagae, G., Suzuki, H., et al. (2013). Integrated molecular analysis of clear-cell renal cell carcinoma. Nat. Genet. 45, 860-867. 10.1038/ng.2699.
18. Cancer Genome Atlas Research Network (2013). Comprehensive molecular characterization of clear cell renal cell carcinoma. Nature 499, 43-49. 10.1038/nature12222.
19. Davis, C. F., Ricketts, C. J., Wang, M., Yang, L., Cherniack, A. D., Shen, H., Buhay, C., Kang, H., Kim, S. C., Fahey, C. C., et al. (2014). The somatic genomic landscape of chromophobe renal cell carcinoma. Cancer Cell 26, 319-330. 10.1016/j.ccr.2014.07.014.
20. Cancer Genome Atlas Research Network, Weinstein, J. N., Collisson, E. A., Mills, G. B., Shaw, K.R.M., Ozenberger, B. A., Ellrott, K., Shmulevich, I., Sander, C., and Stuart, J. M. (2013). The Cancer Genome Atlas Pan-Cancer analysis project. Nat. Genet. 45, 1113-1120. 10.1038/ng.2764.
21. Linehan, W. M., and Ricketts, C. J. (2019). The Cancer Genome Atlas of renal cell carcinoma: findings and clinical implications. Nat. Rev. Urol. 16, 539-552. 10.1038/s41585-019-0211-5.
22. Ricketts, C. J., De Cubas, A. A., Fan, H., Smith, C. C., Lang, M., Reznik, E., Bowlby, R., Gibb, E. A., Akbani, R., Beroukhim, R., et al. (2018). The Cancer Genome Atlas Comprehensive Molecular Characterization of Renal Cell Carcinoma. Cell Rep. 23, 313-326.e5. 10.1016/j.celrep.2018.03.075.
23. Oosterwijk, E., Rathmell, W. K., Junker, K., Brannon, A. R., Pouliot, F., Finley, D. S., Mulders, P.F.A., Kirkali, Z., Uemura, H., and Belldegrun, A. (2011). Basic research in kidney cancer. Eur. Urol. 60, 622-633. 10.1016/j.eururo.2011.06.048.
24. Young, M. D., Mitchell, T. J., Vieira Braga, F. A., Tran, M.G.B., Stewart, B. J., Ferdinand, J. R., Collord, G., Botting, R. A., Popescu, D.-M., Loudon, K. W., et al. (2018). Single-cell transcriptomes from human kidneys reveal the cellular identity of renal tumors. Science 367, 594-599. 10.1126/science.aat1699.
25. Zhang, Y., Narayanan, S. P., Mannan, R., Raskind, G., Wang, X., Vats, P., Su, F., Hosseini, N., Cao, X., Kumar-Sinha, C., et al. (2021). Single-cell analyses of renal cell cancers reveal insights into tumor microenvironment, cell of origin, and therapy response. Proc. Natl. Acad. Sci. U.S.A 118, e2103240118. 10.1073/pnas.2103240118.
26. Motzer, R. J., Robbins, P. B., Powles, T., Albiges, L., Haanen, J. B., Larkin, J., Mu, X. J., Ching, K. A., Uemura, M., Pal, S. K., et al. (2020). Avelumab plus axitinib versus sunitinib in advanced renal cell carcinoma: biomarker analysis of the phase 3 JAVELIN Renal 101 trial. Nat. Med. 26, 1733-1741. 10.1038/s41591-020-1044-8.
27. Motzer, R. J., Tannir, N. M., McDermott, D. F., Aren Frontera, O., Melichar, B., Choueiri, T. K., Plimack, E. R., Barthélémy, P., Porta, C., George, S., et al. (2018). Nivolumab plus Ipilimumab versus Sunitinib in Advanced Renal-Cell Carcinoma. N. Engl. J. Med. 378, 1277-1290. 10.1056/NEJMoa1712126.
28. Motzer, R. J., Banchereau, R., Hamidi, H., Powles, T., McDermott, D., Atkins, M. B., Escudier, B., Liu, L.-F., Leng, N., Abbas, A. R., et al. (2020). Molecular Subsets in Renal Cancer Determine Outcome to Checkpoint and Angiogenesis Blockade. Cancer Cell 38, 803-817.e4. 10.1016/j.ccell.2020.10.011.
29. Joshi, S., Tolkunov, D., Aviv, H., Hakimi, A. A., Yao, M., Hsieh, J. J., Ganesan, S., Chan, C. S., and White, E. (2015). The Genomic Landscape of Renal Oncocytoma Identifies a Metabolic Barrier to Tumorigenesis. Cell Rep. 13, 1895-1908. 10.1016/j.celrep.2015.10.059.
30. Chen, F., Zhang, Y., Şenbabaoğlu, Y., Ciriello, G., Yang, L., Reznik, E., Shuch, B., Micevic, G., De Velasco, G., Shinbrot, E., et al. (2016). Multilevel Genomics-Based Taxonomy of Renal Cell Carcinoma. Cell Rep. 14, 2476-2489. 10.1016/j.celrep.2016.02.024.
31. Jorge, S., Capelo, J. L., LaFramboise, W., Satturwar, S., Korentzelos, D., Bastacky, S., Quiroga-Garza, G., Dhir, R., Wiśniewski, J. R., Lodeiro, C., et al. (2021). Absolute quantitative proteomics using the total protein approach to identify novel clinical immunohistochemical markers in renal neoplasms. BMC Med. 19, 196. 10.1186/s12916-021-02071-9.
32. Drendel, V., Heckelmann, B., Schell, C., Kook, L., Biniossek, M. L., Werner, M., Jilg, C. A., and Schilling, O. (2018). Proteomic distinction of renal oncocytomas and chromophobe renal cell carcinomas. Clin. Proteomics 15, 25. 10.1186/s12014-018-9200-6.
33. Al Ahmad, A., Paffrath, V., Clima, R., Busch, J. F., Rabien, A., Kilic, E., Villegas, S., Timmermann, B., Attimonelli, M., Jung, K., et al. (2019). Papillary Renal Cell Carcinomas Rewire Glutathione Metabolism and Are Deficient in Both Anabolic Glucose Synthesis and Oxidative Phosphorylation. Cancers 11, E1298. 10.3390/cancers11091298.
34. Xu, H., Li, W., Zhu, C., Cheng, N., Li, X., Hao, F., Zhu, J., Huang, L., Wang, R., Wang, L., et al. (2022). Proteomic profiling identifies novel diagnostic biomarkers and molecular subtypes for mucinous tubular and spindle cell carcinoma of the kidney. J. Pathol. 257, 53-67. 10.1002/path.5869.
35. Clark, D. J., Dhanasekaran, S. M., Petralia, F., Pan, J., Song, X., Hu, Y., da Veiga Leprevost, F., Reva, B., Lih, T.-S. M., Chang, H.-Y., et al. (2019). Integrated Proteogenomic Characterization of Clear Cell Renal Cell Carcinoma. Cell 179, 964-983.e31. 10.1016/j.cell.2019.10.007.
36. Mertins, P., Tang, L. C., Krug, K., Clark, D. J., Gritsenko, M. A., Chen, L., Clauser, K. R., Clauss, T. R., Shah, P., Gillette, M. A., et al. (2018). Reproducible workflow for multiplexed deep-scale proteome and phosphoproteome analysis of tumor tissues by liquid chromatography-mass spectrometry. Nat. Protoc. 13, 1632-1661. 10.1038/s41596-018-0006-9.
37. Khaleel, S., Ricketts, C., Linehan, W. M., Ball, M., Manley, B., Turajilic, S., Brugarolas, J., and Hakimi, A. (2022). Genetics and Tumor Microenvironment of Renal Cell Carcinoma. Société Int. Urol. J. 3, 386-396. 10.48083/BLPV3411.
38. Calinawan, A. P., Song, X., Ji, J., Dhanasekaran, S. M., Petralia, F., Wang, P., and Reva, B. (2020). ProTrack: An Interactive Multi-Omics Data Browser for Proteogenomic Studies. Proteomics 20, e1900359. 10.1002/pmic.201900359.
39. Bacigalupa, Z. A., and Rathmell, W. K. (2020). Beyond glycolysis: Hypoxia signaling as a master regulator of alternative metabolic pathways and the implications in clear cell renal cell carcinoma. Cancer Lett. 489, 19-28. 10.1016/j.canlet.2020.05.034.
40. Hanahan, D., and Weinberg, R. A. (2011). Hallmarks of cancer: the next generation. Cell 144, 646-674. 10.1016/j.cell.2011.02.013.
41. Drews, R. M., Hernando, B., Tarabichi, M., Haase, K., Lesluyes, T., Smith, P. S., Morrill Gavarró, L., Couturier, D.-L., Liu, L., Schneider, M., et al. (2022). A pan-cancer compendium of chromosomal instability. Nature 606, 976-983. 10.1038/s41586-022-04789-9.
42. Ricketts, C. J., and Linehan, W. M. (2018). Multi-regional Sequencing Elucidates the Evolution of Clear Cell Renal Cell Carcinoma. Cell 173, 540-542. 10.1016/j.cell.2018.03.077.
43. Turajlic, S., Xu, H., Litchfield, K., Rowan, A., Horswell, S., Chambers, T., O'Brien, T., Lopez, J. I., Watkins, T.B.K., Nicol, D., et al. (2018). Deterministic Evolutionary Trajectories Influence Primary Tumor Growth: TRACERx Renal. Cell 173, 595-610.e11. 10.1016/j.cell.2018.03.043.
44. Tan, V.Y.F., and Févotte, C. (2013). Automatic relevance determination in nonnegative matrix factorization with the B-divergence. IEEE Trans. Pattern Anal. Mach. Intell. 35, 1592-1605. 10.1109/TPAMI.2012.240.
45. Burrell, R. A., McClelland, S. E., Endesfelder, D., Groth, P., Weller, M.-C., Shaikh, N., Domingo, E., Kanu, N., Dewhurst, S. M., Gronroos, E., et al. (2013). Replication stress links structural and numerical cancer chromosomal instability. Nature 494, 492-496. 10.1038/nature11935.
46. Lin, Y.-W., Weng, X.-F., Huang, B.-L., Guo, H.-P., Xu, Y.-W., and Peng, Y.-H. (2021). IGFBP-1 in cancer: expression, molecular mechanisms, and potential clinical implications. Am. J. Transl. Res. 13, 813-832.
47. Xu, T., Gao, S., Liu, J., Huang, Y., Chen, K., and Zhang, X. (2021). MMP9 and IGFBP1 Regulate Tumor Immune and Drive Tumor Progression in Clear Cell Renal Cell Carcinoma. J. Cancer 12, 2243-2257. 10.7150/jca.48664.
48. Cai, F., Miao, Y., Liu, C., Wu, T., Shen, S., Su, X., and Shi, Y. (2018). Pyrroline-5-carboxylate reductase 1 promotes proliferation and inhibits apoptosis in non-small cell lung cancer. Oncol. Lett. 15, 731-740. 10.3892/ol.2017.7400.
49. Ding, J., Kuo, M.-L., Su, L., Xue, L., Luh, F., Zhang, H., Wang, J., Lin, T. G., Zhang, K., Chu, P., et al. (2017). Human mitochondrial pyrroline-5-carboxylate reductase 1 promotes invasiveness and impacts survival in breast cancers. Carcinogenesis 38, 519-531. 10.1093/carcin/bgx022.
50. Zhuang, J., Song, Y., Ye, Y., He, S., Ma, X., Zhang, M., Ni, J., Wang, J., and Xia, W. (2019). PYCR1 interference inhibits cell growth and survival via c-Jun N-terminal kinase/insulin receptor substrate 1 (JNK/IRS1) pathway in hepatocellular cancer. J. Transl. Med. 17, 343. 10.1186/s12967-019-2091-0.
51. Zhang, Y., Zhao, X., Deng, L., Li, X., Wang, G., Li, Y., and Chen, M. (2019). High expression of FABP4 and FABP6 in patients with colorectal cancer. World J. Surg. Oncol. 17, 171. 10.1186/s12957-019-1714-5.
52. Eskandari, E., Mahjoubi, F., and Motalebzadeh, J. (2018). An integrated study on TFs and miRNAs in colorectal cancer metastasis and evaluation of three co-regulated candidate genes as prognostic markers. Gene 679, 150-159. 10.1016/j.gene.2018.09.003.
53. Li, K., Huang, G., Wang, Z., Yang, R., Zhang, W., Ni, B., Guan, J., Yi, G., Li, Z., Zhu, Q., et al. (2022). IKBIP, a novel glioblastoma biomarker, maintains abnormal proliferation of tumor cells by inhibiting the ubiquitination and degradation of CDK4. Biochim. Biophys. Acta Mol. Basis Dis. 1869, 166571. 10.1016/j.bbadis.2022.166571.
54. Hiroshima, Y., Nakamura, F., Miyamoto, H., Mori, R., Taniguchi, K., Matsuyama, R., Akiyama, H., Tanaka, K., Ichikawa, Y., Kato, S., et al. (2013). Collapsin response mediator protein 4 expression is associated with liver metastasis and poor survival in pancreatic cancer. Ann. Surg. Oncol. 20 Suppl 3, S369-378. 10.1245/s10434-012-2491-3.
55. Matsunuma, R., Chan, D. W., Kim, B.-J., Singh, P., Han, A., Saltzman, A. B., Cheng, C., Lei, J. T., Wang, J., Roberto da Silva, L., et al. (2018). DPYSL3 modulates mitosis, migration, and epithelial-to-mesenchymal transition in claudin-low breast cancer. Proc. Natl. Acad. Sci. U.S.A 115, E11978-E11987. 10.1073/pnas.1810598115.
56. Wang, Q.-L., and Liu, L. (2019). PYCR1 is Associated with Papillary Renal Cell Carcinoma Progression. Open Med. Wars. Pol. 14, 586-592. 10.1515/med-2019-0066.
57. Kay, E. J., Paterson, K., Riera-Domingo, C., Sumpton, D., Däbritz, J.H.M., Tardito, S., Boldrini, C., Hernandez-Fernaud, J. R., Athineos, D., Dhayade, S., et al. (2022). Cancer-associated fibroblasts require proline synthesis by PYCR1 for the deposition of pro-tumorigenic extracellular matrix. Nat. Metab. 4, 693-710. 10.1038/s42255-022-00582-0.
58. Milne, K., Sun, J., Zaal, E. A., Mowat, J., Celie, P.H.N., Fish, A., Berkers, C. R., Forlani, G., Loayza-Puch, F., Jamieson, C., et al. (2019). A fragment-like approach to PYCR1 inhibition. Bioorg. Med. Chem. Lett. 29, 2626-2631. 10.1016/j.bmcl.2019.07.047.
59. Turajlic, S., Xu, H., Litchfield, K., Rowan, A., Chambers, T., Lopez, J.I., Nicol, D., O'Brien, T., Larkin, J., Horswell, S., et al. (2018). Tracking Cancer Evolution Reveals Constrained Routes to Metastases: TRACERx Renal. Cell 173, 581-594.e12. 10.1016/j.cell.2018.03.057.
60. Gonçalves, A. F., Adlesic, M., Brandt, S., Hejhal, T., Harlander, S., Sommer, L., Shakhova, O., Wild, P. J., and Frew, I. J. (2017). Evidence of renal angiomyolipoma neoplastic stem cells arising from renal epithelial cells. Nat. Commun. 8, 1466. 10.1038/s41467-017-01514-3.
61. Martignoni, G., Pea, M., Zampini, C., Brunelli, M., Segala, D., Zamboni, G., and Bonetti, F. (2015). PEComas of the kidney and of the genitourinary tract. Semin. Diagn. Pathol. 32, 140-159. 10.1053/j.semdp.2015.02.006.
62. Lake, B. B., Chen, S., Hoshi, M., Plongthongkum, N., Salamon, D., Knoten, A., Vijayan, A., Venkatesh, R., Kim, E. H., Gao, D., et al. (2019). A single-nucleus RNA-sequencing pipeline to decipher the molecular anatomy and pathophysiology of human kidneys. Nat. Commun. 10, 2832. 10.1038/s41467-019-10861-2.
63. Clark, D. J., Dhanasekaran, S. M., Petralia, F., Pan, J., Song, X., Hu, Y., da Veiga Leprevost, F., Reva, B., Lih, T.-S.M., Chang, H.-Y., et al. (2019). Integrated Proteogenomic Characterization of Clear Cell Renal Cell Carcinoma. Cell 179, 964-983.e31. 10.1016/j.cell.2019.10.007.
64. Keranen, L. M., Dutil, E. M., and Newton, A. C. (1995). Protein kinase C is regulated in vivo by three functionally distinct phosphorylations. Curr. Biol. CB 5, 1394-1403. 10.1016/s0960-9822 (95) 00277-6.
65. Li, W., Zhang, J., Bottaro, D. P., and Pierce, J. H. (1997). Identification of serine 643 of protein kinase C-delta as an important autophosphorylation site for its enzymatic activity. J. Biol. Chem. 272, 24550-24555. 10.1074/jbc.272.39.24550.
66. Procaccini, C., Lourenco, E. V., Matarese, G., and La Cava, A. (2009). Leptin signaling: A key pathway in immune responses. Curr. Signal Transduct. Ther. 4, 22-30. 10.2174/157436209787048711.
67. Lam, Q.L.K., and Lu, L. (2007). Role of leptin in immunity. Cell. Mol. Immunol. 4, 1-13.
68. Peng, C., Zeng, W., Su, J., Kuang, Y., He, Y., Zhao, S., Zhang, J., Ma, W., Bode, A. M., Dong, Z., et al. (2016). Cyclin-dependent kinase 2 (CDK2) is a key mediator for EGF-induced cell transformation mediated through the ELK4/c-Fos signaling pathway. Oncogene 35, 1170-1179. 10.1038/one.2015.175.
69. Liao, H., Ji, F., and Ying, S. (2017). CDK1: beyond cell cycle regulation. Aging 9, 2465-2466. 10.18632/aging.101348.
70. Fagundes, R., and Teixeira, L. K. (2021). Cyclin E/CDK2: DNA Replication, Replication Stress and Genomic Instability. Front. Cell Dev. Biol. 9, 774845. 10.3389/fcell.2021.774845.
71. Liberzon, A., Subramanian, A., Pinchback, R., Thorvaldsdóttir, H., Tamayo, P., and Mesirov, J. P. (2011). Molecular signatures database (MSigDB) 3.0. Bioinforma. Oxf. Engl. 27, 1739-1740. 10.1093/bioinformatics/btr260.
72. Zarkowska, T., and Mittnacht, S. (1997). Differential phosphorylation of the retinoblastoma protein by G1/S cyclin-dependent kinases. J. Biol. Chem. 272, 12738-12746. 10.1074/jbc.272.19.12738.
73. Connell-Crowley, L., Harper, J. W., and Goodrich, D. W. (1997). Cyclin D1/Cdk4 regulates retinoblastoma protein-mediated cell cycle arrest by site-specific phosphorylation. Mol. Biol. Cell 8, 287-301. 10.1091/mbc.8.2.287.
74. Beck, T. N., Kaczmar, J., Handorf, E., Nikonova, A., Dubyk, C., Peri, S., Lango, M., Ridge, J. A., Serebriiskii, I. G., Burtness, B., et al. (2015). Phospho-T356RB1 predicts survival in HPV-negative squamous cell carcinoma of the head and neck. Oncotarget 6, 18863-18874. 10.18632/oncotarget.4321.
75. Burke, J. R., Deshong, A. J., Pelton, J. G., and Rubin, S. M. (2010). Phosphorylation-induced conformational changes in the retinoblastoma protein inhibit E2F transactivation domain binding. J. Biol. Chem. 285, 16286-16293. 10.1074/jbc.M110.108167.
76. Chau, B. N., and Wang, J.Y.J. (2003). Coordinated regulation of life and death by RB. Nat. Rev. Cancer 3, 130-138. 10.1038/nrc993.
77. Yuan, Z. M., Huang, Y., Kraeft, S. K., Chen, L. B., Kharbanda, S., and Kufe, D. (1996). Interaction of cyclin-dependent kinase 2 and the Lyn tyrosine kinase in cells treated with 1-beta-D-arabinofuranosylcytosine. Oncogene 13, 939-946.
78. Kamburov, A., Lawrence, M. S., Polak, P., Leshchiner, I., Lage, K., Golub, T. R., Lander, E. S., and Getz, G. (2015). Comprehensive assessment of cancer missense mutation clustering in protein structures. Proc. Natl. Acad. Sci. U.S.A 112, E5486-5495. 10.1073/pnas. 1516373112.
79. McDermott, J. E., Arshad, O. A., Petyuk, V. A., Fu, Y., Gritsenko, M. A., Clauss, T. R., Moore, R. J., Schepmoes, A. A., Zhao, R., Monroe, M. E., et al. (2020). Proteogenomic Characterization of Ovarian HGSC Implicates Mitotic Kinases, Replication Stress in Observed Chromosomal Instability. Cell Rep. Med. 1, 100004. 10.1016/j.xcrm.2020.100004.
80. Gu, Y., Rosenblatt, J., and Morgan, D.O. (1992). Cell cycle regulation of CDK2 activity by phosphorylation of Thr160 and Tyr15. EMBO J. 11, 3995-4005. 10.1002/j.1460-2075.1992.tb05493.x. 81. Hughes, B. T., Sidorova, J., Swanger, J., Monnat, R. J., and Clurman, B. E. (2013). Essential role for Cdk2 inhibitory phosphorylation during replication stress revealed by a human Cdk2 knockin mutation. Proc. Natl. Acad. Sci. U.S.A 110, 8954-8959. 10.1073/pnas. 1302927110.
82. Teixeira, L. K., and Reed, S. I. (2017). Cyclin E Deregulation and Genomic Instability. Adv. Exp. Med. Biol. 1042, 527-547. 10.1007/978-981-10-6955-O_22.
83. Echarti, A., Hecht, M., Büttner-Herold, M., Haderlein, M., Hartmann, A., Fietkau, R., and Distel, L. (2019). CD8+ and Regulatory T cells Differentiate Tumor Immune Phenotypes and Predict Survival in Locally Advanced Head and Neck Cancer. Cancers 11, E1398. 10.3390/cancers11091398.
84. Taylor, E. M., and Lindsay, H. D. (2016). DNA replication stress and cancer: cause or cure? Future Oncol. Lond. Engl. 12, 221-237. 10.2217/fon.15.292.
85. Hong, S., Freeberg, M. A., Han, T., Kamath, A., Yao, Y., Fukuda, T., Suzuki, T., Kim, J. K., and Inoki, K. (2017). LARP1 functions as a molecular switch for mTORC1-mediated translation of an essential class of mRNAs. eLife 6, e25237. 10.7554/eLife.25237.
86. Michels, A. A., Robitaille, A. M., Buczynski-Ruchonnet, D., Hodroj, W., Reina, J. H., Hall, M. N., and Hernandez, N. (2010). mTORC1 directly phosphorylates and regulates human MAF1. Mol. Cell. Biol. 30, 3749-3757. 10.1128/MCB.00319-10.
87. Kim, Y.-M., Jung, C. H., Seo, M., Kim, E. K., Park, J.-M., Bae, S. S., and Kim, D.-H. (2015). mTORC1 phosphorylates UVRAG to negatively regulate autophagosome and endosome maturation. Mol. Cell 57, 207-218. 10.1016/j.molcel.2014.11.013.
88. Odle, R.I., Florey, O., Ktistakis, N. T., and Cook, S. J. (2021). CDK1, the Other “Master Regulator” of Autophagy. Trends Cell Biol. 31, 95-107. 10.1016/j.tcb.2020.11.001.
89. Armstrong, A. J., Halabi, S., Eisen, T., Broderick, S., Stadler, W. M., Jones, R. J., Garcia, J. A., Vaishampayan, U. N., Picus, J., Hawkins, R. E., et al. (2016). Everolimus versus sunitinib for patients with metastatic non-clear cell renal cell carcinoma (ASPEN): a multicentre, open-label, randomised phase 2 trial. Lancet Oncol. 17, 378-388. 10.1016/S1470-2045 (15) 00515-X.
90. Keeley, T. S., Yang, S., and Lau, E. (2019). The Diverse Contributions of Fucose Linkages in Cancer. Cancers 11, E1241. 10.3390/cancers11091241.
91. Pinho, S. S., and Reis, C. A. (2015). Glycosylation in cancer: mechanisms and clinical implications. Nat. Rev. Cancer 15, 540-555. 10.1038/nrc3982.
92. Bagdonaite, I., Malaker, S. A., Polasky, D. A., Riley, N. M., Schjoldager, K., Vakhrushev, S. Y., Halim, A., Aoki-Kinoshita, K. F., Nesvizhskii, A. I., Bertozzi, C. R., et al. (2022). Glycoproteomics. Nat. Rev. Methods Primer 2, 48. 10.1038/s43586-022-00128-4.
93. Costa, A. F., Campos, D., Reis, C. A., and Gomes, C. (2020). Targeting Glycosylation: A New Road for Cancer Drug Discovery. Trends Cancer 6, 757-766. 10.1016/j.trecan.2020.04.002.
94. Reily, C., Stewart, T. J., Renfrow, M. B., and Novak, J. (2019). Glycosylation in health and disease. Nat. Rev. Nephrol. 15, 346-366. 10.1038/s41581-019-0129-4.
95. Zhou, Y., Lih, T.-S.M., Yang, G., Chen, S.-Y., Chen, L., Chan, D. W., Zhang, H., and Li, Q.K. (2020). An Integrated Workflow for Global, Glyco-, and Phospho-proteomic Analysis of Tumor Tissues. Anal. Chem. 92, 1842-1849. 10.1021/acs.analchem.9b03753.
96. Polasky, D.A., Yu, F., Teo, G. C., and Nesvizhskii, A.I. (2020). Fast and comprehensive N-and O-glycoproteomics analysis with MSFragger-Glyco. Nat. Methods 17, 1125-1132. 10.1038/s41592-020-0967-9.
97. Polasky, D.A., Geiszler, D.J., Yu, F., and Nesvizhskii, A.I. (2022). Multiattribute Glycan Identification and FDR Control for Glycoproteomics. Mol. Cell. Proteomics MCP 21, 100205. 10.1016/j.mcpro.2022.100205.
98. Hu, Y., Shah, P., Clark, D.J., Ao, M., and Zhang, H. (2018). Reanalysis of Global Proteomic and Phosphoproteomic Data Identified a Large Number of Glycopeptides. Anal. Chem. 90, 8065-8071. 10.1021/acs.analchem.8b01137.
99. Robin, T., Mariethoz, J., and Lisacek, F. (2020). Examining and Fine-tuning the Selection of Glycan Compositions with GlyConnect Compozitor. Mol. Cell. Proteomics MCP 19, 1602-1618. 10.1074/mcp.RA120.002041.
100. Kürschner, G., Zhang, Q., Clima, R., Xiao, Y., Busch, J.F., Kilic, E., Jung, K., Berndt, N., Bulik, S., Holzhütter, H.-G., et al. (2017). Renal oncocytoma characterized by the defective complex I of the respiratory chain boosts the synthesis of the ROS scavenger glutathione. Oncotarget 8, 105882-105904. 10.18632/oncotarget.22413.
101. Thapa, R., and Wilson, G.D. (2016). The Importance of CD44 as a Stem Cell Biomarker and Therapeutic Target in Cancer. Stem Cells Int. 2016, 2087204. 10.1155/2016/2087204.
102. Thul, P. J., and Lindskog, C. (2018). The human protein atlas: A spatial map of the human proteome. Protein Sci. Publ. Protein Soc. 27, 233-244. 10.1002/pro.3307.
103. Huang, Y.-F., Aoki, K., Akase, S., Ishihara, M., Liu, Y.-S., Yang, G., Kizuka, Y., Mizumoto, S., Tiemeyer, M., Gao, X.-D., et al. (2021). Global mapping of glycosylation pathways in human-derived cells. Dev. Cell 56, 1195-1209.e7. 10.1016/j.devcel.2021.02.023.
104. Bastian, K., Scott, E., Elliott, D. J., and Munkley, J. (2021). FUT8 Alpha-(1,6)-Fucosyltransferase in Cancer. Int. J. Mol. Sci. 22, E455. 10.3390/ijms22010455.
105. Agrawal, P., Fontanals-Cirera, B., Sokolova, E., Jacob, S., Vaiana, C.A., Argibay, D., Davalos, V., McDermott, M., Nayak, S., Darvishian, F., et al. (2017). A Systems Biology Approach Identifies FUT8 as a Driver of Melanoma Metastasis. Cancer Cell 31, 804-819.e7. 10.1016/j.ccell.2017.05.007.
106. Tu, C.-F., Li, F.-A., Li, L.-H., and Yang, R.-B. (2022). Quantitative glycoproteomics analysis identifies novel FUT8 targets and signaling networks critical for breast cancer cell invasiveness. Breast Cancer Res. BCR 24, 21. 10.1186/s13058-022-01513-3.
107. Cancer Genome Atlas Research Network, Linehan, W.M., Spellman, P.T., Ricketts, C.J., Creighton, C.J., Fei, S.S., Davis, C., Wheeler, D.A., Murray, B.A., Schmidt, L., et al. (2016). Comprehensive Molecular Characterization of Papillary Renal-Cell Carcinoma. N. Engl. J. Med. 374, 135-145. 10.1056/NEJMoa1505917.
108. Rhoades Smith, K. E., and Bilen, M.A. (2019). A Review of Papillary Renal Cell Carcinoma and MET Inhibitors. Kidney Cancer Clifton Va 3, 151-161. 10.3233/KCA-190058.
109. Jeon, H.-M., and Lee, J. (2017). MET: roles in epithelial-mesenchymal transition and cancer stemness. Ann. Transl. Med. 5, 5. 10.21037/atm.2016.12.67.
110. Saitou, A., Hasegawa, Y., Fujitani, N., Ariki, S., Uehara, Y., Hashimoto, U., Saito, A., Kuronuma, K., Matsumoto, K., Chiba, H., et al. (2022). N-glycosylation regulates MET processing and signaling. Cancer Sci. 113, 1292-1304. 10.1111/cas.15278.
111. Chen, R., Li, J., Feng, C.-H., Chen, S.-K., Liu, Y.-P., Duan, C.-Y., Li, H., Xia, X.-M., He, T., Wei, M., et al. (2013). c-Met function requires N-linked glycosylation modification of pro-Met. J. Cell. Biochem. 114, 816-822. 10.1002/jcb.24420.
112. Wang, Y., Fukuda, T., Isaji, T., Lu, J., Gu, W., Lee, H.-H., Ohkubo, Y., Kamada, Y., Taniguchi, N., Miyoshi, E., et al. (2015). Loss of a1,6-fucosyltransferase suppressed liver regeneration: implication of core fucose in the regulation of growth factor receptor-mediated cellular signaling. Sci. Rep. 5, 8264. 10.1038/srep08264.
113. Wang, Y., Fukuda, T., Isaji, T., Lu, J., Im, S., Hang, Q., Gu, W., Hou, S., Ohtsubo, K., and Gu, J. (2015). Loss of a1,6-fucosyltransferase inhibits chemical-induced hepatocellular carcinoma and tumorigenesis by down-regulating several cell signaling pathways. FASEB J. Off. Publ. Fed. Am. Soc. Exp. Biol. 29, 3217-3227. 10.1096/fj.15-270710.
114. Capone, E., Iacobelli, S., and Sala, G. (2021). Role of galectin 3 binding protein in cancer progression: a potential novel therapeutic target. J. Transl. Med. 19, 405. 10.1186/s12967-021-03085-w.
115. Kudo-Saito, C., Ishida, A., Shouya, Y., Teramoto, K., Igarashi, T., Kon, R., Saito, K., Awada, C., Ogiwara, Y., and Toyoura, M. (2018). Blocking the FSTL1-DIP2A Axis Improves Anti-tumor Immunity. Cell Rep. 24, 1790-1801. 10.1016/j.celrep.2018.07.043.
116. Agrawal, P., Fontanals-Cirera, B., Sokolova, E., Jacob, S., Vaiana, C. A., Argibay, D., Davalos, V., McDermott, M., Nayak, S., Darvishian, F., et al. (2017). A Systems Biology Approach Identifies FUT8 as a Driver of Melanoma Metastasis. Cancer Cell 31, 804-819.e7. 10.1016/j.ccell.2017.05.007.
117. Santiago, L., Castro, M., Sanz-Pamplona, R., Garzón, M., Ramirez-Labrada, A., Tapia, E., Moreno, V., Layunta, E., Gil-Gómez, G., Garrido, M., et al. (2020). Extracellular Granzyme A Promotes Colorectal Cancer Development by Enhancing Gut Inflammation. Cell Rep. 32, 107847. 10.1016/j.celrep.2020.107847.
118. Huelse, J. M., Fridlyand, D. M., Earp, S., DeRyckere, D., and Graham, D. K. (2020). MERTK in cancer therapy: Targeting the receptor tyrosine kinase in tumor cells and the immune system. Pharmacol. Ther. 213, 107577. 10.1016/j.pharmthera.2020.107577.
119. Fu, Q., Colgan, S. P., and Shelley, C.S. (2016). Hypoxia: The Force that Drives Chronic Kidney Disease. Clin. Med. Res. 14, 15-39. 10.3121/cmr.2015.1282.
120. Rathmell, W. K., Rathmell, J. C., and Linehan, W. M. (2018). Metabolic Pathways in Kidney Cancer: Current Therapies and Future Directions. J. Clin. Oncol. Off. J. Am. Soc. Clin. Oncol., JCO2018792309. 10.1200/JCO.2018.79.2309.
121. Merino, M. J., Torres-Cabala, C., Pinto, P., and Linehan, W. M. (2007). The morphologic spectrum of kidney tumors in hereditary leiomyomatosis and renal cell carcinoma (HLRCC) syndrome. Am. J. Surg. Pathol. 37, 1578-1585. 10.1097/PAS.0b013e31804375b8.
122. Yang, Y., Valera, V., Sourbier, C., Vocke, C. D., Wei, M., Pike, L., Huang, Y., Merino, M. A., Bratslavsky, G., Wu, M., et al. (2012). A novel fumarate hydratase-deficient HLRCC kidney cancer cell line, UOK268: a model of the Warburg effect in cancer. Cancer Genet. 205, 377-390. 10.1016/j.cancergen.2012.05.001.
123. Sourbier, C., Valera-Romero, V., Giubellino, A., Yang, Y., Sudarshan, S., Neckers, L., and Linehan, W. M. (2010). Increasing reactive oxygen species as a therapeutic approach to treat hereditary leiomyomatosis and renal cell carcinoma. Cell Cycle Georget. Tex 9, 4183-4189. 10.4161/cc.9.20.13458.
124. Crooks, D. R., and Linehan, W. M. (2018). The Warburg effect in hominis: isotope-resolved metabolism in ccRCC. Nat. Rev. Urol. 15, 731-732. 10.1038/s41585-018-0110-1.
125. Weiss, R. H. (2018). Metabolomics and Metabolic Reprogramming in Kidney Cancer. Semin. Nephrol. 38, 175-182. 10.1016/j.semnephrol.2018.01.006.
126. Linehan, W. M., Srinivasan, R., and Schmidt, L. S. (2010). The genetic basis of kidney cancer: a metabolic disease. Nat. Rev. Urol. 7, 277-285. 10.1038/nrurol.2010.47.
127. Martínez-Reyes, I., and Chandel, N. S. (2020). Mitochondrial TCA cycle metabolites control physiology and disease. Nat. Commun. 11, 102. 10.1038/s41467-019-13668-3.
128. Patel, R., Raj, A. K., Lokhande, K. B., Almasri, M. A., Alzahrani, K. J., Almeslet, A. S., Swamy, K. V., Sarode, G. S., Sarode, S. C., Patil, S., et al. (2021). Detection of Nail Oncometabolite SAICAR in Oral Cancer Patients and Its Molecular Interactions with PKM2 Enzyme. Int. J. Environ. Res. Public. Health 18, 11225. 10.3390/ijerph182111225.
129. Yusenko, M. V. (2010). Molecular pathology of renal oncocytoma: a review. Int. J. Urol. Off. J. Jpn. Urol. Assoc. 17, 602-612. 10.1111/j.1442-2042.2010.02574.x.
130. Westbrook, R. L., Bridges, E., Roberts, J., Escribano-Gonzalez, C., Eales, K. L., Vettore, L. A., Walker, P. D., Vera-Siguenza, E., Rana, H., Cuozzo, F., et al. (2022). Proline synthesis through PYCR1 is required to support cancer cell proliferation and survival in oxygen-limiting conditions. Cell Rep. 38, 110320. 10.1016/j.celrep.2022.110320.
131. Tehlivets, O., Malanovic, N., Visram, M., Pavkov-Keller, T., and Keller, W. (2013). S-adenosyl-L-homocysteine hydrolase and methylation disorders: yeast as a model system. Biochim. Biophys. Acta 1832, 204-215. 10.1016/j.bbadis.2012.09.007.
132. Löffler, M., Carrey, E. A., and Zameitat, E. (2015). Orotic Acid, More Than Just an Intermediate of Pyrimidine de novo Synthesis. J. Genet. Genomics Yi Chuan Xue Bao 42, 207-219. 10.1016/j.jgg.2015.04.001.
133. Walaszek, Z. (1990). Potential use of D-glucaric acid derivatives in cancer prevention. Cancer Lett. 54, 1-8. 10.1016/0304-3835 (90) 90083-a.
134. Wang, L.-S., Chen, S.-J., Zhang, J.-F., Liu, M.-N., Zheng, J.-H., and Yao, X.-D. (2017). Anti-proliferative potential of Glucosamine in renal cancer cells via inducing cell cycle arrest at G0/G1 phase. BMC Urol. 17, 38. 10.1186/s12894-017-0221-7.
135. Oliveri, V., and Vecchio, G. (2016). 8-Hydroxyquinolines in medicinal chemistry: A structural perspective. Eur. J. Med. Chem. 120, 252-274. 10.1016/j.ejmech.2016.05.007.
136. Vila Justribo, M., Duce Gracia, F., Bello Dronda, S., and Larrad Mur, L. (1986). [Study of helper T lymphocytes and suppressor T lymphocytes in respiratory allergy and their modifications in relation to symptomatology]. Allergol. Immunopathol. (Madr.) 14, 319-324.
137. Blish, K. R., Wang, W., Willingham, M. C., Du, W., Birse, C. E., Krishnan, S. R., Brown, J. C., Hawkins, G. A., Garvin, A. J., D'Agostino, R. B., et al. (2008). A human bone morphogenetic protein antagonist is down-regulated in renal cancer. Mol. Biol. Cell 19, 457-464. 10.1091/mbc.e07-05-0433.
138. Gherardi, E., Birchmeier, W., Birchmeier, C., and Vande Woude, G. (2012). Targeting MET in cancer: rationale and progress. Nat. Rev. Cancer 12, 89-103. 10.1038/nrc3205.
139. Ishibe, S., Karihaloo, A., Ma, H., Zhang, J., Marlier, A., Mitobe, M., Togawa, A., Schmitt, R., Czyczk, J., Kashgarian, M., et al. (2009). Met and the epidermal growth factor receptor act cooperatively to regulate final nephron number and maintain collecting duct morphology. Dev. Camb. Engl. 136, 337-345. 10.1242/dev.024463.
140. Gerlinger, M., Horswell, S., Larkin, J., Rowan, A. J., Salm, M. P., Varela, I., Fisher, R., McGranahan, N., Matthews, N., Santos, C. R., et al. (2014). Genomic architecture and evolution of clear cell renal cell carcinomas defined by multiregion sequencing. Nat. Genet. 46, 225-233. 10.1038/ng.2891.
141. Miao, D., Margolis, C. A., Gao, W., Voss, M. H., Li, W., Martini, D. J., Norton, C., Bossé, D., Wankowicz, S. M., Cullen, D., et al. (2018). Genomic correlates of response to immune checkpoint therapies in clear cell renal cell carcinoma. Science 359, 801-806. 10.1126/science.aan5951.
142. Durinck, S., Stawiski, E.W., Pavía-Jiménez, A., Modrusan, Z., Kapur, P., Jaiswal, B. S., Zhang, N., Toffessi-Tcheuyap, V., Nguyen, T. T., Pahuja, K. B., et al. (2015). Spectrum of diverse genomic alterations define non-clear cell renal carcinoma subtypes. Nat. Genet. 47, 13-21. 10.1038/ng.3146.
143. Xiao, Y., Clima, R., Busch, J., Rabien, A., Kilic, E., Villegas, S. L., Timmermann, B., Attimonelli, M., Jung, K., and Meierhofer, D. (2020). Decreased Mitochondrial DNA Content Drives OXPHOS Dysregulation in Chromophobe Renal Cell Carcinoma. Cancer Res. 80, 3830-3840. 10.1158/0008-5472.CAN-20-0754.
144. Aibar, S., González-Blas, C. B., Moerman, T., Huynh-Thu, V. A., Imrichova, H., Hulselmans, G., Rambow, F., Marine, J.-C., Geurts, P., Aerts, J., et al. (2017). SCENIC: single-cell regulatory network inference and clustering. Nat. Methods 14, 1083-1086. 10.1038/nmeth.4463.
145. Lindgren, D., Eriksson, P., Krawczyk, K., Nilsson, H., Hansson, J., Veerla, S., Sjölund, J., Höglund, M., Johansson, M. E., and Axelson, H. (2017). Cell-Type-Specific Gene Programs of the Normal Human Nephron Define Kidney Cancer Subtypes. Cell Rep. 20, 1476-1489. 10.1016/j.celrep.2017.07.043.
146. Liu, J., Huang, Z., Chen, H.-N., Qin, S., Chen, Y., Jiang, J., Zhang, Z., Luo, M., Ye, Q., Xie, N., et al. (2021). ZNF37A promotes tumor metastasis through transcriptional control of THSD4/TGF-β axis in colorectal cancer. Oncogene 40, 3394-3407. 10.1038/s41388-021-01713-9.
147. Saito, M., Kurokawa, M., Oda, M., Oshima, M., Tsutsui, K., Kosaka, K., Nakao, K., Ogawa, M., Manabe, R.-I., Suda, N., et al. (2011). ADAMTSL6ß protein rescues fibrillin-1 microfibril disorder in a Marfan syndrome mouse model through the promotion of fibrillin-1 assembly. J. Biol. Chem. 286, 38602-38613. 10.1074/jbc.M111.243451.
148. Cohen, H., Ben-Hamo, R., Gidoni, M., Yitzhaki, I., Kozol, R., Zilberberg, A., and Efroni, S. (2014). Shift in GATA3 functions, and GATA3 mutations, control progression and clinical presentation in breast cancer. Breast Cancer Res. BCR 16, 464. 10.1186/s13058-014-0464-0.
149. Adamski, V., Hattermann, K., Kubelt, C., Cohrs, G., Lucius, R., Synowitz, M., Sebens, S., and Held-Feindt, J. (2020). Entry and exit of chemotherapeutically-promoted cellular dormancy in glioblastoma cells is differentially affected by the chemokines CXCL12, CXCL16, and CX3CL1. Oncogene 39, 4421-4435. 10.1038/s41388-020-1302-8.
150. Bailey, M. H., Tokheim, C., Porta-Pardo, E., Sengupta, S., Bertrand, D., Weerasinghe, A., Colaprico, A., Wendl, M. C., Kim, J., Reardon, B., et al. (2018). Comprehensive Characterization of Cancer Driver Genes and Mutations. Cell 173, 371-385.e18. 10.1016/j.cell.2018.02.060.
151. Jia, P., and Zhao, Z. (2019). Characterization of Tumor-Suppressor Gene Inactivation Events in 33 Cancer Types. Cell Rep. 26, 496-506.e3. 10.1016/j.celrep.2018.12.066.
152. Mehra, R., Vats, P., Cao, X., Su, F., Lee, N. D., Lonigro, R., Premkumar, K., Trpkov, K., McKenney, J. K., Dhanasekaran, S. M., et al. (2018). Somatic Bi-allelic Loss of TSC Genes in Eosinophilic Solid and Cystic Renal Cell Carcinoma. Eur. Urol. 74, 483-486. 10.1016/j.eururo.2018.06.007.
153. Vocke, C. D., Ricketts, C. J., Merino, M. J., Srinivasan, R., Metwalli, A. R., Middelton, L. A., Peterson, J., Yang, Y., and Linehan, W. M. (2017). Comprehensive genomic and phenotypic characterization of germline FH deletion in hereditary leiomyomatosis and renal cell carcinoma. Genes. Chromosomes Cancer 56, 484-492. 10.1002/gcc.22452.
154. Yoo, A., Tang, C., Zucker, M., Fitzgerald, K., DiNatale, R. G., Rappold, P. M., Weiss, K., Freeman, B., Lee, C.-H., Schultz, N., et al. (2022). Genomic and Metabolic Hallmarks of SDH-and FH-deficient Renal Cell Carcinomas. Eur. Urol. Focus, S2405-4569 (21) 00312-6. 10.1016/j.euf.2021.12.002.
155. Al-Obaidy, K. I., Saleeb, R. M., Trpkov, K., Williamson, S. R., Sangoi, A. R., Nassiri, M., Hes, O., Montironi, R., Cimadamore, A., Acosta, A. M., et al. (2022). Recurrent KRAS mutations are early events in the development of papillary renal neoplasm with reverse polarity. Mod. Pathol. Off. J. U. S. Can. Acad. Pathol. Inc 35, 1279-1286. 10.1038/s41379-022-01018-6.
156. Lee, H. J., Shin, D. H., Park, J. Y., Kim, S. Y., Hwang, C. S., Lee, J. H., Kim, J. Y., Sol, M. Y., and Nam, J. K. (2020). Unilateral synchronous papillary renal neoplasm with reverse polarity and clear cell renal cell carcinoma: a case report with KRAS and PIK3CA mutations. Diagn. Pathol. 15, 123. 10.1186/s13000-020-01042-7.
157. Braun, D. A., Street, K., Burke, K. P., Cookmeyer, D. L., Denize, T., Pedersen, C. B., Gohil, S. H., Schindler, N., Pomerance, L., Hirsch, L., et al. (2021). Progressive immune dysfunction with advancing disease stage in renal cell carcinoma. Cancer Cell 39, 632-648.e8. 10.1016/j.ccell.2021.02.013.
158. Borcherding, N., Vishwakarma, A., Voigt, A. P., Bellizzi, A., Kaplan, J., Nepple, K., Salem, A. K., Jenkins, R. W., Zakharia, Y., and Zhang, W. (2021). Mapping the immune environment in clear cell renal carcinoma by single-cell genomics. Commun. Biol. 4, 122. 10.1038/s42003-020-01625-6.
159. Bi, K., He, M. X., Bakouny, Z., Kanodia, A., Napolitano, S., Wu, J., Grimaldi, G., Braun, D. A., Cuoco, M. S., Mayorga, A., et al. (2021). Tumor and immune reprogramming during immunotherapy in advanced renal cell carcinoma. Cancer Cell 39, 649-661.e5. 10.1016/j.ccell.2021.02.015.
160. Khadirnaikar, S., Kumar, P., Pandi, S. N., Malik, R., Dhanasekaran, S. M., and Shukla, S. K. (2019). Immune associated LncRNAs identify novel prognostic subtypes of renal clear cell carcinoma. Mol. Carcinog. 58, 544-553. 10.1002/mc.22949.
161. Roldán, F. L., Izquierdo, L., Ingelmo-Torres, M., Lozano, J.J., Carrasco, R., Cuñado, A., Reig, O., Mengual, L., and Alcaraz, A. (2022). Prognostic Gene Expression-Based Signature in Clear-Cell Renal Cell Carcinoma. Cancers 14, 3754. 10.3390/cancers 14153754.
162. Huang, Y., Goel, S., Duda, D.G., Fukumura, D., and Jain, R.K. (2013). Vascular normalization as an emerging strategy to enhance cancer immunotherapy. Cancer Res. 73, 2943-2948. 10.1158/0008-5472.CAN-12-4354.
163. Tian, L., Goldstein, A., Wang, H., Ching Lo, H., Sun Kim, I., Welte, T., Sheng, K., Dobrolecki, L.E., Zhang, X., Putluri, N., et al. (2017). Mutual regulation of tumour vessel normalization and immunostimulatory reprogramming. Nature 544, 250-254. 10.1038/nature21724.
164. Yasuda, S., Sho, M., Yamato, I., Yoshiji, H., Wakatsuki, K., Nishiwada, S., Yagita, H., and Nakajima, Y. (2013). Simultaneous blockade of programmed death 1 and vascular endothelial growth factor receptor 2 (VEGFR2) induces synergistic anti-tumour effect in vivo. Clin. Exp. Immunol. 172, 500-506. 10.1111/cei.12069.
165. Movahedi, K., Laoui, D., Gysemans, C., Baeten, M., Stange, G., Van den Bossche, J., Mack, M., Pipeleers, D., In′t Veld, P., De Baetselier, P., et al. (2010). Different tumor microenvironments contain functionally distinct subsets of macrophages derived from Ly6C (high) monocytes. Cancer Res. 70, 5728-5739. 10.1158/0008-5472.CAN-09-4672.
166. Chen, C.-Y., Jan, Y.-H., Juan, Y.-H., Yang, C.-J., Huang, M.-S., Yu, C.-J., Yang, P.-C., Hsiao, M., Hsu, T.-L., and Wong, C.-H. (2013). Fucosyltransferase 8 as a functional regulator of nonsmall cell lung cancer. Proc. Natl. Acad. Sci. U.S.A 110, 630-635. 10.1073/pnas.1220425110.
167. Corsello, S.M., Bittker, J.A., Liu, Z., Gould, J., McCarren, P., Hirschman, J.E., Johnston, S.E., Vrcic, A., Wong, B., Khan, M., et al. (2017). The Drug Repurposing Hub: a next-generation drug library and information resource. Nat. Med. 23, 405-408. 10.1038/nm.4306.
168. da Veiga Leprevost, F., Haynes, S.E., Avtonomov, D.M., Chang, H.-Y., Shanmugam, A.K., Mellacheruvu, D., Kong, A. T., and Nesvizhskii, A.I. (2020). Philosopher: a versatile toolkit for shotgun proteomics data analysis. Nat. Methods 17, 869-870. 10.1038/s41592-020-0912-y.
169. Kong, A.T., Leprevost, F.V., Avtonomov, D.M., Mellacheruvu, D., and Nesvizhskii, A.I. (2017). MSFragger: ultrafast and comprehensive peptide identification in mass spectrometry-based proteomics. Nat. Methods 14, 513-520. 10.1038/nmeth.4256.
170. Geiszler, D.J., Kong, A.T., Avtonomov, D.M., Yu, F., Leprevost, F. da V., and Nesvizhskii, A.I. (2021). PTM-Shepherd: Analysis and Summarization of Post-Translational and Chemical Modifications From Open Search Results. Mol. Cell. Proteomics MCP 20, 100018. 10.1074/mcp.TIR120.002216.
171. Djomehri, S.I., Gonzalez, M.E., da Veiga Leprevost, F., Tekula, S.R., Chang, H.-Y., White, M.J., Cimino-Mathews, A., Burman, B., Basrur, V., Argani, P., et al. (2020). Quantitative proteomic landscape of metaplastic breast carcinoma pathological subtypes and their relationship to triple-negative tumors. Nat. Commun. 11, 1723. 10.1038/s41467-020-15283-z.
172. Xu, T., Le, T.D., Liu, L., Su, N., Wang, R., Sun, B., Colaprico, A., Bontempi, G., and Li, J. (2017). CancerSubtypes: an R/Bioconductor package for molecular cancer subtype identification, validation and visualization. Bioinforma. Oxf. Engl. 33, 3131-3133. 10.1093/bioinformatics/btx378.
173. Ritchie, M.E., Phipson, B., Wu, D., Hu, Y., Law, C.W., Shi, W., and Smyth, G.K. (2015). limma powers differential expression analyses for RNA-sequencing and microarray studies. Nucleic Acids Res. 43, e47-e47. 10.1093/nar/gkv007.
174. Krug, K., Mertins, P., Zhang, B., Hornbeck, P., Raju, R., Ahmad, R., Szucs, M., Mundt, F., Forestier, D., Jane-Valbuena, J., et al. (2019). A Curated Resource for Phosphosite-specific Signature Analysis. Mol. Cell. Proteomics MCP 18, 576-593. 10.1074/mcp.TIR118.000943.
175. Kamburov, A., Cavill, R., Ebbels, T.M.D., Herwig, R., and Keun, H.C. (2011). Integrated pathway-level analysis of transcriptomics and metabolomics data with IMPaLA. Bioinforma. Oxf. Engl. 27, 2917-2918. 10.1093/bioinformatics/btr499.
176. Yu, G., Wang, L.-G., Han, Y., and He, Q.-Y. (2012). clusterProfiler: an R package for comparing biological themes among gene clusters. Omics J. Integr. Biol. 16, 284-287. 10.1089/omi.2011.0118.
177. Van de Sande, B., Flerin, C., Davie, K., De Waegeneer, M., Hulselmans, G., Aibar, S., Seurinck, R., Saelens, W., Cannoodt, R., Rouchon, Q., et al. (2020). A scalable SCENIC workflow for single-cell gene regulatory network analysis. Nat. Protoc. 15, 2247-2276. 10.1038/s41596-020-0336-2.
178. Petralia, F., Krek, A., Calinawan, A.P., Feng, S., Gosline, S., Pugliese, P., Ceccarelli, M., and Wang, P. (2021). BayesDeBulk: A Flexible Bayesian Algorithm for the Deconvolution of Bulk Tumor Data (Bioinformatics) 10.1101/2021.06.25.449763.
179. Ma, W., Kim, S., Chowdhury, S., Li, Z., Yang, M., Yoo, S., Petralia, F., Jacobsen, J., Li, J.J., Ge, X., et al. (2020). DreamAI: algorithm for the imputation of proteomics data (Bioinformatics) 10.1101/2020.07.21.214205.
180. Fisher, S., Barry, A., Abreu, J., Minie, B., Nolan, J., Delorey, T.M., Young, G., Fennell, T.J., Allen, A., Ambrogio, L., et al. (2011). A scalable, fully automated process for construction of sequence-ready human exome targeted capture libraries. Genome Biol. 12, R1. 10.1186/gb-2011-12-1-r1.
181. Cao, L., Huang, C., Cui Zhou, D., Hu, Y., Lih, T.M., Savage, S.R., Krug, K., Clark, D.J., Schnaubelt, M., Chen, L., et al. (2021). Proteogenomic characterization of pancreatic ductal adenocarcinoma. Cell 184, 5031-5052.e26. 10.1016/j.cell.2021.08.023.
182. Ross, P.L., Huang, Y.N., Marchese, J.N., Williamson, B., Parker, K., Hattan, S., Khainovski, N., Pillai, S., Dey, S., Daniels, S., et al. (2004). Multiplexed Protein Quantitation in Saccharomyces cerevisiae Using Amine-reactive Isobaric Tagging Reagents. Mol. Cell. Proteomics 3, 1154-1169. 10.1074/mcp.M400129-MCP200.
183. Elgogary, A., Xu, Q., Poore, B., Alt, J., Zimmermann, S.C., Zhao, L., Fu, J., Chen, B., Xia, S., Liu, Y., et al. (2016). Combination therapy with BPTES nanoparticles and metformin targets the metabolic heterogeneity of pancreatic cancer. Proc. Natl. Acad. Sci. U.S.A 113, E5328-5336. 10.1073/pnas.1611406113.
184. Nguyen, T., Kirsch, B.J., Asaka, R., Nabi, K., Quinones, A., Tan, J., Antonio, M.J., Camelo, F., Li, T., Nguyen, S., et al. (2019). Uncovering the Role of N-Acetyl-Aspartyl-Glutamate as a Glutamate Reservoir in Cancer. Cell Rep. 27, 491-501.e6. 10.1016/j.celrep.2019.03.036.
185. Udupa, S., Nguyen, S., Hoang, G., Nguyen, T., Quinones, A., Pham, K., Asaka, R., Nguyen, K., Zhang, C., Elgogary, A., et al. (2019). Upregulation of the Glutaminase II Pathway Contributes to Glutamate Production upon Glutaminase 1 Inhibition in Pancreatic Cancer. Proteomics 19, e1800451. 10.1002/pmic.201800451.
186. Robinson, D.R., Wu, Y.-M., Lonigro, R.J., Vats, P., Cobain, E., Everett, J., Cao, X., Rabban, E., Kumar-Sinha, C., Raymond, V., et al. (2017). Integrative clinical genomics of metastatic cancer. Nature 548, 297-303. 10.1038/nature23306.
187. Wu, Y.-M., Cieślik, M., Lonigro, R.J., Vats, P., Reimers, M.A., Cao, X., Ning, Y., Wang, L., Kunju, L.P., de Sarkar, N., et al. (2018). Inactivation of CDK12 Delineates a Distinct Immunogenic Class of Advanced Prostate Cancer. Cell 173, 1770-1782.e14. 10.1016/j.cell.2018.04.034.
188. Pierre-Jean, M., Rigaill, G., and Neuvial, P. (2015). Performance evaluation of DNA copy number segmentation methods. Brief. Bioinform. 16, 600-615. 10.1093/bib/bbu026.
189. Carter, S.L., Cibulskis, K., Helman, E., McKenna, A., Shen, H., Zack, T., Laird, P.W., Onofrio, R.C., Winckler, W., Weir, B. A., et al. (2012). Absolute quantification of somatic DNA alterations in human cancer. Nat. Biotechnol. 30, 413-421. 10.1038/nbt.2203.
190. Riester, M., Singh, A.P., Brannon, A.R., Yu, K., Campbell, C.D., Chiang, D. Y., and Morrissey, M.P. (2016). PureCN: copy number calling and SNV classification using targeted short read sequencing. Source Code Biol. Med. 11, 13. 10.1186/s13029-016-0060-z.
191. Xi, R., Lee, S., Xia, Y., Kim, T.-M., and Park, P.J. (2016). Copy number analysis of whole-genome data using BIC-seq2 and its application to detection of cancer susceptibility variants. Nucleic Acids Res. 44, 6274-6286. 10.1093/nar/gkw491.
192. Robinson, M.D., McCarthy, D. J., and Smyth, G.K. (2010). edgeR: a Bioconductor package for differential expression analysis of digital gene expression data. Bioinforma. Oxf. Engl. 26, 139-140. 10.1093/bioinformatics/btp616.
193. Griffiths, J.A., Richard, A.C., Bach, K., Lun, A. T. L., and Marioni, J.C. (2018). Detection and removal of barcode swapping in single-cell RNA-seq data. Nat. Commun. 9, 2667. 10.1038/s41467-018-05083-x.
194. participants in the 1st Human Cell Atlas Jamboree, Lun, A.T.L., Riesenfeld, S., Andrews, T., Dao, T.P., Gomes, T., and Marioni, J.C. (2019). EmptyDrops: distinguishing cells from empty droplets in droplet-based single-cell RNA sequencing data. Genome Biol. 20, 63. 10.1186/s13059-019-1662-y.
195. Young, M.D., and Behjati, S. (2020). SoupX removes ambient RNA contamination from droplet-based single-cell RNA sequencing data. GigaScience 9, giaa151. 10.1093/gigascience/giaa151.
196. Käll, L., Canterbury, J.D., Weston, J., Noble, W.S., and MacCoss, M.J. (2007). Semi-supervised learning for peptide identification from shotgun proteomics datasets. Nat. Methods 4, 923-925. 10.1038/nmeth1113.
197. Shteynberg, D.D., Deutsch, E.W., Campbell, D.S., Hoopmann, M.R., Kusebauch, U., Lee, D., Mendoza, L., Midha, M.K., Sun, Z., Whetton, A. D., et al. (2019). PTMProphet: Fast and Accurate Mass Modification Localization for the Trans-Proteomic Pipeline. J. Proteome Res. 18, 4262-4272. 10.1021/acs.jproteome.9b00205.
198. Nesvizhskii, A.I., Keller, A., Kolker, E., and Aebersold, R. (2003). A Statistical Model for Identifying Proteins by Tandem Mass Spectrometry. Anal. Chem. 75, 4646-4658. 10.1021/ac0341261.
199. Huang, K.-L., Li, S., Mertins, P., Cao, S., Gunawardena, H.P., Ruggles, K.V., Mani, D.R., Clauser, K.R., Tanioka, M., Usary, J., et al. (2017). Proteogenomic integration reveals therapeutic targets in breast cancer xenografts. Nat. Commun. 8, 14864. 10.1038/ncomms14864.
200. Gu, Z., Eils, R., and Schlesner, M. (2016). Complex heatmaps reveal patterns and correlations in multidimensional genomic data. Bioinforma. Oxf. Engl. 32, 2847-2849. 10.1093/bioinformatics/btw313.
201. Newman, A.M., Liu, C.L., Green, M.R., Gentles, A.J., Feng, W., Xu, Y., Hoang, C.D., Diehn, M., and Alizadeh, A.A. (2015). Robust enumeration of cell subsets from tissue expression profiles. Nat. Methods 12, 453-457. 10.1038/nmeth.3337.
202. Petralia, F., Wang, L., Peng, J., Yan, A., Zhu, J., and Wang, P. (2018). A new method for constructing tumor specific gene co-expression networks based on samples with tumor purity heterogeneity. Bioinforma. Oxf. Engl. 34, i528-i536. 10.1093/bioinformatics/bty280.
203. Zhou, W., Triche, T.J., Laird, P. W., and Shen, H. (2018). SeSAMe: reducing artifactual detection of DNA methylation by Infinium BeadChips in genomic deletions. Nucleic Acids Res. 46, e123. 10.1093/nar/gky691.
204. Hao, Y., Hao, S., Andersen-Nissen, E., Mauck, W.M., Zheng, S., Butler, A., Lee, M.J., Wilk, A.J., Darby, C., Zager, M., et al. (2021). Integrated analysis of multimodal single-cell data. Cell 184, 3573-3587.e29. 10.1016/j.cell.2021.04.048.
205. McInnes, L., Healy, J., and Melville, J. (2018). UMAP: Uniform Manifold Approximation and Projection for Dimension Reduction. 10.48550/ARXIV.1802.03426.
206. Zhang, K., Lee, H.-M., Wei, G.-H., and Manninen, A. (2016). Meta-analysis of gene expression and integrin-associated signaling pathways in papillary renal cell carcinoma subtypes. Oncotarget 7, 84178-84189. 10.18632/oncotarget.12390.
207. Chen, J., Harbert, J., Bhalla, R., and Dewenter, T. (2022). Eosinophilic Solid and Cystic Renal Cell Carcinoma with Non-typical Immunophenotype: A Series of Two Cases. Am. J. Clin. Pathol. 158, S75-S76. 10.1093/ajcp/aqac126.155.
208. Tirosh, I., Izar, B., Prakadan, S.M., Wadsworth, M.H., Treacy, D., Trombetta, J.J., Rotem, A., Rodman, C., Lian, C., Murphy, G., et al. (2016). Dissecting the multicellular ecosystem of metastatic melanoma by single-cell RNA-seq. Science 352, 189-196. 10.1126/science.aad0501.
209. Law, C.W., Chen, Y., Shi, W., and Smyth, G.K. (2014). voom: precision weights unlock linear model analysis tools for RNA-seq read counts. Genome Biol. 15, R29. 10.1186/gb-2014-15-2-129.
210. Fabregat, A., Jupe, S., Matthews, L., Sidiropoulos, K., Gillespie, M., Garapati, P., Haw, R., Jassal, B., Korninger, F., May, B., et al. (2018). The Reactome Pathway Knowledgebase. Nucleic Acids Res. 46, D649-D655. 10.1093/nar/gkx1132.
211. Kanehisa, M., and Goto, S. (2000). KEGG: kyoto encyclopedia of genes and genomes. Nucleic Acids Res. 28, 27-30. 10.1093/nar/28.1.27.
212. Romero, P., Wagg, J., Green, M.L., Kaiser, D., Krummenacker, M., and Karp, P.D. (2005). Computational prediction of human metabolic pathways from the complete human genome. Genome Biol. 6, R2. 10.1186/gb-2004-6-1-r2.

### Example 4

It was investigated if combining UCHL1 and IGF2BP3 identifies the 3 molecular classes of ccRCC that are associated with poor prognosis, namely BAP1 mutants, high wGII and DNA methylation-1 category. The scatter plot in FIG. 35 shows that both these biomarkers collectively detect most all of the tumors with these adverse molecular features.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of treating renal cell carcinoma (RCC), comprising:
a) assaying the level of expression of UCHL1 ubiquitin C-terminal hydrolase L1 (UCHL1) in a sample from a subject diagnosed with RCC;
b) administering an UCHL1 inhibitor to a subject identified as having increased levels of expression of UCHL1.

2. A method of characterizing or prognosing RCC, comprising:
a) assaying the level of expression UCHL1 in a sample from a subject diagnosed with RCC; and
b) identifying said subject as at an increased risk of death or metastatic RCC when said subject is identified as having increased levels of expression of UCHL1 in said sample.

3. The method of claim 2, further comprising administering an UCHL1 inhibitor to said subject.

4. A method of treating RCC, comprising:
a) assaying the level of expression of Pyrroline-5-carboxylate reductase 1 (PYCR1) and/or Insulin-like growth factor 2 mRNA-binding protein 3 (IGF2BP3) in a sample from a subject diagnosed with RCC;
b) administering a PYCR1 and/or IGF2BP3 inhibitor to a subject identified as having increased levels of expression of PYCR1 and/or IGF2BP3.

5. A method of characterizing or prognosing RCC, comprising:
a) assaying the level of expression PYCR1 and/or IGF2BP3 in a sample from a subject diagnosed with RCC; and
b) identifying said subject as at an increased risk of death or metastatic RCC when said subject is identified as having increased levels of expression of PYCR1 and/or IGF2BP3 in said sample.

6. The method of claim 4 or 5, further comprising detecting the level of expression of one or more additional markers selected from the group consisting of DPYSL3, IKBIP, and FABP6.

7. The method of any of the preceding claims, wherein said UCHL1, IGF2BP3, and/or said PYCR1 inhibitor is selected from the group consisting of a small molecule, a nucleic acid, and an antibody.

8. The method of claim 7, wherein said small molecule is CAS-668467-91-2.

9. The method of any of the preceding claims, further comprising administering a MEK inhibitor to said subject.

10. The method of claim 9, wherein said MEK inhibitor is trametinib.

11. The method of any of the preceding claims, further comprising administering adjuvant chemotherapy to said subject.

12. The method of any of the preceding claims, wherein said RCC is clear cell RCC (ccRCC).

13. The method of any of the preceding claims, wherein said RCC is non-clear cell RCC (non-ccRCC).

14. The method of any of the preceding claims, wherein said RCC exhibits high genome instability.

15. The method of any of the preceding claims, wherein said expression is the level of mRNA or protein expressed by a UCHL1, IGF2BP3 or PYCR1 gene.

16. The method of any of the preceding claims, wherein said sample is selected from the group consisting of urine, tissue, blood, plasma, serum, kidney tissue, kidney cells, and renal cancer cells.

17. The method of any of the preceding claims, wherein said assaying is carried out utilizing a method selected from the group consisting of an immunological technique, a sequencing technique, a nucleic acid hybridization technique, and a nucleic acid amplification technique.

18. The method of claim 17, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence-based amplification.

19. The method of claim 17, wherein said immunological technique is selected from the group consisting of immunohistochemistry, ELISA, and a Western blot.

20. The method of claim 17, wherein said assaying comprises the use of a reagent selected from the group consisting of one or more antibodies, a pair of amplification oligonucleotides, a sequencing primer, and an oligonucleotide probe.

21. The method of claim 20, wherein said reagent comprises one or more labels.

22. The use of a UCHL1 inhibitor to treat RCC in a subject identified as having increased levels of expression of UCHL1.

23. The use of a PYCR1 inhibitor to treat RCC in a subject identified as having increased levels of expression of PYCR1.

24. The use of an IGF2BP3 inhibitor to treat RCC in a subject identified as having increased levels of expression of IGF2BP3.

25. A method of characterizing or prognosing RCC, comprising:
a) assaying the level of expression of UCHL1 and IGF2BP3 in a sample from a subject diagnosed with RCC; and
b) identifying said subject as at an increased risk of death or metastatic RCC when said subject is identified as having increased levels of expression of UCHL1 and IGF2BP3 in said sample.
